(19) United States
(12) Patent Application Publication
(10) Pub. No.: US 2024/0327918 A1
(43) Pub. Date: Oct. 3, 2024

(54) A BASELINE GENE EXPRESSION-BASED PROGNOSTIC FOR ANTI-INF ALPHA THERAPY RESPONSE NI PATIENTS WITH INFLAMMATORY BOWEL DISEASE

(71) Applicant: Inflammatix, Inc., Sunnyvale, CA (US)

(72) Inventors: Timothy Elisha Sweeney, Truckee, CA (US); Yudong He, Millbrae, CA (US); Suraj Sakaram, Sunnyvale, CA (US)

(73) Assignee: Inflammatix, Inc., Sunnyvale, CA (US)

(21) Appl. No.: 18/682,210

(22) PCT Filed: Aug. 25, 2022
(86) PCT No.: PCT/US2022/041492

§ 371 Date: Feb. 8, 2024

Related U.S. Application Data
(60) Provisional application No. 63/238,744, filed on Aug. 30, 2021.

(51) Int. Cl. C12Q 1/6883; C12Q 1/6851

(52) U.S. Cl. CPC C12Q 1/6883; C12Q 1/6851; C12Q 2600/106

(57) ABSTRACT

Systems, methods, compositions, apparatuses, and kits for determining the responsiveness of subjects with inflammatory bowel disease (IBD) to anti-TNF alpha therapy using biological samples obtained from the subjects, are provided herein. The disclosed methods and compositions involve biomarkers identified from the application of a machine learning workflow to TNF alpha response training data from biological samples. The biomarkers allow the calculation of a score that can be used to determine the TNF alpha responder status of the subjects.

### CROSS-REFERENCES TO RELATED APPLICATIONS

This application claims priority to U.S. Provisional Application No. 63/238,744, filed 30 Aug. 2021, the disclosure of which is hereby incorporated by reference in its entirety for all purposes.

### STATEMENT REGARDING FEDERALLY SPONSORED RESEARCH OR DEVELOPMENT

This invention was made with government support under Contract No. 1R43DK127578-01 awarded by the National Institutes of Health (NIH, Small Business Innovation Research Program). The government has certain rights in the invention.

### BACKGROUND

Inflammatory Bowel Disease (IBD), a chronic inflammation of the digestive system consisting of two main subtypes, Crohn's Disease (CD) and Ulcerative Colitis (UC), afflicts 6.8 million people globally with age-standardized prevalence rates having increased substantially in the past 30 years.

Although anti-TNF alpha therapies have revolutionized the management and care of IBD, their administration and usage remain suboptimal for the following reasons: 1) greater than 50% of patients do not have a lasting therapeutic response, 2) their usage increases the risk of infections, liver problems, arthritis, and lymphoma, and 3) they are expensive for the patient and healthcare provider. With approximately 1.6 million people suffering from IBD in the US and global prevalence of IBD on the rise, a predictive test for anti-TNF alpha response would greatly improve the efficacy and cost-to-benefit ratio of these biologics.

To date, there is no clear predictive factor of response or loss of response to these therapies. Although several studies have identified single biomarkers of response, none are robust enough to translate to clinical practice. Overcoming this gap in the administration of anti-TNF alpha therapies is an important next step in improving their utility and reducing overall healthcare costs, morbidity, and mortality.

There is thus a need for new, accurate, powerful, and affordable methods for identifying IBD patients who are most likely to benefit from anti-TNF alpha therapy. The present disclosure satisfies this need and provides other advantages as well.

### BRIEF SUMMARY

In one aspect, the present disclosure provides a method of administering medical care to a subject presenting one or more symptoms of Inflammatory Bowel Disease (IBD), the method comprising: (i) receiving a biological sample obtained from the subject; (ii) measuring expression levels of one or more biomarkers in the sample, wherein the one or more biomarkers comprise at least one biomarker from Table 3 or one pair of biomarkers from Table 4; and (iii) generating a TNF alpha prognostic score based on the measured expression levels of the biomarkers in the sample, wherein a TNF alpha prognostic score that exceeds a threshold value indicates that the subject is a candidate for anti-TNF alpha therapy.

In some embodiments, the method further comprises (iv) determining the subject is a candidate for anti-TNF alpha therapy based on the TNF alpha prognostic score exceeding the threshold value; and (v) administering anti-TNF alpha therapy to the subject to treat the IBD. In some embodiments, the method further comprises (iv) determining the subject is not a candidate for anti-TNF alpha therapy based on the TNF alpha prognostic score not exceeding the threshold. In some embodiments, the biological sample is an intestinal biopsy (e.g., colonic or ileal mucosal biopsy), buccal swab, blood, or stool. In some embodiments, the IBD is Crohn's Disease (CD). In some embodiments, the IBD is Ulcerative Colitis (UC). In some embodiments, the IBD is another unclassified IBD. In some embodiments, the expression of the biomarkers is detected using qRT-PCR or isothermal amplification. In some embodiments, the isothermal amplification method is qRT-LAMP. In some embodiments, the expression of the biomarkers is detected using a NanoString nCounter. In some embodiments, the method comprises measuring the expression of 1, 2, 3, 4, 5, 6, 7, 8, 9, or 10 biomarkers in the sample. In some embodiments, the one or more biomarkers comprise WNK2, OCRL, ASB7, PCBP3, AMPD2, FAM155A, and IL13RA2. In some embodiments, the TNF alpha prognostic score exceeds the threshold value and the measured expression levels of at least one of WNK2, OCRL, and ASB7 are elevated relative to a reference level representative of an individual with IBD who is not a candidate for anti-TNF alpha therapy. In some embodiments, the TNF alpha prognostic score exceeds the threshold value and the measured expression levels of at least one of PCBP3, AMPD2, FAM155A, and IL13RA2 are reduced relative to a reference level representative of an individual with IBD who is not a candidate for anti-TNF alpha therapy. In some embodiments, the anti-TNF alpha therapy comprises administering a drug selected from the group consisting of infliximab, Remicade, adalimumab, Humira, certolizumab, Cimzia, golimumab, Simponi, etanercept, Enbrel, and a biosimilar of any drug on this list. In some embodiments, the anti-TNF alpha therapy is another therapy capable of inhibiting one or more TNF alpha activities.

In another aspect, the present disclosure provides a test kit for detecting the expression levels of one or more biomarkers in a biological sample from a subject with Inflammatory Bowel Disease (IBD), wherein the biomarkers comprise at least one biomarker from Table 3, or one pair of biomarkers from Table 4.

In some embodiments, the kit comprises a microarray. In some embodiments, the kit comprises an oligonucleotide for each of the one or more biomarkers, wherein each oligonucleotide hybridizes to one of the biomarkers. In some embodiments, the biomarkers comprise WNK2, OCRL, ASB7, PCBP3, AMPD2, FAM155A, and IL13RA2. In some embodiments, the kit comprises an oligonucleotide that hybridizes to WNK2, an oligonucleotide that hybridizes to OCRL, an oligonucleotide that hybridizes to ASB7, an oligonucleotide that hybridizes to PCBP3, an oligonucleotide that hybridizes to AMPD2, an oligonucleotide that hybridizes to FAM155A, and an oligonucleotide that hybridizes to IL13RA2. In some embodiments, the kit is for detecting 1, 2, 3, 4, 5, 6, 7, 8, 9, 10, or more biomarkers. In some embodiments, the kit further comprises one or more reagents for performing q-RT-PCR, qRT-LAMP, or NanoString nCounter analysis. In some embodiments, the IBD is Crohn's Disease (CD). In some embodiments, the IBD is Ulcerative Colitis (UC). In some embodiments, the IBD is another unclassified IBD. In some embodiments, the biological sample is a mucosal biopsy such as an intestinal biopsy (e.g., colonic or ileal mucosal biopsy). In some embodiments, the biological sample is a buccal swab, blood, or stool. In some embodiments, the test kit further comprises instructions to calculate a TNF alpha prognostic score based on the levels of expression of the biomarkers in the biological sample from the subject, the score reporting the likelihood that the subject will respond to an anti-TNF alpha therapy. In some embodiments, the anti-TNF alpha therapy comprises administering a drug selected from the group consisting of infliximab, Remicade, adalimumab, Humira, certolizumab, Cimzia, golimumab, Simponi, etanercept, Enbrel, and biosimilars of any drug on this list. In some embodiments, the anti-TNF alpha therapy is another therapy capable of inhibiting one or more TNF alpha activities.

In another aspect, the present disclosure provides a computer product comprising a non-transitory computer readable medium storing a plurality of instructions that when executed cause a computer system to perform any one of the herein-described methods.

In another aspect, the present disclosure provides a system comprising: any of the herein-described computer products, and one or more processors for executing instructions stored on the computer readable medium.

In another aspect, the present disclosure provides a system comprising means for performing any one of the herein-described methods.

In another aspect, the present disclosure provides a system comprising one or more processors configured to perform any one of the herein-described methods.

In another aspect, the present disclosure provides a system comprising modules that respectively perform the steps of the herein-described methods.

A better understanding of the nature and advantages of embodiments of the present disclosure may be gained with reference to the following detailed description and the accompanying drawings.

### BRIEF DESCRIPTION OF THE DRAWINGS

FIGS. 1A-1B: A leave-one-study-out (LOSO) multicohort analysis of IBD cohorts reveals 324 significant DE genes. FIG. 1A: Boxplots of meta-scores based on the 324 significant genes (selected by |effect size|≥0.8 and FDR p-value≤10%) identified by comparing anti-TNF alpha responders vs. non-responders in 4 individual studies.

FIG. 1B: ROC curves shown for discriminating responders from non-responders in the discovery datasets obtained with LOSO approach.

FIGS. 2A-2B: Effect sizes and discriminatory performance of the 7-gene signature. FIG. 2A: Forest plots for random effects model estimates of effect size of the 7 genes, comparing anti-TNF alpha responders vs. non-responders. FIG. 2B: ROC curves shown for discriminatory performance of 7-gene signature in discovery datasets obtained with LOSO approach.

FIGS. 3A-3B. Cross-validated training performance of signature gene sets with ML models. FIG. 3A: ROC curves shown for discriminating anti-TNF alpha responders vs. non-responders using all 324 significant DE genes as the feature set to train 8 ML classifiers. FIG. 3B: ROC curves shown for discriminating anti-TNF alpha responders vs. non-responders using 7-gene signature as the feature set to train 8 ML classifiers.

FIGS. 4A-4F. Area under curve (AUC) distribution for single gene and 2-gene combinations. AUCs were calculated to evaluate the performance of predicting responder vs. non-responder samples from the 4 studies. FIG. 4A: Background AUC distribution using each of 15,116 genes measured across all 4 datasets. FIG. 4B: AUC distribution using each of 58 up-regulated or (FIG. 4C) 266 down-regulated genes selected by absolute effect size ≥0.8, FDR value ≤10%. FIG. 4D: AUC distribution for all 2-gene combinations from 324 significant DE mRNAs (total 52,326 pairs). FIG. 4E: AUC distribution for 10,000 randomly selected 2-gene pairs from 52,326 possible pairs with 324 DE genes where samples adjudications were scrambled (50 different permutations collated into one). FIG. 4F: AUC distribution for 10,000 randomly selected 2-gene from 15,116 genes measured across all 4 datasets.

FIG. 5 illustrates a measurement system 500 according to an embodiment of the present disclosure.

FIG. 6 shows a block diagram of an example computer system usable with systems and methods according to embodiments of the present disclosure.

### TERMS

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to primers, probes, oligonucleotides, template RNA or cDNA, genomic DNA, amplified subsequences of biomarker genes, or any polynucleotide composed of deoxyribonucleic acids (DNA), ribonucleic acids (RNA), or any other type of polynucleotide which is an N-glycoside of a purine or pyrimidine base, or modified purine or pyrimidine bases in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). “Nucleic acid”, “DNA” “polynucleotides, and similar terms also include nucleic acid analogs. The polynucleotides are not necessarily physically derived from any existing or natural sequence, but can be generated in any manner, including chemical synthesis, DNA replication, reverse transcription or a combination thereof.

“Primer” as used herein refers to an oligonucleotide, whether occurring naturally or produced synthetically, that is capable of acting as a point of initiation of synthesis when placed under conditions in which synthesis of a primer extension product which is complementary to a nucleic acid strand is induced i.e., in the presence of nucleotides and an agent for polymerization such as DNA polymerase and at a suitable temperature and buffer. Such conditions include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.), and at a suitable temperature. The primer is preferably single-stranded for maximum efficiency in amplification such as a TaqMan real-time quantitative RT-PCR as described herein. The primers herein are selected to be substantially complementary to the different strands of each specific sequence to be amplified, and a given set of primers will act together to amplify a subsequence of the corresponding biomarker gene.

The term “gene” refers to the segment of DNA involved in producing a polypeptide chain. It can include regions preceding and following the coding region (leader and trailer) as well as intervening sequences (introns) between individual coding segments (exons).

“Inflammatory Bowel Disease” or “IBD” refers to a chronic autoimmune condition involving long-term inflammation of the digestive tract, particularly the lower intestine. Most cases of IBD are either Crohn's disease (CD) or ulcerative colitis (UC), together with some IBDs that are unclassified. For the purposes of the present invention, a subject with IBD has received a diagnosis of IBD from a medical professional, and/or has one or more symptoms of IBD, e.g., diarrhea, constipation, fatigue, abdominal pain, cramping, intestinal gas, swelling, or bloating, blood in the stool, mucus in the stool, fever, perianal disease, reduced appetite, or unintended weight loss. Typically, the diagnosis is also based on test results or other clinical analyses such as tests for anemia, stool studies, colonoscopy, flexible sigmoidoscopy, upper endoscopy, capsule endoscopy, balloon-assisted enteroscopy, X-ray, CT scan, MRI, or others. The IBD symptoms in a subject can be mild, moderate, or severe, and can result from any form of IBD, including any form of Crohn's disease (including, e.g., ileocolitis/ileoceceal Crohn's disease), ileitis, gastroduodenal Crohn's disease, jejunoileitis, Crohn's granulomatous colitis) or Ulcerative colitis, and including other forms such as microscopic colitis, indeterminate colitis, diversion colitis, pancolitis, or ulcerative proctitis

“Tumor necrosis factor alpha,” or “TNF alpha” (see, e.g., NCBI Gene ID 7124, or UniProt ID P01375, the entire disclosures of which are herein incorporated by reference) refers to a cytokine released by immune cells such as macrophages as part of an inflammatory response. TNF alpha exists as both a transmembrane form and a soluble form. TNF alpha plays a role in the pathogenesis of IBD, and anti-TNF alpha drugs (also called, e.g., TNF alpha blockers or inhibitors) have been developed for the treatment of IBD and other inflammatory conditions. “Anti-TNF alpha therapy” refers to the administration of one or more anti-TNF alpha drugs, i.e., a drug that can inhibit the activity, levels, or stability of TNF alpha in a subject. In particular embodiments, anti-TNF alpha therapy refers to the administration of a biologic such as infliximab, Remicaade, adalimumab, Humira, certolizumab, Cimzia, golimumab, Simponi, etanercept, Enbrel, or a biosimilar of any drug on this list, or another therapy capable of inhibiting the TNF alpha activities. The therapy can comprise the administration of any one or more TNF alpha inhibitors, for any duration and at any dose. For example, the patient may receive 1 dose, or 2, 3, 4, 5, 6, 7, 8, 9, 10, 15, 20, 25, 30, 40, 50 or more doses, over, e.g., 1, 2, 3, 4, 5, 6, or 7 days, 1, 2, 3, or 4 weeks, 1, 2, 3, 4, 5, 6, 7, 8, 9, 10, 11, 12 or more months.

A “response” to an anti-TNF alpha therapy can refer to any lasting, detectable improvement in any symptom of IBD. A patient showing a response to a therapy means that the patient is a “responder” to the treatment. In particular embodiments, a determination that a patient is a “responder” to an anti-TNF alpha therapy means that the patient has satisfied one or more of the criteria indicated in Table 1, e.g., having an SES-CD score≤2, a Mayo endoscopic subscore of 1, a grade 0 or 1 on the histological score for ulcerative colitis, a decrease of at least 3 points on the histological score, a clear improvement of ulcerations and a decrease in the histological score, a decrease of at least 3 points and 30% in baseline for the total Mayo score, a decrease in the subscore for rectal bleeding of at least 1 point or an absolute subscore for rectal bleeding of 0 or 1, or any combination of any of the above. An IBD patient that is determined to likely be a responder to an anti-TNF alpha treatment using the herein-described methods is considered a “candidate” for anti-TNF alpha therapy.

A “biological sample” refers to a biological specimen obtained from a subject containing, e.g., cells, tissues, or fluids from the subject. For the purposes of the present methods and compositions, a biological sample is taken from a subject with IBD, and in particular embodiments the sample is obtained from or comprises nucleated cells from a tissue affected by IBD, e.g., from the lower GI tract. In particular embodiments, the sample comprises cells of the small intestine (duodenum jejunum, ileum) and/or large intestine (colon). For example, in some embodiments, the sample is an intestinal biopsy (e.g., colonic mucosal biopsy or ileal mucosal biopsy). In some embodiments, the sample is a buccal swab. In some embodiments, the sample is a stool sample. In other embodiments, the sample is a blood sample, e.g., plasma, serum or whole blood sample. Other potential samples that can be used include, urine, ascites, seminal fluid, vaginal secretions, cerebrospinal fluid (CSF), synovial fluid, pleural fluid (pleural lavage), pericardial fluid, peritoneal fluid, amniotic fluid, saliva, nasal fluid, otic fluid, gastric fluid, breast milk, amniotic fluid, bile, gastric juice, lymph, mucus, pericardial fluid, peritoneal fluid, pleural fluid, pus, saliva, sebum, serous fluid, sputum, sweat, tears, and others.

As used herein, a “biomarker gene”, “biomarker mRNA”, or “biomarker” refers to a gene whose expression in cells of a subject (e.g., cells from an intestinal biopsy such as a colonic or ileal mucosal biopsy) is not only correlated with being a responder or a non-responder to an anti-TNF alpha therapy in a patient with IBD (also referred to as the patient's “TNF alpha responder status”), but also of a prognostic value. The expression level of each of the genes need not be correlated with the TNF alpha response status (i.e., being a responder or non-responder) in all patients; rather, a correlation will exist at the population level, such that the level of expression is sufficiently correlated within the overall population of individuals with IBD and with known TNF alpha response status (i.e., responder or non-responder) that it can be combined with the expression levels of other biomarker genes, in any of a number of ways, as described elsewhere herein, and used to calculate a biomarker or TNF alpha prognostic score. The values used for the measured expression level of the individual biomarker genes can be determined in any of a number of ways, including direct readouts from relevant instruments or assay systems, or values determined using methods including, but not limited to, forms of linear or non-linear transformation, rescaling, normalizing, z-scores, ratios against a common reference value, or any other means known to those of skill in the art. In some embodiments, the readout values of the biomarkers are compared to the readout value of a reference or control, e.g., a housekeeping gene whose expression is measured at the same time as the biomarkers. For example, the ratio or log ratio of the biomarkers to the reference gene can be determined. Preferred biomarker genes for the purposes of the present methods include WNK2, OCRL, ASB7, PCBP3, AMPD2, FAM155A, and IL13RA2, but others can be used as well, e.g., other biomarkers identified using the machine learning methods described herein, or other markers presented in Table 3, or any one or any number of the pairs of biomarkers presented in Table 4.

A “biomarker score” or “composite score” or “TNF alpha prognostic score”, terms which can be used interchangeably, refers to a value allowing a determination of the TNF alpha responder status (i.e., being a responder or a non-responder) or the probability of being a responder or non-responder to a TNF alpha inhibitor in a subject with IBD. The biomarker or TNF alpha prognostic score is calculated from the measured expression levels of one or a plurality of biomarker genes, e.g., 1, 2, 3, 4, 5, 6, 7, 8, 9 10 or more individual biomarker genes, in cells (i.e., cells from the subject that are present in the biological sample, e.g., intestinal biopsy) from a subject. In some embodiments, the TNF alpha prognostic score is determined by applying a mathematical formula, or a series of mathematical formulae with specified interconnections, or a machine learning algorithm with optimized hyperparameters, or another parameter-based method by which the measured expression values of the biomarker genes can be used to generate a single “TNF alpha prognostic score”, including, e.g., arithmetic or geometric means with or without weights, linear regression, logistic regression, neural nets, or any other method known in the art. In particular embodiments, the “TNF alpha prognostic score” is used to determine the TNF alpha responder status (i.e., being a responder or non-responder) or the probability of being a responder or non-responder to an anti-TNF alpha therapy in a subject with IBD, by virtue of the score surpassing or not a given threshold value for the outcome in question, as described in more detail elsewhere herein. In some embodiments, the TNF alpha prognostic score is and can be further combined with other factors, such as the presence or severity of specific symptoms, patient factors (e.g. age, sex, vital signs, comorbidities, prior treatment history, or other relevant clinical parameters), e.g., to improve the performance of the TNF alpha prognostic score in determining their TNF alpha responder status.

The term “correlating” generally refers to determining a relationship between one random variable with another. In various embodiments, correlating a given biomarker level or score with the presence or absence of a condition or outcome (e.g., responder or non-responder to an anti-TNF alpha therapy) comprises determining the presence, absence or amount of at least one biomarker in a subject with the same outcome. In specific embodiments, a set of biomarker levels or their score is correlated to the presence or absence of a particular outcome, using receiver operating characteristic (ROC) curves.

“Conservatively modified variants” refers to nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants can have an increased stability, assembly, or activity.

As used in herein, the terms “identical” or percent “identity,” in the context of describing two or more polynucleotide sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. The identity can exist over a region that is at least about 10, 15, 20, 25, 30, 35, 40, 45, 50, or more nucleotides in length. In some embodiments, percent identity is determined over the full-length of the nucleic acid sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm with, e.g., the default parameters can be used. See, e.g., Altschul et al., (1990) J. Mol. Biol. 215:403-410 and the National Center for Biotechnology Information website, ncbi.nlm.nih.gov.

### DETAILED DESCRIPTION

The present disclosure provides methods and compositions for determining whether or not (or the likelihood that) subjects with inflammatory bowel disease (IBD) will respond to an anti-TNF alpha therapy using biological samples from the subjects, and for determining effective treatment strategies for such subjects. The present methods and compositions involve biomarkers identified from the application of a machine learning workflow to TNF alpha responder data, i.e., gene expression data from biological samples taken from patients with IBD taken prior to the treatment, where the patients are known to respond or to not respond to an anti-TNF alpha therapy, and where the status of responder or non-responder is adjudicated after a certain period of time (typically three months or another time interval) post the treatment. Using these data, biomarkers have been identified that allow the generation of a TNF alpha prognostic score that can be used to determine the TNF alpha responder status (i.e., being a responder or non-responder to an anti-TNF alpha treatment) of a subject with IBD, or the probability of being a responder or non-responder.

### I. Subjects

The present methods and compositions can be used to determine whether a patient with inflammatory bowel disease (IBD) is likely to respond to (i.e., is a candidate for) an anti-TNF alpha therapy, i.e., to show lasting, detectable improvement in one or more symptoms or features of IBD, or to satisfy one or more of the criteria for responding shown in Table 1. In various embodiments, the subject may be an adult of any age, a child, or an adolescent. The subject may be male or female.

The subject has one or more symptoms of IBD. A non-limiting list of IBD symptoms includes diarrhea, constipation, fatigue, abdominal pain, cramping, intestinal gas, swelling, or bloating, blood in the stool, mucus in the stool, fever, perianal disease, reduced appetite, unintended weight loss, and others. The diagnosis can also be informed by test results or clinical analyses such as tests for anemia, stool studies, colonoscopy, flexible sigmoidoscopy, upper endoscopy, capsule endoscopy, balloon-assisted enteroscopy, X-ray, CT scan, MRI, or others. The symptoms can be mild, moderate, or severe. The patient can have any form of IBD, including any form of Crohn's disease (including, e.g., ileocolitis/ileoceceal Crohn's disease), ileitis, gastroduodenal Crohn's disease, jejunoileitis, Crohn's granulomatous colitis) or Ulcerative colitis, and including other forms such as microscopic colitis, indeterminate colitis, diversion colitis, pancolitis, ulcerative proctitis, and other IBDs that are unclassified.

In particular embodiments, the subject is present in a medical context, e.g., a clinical setting where IBD diagnosis and/or treatment may take place. A clinical setting does not necessarily indicate that the patient is physically present in a hospital or clinical facility, however. For example, the patient may be at home but has been in communication with a health care provider about his or her condition and its treatment. The results of the methods described herein can allow a determination of the optimal next step or plan of action for the subject's care. In particular, a determination that a subject is likely to be a responder to an anti-TNF alpha treatment, i.e. is a candidate for anti-TNF alpha therapy for a good outcome, can indicate the initiation or continuation of an anti-TNF alpha therapy (alone or together with other treatments for IBD). In contrast, a determination that a subject is not likely to be a responder to an anti-TNF alpha treatment, i.e. is not a candidate for anti-TNF alpha therapy for a good outcome, can indicate that an anti-TNF alpha treatment should not be initiated or, if it has already been initiated, should be discontinued, and that other IBD therapeutic approaches should be explored.

### II. Biological Samples

To assess the biomarker status of the IBD patient, a biological sample is obtained. In particular embodiments, the sample is obtained from or comprises cells or tissue from a tissue affected by IBD, e.g., a mucosal biopsy. In particular, the sample will comprise cells of the lower gastrointestinal tract, i.e., the small intestine (duodenum jejunum, ileum) and large intestine (colon). For example, in some embodiments, the sample is an intestinal biopsy, such as a colonic mucosal biopsy or an ileal mucosal biopsy. In some embodiments, the sample is a buccal swab. In some embodiments, the sample is a stool sample. In other embodiments, the sample is a blood sample, e.g., plasma, serum or whole blood sample. Other potential samples that can be used include, urine, ascites, seminal fluid, vaginal secretions, cerebrospinal fluid (CSF), synovial fluid, pleural fluid (pleural lavage), pericardial fluid, peritoneal fluid, amniotic fluid, saliva, nasal fluid, otic fluid, gastric fluid, breast milk, amniotic fluid, bile, gastric juice, lymph, mucus, pericardial fluid, peritoneal fluid, pleural fluid, pus, saliva, sebum, serous fluid, sputum, sweat, tears, and others. The biological sample, e.g., mucosal biopsy, can be obtained from the subject using conventional techniques known in the art. In some embodiments, the sample is obtained for the purposes of assessing the subject's biomarker status using the herein-described methods, and is, e.g., obtained in connection with and close to the time that the present methods are performed. In other embodiments, the sample has been obtained previously, e.g., for another purpose, and is now being used for the herein-described methods. The sample can be obtained by the same caregiver or clinical facility as that carrying out the herein-described methods, or can be obtained from a different source (e.g., different caregiver or clinical facility).

### III. Selection of Biomarkers

The likelihood of being a responder to an anti-TNF alpha treatment in an IBD subject is determined by calculating a score (“TNF alpha prognostic score” or “biomarker score”) based on the expression levels of biomarkers in a biological sample taken prior to the treatment decision. In some embodiments, a panel of seven biomarkers is used to calculate the score. In particular embodiments, the biomarker genes are WNK2, OCRL, ASB7, PCBP3, AMPD2, FAM155A, and IL13RA2. WNK2 refers to Lysine deficient protein kinase 2 (see, e.g., NCBI Entrez gene ID 65268, the entire disclosure of which is herein incorporated by reference). OCRL refers to Inositol polyphosphate-5-phosphatase (see, e.g., NCBI Entrez gene ID 4952, the entire disclosure of which is herein incorporated by reference). ASB7 refers to Ankyrin repeat and SOCS box containing 7 (see, e.g., NCBI Entrez gene ID 140460, the entire disclosure of which is herein incorporated by reference). PCBP3 refers to Poly (RC) binding protein 3 (see, e.g., NCBI Entrez gene ID 54039, the entire disclosure of which is herein incorporated by reference), AMPD2 refers to Adenosine monophosphate deaminase 2 (see, e.g., NCBI Entrez gene ID 271, the entire disclosure of which is herein incorporated by reference). FAM155A (or NALF1) refers to NALCN channel auxiliary factor 1 (see, e.g., NCBI Entrez gene ID 728215, the entire disclosure of which is herein incorporated by reference). IL13RA2 refers to Interleukin 13 receptor subunit alpha 2 (see, e.g., NCBI Entrez gene ID 3598, the entire disclosure of which is herein incorporated by reference).

However, other biomarkers can be used, e.g., in place of or in addition to any one or more of WNK2, OCRL, ASB7, PCBP3, AMPD2, FAM155A, and IL13RA2. For example, in some embodiments, biomarkers used in the methods include, but are not limited to, any one or more of the 324 biomarkers listed in Table 3. In some embodiments, the biomarkers include any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more biomarkers listed in Table 3. In some embodiments, the biomarkers include any one or more pairs of biomarkers listed in Table 4. For example, in some embodiments, the biomarkers include any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more pairs of biomarkers listed in Table 4. Any number of total biomarkers can be assessed in the methods, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 90, 95, 100, 200, 300, 400, 500, or more total biomarkers. It will be appreciated that any one or more of the herein-disclosed biomarkers can be used in combination with any other biomarkers, i.e., as subsets of a broader panel.

The biomarkers used in the present methods correspond to genes whose expression levels in cells within the biological sample from the subject correlate with the likelihood of the subject being a responder to anti-TNF alpha treatment, e.g., to an improvement or resolution of one or more symptoms or features of IBD in the subject following administration of the anti-TNF alpha therapy. The expression level of the individual biomarkers can be elevated or depressed in individuals responding to the anti-TNF alpha treatment relative to the level in non-responding individuals. For example, in particular embodiments, the expression levels of WNK2, OCRL, and/or ASB7 are elevated in responder subjects as compared to in subjects that are non-responders to an anti-TNF alpha therapy. In particular embodiments, the expression levels of PCBP3, AMPD2, FAM155A, and/or IL13RA2 are reduced in responder subjects as compared to in subjects that are non-responders to an anti-TNF alpha therapy. What is important is that the expression level of the biomarker is positively or inversely correlated with being a responder or non-responder, allowing the determination of an overall score, e.g., a TNF alpha prognostic, or biomarker score, that can be used to inform a treatment decision (i.e., to administer or not administer an anti-TNF alpha treatment).

Additional biomarkers that can be used in the present methods can be assessed and identified using any standard analysis method or metric, e.g., by analyzing data from samples taken from subjects with IBD prior to the treatment and known to respond or not respond to an anti-TNF alpha therapy evaluated post the treatment a certain period, as described in more detail elsewhere herein and as illustrated, e.g., in the Examples. In some methods, the specific form of IBD (e.g., Crohn's disease, Ulcerative Colitis), the specific anti-TNF alpha therapy administered (e.g., infliximab, Remicade, adalimumab, Humira, certolizumab, Cimzia, golimumab, Simponi, etanercept, Enbrel), and/or the specific criteria used to determine the responder status of the training data include that of the subject, but this is not required. Suitable metrics and methods include Pearson correlation, Kendall rank correlation, Spearman rank correlation, t-test, other non-parametric measures, over-sampling of the TNF alpha responding group, under-sampling of the TNF alpha non-responding group, and others including linear regression, non-linear regression, random forest and other tree-based methods, artificial neural networks, etc. In one embodiment, the feature selection uses univariate ranking with the absolute value of the Pearson correlation between the gene expression and outcome as the ranking metric. In some embodiments, features (genes) are selected using metrics that measure the effect size between the responder group and non-responder group. In some embodiments, features (genes) are further selected via greedy forward search optimized on training accuracy for a parsimonious set of genes. In some embodiments, features (genes) are selected via greedy forward search optimized on Area Under Operator Receiver Characteristic.

In some embodiments, data from multiple sources is inputted to a multi-cohort analysis using appropriate software, e.g., the MetaIntegrator package. In some embodiments, effect size is calculated for each mRNA within a study between anti-TNF alpha responding individuals and non-responding controls, e.g., as Hedges' g. In some embodiments, the pooled or summary effect size across all of the datasets is then computed, e.g., using DerSimonian and Laird's random effects model. In some embodiments, the effect size is then summarized and p values across all mRNAs corrected for multiple testing, e.g., based on Benjamini-Hochberg false discovery rate (FDR). In some embodiments, the p-values across the studies are then combined, e.g., using Fisher's sum of logs method, and the log-sum of p values that each mRNA is up- or downregulated is computed, along with corresponding p values. In some embodiments, meta-analysis is performed, e.g., by performing leave one-study out (LOO) analysis by removing one dataset at a time. In some embodiments, a greedy forward search can be used to identify a parsimonious set of genes with the greatest discriminatory power to distinguish samples from anti-TNF alpha responders and non-responders.

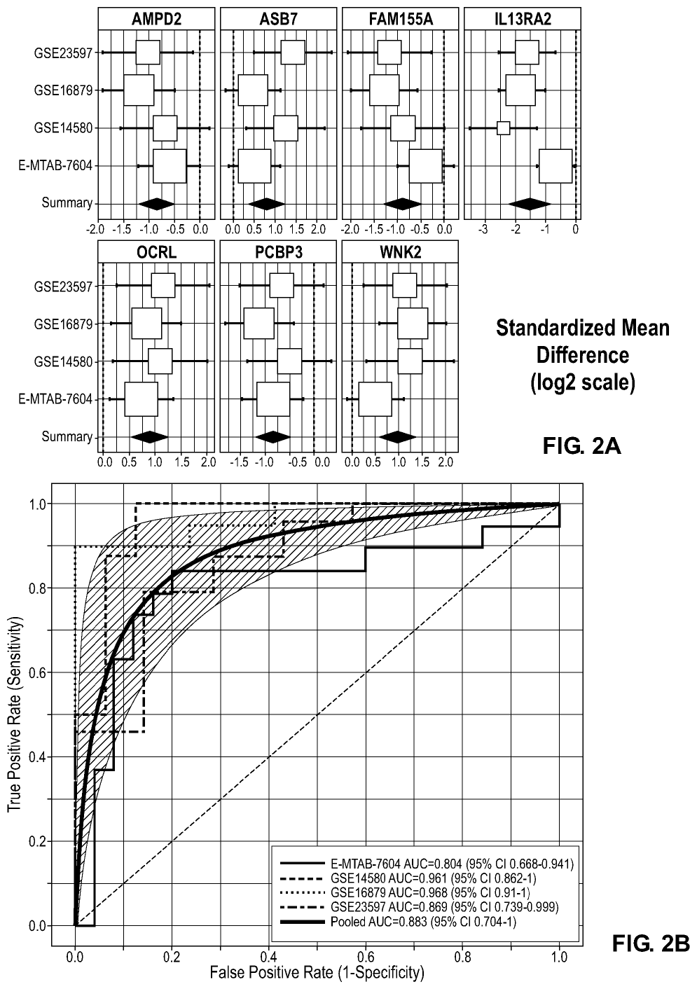

In particular embodiments, a machine learning workflow is applied to the training data, e.g., using a separate validation set or using cross-validation. For example, hyperparameter tuning can be used over a search space of parameters, e.g., parameters known to be effective for model optimization for infectious disease diagnosis. Examples of classifiers that can be used include linear classifiers such as Support Vector Machine (SVM) with linear kernel, logistic regression, and multi-layer perceptron with linear activation function, and non-linear classifiers such as SVM with non-linear kernel. Feature selection can be performed using the gene expression data for the candidate biomarkers as independent variables and using the known outcome as the dependent variable. The different models can be evaluated, e.g., using plots based on sensitivity and false-positive rates for each model, and the decision threshold evaluated during the hyperparameter search, and using ROC-like plots based on pooled cross-validated probabilities for the best models. (See, e.g., Ramkumar et al., Development of a Novel Proteomic Risk-Classifier for Prognostication of Patients with Early-Stage Hormone Receptor-Positive Breast Cancer. Biomarker Insights, Vol. 13, 1-9, 2018, FIG. 2A). Any of a number of different variants of cross-validation (CV) can be used, such as 5-fold random CV, 5-fold grouped CV, where each fold comprises multiple studies, and each study is assigned to exactly one CV fold, and leave-one-study-out (LOSO), where each study forms a CV fold. In some embodiments, the number of genes included in the final model can be limited, e.g., to 5, 6, 7, or 8, to facilitate translation to a rapid molecular assay.

### IV. Detecting Biomarker Expression

As described in more detail below, data sets corresponding to the biomarker gene expression levels as described herein are used to create a diagnostic or predictive rule or model based on the application of a statistical and machine learning algorithm, in order to produce a TNF alpha prognostic score. Such an algorithm uses relationships between a biomarker profile and an outcome, e.g., being a responder or non-responder to an anti-TNF alpha therapy (sometimes referred to as training data). The data are used to infer relationships that are then used to predict the TNF alpha responder status of a subject, e.g. the likelihood of being a responder or non-responder to an anti-TNF alpha therapy.

The expression levels of the biomarkers can be assessed in any of a number of ways. In particular embodiments, the expression levels of the biomarkers are determined by measuring polynucleotide levels of the biomarkers. For example, once the biological sample has been collected and preserved, RNA can be extracted using any method, so long that it permits the preservation of the RNA for subsequent quantification of the expression levels of the biomarker genes and of any control genes to be used, e.g., housekeeping genes used as reference values for the biomarkers. RNA can be extracted, e.g., from preserved cells manually, or using a robotic apparatus, such as Qiacube (QIAGEN) with a commercial RNA extraction kit. In some embodiments, RNA extraction is not performed, e.g., for isothermal amplification methods. In such methods, expression levels can be determined directly through lysis of cells, and then, e.g., reverse transcription and amplification of mRNA.

In some embodiments, the reference nucleic acid is a housekeeping gene or a product thereof, such as a corresponding mRNA transcript. In some embodiments, the reference nucleic acid includes an mRNA transcript that is a pre-mRNA molecule, a 5′ capped mRNA molecule, a 3′ adenylated mRNA molecule, or a mature mRNA molecule. In particular embodiments, the reference nucleic acid is a mature mRNA molecule obtained from a mammalian host that is also the source of the test sample. In some embodiments, the housekeeping gene or product thereof is expressed at a relatively constant rate by a cell of the host, such that the expression rate of the housekeeping gene can be used as a reference point against the expression of other host genes or gene products thereof.

In some embodiments, the reference nucleic acid is a human housekeeping gene. Exemplary human housekeeping genes suitable for use with the present methods include, but are not limited to, YWHAB, Chromosome 1 open reading frame 43 (C10rf43), Charged multivesicular body protein 2A (CHMP2A), ER membrane protein complex subunit 7 (EMC7), Glucose-6-phosphate isomerase (GPI), Proteasome subunit, beta type, 2 (PSMB2), Proteasome subunit, beta type, 4 (PSMB4), Member RAS oncogene family (RAB7A), Receptor accessory protein 5 (REEP5), small nuclear ribonucleoprotein D3 (SNRPD3), Valosin containing protein (VCP) and vacuolar protein sorting 29 homolog (VPS29). In some embodiments, any housekeeping gene provided at www/tau/ac/il˜elieis/HKG/may be used (see, Eisenberg and Levanon., Trends Genet. (2013), 10:569-74). Other suitable housekeeping genes include, e.g., GAPDH, ubiquitin, 18S (18S rRNA, e.g., HGNC (Human Genome Nomenclature Committee) nos. 44278-44281, 37657), ACTB (Actin beta, e.g., HGNC no. 132)), KPNA6 (Karyopherin subunit alpha 6, e.g., HGNC no. 6399), or RREB1 (ras-responsive element binding protein 1, e.g., HGNC no. 10449).

The levels of transcripts of the biomarker genes, or their levels relative to one another, and/or their levels relative to a reference gene such as a housekeeping gene, can be determined from the amount of mRNA, or polynucleotides derived therefrom, present in a biological sample. Polynucleotides can be detected and quantified by a variety of methods including, but not limited to, NanoString (e.g., nCounter analysis), microarray analysis, polymerase chain reaction (PCR), reverse transcriptase polymerase chain reaction (RT-PCR), quantitative RT-PCR (qRT-PCR), serial analysis of gene expression (SAGE), isothermal amplification methods such as qRT-LAMP, internal DNA detection switch, northern blotting, RNA fingerprinting, sequencing methods, ligase chain reaction, Qbeta replicase, strand displacement amplification, transcription based amplification systems, nuclease protection (Si nuclease or RNAse protection assays), as well as methods disclosed in International Publication Nos. WO 88/10315 and WO 89/06700, and International Applications Nos. PCT/US87/00880 and PCT/US89/01025; herein incorporated by reference in their entireties, and methods using MacMan probes, flip probes, and TaqMan probes (see, e.g., Murray et al. (2014) J. Mol Diag. 16:6, pp 627-638). See, e.g., Draghici, Data Analysis Tools for DNA Microarrays, Chapman and Hall/CRC, 2003; Simon et al., Design and Analysis of DNA Microarray Investigations, Springer, 2004; Real-Time PCR: Current Technology and Applications, Logan, Edwards, and Saunders eds., Caister Academic Press, 2009; Bustin, A-Z of Quantitative PCR (IUL Biotechnology, No. 5), International University Line, 2004; Velculescu et al. (1995) Science 270:484-487; Matsumura et al. (2005) Cell. Microbiol. 7:11-18; Serial Analysis of Gene Expression (SAGE): Methods and Protocols (Methods in Molecular Biology), Humana Press, 2008; each of which is herein incorporated by reference in its entirety.

In some embodiments, the biomarker gene expression is detected using a gene expression panel such as a NanoString nCounter, which allows the quantification of biomarker gene expression without the need for amplification or cDNA conversion. In such methods, RNA obtained from the blood or other biological sample from the subject is hybridized in solution to probes, e.g., a labeled reporter probe and a capture probe for each biomarker and control sequence. The target RNA-probe complexes are then purified and immobilized on a solid support, and then quantified, with each marker-specific probe having a specific fluorescent signature that allows the quantification of the specific marker. Such methods and the generation of probes, e.g., capture probes and reporter probes, for such applications are known in the art and are described, e.g., on the website nanostring.com.

For amplification-based methods such as qRT-PCR or qRT-LAMP, the primers can be obtained in any of a number of ways. For example, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

Computer programs that are well known in the art are useful in the design of primers with the required specificity and optimal amplification properties, such as Oligo version 5.0 (National Biosciences). PCR methods are well known in the art, and are described, for example, in Innis et al., eds., PCR Protocols: A Guide To Methods And Applications, Academic Press Inc., San Diego, Calif. (1990); herein incorporated by reference in its entirety.

In some embodiments, microarrays are used to measure the levels of biomarkers. An advantage of microarray analysis is that the expression of each of the biomarkers can be measured simultaneously, and microarrays can be specifically designed to provide a diagnostic expression profile for a particular disease or condition (e.g., IBD). Microarrays are prepared by selecting probes which comprise a polynucleotide sequence, and then immobilizing such probes to a solid support or surface. For example, the microarray may comprise a support or surface with an ordered array of binding (e.g., hybridization) sites or “probes” each representing one of the biomarkers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site. Conditions for preparing microarrays, for hybridization conditions, and for detection of bound probes are well known in the art (see, e.g., Sambrook, et al., Molecular Cloning: A Laboratory Manual (3rd Edition, 2001); Ausubel et al., Current Protocols In Molecular Biology, vol. 2, Current Protocols Publishing, New York (1994); Shalon et al., 1996, Genome Research 6:639-645; Schena et al., Genome Res. 6:639-645 (1996); and Ferguson et al., Nature Biotech. 14:1681-1684 (1996)).

As noted above, the “probe” to which a particular polynucleotide molecule specifically hybridizes contains a complementary polynucleotide sequence. The probes of the microarray typically consist of nucleotide sequences of, e.g., no more than 1,000 nucleotides, or of 10 to 1,000 nucleotides or 10-200, 10-30, 10-40, 20-50, 40-80, 50-150, or 80-120 nucleotides in length. The probes may comprise DNA sequences, RNA sequences, or copolymer sequences of DNA and RNA. The polynucleotide sequences of the probes may also comprise DNA and/or RNA analogs, derivatives, or combinations thereof. For example, the probes can be modified at the base moiety, at the sugar moiety, or at the phosphate backbone (e.g., phosphorothioates). The polynucleotide sequences of the probes may be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes are preferably selected using an algorithm that takes into account binding energies, base composition, sequence complexity, cross-hybridization potential based on probe similarities with other genes in the genome, and secondary structure. See Friend et al., International Patent Publication WO 01/05935, published Jan. 25, 2001; Hughes et al., Nat. Biotech. 19:342-7 (2001). An array will include both positive control probes, e.g., probes known to be complementary and hybridizable to sequences in the target polynucleotide molecules, and negative control probes, e.g., probes known to not be complementary and hybridizable to sequences in the target polynucleotide molecules. In addition, the present methods will include probes to both the biomarkers themselves, as well as to internal control sequences such as housekeeping genes, as described in more detail elsewhere herein.

In one embodiment, the disclosure provides a microarray comprising an oligonucleotide that hybridizes to a WNK2 polynucleotide, an oligonucleotide that hybridizes to an OCRL polynucleotide, an oligonucleotide that hybridizes to an ASB7 polynucleotide, an oligonucleotide that hybridizes to a PCBP3 polynucleotide, an oligonucleotide that hybridizes to an AMPD2 polynucleotide, an oligonucleotide that hybridizes to a FAM155A polynucleotide, and an oligonucleotide that hybridizes to an IL13RA2 polynucleotide. In some embodiments, the disclosure includes a microarray comprising an oligonucleotide that hybridizes to any of the biomarker genes listed in Table 3. In some embodiments, the disclosure includes a microarray comprising two oligonucleotides that hybridize to any of the biomarker pairs listed in Table 4.

In some embodiments, quantitative reverse transcriptase PCR (qRT-PCR) is used to determine the expression profiles of biomarkers (see, e.g., U.S. Patent Application Publication No. 2005/0048542A1; herein incorporated by reference in its entirety). The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

In some embodiments, the PCR employs the Taq DNA polymerase, which has a 5′-3′ nuclease activity but lacks a 3′-5′ proofreading endonuclease activity. TAQMAN PCR typically utilizes the 5′-nuclease activity of Taq or Tth polymerase to hydrolyze a hybridization probe bound to its target amplicon, but any enzyme with equivalent 5′ nuclease activity can be used. In such methods, two oligonucleotide primers are used to generate an amplicon typical of a PCR reaction, and a third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 sequence detection system. The system consists of a thermocycler, laser, charge-coupled device (CCD), camera and computer. The system includes software for running the instrument and for analyzing the data. 5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs that can be used to normalize patterns of gene expression include mRNAs for the housekeeping genes glyceraldehyde-3-phosphate-dehydrogenase (GAPDH) and beta-actin.

In particular embodiments, the biomarker gene expression is determined using isothermal amplification. Isothermal amplification is a process in which a target nucleic acid is amplified using a constant, single, amplification temperature (e.g., from about 30° C. to about 95° C.). Unlike standard PCR, an isothermal amplification reaction does not include multiple cycles of denaturation, hybridization, and extension, of an annealed oligonucleotide to form a population of amplified target nucleic molecules (i.e., amplicons). There are various types of isothermal application known in the art, including but not limited to, loop-mediated isothermal amplification (LAMP), nucleic acid sequence based amplification NASBA, recombinase polymerase amplification (RPA), rolling circle amplification (RCA), nicking enzyme amplification reaction (NEAR), and helicase dependent amplification (HDA).

In particular embodiments, the isothermal amplification is real-time quantitative isothermal amplification, in which a target nucleic acid is amplified at a constant temperature and the target nucleic acid rate of amplification is monitored by fluorescence, turbidity, or similar measures (e.g., NEAR or LAMP). In some cases, RNA (e.g., mRNA) is isolated from a biological sample and is used as a template to synthesize cDNA by reverse-transcription, cDNA molecules are amplified under isothermal amplification conditions such that the production of amplified target nucleic acid can be detected and quantitated.

In particular embodiments, the isothermal amplification is Loop-Mediated Isothermal Amplification (LAMP). LAMP offers selectivity and employs a polymerase and a set of specially designed primers that recognize distinct sequences in the target nucleic acid (see, e.g., Nixon et al., (2014) Bimolecular Detection and Quantitation, 2:4-10; Schuler et al., (2016) Anal Methods., 8:2750-2755; and Schoepp et al., (2017) Sci. Transl. Med., 9: eaal3693). Unlike PCR, the target nucleic acid is amplified at a constant temperature (e.g., 60-65° C.) using multiple inner and outer primers and a polymerase having strand displacement activity. In some instances, an inner primer pair containing a nucleic acid sequence complementary to a portion of the sense and antisense strands of the target nucleic acid initiate LAMP. Following strand displacement synthesis by the inner primers, strand displacement synthesis primed by an outer primer pair can cause release of a single-stranded amplicon. The single-stranded amplicon may serve as a template for further synthesis primed by a second inner and second outer primer that hybridize to the other end of the target nucleic acid and produce a stem-loop nucleic acid structure. In subsequent LAMP cycling, one inner primer hybridizes to the loop on the product and initiates displacement and target nucleic acid synthesis, yielding the original stem-loop product and a new stem-loop product with a stem twice as long. Additionally, the 3′ terminus of an amplicon loop structure serves as initiation site for self-templating strand synthesis, yielding a hairpin-like amplicon that forms an additional loop structure to prime subsequent rounds of self-templated amplification. The amplification continues with accumulation of many copies of the target nucleic acid. The final products of the LAMP process are stem-loop nucleic acids with concatenated repeats of the target nucleic acid in cauliflower-like structures with multiple loops formed by annealing between alternately inverted repeats of a target nucleic acid sequence in the same strand.

In some embodiments, the isothermal amplification assay comprises a digital reverse-transcription loop-mediated isothermal amplification (dRT-LAMP) reaction for quantifying the target nucleic acid (see, e.g., Khorosheva et al., (2016) Nucleic Acid Research, 44:2 e10). Typically, LAMP assays produce a detectable signal (e.g., fluorescence) during the amplification reaction. In some embodiments, fluorescence can be detected and quantified. Any suitable method for detecting and quantifying florescence can be used. In some instances, a device such as Applied Biosystem's QuantStudio can be used to detect and quantify fluorescence from the isothermal amplification assay.

Any suitable method for detecting amplification of a target nucleic acid in a test sample by quantitative real-time isothermal amplification may be used to practice the present methods. In some embodiments, quantitative real-time isothermal amplification of a target nucleic acid in a test sample is determined by detecting of one or more different (distinct) fluorescent labels attached to nucleotides or nucleotide analogs incorporated during isothermal amplification of the target nucleic acid (e.g., 5-FAM (522 nm), ROX (608 nm), FITC (518 nm) and Nile Red (628 nm). In another embodiment, quantitative real-time isothermal amplification of a target nucleic acid in a test sample can be determined by detection of a single fluorophore species (e.g., ROX (608 nm)) attached to nucleotides or nucleotide analogs incorporated during isothermal amplification of the target nucleic acid. In some embodiments, each fluorophore species used emits a fluorescent signal that is distinct from any other fluorophore species, such that each fluorophore can be readily detected among other fluorophore species present in the assay.

In some embodiments, methods of detecting amplification of a target nucleic acid in a test sample by quantitative real-time isothermal amplification can include using intercalating fluorescent dyes, such as SYTO dyes (SYTO 9 or SYTO 82). In some embodiments, methods of detecting amplification of a target nucleic acid in a test sample by quantitative real-time isothermal amplification can include using unlabeled primers to isothermally amplify the target nucleic acid in the test sample, and a labeled probe (e.g., having a fluorophore) to detect isothermal amplification of the target nucleic acid in the test sample. In some embodiments, unlabeled primers are used to isothermally amplify a target nucleic acid present in the test sample, and a probe is used having a 5-FAM dye label on the 5′ end and a minor groove binder (MGB) and non-fluorescent quencher on the 3′ end to detect isothermal amplification of the target nucleic acid (e.g., TaqMan Gene Expression Assays from ThermoFisher Scientific).

In some embodiments, detecting amplification of the target nucleic acid in the test sample is performed using a one-step, or two-step, quantitative real-time isothermal amplification assay. In a one-step quantitative real-time isothermal amplification assay, reverse transcription is combined with quantitative isothermal amplification to form a single quantitative real-time isothermal amplification assay. A one-step assay reduces the number of hands-on manipulations as well as the total time to process a test sample. A two-step assay comprises a first-step, where reverse transcription is performed, followed by a second-step, where quantitative isothermal amplification is performed. It is within the scope of the skilled artisan to determine whether a one-step or two-step assay should be performed.

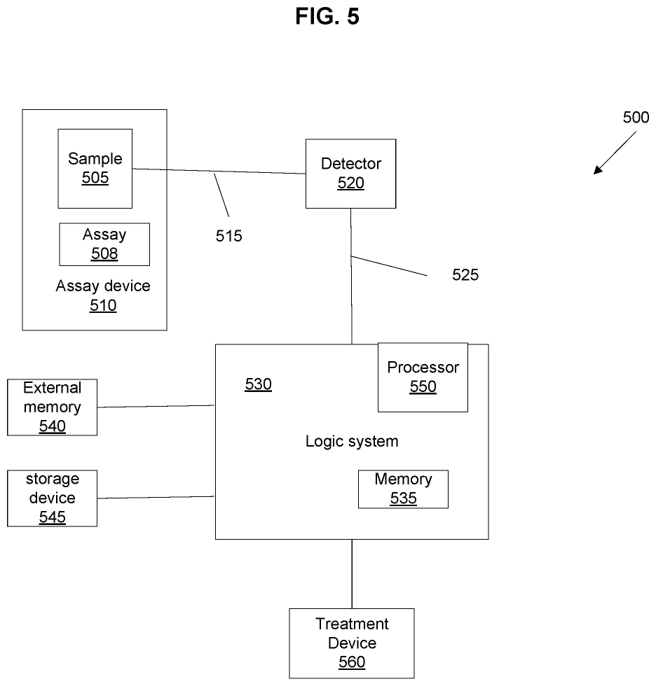

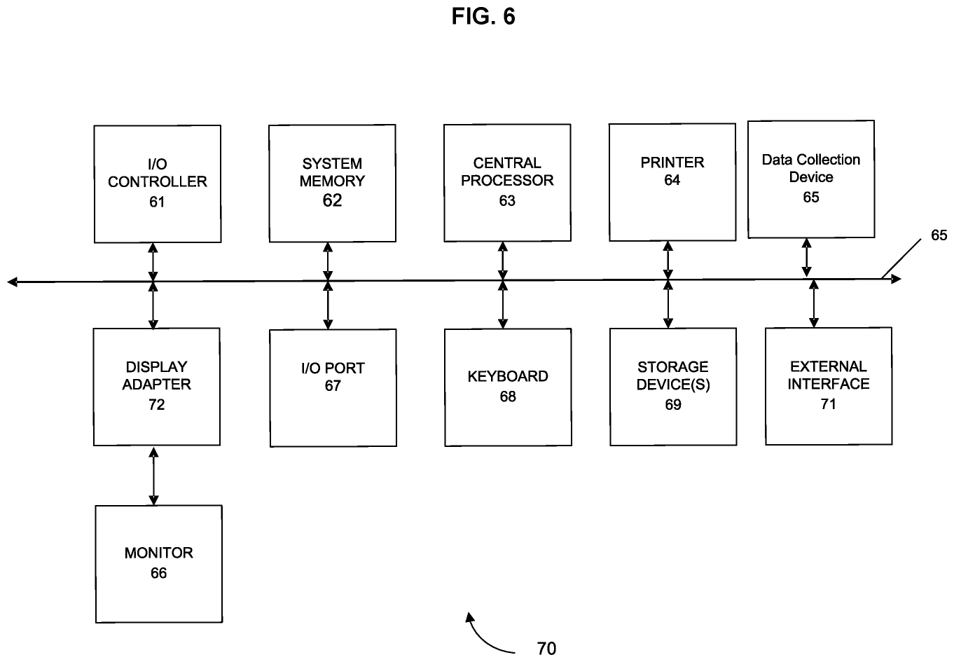

In some embodiments, the amplification and/or detection is carried out in whole or in part using an integrated measurement system, as illustrated in FIG. 5, which may also comprise a computer system as described elsewhere herein (see, e.g., FIG. 6).

In some embodiments, TNF alpha prognostic or biomarker scores are calculated based on the Tt (time to threshold) values or a parameter that measures the rapidity of detected signal rise for each of the tested biomarkers. This may be accomplished by, e.g., establishing standard curves for the isothermal or other amplification of the target nucleic acid (e.g., biomarker) and the reference nucleic acid (e.g., housekeeping gene). The standard curves can be obtained by performing real-time isothermal amplification assays using quantitated calibrator samples with multiple known input concentrations. Appropriate methods are provided in, e.g., PCT Publication No. WO 2020/061217, the entire disclosure of which is herein incorporated by reference.

For example, in some embodiments, to generate a standard curve, quantitated calibrator samples are obtained by performing serial dilutions of a quantitated material. For example, a template is serially diluted in a buffer at 10-fold concentration intervals yielding templates covering a range of concentrations from, e.g., approximately 109 copies/microliter to approximately 102 copies/microliter. The precise concentration of each calibrator sample can be determined using methods known in the art.

To obtain a standard curve, a real-time amplification assay is performed for each aliquot with a known quantity (e.g., 1 microliter L) of a respective calibrator sample with a respective concentration of the target nucleic acid. In a real-time amplification assay for each respective calibrator sample, the intensity of the fluorescence emitted by intercalating fluorescent dyes (e.g., dsDNA dyes) or fluorescent labels for the target nucleic acid is measured as a function of time. For example, a plot can be generated of fluorescence intensity as a function of time in a real-time quantitative amplification assay. A dashed line can be used to represent a pre-determined threshold intensity, and the elapsed time from the moment when the amplification is started is the time-to-threshold Tt. A respective time-to-threshold value can be determined from each respective fluorescence curve as a function of time. Thus, time-to-threshold values Tₜₙ, Tₜₙ₊₁, Tₜₙ₊₂, etc., are obtained for the different calibrator samples.

For exponential amplifications, the time-to-threshold is linearly proportional to the logarithm (e.g., logarithm to base 10) of the starting copy number (also referred to as template abundance). A scatter plot of data points can be generated from the fluorescence curves. Each data point represents a data pair [Log₁₀(CopyNumber), Tₜ] (note that CopyNumber refers to starting number of copies of a nucleic acid in an amplification assay). In some embodiments, the data points fall approximately on a straight line. A linear regression is then performed on the data points in the plot to obtain the straight line that best fits the data points with the least amount of total deviations. The result of the linear regression is a straight line represented by the following equation,

$${T}_{t} = m \times {Log}_{10} ( CopyNumber ) + b , \qquad ( 1 )$$

where m is the slope of the line, and b is y-intercept. The slope m represents the efficiency of the isothermal amplification of the target nucleic acid; b represents a time-to-threshold as template copy number approaches zero. The straight line represented by Equation (1) is referred to as the standard curve.

In some embodiments, replicates (e.g., triplicates) of isothermal amplification assays may be run for each sample in order to gain a higher level of confidence in the data. Replicate time-to-threshold values can be averaged, and standard deviations can be calculated.

Once the standard curve is established for a given isothermal amplification assay, the standard curve can be used to convert a time-to-threshold value to a starting copy number for future runs of the amplification assay of unknown starting numbers of copies of the target nucleic acid, using the following equation,

$$CopyNumber = {10}^{\frac{T t - b}{m}} . \qquad ( 2 )$$

Normally, the data points for low copy numbers or very high copy numbers may fall off of the straight line. The range of copy numbers within which the data points can be represented by the straight line is referred to as the dynamic range of the standard curve. The linear relationship between the time-to-threshold and the logarithmic of copy number represented by the standard curve would be valid only within the dynamic range.

If the amplification efficiencies for a target nucleic acid and a reference nucleic acid are different for a given isothermal amplification assay, it may be necessary to obtain separate standard curves for the target nucleic acid and the reference nucleic acid. Thus, two sets of real-time isothermal amplification assays may be performed, one set for establishing the standard curve for the target nucleic acid, the other set for establishing the standard curve for the reference nucleic acid. In cases where multiple target nucleic acids are considered (e.g., for a panel of seven biomarkers as described herein), a standard curve for each target nucleic acid may be obtained.

In some embodiments, the standard curves are generated prior to obtaining a test sample. That is, the standard curves are not generated on-board with the quantitative isothermal amplification of the test sample. Such standard curves may be referred to as off-board standard curves. Off-board standard curves may be used for estimating relative abundance values. For example, for a test sample of unknown input concentration of a target nucleic acid, a first real-time amplification assay is performed for a first aliquot of the test sample to obtain a first time-to-threshold value with respect to the target nucleic acid. A second real-time isothermal amplification assay is then performed for a second aliquot of the test sample to obtain a second time-to-threshold value with respect to a reference nucleic acid. The first aliquot and the second aliquot contain substantially the same amount of the test sample. The first time-to-threshold value may then be converted into starting number of copies of the target nucleic acid using the standard curve of the target nucleic acid. Similarly, the second time-to-threshold value may be converted into starting number of copies of the reference nucleic acid using the standard curve of the reference nucleic. The starting number of copies of the target nucleic acid is then normalized against that of the reference nucleic acid to obtain a relative abundance value.

In cases where the amplification efficiencies for a target nucleic acid and a reference nucleic acid have approximately the same value that is known, relative abundance may be obtained directly from time-to-threshold values without using standard curves.

### V. Calculating Biomarker (or TNF Alpha-Prognostic) Scores

To determine the likelihood that a subject with IBD will respond to an anti-TNF alpha treatment, a model (e.g., the model with the hyperparameter configuration providing the maximum AUC) is applied to the biomarker expression data from the subject to determine a score, e.g., a “TNF alpha prognostic score” or “biomarker score”, that is indicative of the probability of the subject responding to the anti-TNF alpha therapy. This score can be used, e.g., to classify the subject into any of a number of bins, e.g., 2 bins corresponding to being a responder or non-responder to an anti-TNF alpha therapy, or 3 bins with a “low”, “intermediate” or “indeterminate”, and “high” likelihood of being a responder to an anti-TNF alpha therapy, or 4 bins with “very likely”, “likely”, “unlikely”, and “very unlikely” characterizing the likelihood of responding to an anti-TNF alpha treatment. In a particular embodiment, the model uses logistic regression and the selected biomarker genes, e.g., WNK2, OCRL, ASB7, PCBP3, AMPD2, FAM155A, IL13RA2, to calculate the score. The probability of the subject responding to the anti-TNF alpha therapy as determined using the model is then used to determine the optimal treatment of the subject, as described in more detail elsewhere herein.

The TNF alpha prognostic or biomarker score can be calculated, e.g., by taking the sum, product, or quotient of the gene expression levels (as used herein, “gene levels”, “expression levels”, and “gene expression levels” are interchangeable), taken in terms of their absolute levels or their relative levels as compared to control genes, e.g., housekeeping genes, or by inputting them into a linear or nonlinear algorithm that incorporates at least the measured gene levels, e.g., the measured levels of 2, 3, 4, 5, 6, 7, 8, 9, 10 or more biomarker genes, into an interpretable score. In a particular embodiment, the score is calculated based on the expression data obtained for a panel of seven biomarkers.

In semi-quantitative methods, a threshold or cut-off value is suitably determined, and is optionally a predetermined value. In particular embodiments, the threshold value is predetermined in the sense that it is fixed, for example, based on previous experience with the assay and/or a population of subjects with a given outcome or outcomes, e.g., with a population of 50, 100, 150, 200, 250, 300, 350, 400, 450, 500, or more subjects with IBD responding or not responding to an anti-TNF alpha therapy. Alternatively, the predetermined value can also indicate that the method of arriving at the threshold is predetermined or fixed even if the particular value varies among assays or can even be determined for every assay run.

For the statistical analyses described herein, e.g., for the selection of biomarkers to be included in the calculation of a score or in the calculation of a probability or likelihood of a particular TNF alpha prognostic status in a patient, as well as for diagnostic or therapeutic assessments made in view of a given TNF alpha prognostic or biomarker score, other relevant information can also be considered, such as clinical data regarding the symptoms presented by each individual. This can include demographic information such as age, race, and sex; information regarding a presence, absence, degree, stage, severity or progression of a condition, phenotypic information, such as details of phenotypic traits, genetic or genetically regulated information, amino acid or nucleotide related genomics information, results of other tests including imaging, biochemical and hematological assays, other physiological scores, or the like.

As described above, the abundance values for the individual biomarker genes in cells of the biological sample can be combined using a mathematical formula or a machine learning or other algorithm to produce a single prognostic score, such as the TNF alpha prognostic score that can indicate the likelihood of a subject with IBD being a responder to an anti-TNF alpha therapy. In these embodiments, the produced score carries more predictive power than any individual gene level alone (e.g., has a greater area under the receiver-operating-characteristic curve for discrimination of responders vs non-responders).

In some embodiments, types of algorithms for integrating multiple biomarkers into a single diagnostic score may include, but not limited to, a difference of geometric means, a difference of arithmetic means, a difference of sums, a simple sum, and the like. In some embodiments, a diagnostic score may be estimated based on the relative abundance values of multiple biomarkers using machine-learning models, such as a regression model, a tree-based machine-learning model, a support vector machine (SVM) model, an artificial neural network (ANN) model, or the like.

Biomarker data may also be analyzed by a variety of methods to determine the statistical significance of differences in observed levels of biomarkers between test and reference expression profiles in order to evaluate the TNF alpha responder status or probability of being a responder to an anti-TNF alpha therapy in a subject with IBD. In certain embodiments, patient data is analyzed by one or more methods including, but not limited to, multivariate linear discriminant analysis (LDA), receiver operating characteristic (ROC) analysis, principal component analysis (PCA), ensemble data mining methods, significance analysis of microarrays (SAM), cell specific significance analysis of microarrays (csSAM), spanning-tree progression analysis of density-normalized events (SPADE), and multi-dimensional protein identification technology (MUDPIT) analysis. (See, e.g., Hilbe (2009) Logistic Regression Models, Chapman & Hall/CRC Press; Mclachlan (2004) Discriminant Analysis and Statistical Pattern Recognition. Wiley Interscience; Zweig et al. (1993) Clin. Chem. 39:561-577; Pepe (2003) The statistical evaluation of medical tests for classification and prediction, New York, N.Y.: Oxford; Sing et al. (2005) Bioinformatics 21:3940-3941; Tusher et al. (2001) Proc. Natl. Acad. Sci. U.S.A. 98:5116-5121; Oza (2006) Ensemble data mining, NASA Ames Research Center, Moffett Field, Calif., USA; English et al. (2009) J. Biomed. Inform. 42 (2): 287-295; Zhang (2007) Bioinformatics 8:230; Shen-Orr et al. (2010) Journal of Immunology 184:144-130; Qiu et al. (2011) Nat. Biotechnol. 29 (10): 886-891; Ru et al. (2006) J. Chromatogr. A. 1111 (2): 166-174, Jolliffe Principal Component Analysis (Springer Series in Statistics, 2.sup.nd edition, Springer, N Y, 2002), Koren et al. (2004) IEEE Trans Vis Comput Graph 10:459-470; herein incorporated by reference in their entireties.)

It is not necessary that all of the biomarkers are elevated or depressed relative to control levels in a biological sample from a given subject to give rise to a determination that a subject will respond to an anti-TNF alpha therapy. For example, for a given biomarker level there can be some overlap between individuals falling into different probability categories. However, collectively the combined levels for all of the biomarker genes included in the assay will give rise to a score that, if it surpasses a threshold, e.g., a threshold derived from at least 50, 100, 150, 200, 250, 300, 350, 400, 500 or more IBD patients who respond to an anti-TNF alpha therapy, and/or of 10, 20, 30, 40, 50, 100, 150, 200, 250, 300, 350, 400, 500 or more control individuals with IBD who do not respond to an anti-TNF alpha therapy, that allows a determination concerning the TNF alpha prognostic status of the subject. For example, for a determination of being a non-responder to an anti-TNF alpha therapy, the threshold could be such that at across a population of at least 100 IBD patients who respond and 100 patients with IBD who do not respond to an anti-TNF alpha therapy, at least 90% of the patients who do not respond to the therapy are above the threshold. It will be appreciated that in any given assay there can be more than one threshold, e.g., a threshold in one direction that indicates that a patient will respond to an anti-TNF alpha therapy, and a threshold in the other direction that indicates that a patient will not respond to an anti-TNF alpha therapy. It will also be appreciated that, in some embodiments, an indication of being a responder to an anti-TNF alpha therapy is not specific to the form of IBD and/or to the specific anti-TNF alpha therapy administered. Further, an indication of being a non-responder to an anti-TNF alpha therapy is independent of other aspects of the subject's condition and/or their likelihood of responding to other therapeutic approaches.

As used herein, the terms “probability,” and “risk” with respect to a given outcome refer to conditional probability that subjects with a particular score actually have the condition (e.g., being a responder to an anti-TNF alpha therapy) based on a given mathematical model. An increased probability or risk for example can be relative or absolute and can be expressed qualitatively or quantitatively. For instance, an increased risk can be expressed as simply determining the subject's score and placing the test subject in an “increased risk” category, based upon previous population studies. Alternatively, a numerical expression of the test subject's increased risk can be determined based upon an analysis of the biomarker or prognostic score.

In some embodiments, likelihood is assessed by comparing the level of a biomarker or TNF alpha prognostic score to one or more preselected or threshold levels. Threshold values can be selected that provide an acceptable ability to predict the TNF alpha responder status of an IBD patient. In illustrative examples, receiver operating characteristic (ROC) curves are calculated by plotting the value of a biomarker or TNF alpha prognostic score in two populations in which a first population has a first condition (e.g., non-responder to anti-TNF alpha therapy) and a second population has a second condition (e.g., responder to anti-TNF alpha therapy).

For any particular biomarker, a distribution of biomarker levels for subjects with and without a disease will likely overlap, and some overlap will be present for biomarker or TNF alpha prognostic scores as well. Under such conditions, a test does not absolutely distinguish a first condition and a second condition with 100% accuracy, and the area of overlap indicates where the test cannot distinguish the first condition and the second condition. A threshold value is selected, above which (or below which, depending on how a biomarker or TNF alpha prognostic score changes with a specified condition or prognosis) the test is considered to be “positive” and below which the test is considered to be “negative.” The area under the ROC curve (AUC) provides the C-statistic, which is a measure of the probability that the perceived measurement will allow correct identification of a condition (see, e.g., Hanley et al., Radiology 143:29-36 (1982)).

In some embodiments, a positive likelihood ratio, negative likelihood ratio, odds ratio, and/or AUC or receiver operating characteristic (ROC) values are used as a measure of a method's ability to predict the TNF alpha responder status. As used herein, the term “likelihood ratio” is the probability that a given test result would be observed in a subject with a condition or outcome of interest divided by the probability that that same result would be observed in a patient without the condition or outcome of interest. Thus, a positive likelihood ratio is the probability of a positive result observed in subjects with the specified condition or outcome divided by the probability of a positive results in subjects without the specified condition or outcome. A negative likelihood ratio is the probability of a negative result in subjects without the specified condition or outcome divided by the probability of a negative result in subjects with specified condition or outcome.

The term “odds ratio,” as used herein, refers to the ratio of the odds of an event occurring in one group (e.g., not responding to an anti-TNF alpha therapy) to the odds of it occurring in another group (e.g., responding to an anti-TNF alpha therapy), or to a data-based estimate of that ratio. The term “area under the curve” or “AUC” refers to the area under the curve of a receiver operating characteristic (ROC) curve, both of which are well known in the art. AUC measures are useful for evaluating the accuracy of a classifier across the complete decision threshold range. Classifiers with a greater AUC have a greater capacity to classify unknowns correctly between two or more groups of interest (e.g., responder or non-responder to an anti-TNF alpha therapy, or a low, intermediate, or high probability of being a responder to an anti-TNF alpha therapy). ROC curves are useful for plotting the performance of a particular feature (e.g., any of the biomarker expression levels or biomarker scores described herein and/or any item of additional biomedical information) in distinguishing or discriminating between two populations (e.g., responders or non-responders to an anti-TNF alpha therapy). Typically, the feature data across the entire population (e.g., the cases and controls) are sorted in ascending order based on the value of a single feature. Then, for each value for that feature, the true positive and false positive rates for the data are calculated. The sensitivity is determined by counting the number of cases above the value for that feature and then dividing by the total number of cases. The specificity is determined by counting the number of controls below the value for that feature and then dividing by the total number of controls.

Although this refers to scenarios in which a feature is elevated in cases compared to controls, it also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to produce a single value, and this single value can be plotted in a ROC curve. Additionally, any combination of multiple features, in which the combination derives a single output value, can be plotted in a ROC curve. These combinations of features can comprise a test. The ROC curve is the plot of the sensitivity of a test against 1-specificity of the test, where sensitivity is traditionally presented on the vertical axis and 1-specificity is traditionally presented on the horizontal axis. Thus, “AUC ROC values” are equal to the probability that a classifier will rank a randomly chosen positive instance higher than a randomly chosen negative one.

In some embodiments, at least two (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30 or more) biomarker genes are selected to discriminate between subjects with a first condition or outcome and subjects with a second condition or outcome with at least about 70%, 75%, 80%, 85%, 90%, 95% accuracy or having a C-statistic of at least about 0.70, 0.75, 0.80, 0.85, 0.90, 0.95.

In the case of a positive likelihood ratio, a value of 1 indicates that a positive result is equally likely among subjects in both the “condition” and “control” groups (e.g., in individuals with IBD that are responders or non-responders to an anti-TNF alpha therapy); a value greater than 1 indicates that a positive result is more likely in the condition group (e.g., in responders); and a value less than 1 indicates that a positive result is more likely in the control group (e.g., in non-responders). In this context, “condition” is meant to refer to a group having one characteristic (e.g., being a responder) and “control” group lacking the same characteristic (e.g., being a non-responder). In the case of a negative likelihood ratio, a value of 1 indicates that a negative result is equally likely among subjects in both the “condition” and “control” groups; a value greater than 1 indicates that a negative result is more likely in the “condition” group; and a value less than 1 indicates that a negative result is more likely in the “control” group.

In certain embodiments, the biomarker or TNF alpha prognostic score is calculated, based on the measured levels of the biomarkers in individuals with IBD that are responders or non-responders to an anti-TNF alpha therapy, such that the likelihood ratio corresponding to the high risk bin is 1.5, 2, 2.5, 3, 3.5, 4, or more, or that the likelihood ratio corresponding to the low risk bin is 0.15, 0.10, 0.05, or lower, for being a responder to an anti-TNF alpha therapy.

In the case of an odds ratio, a value of 1 indicates that a positive result is equally likely among subjects in both the condition” and “control” groups; a value greater than 1 indicates that a positive result is more likely in the “condition” group; and a value less than 1 indicates that a positive result is more likely in the “control” group. In the case of an AUC ROC value, this is computed by numerical integration of the ROC curve. The range of this value can be 0.5 to 1.0. A value of 0.5 indicates that a classifier (e.g., a biomarker level) cannot discriminate between cases and controls (e.g., responders vs non-responders), while 1.0 indicates perfect diagnostic accuracy. In certain embodiments, biomarker gene levels and/or biomarker scores are selected to exhibit a positive or negative likelihood ratio of at least about 1.5 or more or about 0.67 or less, at least about 2 or more or about 0.5 or less, at least about 5 or more or about 0.2 or less, at least about 10 or more or about 0.1 or less, or at least about 20 or more or about 0.05 or less.

In certain embodiments, the biomarker gene levels and/or biomarker scores are selected to exhibit an odds ratio of at least about 2 or more or about 0.5 or less, at least about 3 or more or about 0.33 or less, at least about 4 or more or about 0.25 or less, at least about 5 or more or about 0.2 or less, or at least about 10 or more or about 0.1 or less. In certain embodiments, biomarker gene levels and/or biomarker scores are selected to exhibit an AUC ROC value of greater than 0.5, preferably at least 0.6, more preferably 0.7, still more preferably at least 0.8, even more preferably at least 0.9, and most preferably at least 0.95.

In some cases, multiple thresholds can be determined in so-called “tertile,” “quartile,” or “quintile” analyses. In these methods, the “diseased” and “control groups” (or “high risk” and “low risk”) groups are considered together as a single population, and are divided into 3, 4, or 5 (or more) “bins” having equal numbers of individuals. The boundary between two of these “bins” can be considered “thresholds.” A risk (of a particular diagnosis or prognosis for example) can be assigned based on which “bin” a test subject falls into. In some embodiments of the present methods, subjects are assigned to one of three bins, i.e. “low”, “intermediate”, or “high”, referring to the probability of responding to an anti-TNF alpha therapy based on the TNF alpha prognostic score obtained using the present methods. For example, subjects can be classified according to the estimated probability of responding to an anti-TNF alpha therapy into 3 bins: low likelihood (bin 1), intermediate (bin 2), and high-likelihood (bin 3). The bins are defined, e.g., such that the likelihood ratios are <0.15 in bin 1, from 0.15 to 5 in bin 2, and >5 in bin 3.

The phrases “assessing the likelihood” and “determining the likelihood,” as used herein, refer to methods by which the skilled artisan can predict the presence or absence of a condition (e.g., being a responder to an anti-TNF alpha therapy) in a patient. The skilled artisan will understand that this phrase includes within its scope an increased probability that a condition is present or absent in a patient; that is, that a condition is more likely to be present or absent in a subject. For example, the probability that an individual identified as having a specified condition actually has the condition can be expressed as a “positive predictive value” or “PPV.” Positive predictive value can be calculated as the number of true positives divided by the sum of the true positives and false positives. PPV is determined by the characteristics of the predictive methods of the present methods as well as the prevalence of the condition in the population analyzed. The statistical algorithms can be selected such that the positive predictive value in a population having a condition prevalence is in the range of 70% to 99% and can be, for example, at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In other examples, the probability that an individual identified as not having a specified condition or outcome actually does not have that condition can be expressed as a “negative predictive value” or “NPV.” Negative predictive value can be calculated as the number of true negatives divided by the sum of the true negatives and false negatives. Negative predictive value is determined by the characteristics of the diagnostic or prognostic method, system, or code as well as the prevalence of the disease in the population analyzed. The statistical methods and models can be selected such that the negative predictive value in a population having a condition prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, a subject is determined to have a significant probability of having or not having a specified condition or outcome. By “significant probability” is meant that the subject has a reasonable probability (0.6, 0.7, 0.8, 0.9 or more) of having, or not having, a specified condition or outcome.

In some embodiments, the biomarker score is combined with one or more clinical parameters. For example, a formula is used to combine (i) either the individual gene expression values or the output from a classifier that uses the gene expression values, with (ii) the one or more clinical parameter-based scores or data, to generate (iii) a new score that is useful to the clinician.

### VI. Treatment Decisions

The methods described herein may be used to classify subjects with IBD according to whether they are or non-responders to an anti-TNF alpha therapy, or the probability of their being responders to an anti-TNF alpha therapy. In some embodiments, the subjects are classified as responders or non-responders to an anti-TNF alpha therapy. In some embodiments, subjects are classified as having high, low, or intermediate probability of being a responder to an anti-TNF alpha therapy. A determination of a high probability of being a responder to an anti-TNF alpha therapy in an IBD patient can indicate the delivery of an anti-TNF alpha therapy to the patient, alone or together with other drugs or other forms of medical care appropriate for their form of IBD. For example, in some embodiments, patients identified as being a responder to an anti-TNF alpha therapy could be started (or maintained) on an anti-TNF alpha drug such as infliximab, Remicade, adalimumab, Humira, certolizumab, Cimzia, golimumab, Simponi, etanercept, Enbrel, or a biosimilar of one of these drugs. Such TNF alpha therapy could be of any appropriate duration or administration regimen as determined by a medical professional, and is not to the exclusion of other appropriate therapeutic approaches for IBD, as described in more detail below.

In some embodiments, patients identified as being a non-responder to anti-TNF alpha therapy using the herein-described methods can be treated using IBD therapeutic approaches other than via TNF alpha inhibitors, as described in more detail below, and if an anti-TNF alpha therapy had been initiated it could be discontinued.

In either case, regardless of the determined TNF alpha prognostic status in a patient, the patient is likely to receive other forms of medical care for IBD, either in conjuction with an anti-TNF alpha therapy (i.e., in responders) or without it (i.e., in non-responders). As used herein, “medical care” comprises any action taken with respect to the treatment of the subject, whether in an emergency room, urgent care context, another clinical facility or context, or at home, in order to alleviate, eliminate, slow the progression of, or in any way improve any aspect or symptom of IBD, including, but not limited to, administering a therapeutic drug, performing surgery, and assisting with symptom management. For example, in some embodiments the patient is administered an aminosalicylate, a corticosteroid, an antibiotic, an immunomodulator, or a JAK inhibitor. In some embodiments, the patient undergoes surgery such as bowel resection, strictureplasty, colectomy, proctocolectomy with ostomy, surgery for fistulas, or procedures to drain absesses. In some embodiments, the patient receives or is recommended an over-the-counter medicine or supplement such as an anti-diarrheal, pain reliever, iron supplement, vitamin B12 or D, or calcium.

### VII. Kits and Systems

### A. Kits

In one aspect, kits are provided for the determination of the TNF alpha responder status in a subject with IBD, wherein the kits can be used to detect the biomarkers described herein. For example, the kits can be used to detect any one or more of the biomarkers described herein, which are differentially expressed in biological (e.g., intestinal biopsy such as a colonic or ileal mucosal biopsy) samples from subjects that are responders to an anti-TNF alpha therapy and from subjects that are non-responders to an anti-TNF alpha therapy. The kit may include one or more agents for the detection of biomarkers, a container for holding a biological sample isolated from a human subject with IBD; and printed instructions for reacting agents with the biological sample or a portion of the biological sample to detect the presence or amount of at least one biomarker in the biological sample. The agents may be packaged in separate containers. The kit may further comprise one or more control reference samples and reagents for performing a PCR, isothermal amplification, immunoassay, NanoString, or microarray analysis, e.g., reference samples from TNF alpha responders or non-responders. The kit may also comprise one or more devices or implements for carrying out any of the herein devices, e.g., 96-well plates, microfluidic cartridges, single-well multiplex assays, etc.

In certain embodiments, the kit comprises agents for measuring the levels of at least seven biomarkers of interest. For example, the kit may include agents, e.g., primers and/or probes, for detecting biomarkers of a panel comprising a WNK2 polynucleotide, an OCRL polynucleotide, an ASB7 polynucleotide, a PCBP3 polynucleotide, an AMPD2 polynucleotide, a FAM155A polynucleotide, and an IL13RA2 polynucleotide, or for detecting any one or more biomarkers listed in Table 3, or one or more pairs of biomarkers listed in Table 4.

In certain embodiments, the kit comprises a microarray or other solid support for analysis of a plurality of biomarker polynucleotides. An exemplary microarray or other support included in the kit comprises an oligonucleotide that hybridizes to a WNK2 polynucleotide, an oligonucleotide that hybridizes to an OCRL polynucleotide, an oligonucleotide that hybridizes to an ASB7 polynucleotide, an oligonucleotide that hybridizes to a PCBP3 polynucleotide, an oligonucleotide that hybridizes to a PCBP3 polynucleotide, an oligonucleotide that hybridizes to an AMPD2 polynucleotide, an oligonucleotide that hybridizes to a FAM155A polynucleotide, and an oligonucleotide that hybridizes to an IL13RA2 polynucleotide. In some embodiments, the microarray or other support comprises an oligonucleotide for each of the biomarkers detected using the herein-described methods.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of determining the TNF alpha prognostic status of a subject.

### B. Measurement Systems for Detecting and Recording Biomarker Expression

In one aspect, a measurement system is provided. Such systems allow, e.g., the detection of biomarker gene expression in a sample and the recording of the data resulting from the detection. The stored data can then be analyzed as described elsewhere herein to determine the virus infection status of a subject. Such systems can comprise assay systems (e.g., comprising an assay device and detector), which can transmit data to a logic system (such as a computer or other system or device for capturing, transforming, analyzing, or otherwise processing data from the detector). The logic system can have any one or more of multiple functions, including controlling elements of the overall system such as the assay system, sending data or other information to a storage device or external memory, and/or issuing commands to a treatment device.

An exemplary measurement system is shown in FIG. 5. The system as shown includes a sample 505, an assay device 510, where an assay 508 can be performed on sample 505. For example, sample 505 can be contacted with reagents of assay 508 to provide a signal of a physical characteristic 515. An example of an assay device can be a flow cell that includes probes and/or primers of an assay or a tube through which a droplet moves (with the droplet including the assay). Physical characteristic 515 (e.g., a fluorescence intensity, a voltage, or a current), from the sample is detected by detector 520. Detector 520 can take a measurement at intervals (e.g., periodic intervals) to obtain data points that make up a data signal. In one embodiment, an analog-to-digital converter converts an analog signal from the detector into digital form at a plurality of times. Assay device 510 and detector 520 can form an assay system, e.g., an amplification and detection system that measures biomarker gene expression according to embodiments described herein. A data signal 525 is sent from detector 520 to logic system 530. As an example, data signal 525 can be used to determine expression levels for selected biomarkers. Data signal 525 can include various measurements made at a same time, e.g., different colors of fluorescent dyes or different electrical signals for different molecules of sample 505, and thus data signal 525 can correspond to multiple signals. Data signal 525, either directly or after online processing by Processor 550, may be stored in a local memory 535, an external memory 540, or a storage device 545. System 500 may also include a treatment device 560, which can provide a treatment to the subject. Treatment device 560 can determine a treatment and/or be used to perform a treatment, e.g., an anti-TNF alpha treatment. Logic system 530 may be connected to treatment device 560, e.g., to provide results of a method described herein. The treatment device may receive inputs from other devices, such as an imaging device and user inputs (e.g., to control the treatment, such as controls over a robotic system).

### C. Computer Systems and Diagnostic Systems

Certain aspects of the herein-described methods may be totally or partially performed with a computer system including one or more processors, which can be configured to perform the steps. Thus, embodiments are directed to computer systems configured to perform the steps of methods described herein, potentially with different components performing a respective step or a respective group of steps. The computer systems of the present disclosure can be part of a measuring system as described above, or can be independent of any measuring systems. In some embodiments, the present disclosure provides a computer system that calculates a TNF alpha prognostic score based on inputted biomarker expression (and optionally other) data, and determines the TNF alpha responder status of a subject.

An exemplary computer system is shown in FIG. 6. Any of the computer systems may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. The subsystems shown in FIG. 6 are interconnected via a system bus 65. Additional subsystems such as a printer 64, keyboard 68, storage device(s) 69, monitor 66 (e.g., a display screen, such as an LED), which is coupled to display adapter 72, and others are shown. Peripherals and input/output (I/O) devices, which couple to I/O controller 61, can be connected to the computer system by any number of means known in the art such as input/output (I/O) port 67 (e.g., USB, FireWire®). For example, I/O port 67 or external interface 71 (e.g. Ethernet, Wi-Fi, etc.) can be used to connect computer system 70 to a wide area network such as the Internet, a mouse input device, or a scanner. The interconnection via system bus 65 allows the central processor 63 to communicate with each subsystem and to control the execution of a plurality of instructions from system memory 62 or the storage device(s) 69 (e.g., a fixed disk, such as a hard drive, or optical disk), as well as the exchange of information between subsystems. The system memory 62 and/or the storage device(s) 69 may embody a computer readable medium. Another subsystem is a data collection device 65, such as a camera, microphone, accelerometer, and the like. Any of the data mentioned herein can be output from one component to another component and can be output to the user. A computer system can include a plurality of the same components or subsystems, e.g., connected together by external interface 71, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

In one aspect, the present disclosure provides a computer implemented method for determining the TNF alpha responder status of an IBD patient. The computer performs steps comprising, e.g., receiving inputted patient data comprising values for the levels of one or more biomarkers in a biological sample from the patient; analyzing the levels of one or more biomarkers and optionally comparing them to respective reference values, e.g., to a housekeeping reference gene for normalization; calculating a TNF alpha prognostic score for the patient based on the levels of the biomarkers and comparing the score to one or more threshold values to assign the patient to a TNF alpha responder category; and displaying information regarding the TNF alpha responder status or probability of a TNF alpha response in the patient. In certain embodiments, the inputted patient data comprises values for the levels of a plurality of biomarkers in a biological sample from the patient, e.g., biomarkers comprising one or more biomarkers listed in Table 3 or one or more pairs of biomarkers listed in Table 4. In one embodiment, the inputted patient data comprises values for the levels of WNK2, OCRL, ASB7, PCBP3, AMPD2, FAM155A, and IL13RA2 polynucleotides.

In a further aspect, a diagnostic system is included for performing the computer implemented method, as described. A diagnostic system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for determining the TNF alpha responder status (i.e., being a responder or non-responder to anti-TNF alpha therapy) of the subject. For example, the storage component includes instructions for calculating the TNF alpha prognostic score for the subject based on biomarker expression levels, as described herein. In addition, the storage component may further comprise instructions for performing multivariate linear discriminant analysis (LDA), receiver operating characteristic (ROC) analysis, principal component analysis (PCA), ensemble data mining methods, cell specific significance analysis of microarrays (csSAM), or multi-dimensional protein identification technology (MUDPIT) analysis. The computer processor is coupled to the storage component and configured to execute the instructions stored in the storage component in order to receive patient data and analyze patient data according to one or more algorithms. The display component displays information regarding the diagnosis of the patient. The storage component may be of any type capable of storing information accessible by the processor, such as a hard-drive, memory card, ROM, RAM, DVD, CD-ROM, USB Flash drive, write-capable, and read-only memories.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data. In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may actually comprise a collection of processors which may or may not operate in parallel. In one aspect, computer is a server communicating with one or more client computers. Each client computer may be configured similarly to the server, with a processor, storage component and instructions. Although the client computers and may comprise a full-sized personal computer, many aspects of the system and method are particularly advantageous when used in connection with mobile devices capable of wirelessly exchanging data with a server over a network such as the Internet.

### VIII. EXAMPLES

The following examples are offered to illustrate, but not to limit, the claimed invention.

A. Example 1. A Baseline Gene Expression-Based Prognostic for Anti-TNFa Therapy in Patients with Inflammatory Bowel Disease

### 1. Summary

Anti-TNF alpha therapies transformed the care and management of Inflammatory Bowel Disease (IBD). However, they are costly and ineffective in greater than 50% of patients, and increase the risk of infections, liver issues, arthritis, and lymphoma. With 1.6 million Americans suffering from IBD and global prevalence on the rise, a prognostic for anti-TNF alpha response would greatly improve the efficacy and cost-to-benefit ratio of these biologics. Herein, we describe the results of our biomarker discovery research efforts to identify a robust, prognostic multi-mRNA signature for anti-TNF alpha response in Inflammatory Bowel Disease (IBD) patients. Our survey of public data repositories and further curation identified four transcriptomic datasets (n=136) from colonic and ileal mucosal biopsies of IBD patients collected prior to treatment of anti-TNF alpha therapy with clinical response adjudicated post treatment. We removed platform related batch effects using COCONUT and, with conormalized datasets, performed a leave-one-study-out (LOSO) multicohort analysis of the four datasets and identified 324 significant differentially expressed (DE) genes between responders and non-responders. We evaluated the discriminatory performance of these genes by computing a difference of geometric means-based response score and subsequently computing area under receiver operating characteristic curves (AUROC). To identify a parsimonious gene signature for clinical use, we used a greedy forward search algorithm which yielded 7 genes whose performance we similarly evaluated. To show that any combination of the significant genes has robust performance, we evaluated all gene-pairs possible. In addition, we used both the DE set (324 genes) and the parsimonious set (7 genes) with all 136 samples combined into one dataset to evaluate eight available classifiers using our Inflammatix Machine Learning (IML) platform, which covers a broad spectrum of Machine Learning algorithms commonly used in biomedical fields. Overall, our preliminary work suggests the existence of a robust, generalizable response signal with potential to be translated for clinical use.

| | | Timepoint of | |
|---|---|---|---|
| Accession | Author | assessment | Clinical Adjudication Guidelines |
| EMTAB7604 | Verstockt | 6 months after | In CD patients, response was defined as a Simple |
| | | Infliximab and/or | Endoscopic Disease (SES-CD) score <=2 (cite |
| | | adalimumab | 30137278). In UC patients, response was defined as |
| | | treatment | Mayo endoscopic subscore of <=1 and was considered |
| | | | as endoscopic remission. |
| GSE14580 | Arijs | 4 weeks after | Response was defined as complete mucosal healing |
| | | Infliximab | with a Mayo endoscopic subscore of 0 or 1 (cite PMID: |
| | | treatment | 16339095) and a grade 0 or 1 on the histological score |
| | | | for ulcerative colitis (cite PMID: 10940279). Patients |
| | | | who did not achieve healing were considered non- |
| | | | responders although some of them presented |
| | | | endoscopic and/or histological improvement. |
| GSE16879 | Arijs | 4-6 weeks | For CDc (CD samples extracted from colon), response |
| | | after first | was defined as complete mucosal healing with a |
| | | Infliximab | decrease of at least 3 points on the histological score |
| | | treatment | and decrease to a Mayo endoscopic subscore of 0 or 1. |
| | | | For CDi (CD samples extracted from Ileum), response |
| | | | was defined as patients with a clear improvement of |
| | | | the ulcerations and a decrease on the histological score |
| | | | were considered responders. Patients who did not |
| | | | achieve this healing were considered non-responders |
| | | | although some of them presented endoscopic and/or |
| | | | histological improvement (cite PMID: 9453485). |
| GSE23597 | Toedter | At weeks 8 post | Clinical response was defined as a decrease from |
| | | Infliximab | baselinein the total Mayo score of at least 3 points and |
| | | treatment | at least 30 percent, with an accompanying decrease in |
| | | | the subscore for rectal bleeding of at least 1 point or an |
| | | | absolute subscore for rectal bleeding of 0 or 1. (cite |
| | | | PMID: 16339095) |

TABLE 2
Mucosal biopsy datasets used for multicohort analysis. Responders and non-responders
were labeled based on cohort's annotation criteria as described in Methods.

| | | | | | | | Non- | |
|---|---|---|---|---|---|---|---|---|
| Accession | Author | Center | Platform | Disease | anti-TNFa | Responders | Responders | Total |
| EMTAB7604 | Verstockt | University | Illumina | Inflammatory | Adalimumab/ | 19 | 25 | 44 |
| | | Hospital | HiSeq | Bowel | Infliximab | | | |
| | | Leuven | 4000 | Disease | | | | |
| GSE14580 | Arijs | University | GPL570 | Ulcerative | Infliximab | 8 | 16 | 24 |
| | | Hospital | | Colitis | | | | |
| | | Leuven | | | | | | |
| GSE16879 | Arijs | University | GPL570 | Crohn's | Infliximab | 20 | 17 | 37 |
| | | Hospital | | Disease | | | | |
| | | Leuven | | | | | | |
| GSE23597 | Toedter | Multi- | GPL570 | Ulcerative | Infliximab | 24 | 7 | 31 |
| | | center | | Colitis | | | | |
| | | ACT1 | | | | | | |
| Total | 3 | >2 centers | 2 | 2 major | 2 anti-TNFa | 71 | 65 | 136 |
| | Authors | | platforms | subtypes | therapies | | | |

### 3. Methods

Raw data for the microarray datasets were downloaded as CEL files and normalized using the robust multichip average (RMA) method using the Affy R package (version 1.63.1) in conjunction with a custom CDF from Brain Array, HGU133Plus2 Hs ENTREZG (version 23.0.0, ENTREZG) to leverage updated chip annotations. For E-MTAB-7604, an RNA-Seq dataset, raw data was downloaded as FASTQ files and processed using our internal RNAseq pipeline which uses the STAR aligner in conjunction with GENCODE v32 GTF (transcriptome reference) to map reads to the human genome, GRCh38 build. Mapped reads were quantified as per Ensembl transcript ID as defined in GENCODE v32 annotation. Reads were summed across Ensembl transcript IDs mapping to Entrez gene IDs in order to compare them with the microarray datasets (AnnotationDbi from Bioconductor). Lowly expressed genes with few counts that cannot be measured reliably were filtered using the following cutoff: max counts per million (CPM) less than 5 across all 44 samples. Normalization factors were obtained using edgeR's Trimmed Mean of M values (TMM) method (R package v.3.28.0). The voom method (limma R package v.3.41.18) was then used to transform counts into normalized log 2-CPM.

A modified version of the ComBat empirical Bayes normalization method known as Combat CO-Normalization Using conTrols (COCONUT) was used to conormalize samples across platforms using healthy controls. This approach makes one strong assumption: healthy control samples from different cohorts represent the same distribution. Briefly, healthy controls from each platform will undergo ComBat co-normalization without covariates. RMA normalized expression data was further conormalized across datasets to remove platform batch effects. All three microarray datasets used the Affymetrix Human Genome U133 Plus 2.0 Array (GPL570), thus we combined these datasets into one cohort including the 12 healthy controls from GSE14580 and GSE16879. We then used datasets of the same platform (but not within the scope of this analysis) that have healthy control samples, GSE66099 and EMTAB3162, and pooled those into one cohort. We then used these two cohorts to conormalize across the GPL570 platform. RNAseq data from (Thair et al iScience, GSE15264) was combined with E-MTAB-7604 to make use of the healthy controls (n=24) processed on the Illumina sequencing platforms. This allowed us to conormalize across GPL570 and RNAseq platforms. Conormalized data was used for downstream multicohort analysis.

A leave-one-study-out (LOSO) multicohort analysis with k studies was performed by holding out one study and performing a multicohort analysis on the remaining k-1 cohorts, repeated k times in a round-robin fashion where a different study was held out each time. In each round, Hedges' g effect size was calculated for all genes between anti-TNF alpha responders and non-responders within a study and summarized across all studies using the DerSimonian & Laird random-effects model to obtain a pooled or summary effect size. A p-value based on standard normal distribution was calculated for the pooled effect size with a Benjamini-Hochberg False Discovery Rate (FDR) correction for multiple hypothesis testing (q-value). Only genes that were significant across all LOSO rounds were considered. A q-value threshold of 10% and absolute effect size threshold of 0.8 (1.75-fold) were used to obtain a set of significant differentially expressed (DE) genes.

Gene sets were evaluated on their class discriminatory power using a difference of geometric means-based score:

$$Response Score = Zscore ( GeoMea n ( p o s ) - G eoMean ( n e g ) * ( \frac{Npos}{Nneg} ) )$$

Where GeoMean(pos) and GeoMean(neg) are the geometric mean of the expression of all positive genes (over-expressed in responders) and geometric mean of the expression of all negative genes (under-expressed in responders), respectively, and

$$\frac{Npos}{Nneg}$$

is a ratio of the number of positive to negative genes, respectively. A response score was calculated for each sample in conjunction with its binary response label, and AUROCs were calculated on a dataset basis using the trapezoidal method to assess the discriminatory performance of the genes. A smoothened pooled ROC curve with weighted standard deviation was generated using the Kester and Buntinx Method (Kester and Buntix, 2000).

### 4. Results

a) Identification of Significant DE Genes and their Performance

We performed a LOSO multicohort analysis comparing the transcriptomic profiles of 71 responders vs 65 non-responders to identify DE genes between the two classes. The resulting output underwent a thresholding process where we filtered genes based on effect size >0.8 (positive or over-expressed) and effect size <−0.8 (negative or under-expressed) and false discovery rate (FDR)<10%, yielding a total of 324 DE genes (58 positive genes, 266 negative genes, Table 3).

TABLE 3
Summary statistics of 324 DE significant genes from multicohort analysis of 4
IBD datasets. Thresholding criteria for filtering: absolute (effect size) >0.8
and q-value >10%. The table captures Hedges' g effect size,
effect size standard error, FDR-adjusted p-value, Tau squared (measure of
heterogeneity), and heterogeneity p-value. Genes that are bolded denote the
forward search gene set. Amongst 324 signature genes, seven fold faced genes
are selected for one classifier by using a greedy forward search alrogrithm

| | | | | | Tau- | Het p- |
|---|---|---|---|---|---|---|
| Gene ID | symbol | Effect Size | Std Error | FDR p-val | squared | value |
| 8165 | AKAP1 | 0.995 | 0.211 | 3.97E−04 | 2.75E−02 | 3.16E−01 |
| 65268 | WNK2 | 0.974 | 0.210 | 4.75E−04 | 2.52E−02 | 3.22E−01 |
| 5525 | PPP2R5A | 0.970 | 0.191 | 1.51E−04 | 0.00E+00 | 6.88E−01 |
| 254887 | ZDHHC23 | 0.970 | 0.191 | 1.51E−04 | 0.00E+00 | 8.45E−01 |
| 146223 | CMTM4 | 0.940 | 0.230 | 2.08E−03 | 6.01E−02 | 2.42E−01 |
| 23321 | TRIM2 | 0.931 | 0.248 | 4.55E−03 | 9.32E−02 | 1.84E−01 |
| 4705 | NDUFA10 | 0.929 | 0.191 | 2.67E−04 | 0.00E+00 | 6.08E−01 |
| 4306 | NR3C2 | 0.926 | 0.190 | 2.67E−04 | 0.00E+00 | 8.35E−01 |
| 4642 | MYO1D | 0.925 | 0.190 | 2.67E−04 | 0.00E+00 | 4.80E−01 |
| 1832 | DSP | 0.915 | 0.224 | 2.12E−03 | 5.15E−02 | 2.59E−01 |
| 10422 | UBAC1 | 0.911 | 0.242 | 4.55E−03 | 8.34E−02 | 1.99E−01 |
| 152189 | CMTM8 | 0.910 | 0.190 | 3.23E−04 | 0.00E+00 | 4.69E−01 |
| 54602 | NDFIP2 | 0.908 | 0.190 | 3.28E−04 | 0.00E+00 | 7.38E−01 |
| 55204 | GOLPH3L | 0.901 | 0.269 | 1.16E−02 | 1.35E−01 | 1.28E−01 |
| 4952 | OCRL | 0.899 | 0.190 | 3.69E−04 | 0.00E+00 | 8.54E−01 |
| 1045 | CDX2 | 0.896 | 0.191 | 4.03E−04 | 0.00E+00 | 4.19E−01 |
| 112 | ADCY6 | 0.895 | 0.190 | 4.03E−04 | 0.00E+00 | 5.37E−01 |
| 376267 | RAB15 | 0.893 | 0.190 | 4.03E−04 | 0.00E+00 | 5.60E−01 |
| 5570 | PKIB | 0.884 | 0.190 | 4.66E−04 | 0.00E+00 | 7.06E−01 |
| 6272 | SORT1 | 0.883 | 0.190 | 4.85E−04 | 0.00E+00 | 4.23E−01 |
| 4756 | NEO1 | 0.881 | 0.204 | 1.16E−03 | 2.09E−02 | 3.32E−01 |
| 7384 | UQCRC1 | 0.881 | 0.241 | 5.77E−03 | 8.09E−02 | 2.02E−01 |
| 348110 | ARPIN | 0.878 | 0.190 | 5.08E−04 | 0.00E+00 | 8.83E−01 |
| 79956 | ERMP1 | 0.878 | 0.200 | 9.30E−04 | 1.40E−02 | 3.50E−01 |
| 153769 | SH3RF2 | 0.877 | 0.190 | 5.20E−04 | 0.00E+00 | 4.12E−01 |
| 8991 | SELENBP1 | 0.876 | 0.220 | 2.68E−03 | 4.47E−02 | 2.74E−01 |
| 622 | BDH1 | 0.874 | 0.190 | 5.52E−04 | 0.00E+00 | 4.62E−01 |
| 57017 | COQ9 | 0.873 | 0.189 | 5.24E−04 | 0.00E+00 | 6.94E−01 |
| 4038 | LRP4 | 0.871 | 0.189 | 5.42E−04 | 0.00E+00 | 8.53E−01 |
| 116987 | AGAP1 | 0.868 | 0.189 | 5.69E−04 | 0.00E+00 | 6.28E−01 |
| 26073 | POLDIP2 | 0.868 | 0.189 | 5.60E−04 | 0.00E+00 | 7.37E−01 |
| 5211 | PFKL | 0.861 | 0.244 | 7.73E−03 | 8.68E−02 | 1.92E−01 |
| 1945 | EFNA4 | 0.860 | 0.189 | 6.29E−04 | 0.00E+00 | 5.44E−01 |
| 3268 | AGFG2 | 0.857 | 0.189 | 6.60E−04 | 0.00E+00 | 5.33E−01 |
| 57698 | SHTN1 | 0.855 | 0.189 | 6.60E−04 | 0.00E+00 | 7.88E−01 |
| 22906 | TRAK1 | 0.854 | 0.189 | 6.60E−04 | 0.00E+00 | 8.94E−01 |
| 57648 | KIAA1522 | 0.854 | 0.217 | 2.96E−03 | 4.10E−02 | 2.81E−01 |
| 619279 | ZNF704 | 0.852 | 0.253 | 1.11E−02 | 1.04E−01 | 1.67E−01 |
| 25934 | NIPSNAP3A | 0.851 | 0.189 | 6.82E−04 | 0.00E+00 | 6.41E−01 |
| 93974 | ATP5IF1 | 0.844 | 0.189 | 7.54E−04 | 0.00E+00 | 6.07E−01 |
| 7855 | FZD5 | 0.841 | 0.189 | 7.93E−04 | 0.00E+00 | 7.34E−01 |
| 1352 | COX10 | 0.839 | 0.189 | 8.46E−04 | 0.00E+00 | 4.66E−01 |
| 7105 | TSPAN6 | 0.830 | 0.189 | 9.30E−04 | 0.00E+00 | 5.68E−01 |
| 140803 | TRPM6 | 0.830 | 0.245 | 1.07E−02 | 8.96E−02 | 1.88E−01 |
| 1456 | CSNK1G3 | 0.826 | 0.188 | 9.42E−04 | 0.00E+00 | 9.20E−01 |
| 3291 | HSD11B2 | 0.822 | 0.188 | 9.86E−04 | 0.00E+00 | 7.57E−01 |
| 55620 | STAP2 | 0.822 | 0.189 | 1.02E−03 | 0.00E+00 | 5.47E−01 |
| 79695 | GALNT12 | 0.822 | 0.188 | 9.86E−04 | 0.00E+00 | 6.36E−01 |
| 389084 | SNORC | 0.818 | 0.188 | 1.06E−03 | 0.00E+00 | 6.61E−01 |
| 57186 | RALGAPA2 | 0.818 | 0.189 | 1.07E−03 | 0.00E+00 | 5.12E−01 |
| 10939 | AFG3L2 | 0.817 | 0.188 | 1.04E−03 | 0.00E+00 | 9.11E−01 |
| 26035 | GLCE | 0.817 | 0.232 | 7.82E−03 | 6.66E−02 | 2.26E−01 |
| 26275 | HIBCH | 0.815 | 0.188 | 1.11E−03 | 0.00E+00 | 9.58E−01 |
| 79022 | TMEM106C | 0.812 | 0.188 | 1.16E−03 | 0.00E+00 | 5.52E−01 |
| 8702 | B4GALT4 | 0.811 | 0.188 | 1.14E−03 | 0.00E+00 | 8.56E−01 |
| 140460 | ASB7 | 0.810 | 0.235 | 9.52E−03 | 7.24E−02 | 2.16E−01 |
| 6920 | TCEA3 | 0.809 | 0.188 | 1.16E−03 | 0.00E+00 | 7.80E−01 |
| 283431 | GAS2L3 | 0.808 | 0.188 | 1.18E−03 | 0.00E+00 | 9.58E−01 |
| 56975 | FAM20C | −0.808 | 0.205 | 3.00E−03 | 2.35E−02 | 3.24E−01 |
| 50515 | CHST11 | −0.808 | 0.188 | 1.22E−03 | 0.00E+00 | 6.96E−01 |
| 150372 | NFAM1 | −0.808 | 0.188 | 1.22E−03 | 0.00E+00 | 4.70E−01 |
| 8840 | CCN4 | −0.808 | 0.189 | 1.24E−03 | 0.00E+00 | 4.87E−01 |
| 148741 | ANKRD35 | −0.810 | 0.188 | 1.16E−03 | 0.00E+00 | 9.66E−01 |
| 51296 | SLC15A3 | −0.810 | 0.189 | 1.18E−03 | 0.00E+00 | 7.33E−01 |
| 50856 | CLEC4A | −0.811 | 0.221 | 5.53E−03 | 4.72E−02 | 2.67E−01 |
| 669 | BPGM | −0.812 | 0.208 | 3.18E−03 | 2.66E−02 | 3.16E−01 |
| 11245 | GPR176 | −0.813 | 0.188 | 1.14E−03 | 0.00E+00 | 5.86E−01 |
| 3383 | ICAM1 | −0.813 | 0.189 | 1.15E−03 | 0.00E+00 | 5.67E−01 |
| 27 | ABL2 | −0.815 | 0.219 | 4.87E−03 | 4.44E−02 | 2.73E−01 |
| 23299 | BICD2 | −0.816 | 0.189 | 1.13E−03 | 0.00E+00 | 4.46E−01 |
| 2191 | FAP | −0.819 | 0.189 | 1.13E−03 | 0.00E+00 | 4.16E−01 |
| 10044 | SH2D3C | −0.820 | 0.189 | 1.06E−03 | 0.00E+00 | 5.50E−01 |
| 6696 | SPP1 | −0.820 | 0.189 | 1.07E−03 | 0.00E+00 | 5.14E−01 |
| 222487 | ADGRG3 | −0.820 | 0.188 | 1.02E−03 | 0.00E+00 | 8.21E−01 |
| 7127 | TNFAIP2 | −0.820 | 0.189 | 1.04E−03 | 0.00E+00 | 5.04E−01 |
| 26112 | CCDC69 | −0.821 | 0.205 | 2.57E−03 | 2.33E−02 | 3.25E−01 |
| 1808 | DPYSL2 | −0.821 | 0.222 | 5.21E−03 | 4.95E−02 | 2.62E−01 |
| 90102 | PHLDB2 | −0.823 | 0.215 | 3.96E−03 | 3.80E−02 | 2.88E−01 |
| 54039 | PCBP3 | −0.824 | 0.189 | 9.86E−04 | 0.00E+00 | 7.20E−01 |
| 80014 | WWC2 | −0.825 | 0.189 | 9.86E−04 | 0.00E+00 | 5.11E−01 |
| 6925 | TCF4 | −0.825 | 0.189 | 9.69E−04 | 0.00E+00 | 6.66E−01 |
| 5341 | PLEK | −0.826 | 0.189 | 9.83E−04 | 0.00E+00 | 6.50E−01 |
| 4689 | NCF4 | −0.826 | 0.188 | 9.41E−04 | 0.00E+00 | 9.11E−01 |
| 84243 | ZDHHC18 | −0.826 | 0.188 | 9.41E−04 | 0.00E+00 | 9.76E−01 |
| 11259 | FILIP1L | −0.826 | 0.189 | 9.86E−04 | 0.00E+00 | 4.55E−01 |
| 25966 | C2CD2 | −0.827 | 0.189 | 9.86E−04 | 0.00E+00 | 5.12E−01 |
| 64744 | SMAP2 | −0.828 | 0.188 | 9.30E−04 | 0.00E+00 | 9.16E−01 |
| 118788 | PIK3AP1 | −0.828 | 0.188 | 9.30E−04 | 0.00E+00 | 9.68E−01 |
| 399959 | MIR100HG | −0.828 | 0.188 | 9.30E−04 | 0.00E+00 | 8.26E−01 |
| 285 | ANGPT2 | −0.830 | 0.189 | 9.42E−04 | 0.00E+00 | 4.59E−01 |
| 7099 | TLR4 | −0.830 | 0.189 | 9.42E−04 | 0.00E+00 | 5.07E−01 |
| 221895 | JAZF1 | −0.830 | 0.189 | 9.30E−04 | 0.00E+00 | 5.46E−01 |
| 3002 | GZMB | −0.831 | 0.189 | 9.30E−04 | 0.00E+00 | 6.88E−01 |
| 399 | RHOH | −0.831 | 0.189 | 9.24E−04 | 0.00E+00 | 8.39E−01 |
| 3577 | CXCR1 | −0.831 | 0.189 | 9.30E−04 | 0.00E+00 | 4.30E−01 |
| 23075 | SWAP70 | −0.832 | 0.189 | 9.30E−04 | 0.00E+00 | 6.99E−01 |
| 8877 | SPHK1 | −0.833 | 0.198 | 1.53E−03 | 1.20E−02 | 3.56E−01 |
| 115004 | CGAS | −0.833 | 0.188 | 8.77E−04 | 0.00E+00 | 9.95E−01 |
| 55704 | CCDC88A | −0.835 | 0.209 | 2.58E−03 | 2.90E−02 | 3.10E−01 |
| 27128 | CYTH4 | −0.836 | 0.188 | 8.42E−04 | 0.00E+00 | 9.67E−01 |
| 170954 | PPP1R18 | −0.836 | 0.189 | 8.46E−04 | 0.00E+00 | 8.18E−01 |
| 1191 | CLU | −0.839 | 0.189 | 8.17E−04 | 0.00E+00 | 9.69E−01 |
| 1390 | CREM | −0.839 | 0.234 | 6.99E−03 | 6.95E−02 | 2.23E−01 |
| 3059 | HCLS1 | −0.840 | 0.189 | 8.17E−04 | 0.00E+00 | 8.72E−01 |
| 23466 | CBX6 | −0.840 | 0.189 | 8.03E−04 | 0.00E+00 | 7.77E−01 |
| 6695 | SPOCK1 | −0.841 | 0.189 | 8.17E−04 | 0.00E+00 | 5.05E−01 |
| 7852 | CXCR4 | −0.841 | 0.188 | 7.77E−04 | 0.00E+00 | 9.43E−01 |
| 8869 | ST3GAL5 | −0.844 | 0.189 | 7.46E−04 | 0.00E+00 | 7.68E−01 |
| 170371 | TMEM273 | −0.845 | 0.231 | 5.83E−03 | 6.49E−02 | 2.31E−01 |
| 1378 | CR1 | −0.847 | 0.189 | 7.28E−04 | 0.00E+00 | 6.95E−01 |
| 83593 | RASSF5 | −0.849 | 0.189 | 7.17E−04 | 0.00E+00 | 6.06E−01 |
| 1901 | S1PR1 | −0.849 | 0.227 | 4.70E−03 | 5.75E−02 | 2.46E−01 |
| 271 | AMPD2 | −0.850 | 0.189 | 7.17E−04 | 0.00E+00 | 6.09E−01 |
| 26064 | RAI14 | −0.851 | 0.192 | 8.46E−04 | 2.89E−03 | 3.83E−01 |
| 1880 | GPR183 | −0.851 | 0.203 | 1.57E−03 | 1.84E−02 | 3.39E−01 |
| 9953 | HS3ST3B1 | −0.851 | 0.223 | 3.97E−03 | 4.97E−02 | 2.63E−01 |
| 1960 | EGR3 | −0.851 | 0.189 | 6.77E−04 | 0.00E+00 | 9.29E−01 |
| 9435 | CHST2 | −0.851 | 0.189 | 7.06E−04 | 0.00E+00 | 5.78E−01 |
| 10581 | IFITM2 | −0.851 | 0.189 | 6.77E−04 | 0.00E+00 | 8.00E−01 |
| 2026 | ENO2 | −0.853 | 0.189 | 6.77E−04 | 0.00E+00 | 9.43E−01 |
| 3142 | HLX | −0.853 | 0.217 | 2.94E−03 | 3.98E−02 | 2.84E−01 |
| 3936 | LCP1 | −0.853 | 0.189 | 6.77E−04 | 0.00E+00 | 6.74E−01 |
| 586 | BCAT1 | −0.854 | 0.190 | 6.77E−04 | 0.00E+00 | 4.93E−01 |
| 23406 | COTL1 | −0.854 | 0.232 | 5.45E−03 | 6.58E−02 | 2.29E−01 |
| 7940 | LST1 | −0.854 | 0.222 | 3.68E−03 | 4.84E−02 | 2.65E−01 |
| 4778 | NFE2 | −0.854 | 0.189 | 6.77E−04 | 0.00E+00 | 4.88E−01 |
| 51363 | CHST15 | −0.855 | 0.235 | 6.16E−03 | 7.21E−02 | 2.18E−01 |
| 28984 | RGCC | −0.855 | 0.202 | 1.44E−03 | 1.68E−02 | 3.43E−01 |
| 11031 | RAB31 | −0.856 | 0.189 | 6.64E−04 | 0.00E+00 | 5.38E−01 |
| 64127 | NOD2 | −0.856 | 0.190 | 6.68E−04 | 0.00E+00 | 6.44E−01 |
| 1844 | DUSP2 | −0.857 | 0.189 | 6.64E−04 | 0.00E+00 | 4.41E−01 |
| 84984 | CEP19 | −0.859 | 0.189 | 6.37E−04 | 0.00E+00 | 4.88E−01 |
| 6004 | RGS16 | −0.860 | 0.220 | 3.13E−03 | 4.55E−02 | 2.71E−01 |
| 6777 | STAT5B | −0.861 | 0.189 | 6.09E−04 | 0.00E+00 | 8.19E−01 |
| 4478 | MSN | −0.862 | 0.190 | 6.52E−04 | 0.00E+00 | 4.86E−01 |
| 7832 | BTG2 | −0.862 | 0.189 | 6.06E−04 | 0.00E+00 | 9.28E−01 |
| 57580 | PREX1 | −0.863 | 0.190 | 6.25E−04 | 0.00E+00 | 4.60E−01 |
| 4017 | LOXL2 | −0.864 | 0.198 | 9.81E−04 | 1.04E−02 | 3.61E−01 |
| 6503 | SLA | −0.864 | 0.190 | 6.15E−04 | 0.00E+00 | 5.33E−01 |
| 10287 | RGS19 | −0.864 | 0.209 | 1.85E−03 | 2.79E−02 | 3.13E−01 |
| 79827 | CLMP | −0.866 | 0.231 | 4.65E−03 | 6.43E−02 | 2.33E−01 |
| 730249 | ACOD1 | −0.866 | 0.191 | 6.29E−04 | 6.27E−04 | 3.90E−01 |
| 5270 | SERPINE2 | −0.867 | 0.190 | 6.00E−04 | 0.00E+00 | 4.33E−01 |
| 284119 | CAVIN1 | −0.867 | 0.234 | 5.16E−03 | 6.92E−02 | 2.23E−01 |
| 4327 | MMP19 | −0.868 | 0.189 | 5.65E−04 | 0.00E+00 | 6.95E−01 |
| 85464 | SSH2 | −0.868 | 0.227 | 3.97E−03 | 5.82E−02 | 2.44E−01 |
| 9938 | ARHGAP25 | −0.868 | 0.189 | 5.65E−04 | 0.00E+00 | 5.27E−01 |
| 963 | CD53 | −0.869 | 0.189 | 5.52E−04 | 0.00E+00 | 8.08E−01 |
| 1117 | CHI3L2 | −0.870 | 0.189 | 5.52E−04 | 0.00E+00 | 9.43E−01 |
| 359845 | RFLNB | −0.872 | 0.219 | 2.71E−03 | 4.39E−02 | 2.76E−01 |
| 51050 | PI15 | −0.872 | 0.261 | 1.17E−02 | 1.18E−01 | 1.48E−01 |
| 1903 | S1PR3 | −0.873 | 0.189 | 5.30E−04 | 0.00E+00 | 5.44E−01 |
| 3937 | LCP2 | −0.875 | 0.231 | 4.17E−03 | 6.29E−02 | 2.36E−01 |
| 6374 | CXCL5 | −0.876 | 0.243 | 6.42E−03 | 8.35E−02 | 1.99E−01 |
| 9586 | CREB5 | −0.878 | 0.218 | 2.32E−03 | 4.13E−02 | 2.81E−01 |
| 969 | CD69 | −0.878 | 0.238 | 5.27E−03 | 7.39E−02 | 2.16E−01 |
| 7409 | VAV1 | −0.879 | 0.189 | 4.85E−04 | 0.00E+00 | 9.19E−01 |
| 1809 | DPYSL3 | −0.879 | 0.190 | 5.20E−04 | 0.00E+00 | 5.29E−01 |
| 1306 | COL15A1 | −0.880 | 0.262 | 1.13E−02 | 1.20E−01 | 1.46E−01 |
| 728215 | FAM155A | −0.882 | 0.224 | 2.93E−03 | 5.10E−02 | 2.60E−01 |
| 7431 | VIM | −0.883 | 0.231 | 3.96E−03 | 6.27E−02 | 2.37E−01 |
| 24147 | FJX1 | −0.884 | 0.190 | 4.75E−04 | 0.00E+00 | 5.53E−01 |
| 1846 | DUSP4 | −0.888 | 0.191 | 4.75E−04 | 0.00E+00 | 4.49E−01 |
| 1284 | COL4A2 | −0.889 | 0.252 | 7.76E−03 | 1.01E−01 | 1.73E−01 |
| 7846 | TUBA1A | −0.889 | 0.191 | 4.66E−04 | 2.76E−04 | 3.91E−01 |
| 284996 | RNF149 | −0.892 | 0.190 | 4.11E−04 | 0.00E+00 | 6.37E−01 |
| 3055 | HCK | −0.893 | 0.209 | 1.24E−03 | 2.61E−02 | 3.19E−01 |
| 5328 | PLAU | −0.893 | 0.234 | 3.97E−03 | 6.65E−02 | 2.30E−01 |
| 51278 | IER5 | −0.894 | 0.190 | 4.00E−04 | 0.00E+00 | 6.52E−01 |
| 1230 | CCR1 | −0.894 | 0.190 | 4.03E−04 | 0.00E+00 | 5.80E−01 |
| 9535 | GMFG | −0.894 | 0.190 | 4.03E−04 | 0.00E+00 | 4.53E−01 |
| 4897 | NRCAM | −0.895 | 0.224 | 2.60E−03 | 5.20E−02 | 2.58E−01 |
| 5788 | PTPRC | −0.895 | 0.246 | 6.05E−03 | 8.98E−02 | 1.89E−01 |
| 117289 | TAGAP | −0.896 | 0.200 | 7.28E−04 | 1.32E−02 | 3.53E−01 |
| 9467 | SH3BP5 | −0.896 | 0.212 | 1.45E−03 | 3.18E−02 | 3.04E−01 |
| 51365 | PLA1A | −0.898 | 0.246 | 5.88E−03 | 8.97E−02 | 1.89E−01 |
| 114990 | VASN | −0.899 | 0.209 | 1.16E−03 | 2.59E−02 | 3.19E−01 |
| 3684 | ITGAM | −0.900 | 0.190 | 3.69E−04 | 0.00E+00 | 8.63E−01 |
| 91607 | SLFN11 | −0.900 | 0.231 | 3.24E−03 | 6.23E−02 | 2.38E−01 |
| 83643 | CCDC3 | −0.901 | 0.190 | 3.70E−04 | 0.00E+00 | 6.20E−01 |
| 55529 | PIP4P2 | −0.903 | 0.215 | 1.55E−03 | 3.58E−02 | 2.95E−01 |
| 10979 | FERMT2 | −0.904 | 0.216 | 1.61E−03 | 3.71E−02 | 2.92E−01 |
| 929 | CD14 | −0.905 | 0.190 | 3.51E−04 | 0.00E+00 | 5.87E−01 |
| 1475 | CSTA | −0.907 | 0.190 | 3.34E−04 | 0.00E+00 | 8.37E−01 |
| 50807 | ASAP1 | −0.907 | 0.222 | 2.08E−03 | 4.62E−02 | 2.71E−01 |
| 9945 | GFPT2 | −0.909 | 0.191 | 3.34E−04 | 0.00E+00 | 6.67E−01 |
| 2867 | FFAR2 | −0.909 | 0.190 | 3.28E−04 | 0.00E+00 | 7.97E−01 |
| 1397 | CRIP2 | −0.910 | 0.257 | 7.68E−03 | 1.11E−01 | 1.58E−01 |
| 715 | C1R | −0.910 | 0.202 | 6.77E−04 | 1.51E−02 | 3.48E−01 |
| 25884 | CHRDL2 | −0.910 | 0.190 | 3.22E−04 | 0.00E+00 | 8.43E−01 |
| 4210 | MEFV | −0.910 | 0.255 | 7.07E−03 | 1.06E−01 | 1.65E−01 |
| 4542 | MYO1F | −0.911 | 0.218 | 1.63E−03 | 4.05E−02 | 2.84E−01 |
| 7076 | TIMP1 | −0.912 | 0.190 | 3.22E−04 | 0.00E+00 | 7.73E−01 |
| 8767 | RIPK2 | −0.915 | 0.190 | 3.00E−04 | 0.00E+00 | 4.29E−01 |
| 2321 | FLT1 | −0.915 | 0.253 | 6.38E−03 | 1.03E−01 | 1.70E−01 |
| 11025 | LILRB3 | −0.916 | 0.191 | 3.12E−04 | 0.00E+00 | 4.53E−01 |
| 53829 | P2RY13 | −0.916 | 0.247 | 5.15E−03 | 9.14E−02 | 1.87E−01 |
| 4616 | GADD45B | −0.918 | 0.191 | 2.96E−04 | 0.00E+00 | 6.27E−01 |
| 11009 | IL24 | −0.919 | 0.210 | 9.42E−04 | 2.70E−02 | 3.17E−01 |
| 8794 | TNFRSF10C | −0.920 | 0.191 | 2.86E−04 | 0.00E+00 | 6.50E−01 |
| 4162 | MCAM | −0.920 | 0.246 | 4.60E−03 | 8.83E−02 | 1.92E−01 |
| 9750 | RIPOR2 | −0.920 | 0.190 | 2.78E−04 | 0.00E+00 | 7.28E−01 |
| 9180 | OSMR | −0.922 | 0.191 | 2.78E−04 | 0.00E+00 | 5.92E−01 |
| 1116 | CHI3L1 | −0.922 | 0.191 | 2.78E−04 | 0.00E+00 | 5.13E−01 |
| 8477 | GPR65 | −0.923 | 0.191 | 2.78E−04 | 0.00E+00 | 5.58E−01 |
| 2207 | FCER1G | −0.923 | 0.234 | 2.94E−03 | 6.76E−02 | 2.28E−01 |
| 144423 | GLT1D1 | −0.925 | 0.190 | 2.67E−04 | 0.00E+00 | 6.45E−01 |
| 9120 | SLC16A6 | −0.925 | 0.190 | 2.67E−04 | 0.00E+00 | 5.40E−01 |
| 5396 | PRRX1 | −0.926 | 0.190 | 2.67E−04 | 0.00E+00 | 8.51E−01 |
| 942 | CD86 | −0.927 | 0.260 | 7.32E−03 | 1.16E−01 | 1.52E−01 |
| 23597 | ACOT9 | −0.928 | 0.210 | 8.94E−04 | 2.74E−02 | 3.16E−01 |
| 10395 | DLC1 | −0.929 | 0.191 | 2.67E−04 | 0.00E+00 | 5.69E−01 |
| 6351 | CCL4 | −0.930 | 0.191 | 2.67E−04 | 0.00E+00 | 4.21E−01 |
| 10409 | BASP1 | −0.935 | 0.191 | 2.49E−04 | 0.00E+00 | 6.11E−01 |
| 6648 | SOD2 | −0.939 | 0.235 | 2.57E−03 | 6.80E−02 | 2.28E−01 |
| 953 | ENTPD1 | −0.940 | 0.252 | 4.82E−03 | 9.86E−02 | 1.78E−01 |
| 137835 | TMEM71 | −0.940 | 0.248 | 4.17E−03 | 9.25E−02 | 1.86E−01 |
| 143458 | LDLRAD3 | −0.943 | 0.277 | 1.04E−02 | 1.51E−01 | 1.15E−01 |
| 597 | BCL2A1 | −0.944 | 0.192 | 2.25E−04 | 0.00E+00 | 4.33E−01 |
| 7462 | LAT2 | −0.945 | 0.191 | 1.99E−04 | 0.00E+00 | 9.99E−01 |
| 10859 | LILRB1 | −0.945 | 0.191 | 2.08E−04 | 0.00E+00 | 5.41E−01 |
| 3310 | HSPA6 | −0.947 | 0.192 | 2.08E−04 | 0.00E+00 | 4.07E−01 |
| 2247 | FGF2 | −0.949 | 0.192 | 2.08E−04 | 0.00E+00 | 4.56E−01 |
| 4600 | MX2 | −0.949 | 0.208 | 5.85E−04 | 2.34E−02 | 3.26E−01 |
| 3956 | LGALS1 | −0.950 | 0.253 | 4.55E−03 | 1.01E−01 | 1.74E−01 |
| 84617 | TUBB6 | −0.952 | 0.191 | 1.97E−04 | 0.00E+00 | 5.76E−01 |
| 11010 | GLIPR1 | −0.953 | 0.253 | 4.40E−03 | 1.01E−01 | 1.74E−01 |
| 10439 | OLFM1 | −0.953 | 0.202 | 3.83E−04 | 1.46E−02 | 3.49E−01 |
| 2771 | GNAI2 | −0.954 | 0.223 | 1.26E−03 | 4.76E−02 | 2.69E−01 |
| 1123 | CHN1 | −0.954 | 0.208 | 5.69E−04 | 2.41E−02 | 3.25E−01 |
| 10630 | PDPN | −0.955 | 0.205 | 4.75E−04 | 1.87E−02 | 3.39E−01 |
| 3576 | CXCL8 | −0.958 | 0.192 | 1.94E−04 | 0.00E+00 | 4.12E−01 |
| 5272 | SERPINB9 | −0.960 | 0.191 | 1.65E−04 | 0.00E+00 | 9.16E−01 |
| 3084 | NRG1 | −0.962 | 0.217 | 8.67E−04 | 3.90E−02 | 2.88E−01 |
| 5142 | PDE4B | −0.964 | 0.192 | 1.70E−04 | 0.00E+00 | 4.21E−01 |
| 9934 | P2RY14 | −0.965 | 0.243 | 2.70E−03 | 8.14E−02 | 2.05E−01 |
| 3678 | ITGA5 | −0.965 | 0.192 | 1.65E−04 | 0.00E+00 | 4.40E−01 |
| 7130 | TNFAIP6 | −0.965 | 0.272 | 7.46E−03 | 1.39E−01 | 1.27E−01 |
| 79930 | DOK3 | −0.969 | 0.191 | 1.51E−04 | 0.00E+00 | 9.10E−01 |
| 9448 | MAP4K4 | −0.970 | 0.210 | 5.14E−04 | 2.52E−02 | 3.22E−01 |
| 23452 | ANGPTL2 | −0.970 | 0.192 | 1.51E−04 | 0.00E+00 | 4.91E−01 |
| 80723 | SLC35G2 | −0.971 | 0.230 | 1.51E−03 | 5.88E−02 | 2.46E−01 |
| 3772 | KCNJ15 | −0.973 | 0.192 | 1.51E−04 | 0.00E+00 | 6.96E−01 |
| 140876 | RIPOR3 | −0.974 | 0.192 | 1.51E−04 | 0.00E+00 | 4.08E−01 |
| 26253 | CLEC4E | −0.974 | 0.232 | 1.55E−03 | 6.15E−02 | 2.41E−01 |
| 6347 | CCL2 | −0.976 | 0.249 | 3.09E−03 | 9.27E−02 | 1.87E−01 |
| 4314 | MMP3 | −0.976 | 0.241 | 2.25E−03 | 7.73E−02 | 2.13E−01 |
| 387763 | C11orf96 | −0.979 | 0.251 | 3.22E−03 | 9.62E−02 | 1.82E−01 |
| 5742 | PTGS1 | −0.979 | 0.192 | 1.49E−04 | 0.00E+00 | 4.09E−01 |
| 57214 | CEMIP | −0.981 | 0.192 | 1.45E−04 | 0.00E+00 | 4.43E−01 |
| 2014 | EMP3 | −0.982 | 0.274 | 6.83E−03 | 1.42E−01 | 1.25E−01 |
| 6556 | SLC11A1 | −0.983 | 0.213 | 5.20E−04 | 3.00E−02 | 3.10E−01 |
| 9473 | THEMIS2 | −0.985 | 0.192 | 1.32E−04 | 0.00E+00 | 5.90E−01 |
| 1441 | CSF3R | −0.985 | 0.192 | 1.32E−04 | 0.00E+00 | 5.33E−01 |
| 3557 | IL1RN | −0.991 | 0.193 | 1.32E−04 | 0.00E+00 | 4.30E−01 |
| 8303 | SNN | −0.992 | 0.192 | 1.19E−04 | 0.00E+00 | 6.08E−01 |
| 22918 | CD93 | −0.992 | 0.281 | 7.82E−03 | 1.58E−01 | 1.08E−01 |
| 23643 | LY96 | −0.993 | 0.248 | 2.57E−03 | 9.07E−02 | 1.91E−01 |
| 56603 | CYP26B1 | −0.996 | 0.193 | 1.18E−04 | 0.00E+00 | 4.20E−01 |
| 120892 | LRRK2 | −0.997 | 0.260 | 3.79E−03 | 1.13E−01 | 1.58E−01 |
| 115908 | CTHRC1 | −1.000 | 0.270 | 5.23E−03 | 1.35E−01 | 1.33E−01 |
| 3553 | IL1B | −1.000 | 0.192 | 1.01E−04 | 0.00E+00 | 8.70E−01 |
| 604 | BCL6 | −1.001 | 0.192 | 1.01E−04 | 0.00E+00 | 8.99E−01 |
| 7056 | THBD | −1.001 | 0.241 | 1.74E−03 | 7.72E−02 | 2.13E−01 |
| 5054 | SERPINE1 | −1.002 | 0.193 | 1.06E−04 | 0.00E+00 | 4.55E−01 |
| 85450 | ITPRIP | −1.005 | 0.192 | 9.97E−05 | 0.00E+00 | 9.00E−01 |
| 8572 | PDLIM4 | −1.006 | 0.193 | 1.01E−04 | 0.00E+00 | 4.21E−01 |
| 166336 | PRICKLE2 | −1.006 | 0.255 | 2.94E−03 | 1.03E−01 | 1.73E−01 |
| 2358 | FPR2 | −1.009 | 0.193 | 9.97E−05 | 0.00E+00 | 7.75E−01 |
| 2252 | FGF7 | −1.012 | 0.193 | 9.82E−05 | 0.00E+00 | 4.27E−01 |
| 7474 | WNT5A | −1.021 | 0.260 | 3.08E−03 | 1.13E−01 | 1.61E−01 |
| 1294 | COL7A1 | −1.022 | 0.193 | 8.09E−05 | 0.00E+00 | 6.44E−01 |
| 7107 | GPR137B | −1.024 | 0.212 | 2.78E−04 | 2.65E−02 | 3.20E−01 |
| 112464 | CAVIN3 | −1.025 | 0.253 | 2.25E−03 | 9.95E−02 | 1.78E−01 |
| 81553 | FAM49A | −1.025 | 0.253 | 2.25E−03 | 9.86E−02 | 1.79E−01 |
| 1439 | CSF2RB | −1.025 | 0.193 | 7.78E−05 | 0.00E+00 | 5.71E−01 |
| 7124 | TNF | −1.029 | 0.192 | 6.82E−05 | 0.00E+00 | 8.74E−01 |
| 6283 | S100A12 | −1.033 | 0.241 | 1.25E−03 | 7.70E−02 | 2.14E−01 |
| 1051 | CEBPB | −1.035 | 0.236 | 9.41E−04 | 6.66E−02 | 2.33E−01 |
| 4688 | NCF2 | −1.037 | 0.238 | 1.00E−03 | 7.08E−02 | 2.25E−01 |
| 7096 | TLR1 | −1.044 | 0.271 | 3.66E−03 | 1.33E−01 | 1.36E−01 |
| 728 | C5AR1 | −1.047 | 0.194 | 5.65E−05 | 0.00E+00 | 5.41E−01 |
| 353514 | LILRA5 | −1.048 | 0.193 | 5.43E−05 | 0.00E+00 | 8.92E−01 |
| 2268 | FGR | −1.048 | 0.194 | 5.65E−05 | 0.00E+00 | 4.06E−01 |
| 121260 | SLC15A4 | −1.049 | 0.210 | 1.90E−04 | 2.35E−02 | 3.28E−01 |
| 2153 | F5 | −1.050 | 0.222 | 3.73E−04 | 4.26E−02 | 2.82E−01 |
| 11314 | CD300A | −1.052 | 0.259 | 2.25E−03 | 1.11E−01 | 1.63E−01 |
| 2215 | FCGR3B | −1.054 | 0.194 | 5.43E−05 | 0.00E+00 | 4.26E−01 |
| 6403 | SELP | −1.055 | 0.233 | 6.68E−04 | 6.27E−02 | 2.40E−01 |
| 7837 | PXDN | −1.056 | 0.288 | 5.66E−03 | 1.70E−01 | 1.01E−01 |
| 5552 | SRGN | −1.056 | 0.212 | 1.95E−04 | 2.65E−02 | 3.20E−01 |
| 3624 | INHBA | −1.059 | 0.227 | 4.73E−04 | 5.05E−02 | 2.66E−01 |
| 7133 | TNFRSF1B | −1.059 | 0.194 | 5.38E−05 | 0.00E+00 | 4.17E−01 |
| 2213 | FCGR2B | −1.062 | 0.193 | 4.82E−05 | 0.00E+00 | 9.05E−01 |
| 3371 | TNC | −1.064 | 0.251 | 1.44E−03 | 9.35E−02 | 1.89E−01 |
| 4837 | NNMT | −1.066 | 0.194 | 4.82E−05 | 0.00E+00 | 3.97E−01 |
| 6279 | S100A8 | −1.067 | 0.220 | 2.67E−04 | 3.81E−02 | 2.93E−01 |
| 366 | AQP9 | −1.068 | 0.195 | 4.82E−05 | 0.00E+00 | 3.97E−01 |
| 6402 | SELL | −1.068 | 0.194 | 4.82E−05 | 0.00E+00 | 6.28E−01 |
| 116496 | NIBAN1 | −1.070 | 0.265 | 2.31E−03 | 1.21E−01 | 1.51E−01 |
| 5008 | OSM | −1.070 | 0.194 | 4.82E−05 | 0.00E+00 | 7.87E−01 |
| 51334 | PRR16 | −1.072 | 0.286 | 4.55E−03 | 1.65E−01 | 1.05E−01 |
| 5937 | RBMS1 | −1.074 | 0.288 | 4.86E−03 | 1.69E−01 | 1.01E−01 |
| 201799 | TMEM154 | −1.074 | 0.194 | 4.82E−05 | 0.00E+00 | 8.36E−01 |
| 60675 | PROK2 | −1.078 | 0.195 | 4.82E−05 | 0.00E+00 | 4.88E−01 |
| 6515 | SLC2A3 | −1.082 | 0.195 | 4.82E−05 | 0.00E+00 | 4.01E−01 |
| 1440 | CSF3 | −1.085 | 0.221 | 2.33E−04 | 3.98E−02 | 2.89E−01 |
| 11027 | LILRA2 | −1.093 | 0.236 | 5.08E−04 | 6.59E−02 | 2.35E−01 |
| 50486 | G0S2 | −1.095 | 0.195 | 4.82E−05 | 0.00E+00 | 5.00E−01 |
| 3569 | IL6 | −1.098 | 0.221 | 1.97E−04 | 3.91E−02 | 2.91E−01 |
| 54210 | TREM1 | −1.098 | 0.195 | 4.82E−05 | 0.00E+00 | 6.46E−01 |
| 8875 | VNN2 | −1.102 | 0.271 | 2.14E−03 | 1.32E−01 | 1.40E−01 |
| 89927 | BMERB1 | −1.110 | 0.280 | 2.79E−03 | 1.51E−01 | 1.19E−01 |
| 196383 | RILPL2 | −1.119 | 0.296 | 4.40E−03 | 1.88E−01 | 8.80E−02 |
| 6280 | S100A9 | −1.125 | 0.223 | 1.62E−04 | 4.14E−02 | 2.87E−01 |
| 5743 | PTGS2 | −1.132 | 0.199 | 4.82E−05 | 3.94E−03 | 3.80E−01 |
| 6401 | SELE | −1.132 | 0.280 | 2.25E−03 | 1.50E−01 | 1.21E−01 |
| 3579 | CXCR2 | −1.159 | 0.196 | 2.69E−05 | 0.00E+00 | 5.06E−01 |
| 10288 | LILRB2 | −1.161 | 0.217 | 6.82E−05 | 3.09E−02 | 3.11E−01 |
| 6781 | STC1 | −1.168 | 0.266 | 9.40E−04 | 1.19E−01 | 1.57E−01 |
| 5069 | PAPPA | −1.198 | 0.278 | 1.16E−03 | 1.44E−01 | 1.30E−01 |
| 2357 | FPR1 | −1.211 | 0.198 | 1.48E−05 | 0.00E+00 | 4.26E−01 |
| 3589 | IL11 | −1.226 | 0.247 | 1.99E−04 | 8.01E−02 | 2.15E−01 |
| 3598 | IL13RA2 | −1.541 | 0.385 | 2.53E−03 | 3.99E−01 | 2.18E−02 |

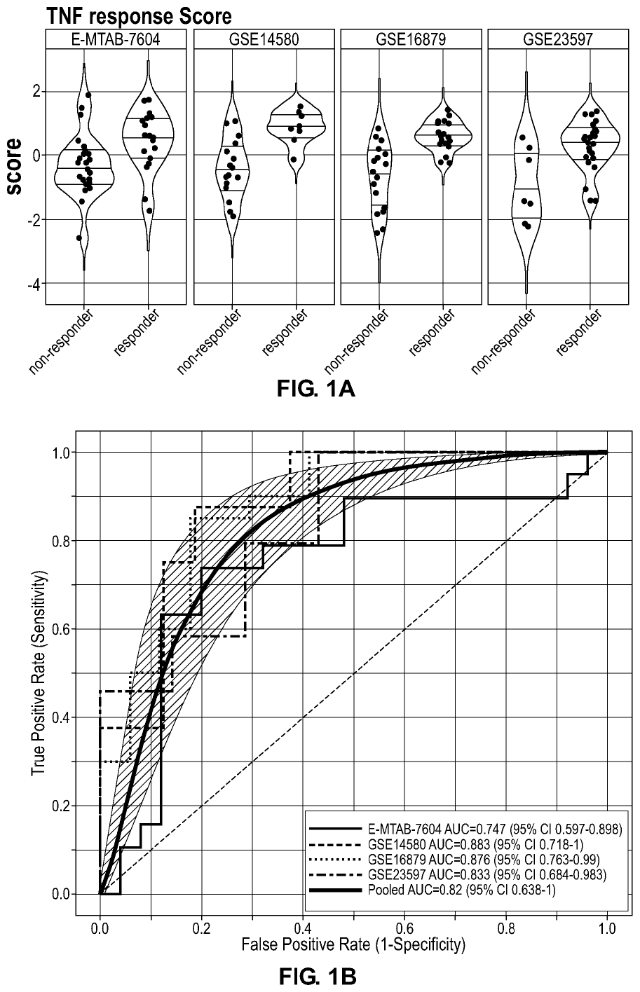

Using the 324 DE genes, we computed a response score for each sample across all studies (FIG. 1A). The median distribution of scores between responders and non-responders shows that these 324 genes are robust in separating the two classes. The 324 DE genes robustly distinguished responders from non-responders (FIG. 1B) with a pooled AUROC of 0.82 (range: 0.64-1).

### b) Identification of Parsimonious Gene Signature and its Performance

We used a greedy forward search to downselect the most predictive combination of genes from the 324 significant DE genes that would be optimized for response prognosis, resulting in seven genes: WNK2, OCRL, ASB7 (positive genes); PCBP3, AMPD2, FAM155A, IL13RA2 (negative genes) (FIG. 2A, Table 3, in bold). Using this 7-gene signature, we similarly computed a response score, showing a 6-point improvement in discriminatory performance compared to 324 DE genes with a pooled AUROC of 0.88 (range 0.74 to 1; FIG. 2B).

### c) Evaluation of Gene Signatures Using IML Platform

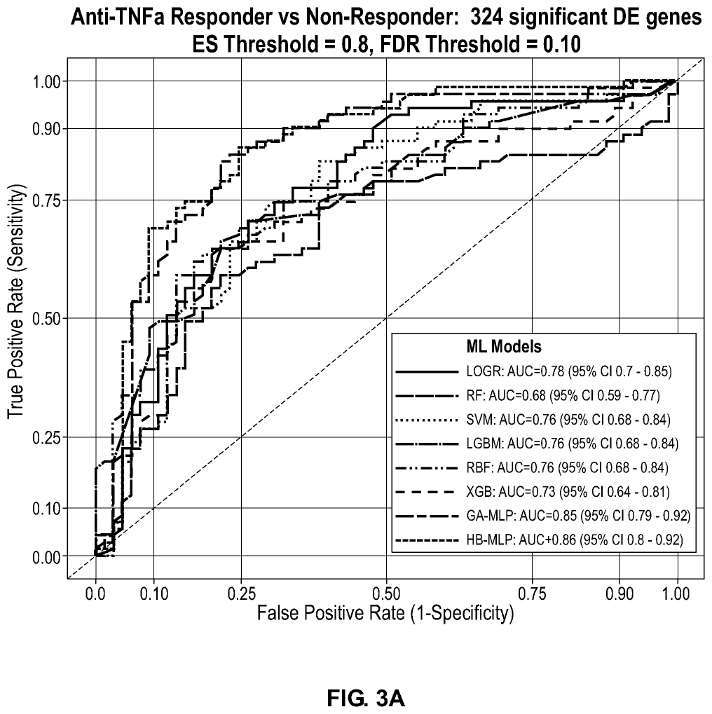

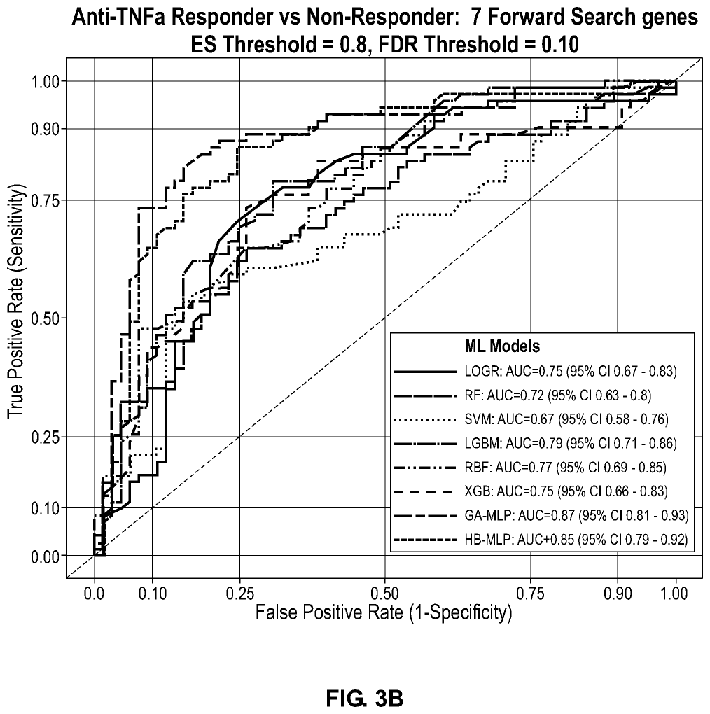

In order to assess the feasibility of translating the signature gene sets to a clinically useful test, we used expression data from all 136 samples for two feature sets-324 DE genes and 7-gene signature—to train a gamut of classifiers [Logistic Regression (LOGR), Random Forest (RF), Support Vector Machines (SVM), Light Gradient Boosting Machine (LGBM), Radial Basis Function (RBF), Extreme Gradient Boosting (XGBoost), Genetic Algorithms-Multilayer Perceptron (GA-MLP), and Hyperband-Multilayer Perceptron (HB-MLP)] using the LOSO cross-validation technique whereby, in each training fold, one dataset is held-out. This list of ML classifiers covers a broad spectrum of Machine Learning algorithms used in today's biomedical applications, ranging from linear to non-linear and simple to complex. The training procedure comprised large-scale hyperparameter searches to find the best model. With 324 DE genes as input, all models had remarkable predictive probabilities with AUCs: 0.78, 0.68, 0.76, 0.76, 0.76, 0.73, 0.85, and 0.86 for LOGR, RF, SVM, RBF, LGBM, XGBoost, GA-MLP, and HB-MLP respectively (FIG. 3A).

Similarly, our 7-gene signature had predictive probabilities with AUCs: 0.75, 0.72, 0.67, 0.79, 0.77, 0.75, 0.87, and 0.85 for LOGR, RF, SVM, RBF, LGBM, XGBoost, GA-MLP, and HB-MLP respectively (FIG. 3B). It is interesting to note that three classifiers (LOGR, RF, and SVM) suffered in performance with the 7-gene signature, whereas the other models maintained similar level of performance with the 324 gene set. Overall, these results suggest it is feasible to develop a pre-trained and tuned “locked” classifier which considers a patient's expression measurements for a small set of genes assayed prior to the treatment and predicts whether that patient is a responder or non-responder.

### d) Performance of Individual Gene and Two-Gene Combinations

Although we have shown robust performance with the 324 DE genes as well as the 7-gene signature, which is a subset of the 324 that constitute a minimal set of genes with optimized performance, it is possible that with a different patient cohort in discovery, other genes may be selected by greedy forward search. To ensure flexibility in marker selection, we evaluated the performance of the 324 genes in single gene and 2-gene pair fashion (52,326 combinations) on a dataset basis and showed that in comparison to a single gene or randomly chosen 2-gene pair from all the genes measured in the datasets (15,116 genes), the mean AUC distribution is significantly higher.

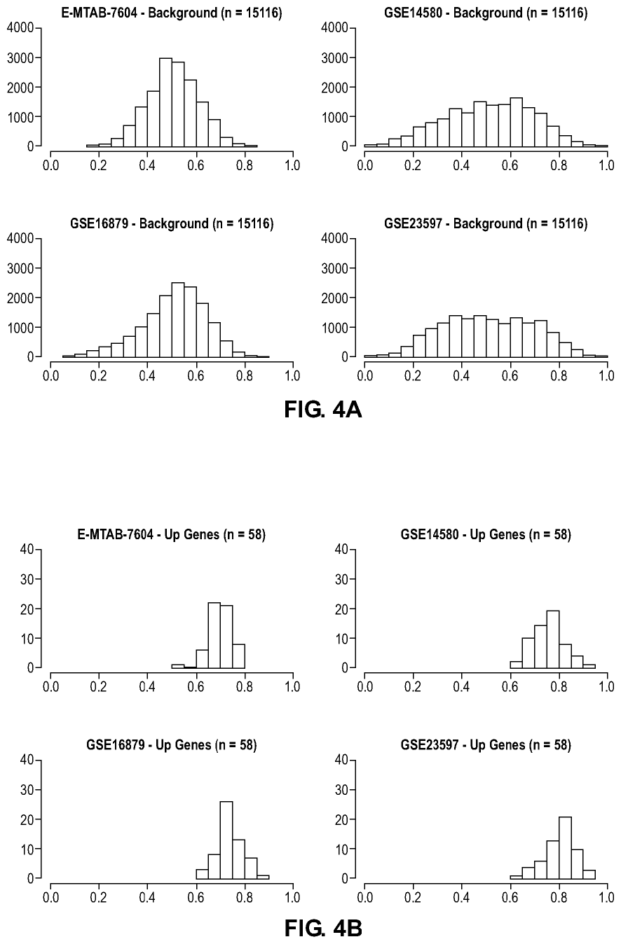

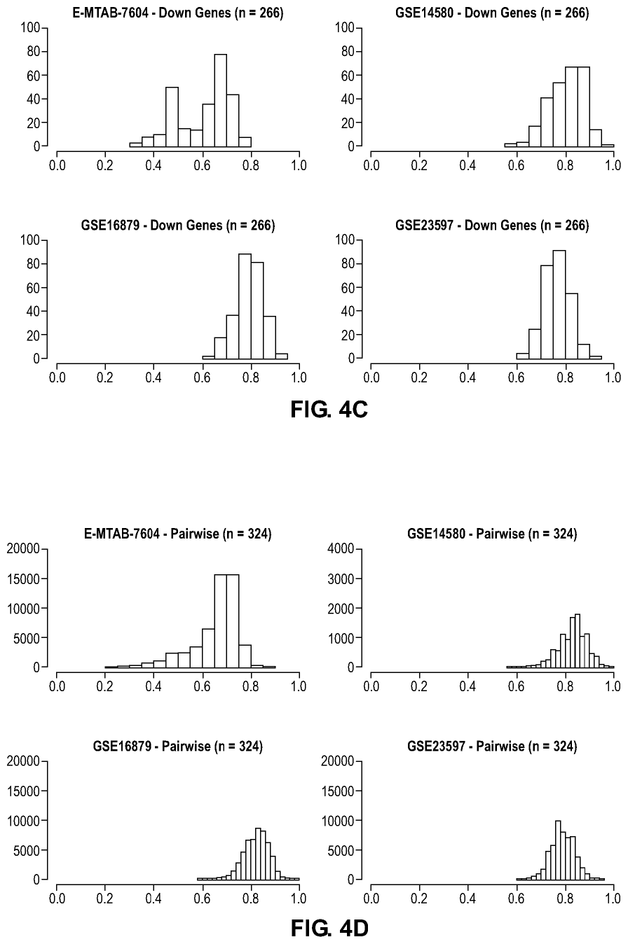

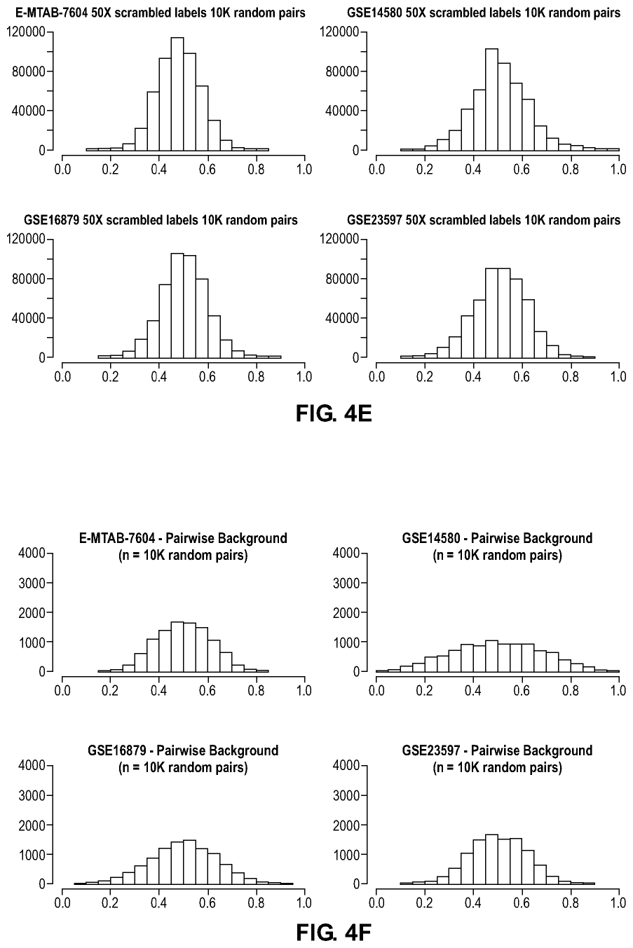

First, we determined the distribution of AUCs for all 15,116 genes as single-gene predictive markers to characterize background behavior (FIG. 4A). Next, we characterized signal for each of the 58 up-regulated DE genes (FIG. 4B) as well as the 266 down-regulated DE genes (FIG. 4C). Evidently, AUC distribution in FIG. 4B and FIG. 4C are significantly higher than in those in FIG. 4A.

TABLE 4
Performance of 10,000 randomly selected 2-gene combinations derived from the 324 DE genes,
representing 19.11% of all possible 2-gene combinations from 324 signature genes. The 2-gene
pairs are ordered from highest to lowest AUC (starting with left-most Mean AUC column).

| | | Mean | | | Mean | | | Mean |
|---|---|---|---|---|---|---|---|---|
| Gene 1 | Gene 2 | AUC | Gene 1 | Gene 2 | AUC | Gene 1 | Gene 2 | AUC |
| FPR2 | SPOCK1 | 0.811 | LGALS1 | NRCAM | 0.769 | LCP1 | SLC16A6 | 0.694 |
| MMP3 | MX2 | 0.799 | FGF2 | SELE | 0.806 | LILRA2 | S1PR1 | 0.758 |
| CXCR2 | PREX1 | 0.772 | ARPIN | SLC2A3 | 0.766 | PFKL | INHBA | 0.810 |
| COL7A1 | MMP19 | 0.792 | CSF3R | GPR183 | 0.722 | TREM1 | LST1 | 0.751 |
| GLIPR1 | COL7A1 | 0.741 | AKAP1 | FPR2 | 0.797 | CSNK1G3 | NRCAM | 0.768 |
| TNF | OSMR | 0.804 | SORT1 | EMP3 | 0.785 | FGR | CHST15 | 0.790 |
| EFNA4 | RGS16 | 0.768 | RAB31 | TMEM71 | 0.782 | LILRB2 | SRGN | 0.806 |
| TMEM154 | ANGPTL2 | 0.783 | FPR2 | NOD2 | 0.738 | SELL | TNFRSF1B | 0.764 |
| CEBPB | PCBP3 | 0.784 | LOXL2 | SLC16A6 | 0.777 | SNORC | LDLRAD3 | 0.809 |
| SMAP2 | ACOD1 | 0.753 | SPOCK1 | CD14 | 0.755 | SELENBP1 | TREM1 | 0.804 |
| RGS16 | SH3BP5 | 0.766 | RHOH | CXCR4 | 0.675 | RHOH | FAM155A | 0.788 |
| RHOH | TLR1 | 0.729 | ANGPT2 | PAPPA | 0.826 | CGAS | SERPINB9 | 0.749 |
| TMEM154 | RBMS1 | 0.754 | EFNA4 | SERPINE2 | 0.792 | TRAK1 | CHST15 | 0.788 |
| FFAR2 | CLEC4A | 0.718 | CHI3L2 | TNFAIP2 | 0.740 | BDH1 | CD69 | 0.778 |
| ASB7 | NFAM1 | 0.722 | COL15A1 | CHST11 | 0.765 | LCP1 | WNT5A | 0.771 |
| PPP2R5A | S100A12 | 0.790 | TMEM273 | GMFG | 0.781 | CEBPB | CD14 | 0.762 |
| FCGR3B | PREX1 | 0.809 | DLC1 | LCP1 | 0.770 | BICD2 | STAT5B | 0.755 |
| CHI3L1 | MEFV | 0.793 | TSPAN6 | NCF2 | 0.811 | DSP | PDLIM4 | 0.791 |
| CMTM8 | FAM155A | 0.792 | CTHRC1 | IER5 | 0.770 | LILRB3 | SLC35G2 | 0.767 |
| LILRB2 | CXCR2 | 0.780 | DPYSL3 | CYP26B1 | 0.811 | GZMB | MMP3 | 0.798 |
| TMEM106C | VASN | 0.760 | CSNK1G3 | GAS2L3 | 0.759 | RIPOR3 | ACOD1 | 0.782 |
| IFITM2 | FPR2 | 0.790 | RALGAPA2 | SSH2 | 0.766 | FGR | ANGPT2 | 0.803 |
| CMTM4 | PXDN | 0.811 | FERMT2 | C1R | 0.769 | CCR1 | TCF4 | 0.770 |
| RILPL2 | CHRDL2 | 0.781 | AGAP1 | PPP1R18 | 0.754 | ABL2 | C1R | 0.763 |
| NEO1 | CHST15 | 0.795 | FCGR3B | MSN | 0.814 | RILPL2 | NCF2 | 0.804 |
| GPR183 | FGF2 | 0.760 | IL11 | VAV1 | 0.818 | LGALS1 | C1R | 0.764 |
| CHRDL2 | TNFAIP2 | 0.756 | BPGM | ZDHHC18 | 0.758 | NDFIP2 | SMAP2 | 0.751 |
| FAP | IER5 | 0.743 | NCF4 | IER5 | 0.738 | EMP3 | PTGS1 | 0.766 |
| FILIP1L | IL1RN | 0.826 | G0S2 | TIMP1 | 0.810 | STAP2 | CXCR1 | 0.744 |
| KCNJ15 | G0S2 | 0.741 | RGS19 | COL7A1 | 0.766 | ADGRG3 | CLEC4A | 0.751 |
| PPP2R5A | PREX1 | 0.790 | CSTA | CLEC4A | 0.739 | ADCY6 | ACOT9 | 0.787 |
| HCK | IL11 | 0.813 | CYTH4 | GNAI2 | 0.758 | GLIPR1 | JAZF1 | 0.734 |
| VASN | STAT5B | 0.718 | CREM | S1PR3 | 0.774 | CSNK1G3 | ST3GAL5 | 0.742 |
| GLCE | TAGAP | 0.749 | SPP1 | CCDC3 | 0.758 | GAS2L3 | GADD45B | 0.776 |
| FJX1 | FAM20C | 0.758 | PAPPA | C5AR1 | 0.823 | LILRB1 | CRIP2 | 0.793 |
| FERMT2 | COTL1 | 0.783 | LRRK2 | BICD2 | 0.753 | SSH2 | THEMIS2 | 0.742 |
| GZMB | HS3ST3B1 | 0.796 | LILRA2 | FILIP1L | 0.794 | DPYSL3 | SLA | 0.732 |
| LILRB2 | LILRB3 | 0.746 | HIBCH | SLC15A4 | 0.795 | HCK | CXCR1 | 0.750 |
| MCAM | TLR1 | 0.793 | OCRL | UQCRC1 | 0.799 | RNF149 | IL13RA2 | 0.864 |
| GLCE | ST3GAL5 | 0.772 | RHOH | CEP19 | 0.665 | LILRB3 | CD93 | 0.757 |
| B4GALT4 | CXCR2 | 0.824 | COTL1 | RGCC | 0.766 | ADCY6 | TMEM106C | 0.785 |
| TRIM2 | ABL2 | 0.788 | CHI3L2 | LGALS1 | 0.752 | GPR183 | CD53 | 0.727 |
| ITGAM | BCAT1 | 0.754 | ATP5IF1 | FILIP1L | 0.788 | VASN | OSMR | 0.736 |
| PPP2R5A | KIAA1522 | 0.782 | AFG3L2 | TNC | 0.795 | CAVIN3 | RIPK2 | 0.792 |
| ANGPT2 | FAM49A | 0.811 | CAVIN3 | RASSF5 | 0.767 | SH3RF2 | CSF3 | 0.800 |
| OCRL | CD14 | 0.775 | AMPD2 | BMERB1 | 0.801 | MX2 | NFE2 | 0.742 |
| CMTM4 | CYP26B1 | 0.820 | NDFIP2 | CXCR2 | 0.834 | WNK2 | C5AR1 | 0.798 |
| OLFM1 | PPP1R18 | 0.773 | ARPIN | SLC35G2 | 0.808 | WNK2 | DPYSL3 | 0.806 |
| IL1RN | SPOCK1 | 0.804 | CMTM4 | FCGR2B | 0.796 | ENO2 | RIPOR2 | 0.768 |
| FCER1G | RNF149 | 0.799 | SERPINB9 | FAM20C | 0.784 | HCLS1 | MMP19 | 0.713 |
| LILRB2 | NCF2 | 0.743 | COX10 | RAB15 | 0.788 | CD300A | RIPOR2 | 0.790 |
| SRGN | STC1 | 0.810 | MIR100HG | TLR4 | 0.722 | CTHRC1 | PIP4P2 | 0.786 |
| TMEM71 | CSF3R | 0.708 | GPR183 | PLEK | 0.756 | CLEC4A | FAM155A | 0.820 |
| FAM20C | SPP1 | 0.770 | ICAM1 | NOD2 | 0.724 | G0S2 | P2RY14 | 0.815 |
| IL13RA2 | CD53 | 0.843 | CMTM4 | GADD45B | 0.780 | CLU | TUBA1A | 0.765 |
| CXCL8 | CCDC3 | 0.797 | CSNK1G3 | SELENBP1 | 0.797 | CDX2 | GZMB | 0.791 |
| PAPPA | ZDHHC18 | 0.840 | BDH1 | HLX | 0.783 | RHOH | SLC35G2 | 0.774 |
| GLT1D1 | HCK | 0.759 | CD93 | SSH2 | 0.805 | SELENBP1 | BCL6 | 0.774 |
| CLEC4E | IL1B | 0.785 | ATP5IF1 | VAV1 | 0.756 | AKAP1 | SERPINE1 | 0.790 |
| POLDIP2 | VASN | 0.756 | ATP5IF1 | CREB5 | 0.782 | MYO1F | PXDN | 0.781 |
| RHOH | PREX1 | 0.701 | SORT1 | VAV1 | 0.757 | COX10 | TRAK1 | 0.789 |
| ASAP1 | ARHGAP25 | 0.763 | HLX | TREM1 | 0.732 | RIPOR3 | CD53 | 0.766 |
| SNORC | STAP2 | 0.768 | UBAC1 | BCAT1 | 0.767 | EGR3 | MMP3 | 0.762 |
| IFITM2 | P2RY14 | 0.796 | DUSP2 | SELE | 0.807 | AGAP1 | LILRA5 | 0.765 |
| LILRA5 | BCL6 | 0.701 | RALGAPA2 | KIAA1522 | 0.773 | FPR1 | BPGM | 0.787 |
| FJX1 | CCDC88A | 0.756 | CCR1 | TMEM71 | 0.809 | SLC15A4 | TNC | 0.802 |
| IL13RA2 | PIP4P2 | 0.865 | PTGS2 | TIMP1 | 0.790 | GPR183 | FCGR2B | 0.762 |
| PPP2R5A | PI15 | 0.779 | GZMB | MAP4K4 | 0.782 | KIAA1522 | ICAM1 | 0.756 |
| NEO1 | DUSP2 | 0.796 | CDX2 | AGAP1 | 0.779 | MYO1D | RGCC | 0.796 |
| GNAI2 | PI15 | 0.755 | PDE4B | MAP4K4 | 0.724 | SERPINE1 | SLC16A6 | 0.801 |
| ERMP1 | CEMIP | 0.812 | CHI3L2 | PAPPA | 0.820 | LILRB3 | SSH2 | 0.731 |
| BCL2A1 | PHLDB2 | 0.786 | S100A9 | TLR4 | 0.741 | SH3RF2 | NDUFA10 | 0.798 |
| TCEA3 | DUSP2 | 0.789 | CGAS | ENO2 | 0.755 | SORT1 | CCN4 | 0.778 |
| HLX | SLC35G2 | 0.754 | LDLRAD3 | RBMS1 | 0.788 | G0S2 | CCL2 | 0.805 |
| RGCC | RIPOR2 | 0.780 | CGAS | FGR | 0.756 | GLIPR1 | VAV1 | 0.679 |
| TMEM71 | GLT1D1 | 0.744 | GLIPR1 | CYP26B1 | 0.747 | CSF3 | GNAI2 | 0.785 |
| AKAP1 | ASAP1 | 0.802 | CTHRC1 | CYTH4 | 0.784 | SERPINE2 | S100A8 | 0.825 |
| CLEC4E | IL13RA2 | 0.843 | KIAA1522 | SLC35G2 | 0.805 | LRP4 | TMEM273 | 0.793 |
| LRRK2 | CAVIN1 | 0.766 | RHOH | SELE | 0.785 | RGCC | MSN | 0.775 |
| AMPD2 | AQP9 | 0.788 | FCER1G | LOXL2 | 0.768 | ATP5IF1 | JAZF1 | 0.774 |
| F5 | GPR137B | 0.794 | CTHRC1 | PROK2 | 0.759 | PCBP3 | P2RY14 | 0.773 |
| LDLRAD3 | S1PR3 | 0.784 | DUSP4 | OSMR | 0.800 | CLU | ICAM1 | 0.775 |
| ADCY6 | TREM1 | 0.781 | CEP19 | TNFRSF10C | 0.716 | CDX2 | GADD45B | 0.795 |
| PIP4P2 | SNN | 0.818 | CLEC4E | SLC16A6 | 0.710 | OLFM1 | PTGS1 | 0.782 |
| SORT1 | MYO1F | 0.768 | FERMT2 | LST1 | 0.780 | LILRA5 | STC1 | 0.806 |
| RAI14 | CYTH4 | 0.779 | NFAM1 | FCGR3B | 0.788 | CEP19 | CHST2 | 0.709 |
| TRIM2 | NNMT | 0.804 | HCK | S100A9 | 0.783 | RILPL2 | PDLIM4 | 0.798 |
| DSP | NRG1 | 0.810 | CDX2 | CSF3 | 0.821 | PPP2R5A | GPR176 | 0.760 |
| IL1B | SPOCK1 | 0.804 | IL6 | CEMIP | 0.805 | SLC15A4 | OSMR | 0.786 |
| STAP2 | CYP26B1 | 0.826 | CHST11 | SLC11A1 | 0.776 | FFAR2 | MX2 | 0.717 |
| HCLS1 | SH3BP5 | 0.676 | ERMP1 | LY96 | 0.801 | MMP19 | TIMP1 | 0.764 |
| CSF3 | CLMP | 0.775 | NIPSNAP3A | TREM1 | 0.812 | SELE | ZDHHC18 | 0.802 |
| MEFV | SLC35G2 | 0.798 | WNK2 | CHST15 | 0.814 | LILRB3 | VASN | 0.732 |
| OSM | PDLIM4 | 0.822 | TLR1 | RIPOR2 | 0.749 | STAP2 | JAZF1 | 0.757 |
| PPP1R18 | PIP4P2 | 0.766 | EFNA4 | MX2 | 0.772 | SWAP70 | SPOCK1 | 0.720 |
| ADCY6 | THEMIS2 | 0.789 | DSP | SLA | 0.760 | TCEA3 | SLC11A1 | 0.793 |
| GLT1D1 | ARHGAP25 | 0.721 | ERMP1 | PTGS2 | 0.798 | CGAS | SLC11A1 | 0.786 |
| PRICKLE2 | CD14 | 0.769 | RNF149 | S100A8 | 0.802 | ZNF704 | GPR176 | 0.780 |
| PIP4P2 | S100A12 | 0.792 | NRG1 | CCDC88A | 0.790 | HCLS1 | CEMIP | 0.753 |
| WNK2 | CEBPB | 0.823 | FGR | GMFG | 0.802 | DUSP2 | NCF2 | 0.767 |
| AGAP1 | LDLRAD3 | 0.784 | NFE2 | CHST2 | 0.810 | SLC15A4 | HCK | 0.772 |
| FCER1G | GPR137B | 0.785 | TMEM71 | LST1 | 0.794 | HSD11B2 | LILRA5 | 0.795 |
| NRCAM | SPHK1 | 0.757 | CD93 | VNN2 | 0.829 | COL7A1 | MEFV | 0.797 |
| LILRA2 | BPGM | 0.776 | NR3C2 | IER5 | 0.781 | TMEM71 | ANKRD35 | 0.808 |
| RIPOR3 | FCER1G | 0.798 | TNF | TUBB6 | 0.800 | STAP2 | CD300A | 0.794 |
| SH2D3C | PRRX1 | 0.773 | TAGAP | CCDC3 | 0.722 | ST3GAL5 | GMFG | 0.769 |
| DUSP2 | RFLNB | 0.770 | CHRDL2 | CAVIN1 | 0.768 | SLC35G2 | CD69 | 0.788 |
| CAVIN1 | SPOCK1 | 0.789 | TRAK1 | NEO1 | 0.785 | RGS19 | PDLIM4 | 0.736 |
| AFG3L2 | MX2 | 0.801 | AGFG2 | IL13RA2 | 0.860 | CD93 | SLC2A3 | 0.748 |
| CR1 | PI15 | 0.755 | S100A9 | GMFG | 0.832 | ADCY6 | TLR4 | 0.734 |
| NDFIP2 | KIAA1522 | 0.778 | COL7A1 | ZDHHC18 | 0.799 | INHBA | VNN2 | 0.823 |
| FCGR2B | MEFV | 0.798 | LILRB3 | LAT2 | 0.755 | LILRB2 | IL1RN | 0.829 |
| AKAP1 | PDLIM4 | 0.796 | CD300A | SLC15A4 | 0.832 | TSPAN6 | IL11 | 0.835 |
| CLEC4E | VIM | 0.755 | PIK3AP1 | PDLIM4 | 0.753 | TRPM6 | IFITM2 | 0.775 |
| ANGPTL2 | LAT2 | 0.772 | POLDIP2 | GPR137B | 0.800 | GLT1D1 | CCL2 | 0.775 |
| FILIP1L | STC1 | 0.812 | AGAP1 | FCER1G | 0.773 | VASN | CXCL8 | 0.751 |
| LILRB2 | CYTH4 | 0.808 | AKAP1 | B4GALT4 | 0.787 | TCEA3 | G0S2 | 0.820 |
| CLEC4A | BCL2A1 | 0.742 | TLR1 | CEP19 | 0.712 | CSF3R | LGALS1 | 0.733 |
| PKIB | FAM20C | 0.775 | INHBA | SLC11A1 | 0.811 | FCGR2B | LST1 | 0.773 |
| STC1 | CREB5 | 0.803 | IL11 | PLAU | 0.809 | SHTN1 | EMP3 | 0.784 |
| CLU | CCDC88A | 0.773 | CDX2 | LGALS1 | 0.799 | PKIB | PROK2 | 0.799 |
| HCLS1 | SPP1 | 0.718 | RAB15 | COQ9 | 0.793 | ASB7 | RBMS1 | 0.789 |
| TCF4 | SLC35G2 | 0.777 | SELENBP1 | ASAP1 | 0.785 | ASAP1 | SLFN11 | 0.765 |
| CHRDL2 | GZMB | 0.787 | BASP1 | CBX6 | 0.771 | TMEM106C | ASAP1 | 0.778 |
| LDLRAD3 | SELP | 0.794 | SNORC | DUSP2 | 0.778 | HCLS1 | WNT5A | 0.750 |
| LRP4 | GPR176 | 0.798 | TAGAP | RFLNB | 0.742 | CDX2 | CCDC69 | 0.775 |
| CLU | CHRDL2 | 0.756 | SH3RF2 | NEO1 | 0.791 | BCAT1 | SELL | 0.759 |
| LCP1 | SELE | 0.786 | DUSP4 | TUBA1A | 0.780 | AMPD2 | MCAM | 0.786 |
| KIAA1522 | LCP1 | 0.758 | UQCRC1 | PXDN | 0.811 | RGS19 | FERMT2 | 0.742 |
| GAS2L3 | CD14 | 0.766 | NRG1 | IL1RN | 0.795 | SH3RF2 | SWAP70 | 0.771 |
| ANKRD35 | TUBB6 | 0.785 | ADCY6 | ST3GAL5 | 0.771 | PPP2R5A | WWC2 | 0.787 |
| OCRL | SLC15A3 | 0.777 | GALNT12 | CHI3L2 | 0.751 | ANKRD35 | SELE | 0.822 |
| S1PR3 | NOD2 | 0.733 | IFITM2 | CD53 | 0.747 | PRICKLE2 | PCBP3 | 0.809 |
| TRAK1 | NCF2 | 0.805 | ZNF704 | ATP5IF1 | 0.794 | FJX1 | CCDC3 | 0.764 |
| SLC11A1 | MAP4K4 | 0.810 | HSD11B2 | RNF149 | 0.787 | COTL1 | ST3GAL5 | 0.765 |
| NRCAM | TLR4 | 0.749 | PI15 | OSMR | 0.768 | NR3C2 | LST1 | 0.789 |
| BCAT1 | TUBB6 | 0.753 | LILRB2 | CD53 | 0.742 | CLEC4A | S100A12 | 0.793 |
| GAS2L3 | GMFG | 0.779 | LGALS1 | VAV1 | 0.753 | AGFG2 | ANKRD35 | 0.763 |
| COQ9 | PTGS2 | 0.815 | NR3C2 | RGS16 | 0.779 | F5 | PLA1A | 0.777 |
| CSF3 | TNFAIP2 | 0.787 | CHST2 | ARHGAP25 | 0.767 | BASP1 | ASAP1 | 0.787 |
| HSD11B2 | STAP2 | 0.752 | CEBPB | CHN1 | 0.790 | SHTN1 | GALNT12 | 0.780 |
| ADCY6 | RGS16 | 0.790 | FJX1 | SRGN | 0.763 | SELENBP1 | HCLS1 | 0.780 |
| LILRB3 | LCP2 | 0.744 | TNFAIP2 | CD69 | 0.731 | CSF3R | P2RY14 | 0.764 |
| CBX6 | PXDN | 0.780 | TCEA3 | CSTA | 0.780 | FAM20C | CCN4 | 0.775 |
| GAS2L3 | TNF | 0.785 | GOLPH3L | PDE4B | 0.783 | IL13RA2 | CEMIP | 0.844 |
| SSH2 | ST3GAL5 | 0.747 | ARPIN | RILPL2 | 0.785 | DSP | PTGS2 | 0.794 |
| ANKRD35 | ST3GAL5 | 0.736 | FGF7 | ASAP1 | 0.783 | CYTH4 | TUBA1A | 0.770 |
| GAS2L3 | FJX1 | 0.756 | CGAS | CBX6 | 0.704 | FPR2 | GPR137B | 0.810 |
| SLC2A3 | VIM | 0.732 | PLA1A | TLR1 | 0.772 | GPR137B | ENTPD1 | 0.810 |
| RASSF5 | CD53 | 0.695 | ANKRD35 | TLR1 | 0.816 | DUSP4 | MCAM | 0.788 |
| C11orf96 | IER5 | 0.759 | VIM | CD69 | 0.745 | EFNA4 | TNFAIP6 | 0.788 |
| ZNF704 | ZDHHC18 | 0.787 | C2CD2 | MMP19 | 0.766 | S1PR1 | PTGS1 | 0.792 |
| HCLS1 | VAV1 | 0.660 | LRRK2 | SLC16A6 | 0.710 | CREM | RAI14 | 0.778 |
| COX10 | COL4A2 | 0.784 | FCGR3B | S100A8 | 0.814 | BCL2A1 | OSMR | 0.785 |
| CEBPB | RFLNB | 0.808 | FERMT2 | LOXL2 | 0.777 | S1PR3 | LY96 | 0.774 |
| FFAR2 | CCL4 | 0.742 | ANGPTL2 | TCF4 | 0.753 | AGFG2 | RGCC | 0.793 |
| HLX | SLFN11 | 0.703 | IL24 | FILIP1L | 0.809 | IFITM2 | LST1 | 0.752 |
| GOLPH3L | BMERB1 | 0.814 | TRPM6 | TMEM154 | 0.802 | ABL2 | LGALS1 | 0.766 |
| KIAA1522 | INHBA | 0.795 | IL1B | TNFAIP6 | 0.780 | CXCR2 | MX2 | 0.775 |
| CHST15 | SLC35G2 | 0.811 | ASB7 | ENO2 | 0.784 | CCR1 | ST3GAL5 | 0.783 |
| SLC11A1 | CD53 | 0.776 | LGALS1 | OSMR | 0.781 | GPR183 | SPHK1 | 0.764 |
| EFNA4 | PLAU | 0.774 | NCF2 | SLC15A3 | 0.764 | MEFV | S100A9 | 0.827 |
| CSF3R | CEMIP | 0.739 | AQP9 | HS3ST3B1 | 0.812 | OSM | LAT2 | 0.809 |
| CCDC88A | VNN2 | 0.780 | NCF2 | RGS16 | 0.749 | PKIB | MEFV | 0.795 |
| NDUFA10 | SWAP70 | 0.741 | LDLRAD3 | SERPINB9 | 0.799 | PKIB | IFITM2 | 0.775 |
| LILRA2 | CXCL5 | 0.755 | NRCAM | SH3BP5 | 0.756 | TRPM6 | COL7A1 | 0.796 |
| S100A12 | TNF | 0.795 | ZDHHC23 | SMAP2 | 0.778 | ANGPTL2 | P2RY14 | 0.779 |
| RBMS1 | PHLDB2 | 0.773 | GLIPR1 | IL1RN | 0.752 | GPR176 | MCAM | 0.762 |
| AGFG2 | NCF4 | 0.752 | PFKL | BTG2 | 0.769 | ASB7 | THBD | 0.805 |
| PDE4B | STC1 | 0.774 | GLIPR1 | STC1 | 0.768 | VIM | CEP19 | 0.714 |
| COL15A1 | TNFRSF10C | 0.774 | GOLPH3L | PTGS1 | 0.804 | NDFIP2 | BPGM | 0.779 |
| FILIP1L | ICAM1 | 0.763 | NFAM1 | SLC16A6 | 0.698 | ASAP1 | ACOD1 | 0.780 |
| SRGN | SLC11A1 | 0.799 | FFAR2 | SERPINE1 | 0.805 | SH2D3C | CCR1 | 0.794 |
| RAB31 | MIR100HG | 0.754 | STAP2 | LAT2 | 0.779 | CHST15 | SERPINE2 | 0.803 |
| ITGAM | NNMT | 0.781 | ERMP1 | HLX | 0.773 | PPP2R5A | TAGAP | 0.779 |
| OLFM1 | IFITM2 | 0.784 | PPP2R5A | HCK | 0.773 | SERPINE2 | VNN2 | 0.809 |
| SLC2A3 | THEMIS2 | 0.738 | AMPD2 | C1R | 0.770 | SERPINE2 | RIPOR2 | 0.772 |
| BCL6 | GPR65 | 0.686 | CXCL5 | P2RY14 | 0.784 | EFNA4 | JAZF1 | 0.772 |
| LILRB3 | THEMIS2 | 0.743 | CTHRC1 | DPYSL3 | 0.782 | HLX | PLEK | 0.717 |
| IL13RA2 | BPGM | 0.866 | RNF149 | BCL6 | 0.719 | CCDC69 | IER5 | 0.718 |
| CXCR4 | WWC2 | 0.740 | MX2 | CD53 | 0.719 | POLDIP2 | VIM | 0.763 |
| DLC1 | S100A9 | 0.836 | S1PR3 | NRCAM | 0.756 | FILIP1L | INHBA | 0.801 |
| STAP2 | SRGN | 0.785 | C5AR1 | LAT2 | 0.790 | NEO1 | B4GALT4 | 0.778 |
| RFLNB | SRGN | 0.812 | COL15A1 | PAPPA | 0.810 | BICD2 | RFLNB | 0.764 |
| LOXL2 | G0S2 | 0.825 | DUSP2 | ENO2 | 0.792 | NRG1 | CHST15 | 0.804 |
| AFG3L2 | LY96 | 0.775 | B4GALT4 | CAVIN1 | 0.751 | PPP1R18 | RNF149 | 0.778 |
| COX10 | S1PR3 | 0.772 | CD300A | PXDN | 0.793 | DSP | RGCC | 0.803 |
| STAP2 | FJX1 | 0.777 | GPR183 | OSM | 0.791 | CSF2RB | HSPA6 | 0.747 |
| CSNK1G3 | MYO1D | 0.771 | NR3C2 | DPYSL2 | 0.794 | TSPAN6 | SLC11A1 | 0.795 |
| SWAP70 | FFAR2 | 0.689 | FAM20C | GPR137B | 0.787 | AGAP1 | MIR100HG | 0.765 |
| GALNT12 | ITGAM | 0.780 | ENTPD1 | ARHGAP25 | 0.782 | HLX | NRCAM | 0.704 |
| CYTH4 | LAT2 | 0.752 | POLDIP2 | SLA | 0.754 | EMP3 | HS3ST3B1 | 0.781 |
| CHST15 | SPP1 | 0.759 | BASP1 | TNFRSF10C | 0.782 | ATP5IF1 | COL7A1 | 0.810 |
| BDH1 | SLC35G2 | 0.816 | FCER1G | CHST15 | 0.783 | NRG1 | TNC | 0.812 |
| CSF3 | LST1 | 0.786 | PKIB | PCBP3 | 0.761 | CSTA | CCDC3 | 0.749 |
| GOLPH3L | CREM | 0.787 | DPYSL2 | P2RY13 | 0.770 | MX2 | SLC11A1 | 0.800 |
| TAGAP | CYP26B1 | 0.748 | PIK3AP1 | CHST11 | 0.678 | FJX1 | PTPRC | 0.758 |
| OLFM1 | LOXL2 | 0.781 | GLIPR1 | TNFRSF10C | 0.697 | CR1 | ITGAM | 0.725 |
| UBAC1 | CHRDL2 | 0.784 | FAP | HCK | 0.737 | CRIP2 | FJX1 | 0.759 |
| TMEM106C | ERMP1 | 0.779 | BASP1 | SPHK1 | 0.779 | UBAC1 | TREM1 | 0.767 |
| CXCL8 | PTPRC | 0.790 | UQCRC1 | FCGR3B | 0.804 | FGF7 | PTPRC | 0.796 |
| SELE | TNFRSF1B | 0.776 | CXCR1 | BMERB1 | 0.778 | TUBA1A | FAM49A | 0.773 |
| SHTN1 | CHRDL2 | 0.756 | IL1RN | SOD2 | 0.784 | IL24 | CXCR1 | 0.797 |
| AMPD2 | GFPT2 | 0.763 | LILRB3 | CXCR4 | 0.686 | PKIB | CAVIN1 | 0.772 |
| PIK3AP1 | DPYSL2 | 0.719 | EGR3 | C1R | 0.764 | SNORC | ENTPD1 | 0.796 |
| SLC15A3 | S100A9 | 0.796 | GLCE | NNMT | 0.808 | CTHRC1 | MIR100HG | 0.796 |
| AQP9 | CD53 | 0.769 | HLX | CCDC3 | 0.720 | ABL2 | TIMP1 | 0.764 |
| TMEM154 | ST3GAL5 | 0.770 | SERPINB9 | SLA | 0.753 | ITGAM | THBD | 0.784 |
| CSF3 | ANKRD35 | 0.807 | TMEM71 | FLT1 | 0.775 | HSPA6 | MMP3 | 0.769 |
| CLEC4E | ZDHHC18 | 0.766 | SELE | GFPT2 | 0.787 | CHST11 | CCL4 | 0.751 |
| CAVIN3 | S1PR1 | 0.769 | CDX2 | TLR1 | 0.807 | GOLPH3L | TMEM106C | 0.752 |
| ENO2 | BTG2 | 0.776 | CAVIN1 | DOK3 | 0.735 | CAVIN1 | MIR100HG | 0.761 |
| POLDIP2 | FAM155A | 0.807 | TCEA3 | CSF3 | 0.790 | NRG1 | OSM | 0.811 |
| RHOH | CD14 | 0.726 | FCER1G | CXCL5 | 0.765 | LOXL2 | BTG2 | 0.780 |
| CLEC4E | CD53 | 0.698 | SLC35G2 | CCDC3 | 0.784 | PLAU | CCDC88A | 0.769 |
| FPR2 | TLR4 | 0.699 | CLU | CSF2RB | 0.764 | CHI3L2 | THBD | 0.782 |
| S1PR1 | TNFRSF10C | 0.741 | ENO2 | ICAM1 | 0.761 | PROK2 | TUBB6 | 0.726 |
| CXCR1 | RHOH | 0.696 | MAP4K4 | GFPT2 | 0.746 | PRICKLE2 | ACOT9 | 0.801 |
| GAS2L3 | AMPD2 | 0.772 | FJX1 | INHBA | 0.797 | TRAK1 | SLC11A1 | 0.788 |
| HSPA6 | TNFAIP6 | 0.774 | SRGN | ZDHHC18 | 0.785 | TAGAP | SLC35G2 | 0.748 |
| SRGN | LST1 | 0.780 | S1PR1 | NCF2 | 0.765 | GAS2L3 | CCN4 | 0.776 |
| ASB7 | BCAT1 | 0.770 | COL7A1 | SLC2A3 | 0.766 | ADGRG3 | FFAR2 | 0.758 |
| CSF2RB | CLMP | 0.777 | TCEA3 | IFITM2 | 0.785 | CMTM4 | CD93 | 0.815 |
| DPYSL3 | SELP | 0.791 | PAPPA | WNT5A | 0.830 | S100A8 | CLMP | 0.813 |
| SELENBP1 | IL24 | 0.812 | CD300A | PTPRC | 0.791 | CHRDL2 | MIR100HG | 0.755 |
| CD300A | PPP1R18 | 0.777 | CMTM4 | SLC16A6 | 0.762 | NCF4 | ASAP1 | 0.756 |
| GOLPH3L | S100A9 | 0.834 | NDFIP2 | GOLPH3L | 0.761 | LILRB1 | IL24 | 0.813 |
| NDFIP2 | CCR1 | 0.789 | ZDHHC23 | MIR100HG | 0.800 | TREM1 | ENTPD1 | 0.781 |
| TMEM154 | S100A12 | 0.795 | TUBB6 | TNFRSF10C | 0.777 | SOD2 | WWC2 | 0.780 |
| AKAP1 | TNF | 0.798 | FGF2 | PHLDB2 | 0.771 | RAB31 | GLT1D1 | 0.742 |
| NIPSNAP3A | SELL | 0.811 | IL11 | PIP4P2 | 0.837 | ANGPTL2 | TNFAIP2 | 0.757 |
| GPR176 | BCL6 | 0.705 | NOD2 | CD14 | 0.721 | CXCR1 | ITGAM | 0.701 |
| ZDHHC23 | RIPK2 | 0.823 | RNF149 | S100A9 | 0.816 | LRRK2 | GLT1D1 | 0.725 |
| CCDC69 | TLR4 | 0.699 | GPR176 | SPP1 | 0.751 | RAB15 | EMP3 | 0.795 |
| STAP2 | MYO1F | 0.777 | ANKRD35 | STC1 | 0.827 | PTGS2 | GFPT2 | 0.765 |
| FLT1 | SH3BP5 | 0.763 | CD300A | SELP | 0.816 | IL6 | BMERB1 | 0.845 |
| COTL1 | RAI14 | 0.779 | CYTH4 | PTGS1 | 0.791 | TNC | CEMIP | 0.792 |
| SORT1 | VIM | 0.765 | SH3RF2 | SNN | 0.795 | PI15 | TNFAIP6 | 0.774 |
| BMERB1 | ARHGAP25 | 0.809 | VASN | SLC2A3 | 0.736 | TRPM6 | PTGS2 | 0.802 |
| EMP3 | BCAT1 | 0.749 | SLC15A4 | TCF4 | 0.759 | AFG3L2 | CD69 | 0.776 |
| CMTM8 | CR1 | 0.750 | TRIM2 | SPHK1 | 0.774 | LCP1 | ASAP1 | 0.744 |
| PKIB | IL24 | 0.803 | BCL2A1 | TUBB6 | 0.786 | LRRK2 | DPYSL2 | 0.776 |
| TRAK1 | IL1B | 0.787 | CXCR1 | SOD2 | 0.726 | LGALS1 | P2RY14 | 0.783 |
| TSPAN6 | IL6 | 0.821 | OSMR | CREB5 | 0.786 | RFLNB | ITGA5 | 0.787 |
| CSF2RB | ENTPD1 | 0.787 | IL13RA2 | BMERB1 | 0.878 | COTL1 | LGALS1 | 0.777 |
| GAS2L3 | WWC2 | 0.779 | COTL1 | SLC15A3 | 0.744 | ANKRD35 | ZDHHC18 | 0.776 |
| RNF149 | SPP1 | 0.765 | TAGAP | BPGM | 0.740 | G0S2 | ST3GAL5 | 0.811 |
| CSTA | WWC2 | 0.744 | ZDHHC23 | OSMR | 0.809 | C2CD2 | CEMIP | 0.771 |
| BDH1 | PTGS2 | 0.804 | ANGPT2 | SERPINE2 | 0.788 | BICD2 | CHST11 | 0.714 |
| CHRDL2 | CXCR2 | 0.827 | TNF | PDLIM4 | 0.796 | IL1RN | BPGM | 0.781 |
| GLT1D1 | FCER1G | 0.756 | FZD5 | PHLDB2 | 0.787 | NIBAN1 | STAT5B | 0.782 |
| PPP2R5A | NFE2 | 0.805 | MMP3 | SPP1 | 0.773 | OCRL | CYP26B1 | 0.825 |
| SLC15A3 | TNFAIP2 | 0.729 | CHI3L2 | FPR2 | 0.788 | SWAP70 | CREB5 | 0.710 |
| CHN1 | F5 | 0.794 | GLCE | ENO2 | 0.784 | B4GALT4 | LAT2 | 0.766 |
| CMTM8 | CREB5 | 0.787 | AQP9 | SLC2A3 | 0.753 | IL1B | BCL6 | 0.728 |
| CCL4 | SLC2A3 | 0.723 | NDUFA10 | CCL2 | 0.776 | CEBPB | CHST11 | 0.771 |
| ZDHHC23 | CCR1 | 0.830 | PKIB | S1PR1 | 0.791 | C5AR1 | ZDHHC18 | 0.784 |
| NDFIP2 | CD53 | 0.758 | SLC15A4 | FAM49A | 0.807 | CMTM8 | MMP19 | 0.744 |
| ASB7 | FCGR3B | 0.809 | MYO1F | PI15 | 0.759 | LILRB1 | FGF7 | 0.818 |
| HSD11B2 | C2CD2 | 0.777 | RAI14 | CCL4 | 0.777 | CEBPB | PLAU | 0.768 |
| SLC2A3 | ST3GAL5 | 0.767 | RALGAPA2 | LILRB2 | 0.812 | CHRDL2 | IL6 | 0.805 |
| RIPOR3 | PTGS1 | 0.799 | NR3C2 | G0S2 | 0.816 | NFAM1 | TNFAIP2 | 0.719 |
| LILRA5 | SOD2 | 0.753 | CMTM8 | BCL6 | 0.735 | C11orf96 | S100A12 | 0.813 |
| CXCR2 | SPP1 | 0.785 | LRP4 | VASN | 0.778 | FCGR3B | FPR1 | 0.771 |
| GZMB | RIPK2 | 0.773 | SH3RF2 | GALNT12 | 0.778 | TMEM106C | NOD2 | 0.742 |
| HIBCH | ATP5IF1 | 0.761 | IL13RA2 | SPP1 | 0.837 | PTGS1 | SLA | 0.750 |
| MYO1F | HS3ST3B1 | 0.782 | HLX | GMFG | 0.758 | CSNK1G3 | ZDHHC23 | 0.768 |
| CSNK1G3 | GLIPR1 | 0.767 | LILRA2 | CCDC3 | 0.759 | CXCR1 | TUBB6 | 0.769 |
| LILRB2 | CCL4 | 0.776 | DSP | TREM1 | 0.769 | WNK2 | CEMIP | 0.806 |
| CHST11 | FAM155A | 0.783 | RFLNB | BCL2A1 | 0.772 | RALGAPA2 | CXCL5 | 0.749 |
| TMEM71 | SPP1 | 0.762 | SHTN1 | PHLDB2 | 0.780 | OSM | PRRX1 | 0.802 |
| LRP4 | PKIB | 0.765 | TLR4 | ARHGAP25 | 0.699 | PHLDB2 | THEMIS2 | 0.773 |
| GOLPH3L | SLA | 0.761 | GOLPH3L | COL15A1 | 0.781 | LOXL2 | PROK2 | 0.725 |
| COL7A1 | ASAP1 | 0.792 | CHN1 | OSM | 0.820 | AMPD2 | DOK3 | 0.743 |
| ARPIN | TNFRSF10C | 0.782 | RAB15 | ABL2 | 0.804 | FILIP1L | PAPPA | 0.837 |
| DPYSL3 | IL1RN | 0.816 | SORT1 | CRIP2 | 0.765 | LGALS1 | SOD2 | 0.772 |
| GPR176 | DUSP2 | 0.755 | EMP3 | CHST11 | 0.730 | CHI3L1 | FCER1G | 0.790 |
| RHOH | SLC15A3 | 0.723 | CDX2 | JAZF1 | 0.778 | HCLS1 | NCF2 | 0.724 |
| WNK2 | ATP5IF1 | 0.811 | SLC15A4 | C11orf96 | 0.802 | CMTM4 | GPR183 | 0.784 |
| CCR1 | PAPPA | 0.830 | CLEC4A | TNF | 0.803 | SH3RF2 | S100A12 | 0.789 |
| GLIPR1 | HLX | 0.707 | BASP1 | LRRK2 | 0.800 | PIK3AP1 | RBMS1 | 0.714 |
| NDFIP2 | FPR1 | 0.801 | SH3RF2 | SMAP2 | 0.766 | NIBAN1 | PAPPA | 0.817 |
| IL11 | TCF4 | 0.825 | PI15 | GPR65 | 0.767 | AGFG2 | RIPK2 | 0.773 |
| CGAS | TNFAIP6 | 0.779 | PKIB | GFPT2 | 0.781 | IL13RA2 | RIPOR2 | 0.859 |
| CLEC4E | SOD2 | 0.746 | CHI3L2 | PI15 | 0.747 | ITPRIP | GMFG | 0.786 |
| FAP | OSM | 0.793 | SMAP2 | ENTPD1 | 0.771 | BCL6 | TNFAIP6 | 0.764 |
| DPYSL2 | NCF4 | 0.752 | MYO1D | RNF149 | 0.804 | CCDC69 | PLA1A | 0.769 |
| PRICKLE2 | RGS16 | 0.794 | NDFIP2 | GPR65 | 0.785 | ANKRD35 | NNMT | 0.808 |
| CHST11 | FAM20C | 0.758 | PXDN | ITPRIP | 0.807 | TNFAIP6 | VNN2 | 0.798 |
| OLFM1 | SELL | 0.785 | LILRB2 | ACOT9 | 0.789 | PREX1 | SLA | 0.717 |
| ACOT9 | NFE2 | 0.768 | FPR2 | TCF4 | 0.779 | SH2D3C | BICD2 | 0.733 |
| LDLRAD3 | ANKRD35 | 0.816 | RGS19 | RBMS1 | 0.724 | TLR1 | SLC35G2 | 0.803 |
| MYO1D | CHST11 | 0.774 | CXCL5 | TIMP1 | 0.770 | AMPD2 | PLEK | 0.772 |
| IL1B | CCN4 | 0.785 | NNMT | CEP19 | 0.728 | PDPN | CRIP2 | 0.790 |
| ZDHHC23 | AGFG2 | 0.798 | DSP | CLEC4E | 0.779 | NR3C2 | SELENBP1 | 0.791 |
| DUSP2 | PI15 | 0.757 | NR3C2 | PTGS2 | 0.808 | CSNK1G3 | LILRB1 | 0.790 |
| OCRL | LCP2 | 0.804 | MCAM | TNFRSF10C | 0.774 | LCP2 | CLEC4A | 0.740 |
| LILRB1 | PPP1R18 | 0.766 | ZNF704 | TCEA3 | 0.781 | C11orf96 | SPOCK1 | 0.788 |
| GNAI2 | TNFRSF1B | 0.726 | GOLPH3L | BPGM | 0.785 | AFG3L2 | CHST15 | 0.776 |
| DSP | PLEK | 0.785 | NDUFA10 | TSPAN6 | 0.765 | CYP26B1 | P2RY14 | 0.788 |
| SNORC | MYO1F | 0.783 | S1PR1 | HLX | 0.712 | PDPN | BCL6 | 0.707 |
| GALNT12 | MEFV | 0.793 | GALNT12 | IFITM2 | 0.773 | TMEM154 | FFAR2 | 0.727 |
| NR3C2 | F5 | 0.785 | PXDN | ST3GAL5 | 0.791 | PTPRC | CCDC3 | 0.762 |
| HSD11B2 | DOK3 | 0.761 | ASB7 | TAGAP | 0.724 | NFAM1 | CEP19 | 0.717 |
| C1R | GPR65 | 0.760 | PPP2R5A | CHRDL2 | 0.777 | WNK2 | CREB5 | 0.819 |
| S100A12 | HS3ST3B1 | 0.808 | CSTA | TLR4 | 0.696 | ZNF704 | RASSF5 | 0.766 |
| NOD2 | THEMIS2 | 0.723 | ITGAM | SELE | 0.802 | GLCE | CCL2 | 0.776 |
| SRGN | CD86 | 0.787 | CSNK1G3 | C5AR1 | 0.802 | C2CD2 | ITPRIP | 0.804 |
| AGAP1 | SERPINB9 | 0.774 | GOLPH3L | ANKRD35 | 0.765 | CHI3L1 | PI15 | 0.773 |
| FCER1G | THEMIS2 | 0.770 | TRIM2 | CLEC4A | 0.812 | TMEM106C | GNAI2 | 0.756 |
| TRPM6 | HS3ST3B1 | 0.773 | B4GALT4 | CSF3 | 0.802 | IFITM2 | CLMP | 0.766 |
| ADCY6 | DPYSL3 | 0.784 | INHBA | GMFG | 0.797 | EMP3 | HCK | 0.762 |
| GOLPH3L | B4GALT4 | 0.752 | RAB15 | PDPN | 0.790 | SH3RF2 | CD14 | 0.785 |
| VASN | MX2 | 0.756 | DUSP2 | TNFAIP2 | 0.749 | LRRK2 | GZMB | 0.780 |
| GPR183 | FAM155A | 0.812 | LDLRAD3 | STC1 | 0.810 | FGR | PROK2 | 0.746 |
| POLDIP2 | SLC11A1 | 0.799 | CHST2 | CD53 | 0.746 | POLDIP2 | CYP26B1 | 0.809 |
| UQCRC1 | FGR | 0.808 | CTHRC1 | DPYSL2 | 0.773 | NRCAM | ST3GAL5 | 0.755 |
| COL7A1 | CXCR2 | 0.830 | BASP1 | PREX1 | 0.770 | IL24 | PTGS1 | 0.803 |
| DUSP2 | CCDC3 | 0.786 | ANGPT2 | RHOH | 0.768 | BASP1 | MMP19 | 0.783 |
| NIPSNAP3A | PPP1R18 | 0.777 | DUSP4 | PXDN | 0.806 | NDUFA10 | BMERB1 | 0.829 |
| NEO1 | LRRK2 | 0.804 | RGS19 | NOD2 | 0.673 | TRPM6 | UQCRC1 | 0.784 |
| BICD2 | PLEK | 0.756 | SHTN1 | C5AR1 | 0.790 | COL4A2 | WWC2 | 0.735 |
| LRP4 | GZMB | 0.782 | SMAP2 | RASSF5 | 0.734 | COTL1 | IL6 | 0.809 |
| MYO1D | SLC15A3 | 0.766 | FCGR3B | RHOH | 0.788 | NFAM1 | CXCR1 | 0.682 |
| FILIP1L | PLAU | 0.769 | HIBCH | NDUFA10 | 0.767 | GLCE | TNFRSF10C | 0.750 |
| ARPIN | CD300A | 0.792 | CEBPB | RASSF5 | 0.779 | CMTM4 | C2CD2 | 0.792 |
| NDFIP2 | RAB31 | 0.777 | CSF2RB | PTGS2 | 0.788 | DUSP4 | PROK2 | 0.715 |
| CREB5 | P2RY14 | 0.787 | FAP | SERPINE1 | 0.763 | HSPA6 | WNT5A | 0.788 |
| EGR3 | ACOD1 | 0.763 | DSP | TNFAIP2 | 0.763 | DUSP2 | THEMIS2 | 0.769 |
| HCLS1 | CXCL8 | 0.749 | S100A9 | SLFN11 | 0.781 | RAB15 | PROK2 | 0.755 |
| AKAP1 | PTGS2 | 0.813 | TRPM6 | LRRK2 | 0.822 | ANKRD35 | TNF | 0.794 |
| SELP | ST3GAL5 | 0.790 | S100A9 | RIPK2 | 0.817 | ENO2 | CLEC4A | 0.815 |
| CSNK1G3 | LGALS1 | 0.786 | CCDC88A | THBD | 0.795 | FJX1 | SLC35G2 | 0.790 |
| TRAK1 | S100A12 | 0.784 | NCF4 | PROK2 | 0.762 | FERMT2 | BCL2A1 | 0.778 |
| INHBA | G0S2 | 0.818 | EGR3 | SLC35G2 | 0.774 | CHN1 | P2RY14 | 0.790 |
| CMTM4 | RIPOR3 | 0.806 | PPP1R18 | PREX1 | 0.732 | PFKL | SELENBP1 | 0.798 |
| IL1RN | ACOD1 | 0.786 | NNMT | CD69 | 0.777 | NIPSNAP3A | LILRA5 | 0.811 |
| IL6 | PRRX1 | 0.780 | LCP2 | SLC11A1 | 0.804 | IL24 | LOXL2 | 0.809 |
| FCER1G | TNFAIP6 | 0.785 | CGAS | ACOD1 | 0.746 | CLU | HLX | 0.729 |
| FGR | SWAP70 | 0.737 | NDUFA10 | BICD2 | 0.805 | TNF | SPHK1 | 0.795 |
| ARPIN | NDUFA10 | 0.773 | LILRB2 | ANKRD35 | 0.832 | NIPSNAP3A | TMEM71 | 0.819 |
| OCRL | MEFV | 0.789 | IL1B | NCF2 | 0.771 | TCEA3 | ENTPD1 | 0.802 |
| BDH1 | GMFG | 0.793 | GPR176 | RIPK2 | 0.767 | CCL2 | SH3BP5 | 0.758 |
| GALNT12 | TMEM154 | 0.792 | CLEC4E | SRGN | 0.767 | CAVIN3 | IL11 | 0.832 |
| OCRL | RAI14 | 0.779 | CAVIN3 | CD69 | 0.769 | POLDIP2 | RASSF5 | 0.772 |
| B4GALT4 | BICD2 | 0.755 | CLEC4E | GADD45B | 0.754 | TREM1 | VIM | 0.769 |
| IL1RN | ENTPD1 | 0.807 | COQ9 | TIMP1 | 0.774 | CMTM8 | FAM20C | 0.778 |
| PIK3AP1 | KCNJ15 | 0.689 | RILPL2 | PTPRC | 0.799 | ADGRG3 | CAVIN1 | 0.744 |
| CSF3R | TMEM154 | 0.713 | NDUFA10 | STAP2 | 0.759 | PLA1A | TUBB6 | 0.777 |
| PREX1 | GMFG | 0.771 | RAB31 | BMERB1 | 0.819 | NR3C2 | CYTH4 | 0.773 |
| FZD5 | DLC1 | 0.794 | FCGR3B | MIR100HG | 0.811 | B4GALT4 | CD53 | 0.738 |
| LST1 | DOK3 | 0.749 | RFLNB | AQP9 | 0.788 | CHI3L1 | WNT5A | 0.823 |
| RAB31 | DUSP2 | 0.762 | CHST11 | FAM49A | 0.729 | CSF2RB | SLC11A1 | 0.813 |
| HSD11B2 | FFAR2 | 0.787 | RNF149 | OSMR | 0.798 | OCRL | LDLRAD3 | 0.805 |
| PPP2R5A | CXCR1 | 0.763 | ENO2 | FPR1 | 0.791 | WNT5A | GFPT2 | 0.779 |
| IL24 | ITGA5 | 0.802 | NDUFA10 | CSTA | 0.760 | TMEM71 | P2RY14 | 0.800 |
| UQCRC1 | LILRB1 | 0.803 | LILRB2 | RILPL2 | 0.820 | PPP1R18 | CLEC4A | 0.771 |
| IL1B | VNN2 | 0.804 | CSF3 | ARHGAP25 | 0.781 | CCDC69 | MMP19 | 0.773 |
| COQ9 | VNN2 | 0.824 | RILPL2 | MYO1F | 0.791 | MCAM | SERPINE1 | 0.791 |
| CSNK1G3 | RILPL2 | 0.775 | TMEM106C | S1PR3 | 0.749 | VNN2 | GFPT2 | 0.783 |
| CMTM4 | GPR137B | 0.795 | SLC15A4 | VIM | 0.787 | CDX2 | SERPINE2 | 0.783 |
| MYO1F | NCF4 | 0.765 | DPYSL3 | P2RY13 | 0.760 | DPYSL3 | OSMR | 0.784 |
| IL1RN | PRR16 | 0.833 | OLFM1 | CTHRC1 | 0.790 | LILRB1 | AQP9 | 0.798 |
| LRP4 | PTGS1 | 0.793 | TRAK1 | LST1 | 0.762 | LRRK2 | SOD2 | 0.783 |
| VAV1 | CREB5 | 0.731 | TREM1 | SELL | 0.791 | PROK2 | MAP4K4 | 0.719 |
| GOLPH3L | NCF4 | 0.753 | CCR1 | DPYSL3 | 0.789 | TCEA3 | PIK3AP1 | 0.733 |
| SPOCK1 | WNT5A | 0.804 | FCER1G | CD93 | 0.800 | FGF2 | MX2 | 0.778 |
| CD93 | SLA | 0.768 | ASB7 | LY96 | 0.797 | CHRDL2 | MMP19 | 0.768 |
| CHI3L1 | GADD45B | 0.808 | ADCY6 | ANGPTL2 | 0.781 | DUSP4 | FCER1G | 0.795 |
| LRP4 | ITPRIP | 0.799 | RGS19 | MAP4K4 | 0.722 | MYO1D | PDE4B | 0.787 |
| COX10 | CBX6 | 0.776 | PPP2R5A | BCAT1 | 0.780 | NDFIP2 | MSN | 0.775 |
| POLDIP2 | CREB5 | 0.793 | JAZF1 | MMP19 | 0.774 | B4GALT4 | CLU | 0.756 |
| CSTA | IL1RN | 0.761 | SHTN1 | MIR100HG | 0.754 | KIAA1522 | CHST15 | 0.782 |
| ANGPT2 | SERPINB9 | 0.802 | STAP2 | LGALS1 | 0.762 | BICD2 | BMERB1 | 0.809 |
| F5 | C11orf96 | 0.800 | RAI14 | LOXL2 | 0.773 | CLU | COL15A1 | 0.773 |
| IL1B | ITPRIP | 0.804 | NIBAN1 | SMAP2 | 0.776 | ASB7 | FGF7 | 0.803 |
| PI15 | DOK3 | 0.757 | ZDHHC23 | CYP26B1 | 0.831 | CHI3L1 | TREM1 | 0.780 |
| AGAP1 | LY96 | 0.789 | SWAP70 | SERPINE2 | 0.721 | TMEM106C | PIP4P2 | 0.778 |
| CGAS | CHRDL2 | 0.770 | KIAA1522 | STC1 | 0.813 | DUSP4 | FAM49A | 0.805 |
| STAP2 | GPR183 | 0.752 | VIM | HS3ST3B1 | 0.774 | COX10 | NOD2 | 0.732 |
| IL6 | RASSF5 | 0.799 | CBX6 | RGCC | 0.776 | SPP1 | ST3GAL5 | 0.745 |
| CD93 | ITGA5 | 0.789 | COL7A1 | C11orf96 | 0.792 | S1PR1 | CLEC4E | 0.728 |
| IER5 | STC1 | 0.783 | UQCRC1 | SELL | 0.808 | CHN1 | CCL2 | 0.767 |
| PKIB | RHOH | 0.791 | BPGM | STAT5B | 0.753 | TUBA1A | ARHGAP25 | 0.752 |
| COQ9 | S100A8 | 0.816 | CSF3 | PRR16 | 0.816 | AKAP1 | MIR100HG | 0.770 |
| CHST2 | P2RY14 | 0.793 | CYTH4 | RASSF5 | 0.768 | EGR3 | PLA1A | 0.769 |
| IL1RN | ITPRIP | 0.804 | CBX6 | SH3BP5 | 0.707 | NNMT | FAM49A | 0.795 |
| PIK3AP1 | MMP19 | 0.733 | UQCRC1 | SELP | 0.798 | GLT1D1 | IL6 | 0.811 |
| CSF2RB | FAP | 0.767 | NDUFA10 | CLEC4E | 0.799 | TRAK1 | LCP2 | 0.791 |
| ASB7 | IL11 | 0.844 | HLX | PCBP3 | 0.723 | PRR16 | PTPRC | 0.807 |
| CCR1 | SELE | 0.823 | PDE4B | PHLDB2 | 0.742 | CXCR4 | SLC16A6 | 0.706 |
| ARPIN | BDH1 | 0.770 | AGAP1 | NFE2 | 0.772 | ARPIN | ANKRD35 | 0.773 |
| FGF2 | AMPD2 | 0.776 | UBAC1 | COL15A1 | 0.774 | S100A9 | CXCL5 | 0.784 |
| CSTA | NFE2 | 0.736 | UQCRC1 | AKAP1 | 0.808 | MCAM | CCL2 | 0.758 |
| CHST11 | PRRX1 | 0.758 | DSP | LILRA5 | 0.794 | PDPN | GADD45B | 0.788 |
| ANKRD35 | ITGA5 | 0.779 | CXCR1 | SPOCK1 | 0.761 | NDFIP2 | COL4A2 | 0.774 |
| DLC1 | CR1 | 0.780 | CCDC69 | SELP | 0.805 | RAB15 | STAT5B | 0.774 |
| FCGR3B | SELP | 0.831 | S100A12 | LAT2 | 0.815 | ADCY6 | LDLRAD3 | 0.793 |
| SRGN | SOD2 | 0.790 | VIM | CXCR4 | 0.713 | VASN | PRICKLE2 | 0.742 |
| UBAC1 | AKAP1 | 0.823 | S1PR1 | RASSF5 | 0.731 | IL11 | CLEC4A | 0.834 |
| ERMP1 | CCDC3 | 0.781 | GALNT12 | CD69 | 0.778 | MEFV | NRCAM | 0.778 |
| PRICKLE2 | THEMIS2 | 0.812 | TCEA3 | CEBPB | 0.801 | ADGRG3 | BTG2 | 0.755 |
| GLT1D1 | OSMR | 0.785 | LILRA2 | NIBAN1 | 0.751 | ADCY6 | FFAR2 | 0.762 |
| COTL1 | SELL | 0.790 | MSN | CHST11 | 0.752 | PDE4B | VIM | 0.743 |
| RAI14 | SLC35G2 | 0.787 | SELL | BPGM | 0.787 | GOLPH3L | CYP26B1 | 0.812 |
| IFITM2 | TNF | 0.770 | TLR1 | TNF | 0.804 | IL24 | CD14 | 0.793 |
| GZMB | PLAU | 0.770 | CLEC4A | CCDC88A | 0.754 | EGR3 | FPR1 | 0.765 |
| ST3GAL5 | CREB5 | 0.789 | CLU | PTPRC | 0.759 | GOLPH3L | ADGRG3 | 0.764 |
| CHI3L2 | HS3ST3B1 | 0.741 | ACOT9 | SELP | 0.806 | NDFIP2 | WWC2 | 0.769 |
| JAZF1 | SELE | 0.806 | CCDC69 | TNC | 0.787 | FILIP1L | VIM | 0.746 |
| TRPM6 | FAM49A | 0.819 | LY96 | CREB5 | 0.788 | PIK3AP1 | CR1 | 0.677 |
| AFG3L2 | PHLDB2 | 0.790 | C2CD2 | C1R | 0.775 | MX2 | SOD2 | 0.797 |
| MMP3 | S100A9 | 0.808 | FLT1 | CLMP | 0.765 | BASP1 | PAPPA | 0.837 |
| FGF2 | IL1B | 0.789 | TCEA3 | GLIPR1 | 0.765 | RALGAPA2 | LRRK2 | 0.808 |
| CRIP2 | CYP26B1 | 0.814 | FERMT2 | DUSP4 | 0.807 | CHI3L2 | CXCL5 | 0.780 |
| ABL2 | THBD | 0.777 | HCK | BPGM | 0.751 | PLA1A | RASSF5 | 0.757 |
| FERMT2 | IL6 | 0.813 | TNF | FAM49A | 0.797 | CBX6 | THEMIS2 | 0.725 |
| NRG1 | S100A8 | 0.810 | ZNF704 | BMERB1 | 0.835 | RGCC | TUBB6 | 0.779 |
| CD93 | ANGPT2 | 0.804 | CYTH4 | SERPINE2 | 0.797 | SLC15A3 | PDLIM4 | 0.781 |
| SWAP70 | GNAI2 | 0.688 | CCR1 | GPR65 | 0.784 | TMEM71 | C11orf96 | 0.808 |
| S1PR1 | FGF7 | 0.777 | RGCC | ST3GAL5 | 0.786 | FFAR2 | CD14 | 0.743 |
| PTGS1 | BCL2A1 | 0.781 | TSPAN6 | PLAU | 0.804 | S1PR3 | SLC35G2 | 0.799 |
| LILRB2 | BCL6 | 0.706 | TNF | TUBA1A | 0.790 | ANGPT2 | MCAM | 0.776 |
| BICD2 | SRGN | 0.798 | IER5 | PRRX1 | 0.739 | RFLNB | SERPINE1 | 0.793 |
| GOLPH3L | PHLDB2 | 0.773 | PTGS2 | SELL | 0.809 | ACOT9 | RHOH | 0.752 |
| SLA | CXCR4 | 0.712 | ZDHHC18 | RIPK2 | 0.778 | ITGA5 | PDE4B | 0.761 |
| UQCRC1 | ICAM1 | 0.768 | PKIB | FZD5 | 0.754 | PKIB | LY96 | 0.812 |
| PTGS2 | GMFG | 0.801 | TRPM6 | MX2 | 0.789 | B4GALT4 | TCF4 | 0.747 |
| PKIB | PDE4B | 0.804 | LILRA5 | LOXL2 | 0.780 | HSD11B2 | SNORC | 0.744 |
| CCR1 | NRCAM | 0.782 | CHN1 | CSF3 | 0.796 | NCF4 | BCAT1 | 0.750 |
| TRAK1 | NRG1 | 0.805 | ANKRD35 | CAVIN1 | 0.788 | SMAP2 | TNFAIP2 | 0.751 |
| TSPAN6 | ITGA5 | 0.796 | SERPINE2 | LST1 | 0.785 | CSF3R | RAI14 | 0.742 |
| UBAC1 | TMEM273 | 0.815 | CHI3L1 | FGF2 | 0.786 | MMP19 | CCDC88A | 0.775 |
| CHN1 | ACOT9 | 0.807 | TMEM273 | RBMS1 | 0.772 | AFG3L2 | ATP5IF1 | 0.749 |
| MEFV | CREB5 | 0.719 | LILRA2 | BCL6 | 0.699 | FPR2 | PDE4B | 0.727 |
| LILRB2 | CR1 | 0.773 | SLC15A4 | C2CD2 | 0.782 | ARPIN | FJX1 | 0.760 |
| S1PR1 | VIM | 0.753 | NDFIP2 | ARHGAP25 | 0.779 | BCL6 | HS3ST3B1 | 0.747 |
| NDFIP2 | HCLS1 | 0.754 | IFITM2 | IL24 | 0.793 | COX10 | MX2 | 0.788 |
| COTL1 | MX2 | 0.770 | GOLPH3L | PRR16 | 0.822 | ADCY6 | NDFIP2 | 0.780 |
| PIK3AP1 | ARHGAP25 | 0.674 | PDPN | SPP1 | 0.768 | TSPAN6 | NFE2 | 0.789 |
| BCL2A1 | TNFRSF1B | 0.737 | TCEA3 | ITPRIP | 0.799 | GPR65 | CHST2 | 0.780 |
| GALNT12 | AQP9 | 0.805 | TMEM154 | CBX6 | 0.738 | PFKL | TUBA1A | 0.780 |
| KIAA1522 | FGF7 | 0.779 | CAVIN1 | P2RY13 | 0.763 | NNMT | IER5 | 0.770 |
| CHN1 | BCL2A1 | 0.780 | BASP1 | SLC16A6 | 0.779 | ADGRG3 | CXCR1 | 0.705 |
| GLIPR1 | CHST11 | 0.685 | LILRA2 | FGR | 0.755 | FJX1 | HCK | 0.752 |
| PAPPA | WWC2 | 0.798 | FGF2 | SWAP70 | 0.723 | SWAP70 | ZDHHC18 | 0.728 |
| FILIP1L | ACOD1 | 0.784 | AFG3L2 | SH3BP5 | 0.779 | CXCR2 | NCF4 | 0.816 |
| RAB15 | STAP2 | 0.782 | EFNA4 | ABL2 | 0.769 | HCLS1 | KCNJ15 | 0.684 |
| LILRB1 | RAI14 | 0.803 | AGAP1 | WNT5A | 0.802 | LDLRAD3 | OSM | 0.822 |
| SELP | THBD | 0.800 | GAS2L3 | IL1RN | 0.769 | GAS2L3 | TAGAP | 0.751 |
| DPYSL2 | SERPINE1 | 0.793 | TRPM6 | PROK2 | 0.765 | MMP19 | CD69 | 0.762 |
| PDPN | CLEC4E | 0.770 | TUBB6 | CCN4 | 0.779 | B4GALT4 | GPR176 | 0.750 |
| LAT2 | BTG2 | 0.762 | CAVIN1 | P2RY14 | 0.757 | SH2D3C | MAP4K4 | 0.734 |
| AGAP1 | P2RY13 | 0.794 | CXCL8 | PTGS1 | 0.799 | PRRX1 | P2RY14 | 0.770 |
| FAM49A | ITPRIP | 0.814 | NEO1 | CXCR2 | 0.809 | LRP4 | NRCAM | 0.776 |
| PLEK | LST1 | 0.740 | SSH2 | CREB5 | 0.738 | TNFAIP6 | CHST2 | 0.788 |
| CMTM4 | RALGAPA2 | 0.785 | SELENBP1 | FJX1 | 0.792 | GNAI2 | IL11 | 0.815 |
| PIP4P2 | CD53 | 0.721 | MCAM | LAT2 | 0.790 | OSM | SELE | 0.816 |
| RAB31 | CLEC4E | 0.756 | PREX1 | TLR1 | 0.736 | PRICKLE2 | IL1RN | 0.825 |
| ACOT9 | NOD2 | 0.731 | COX10 | GLT1D1 | 0.773 | SORT1 | RAB31 | 0.775 |
| SPOCK1 | TLR4 | 0.722 | CLEC4E | BCL6 | 0.693 | LILRA5 | PCBP3 | 0.760 |
| FZD5 | FAM155A | 0.795 | CHN1 | SLFN11 | 0.749 | CGAS | PI15 | 0.754 |
| RALGAPA2 | ENTPD1 | 0.808 | DLC1 | WNT5A | 0.818 | CAVIN3 | TLR1 | 0.781 |
| NEO1 | TSPAN6 | 0.781 | OLFM1 | CR1 | 0.753 | P2RY13 | ITPRIP | 0.814 |
| CRIP2 | S100A8 | 0.833 | PROK2 | LST1 | 0.721 | ICAM1 | NNMT | 0.766 |
| CHN1 | SPOCK1 | 0.784 | BDH1 | DUSP4 | 0.761 | BASP1 | GPR176 | 0.757 |
| CBX6 | IER5 | 0.688 | KCNJ15 | BCL6 | 0.700 | RHOH | C1R | 0.745 |
| PPP2R5A | THBD | 0.797 | SWAP70 | FPR2 | 0.709 | TSPAN6 | G0S2 | 0.838 |
| CYTH4 | NRCAM | 0.771 | GPR183 | CLEC4A | 0.762 | ADGRG3 | SLA | 0.744 |
| LOXL2 | CD86 | 0.784 | MYO1F | ITPRIP | 0.802 | PFKL | CD69 | 0.762 |
| AFG3L2 | TNFRSF10C | 0.770 | ANKRD35 | SLC15A3 | 0.748 | CBX6 | VNN2 | 0.788 |
| CHI3L1 | IL1RN | 0.805 | ADGRG3 | TNFRSF1B | 0.732 | LDLRAD3 | LAT2 | 0.787 |
| RIPOR3 | NNMT | 0.810 | RHOH | TLR4 | 0.686 | DSP | LRRK2 | 0.791 |
| BDH1 | TUBA1A | 0.798 | ASB7 | TCF4 | 0.762 | GLT1D1 | S1PR3 | 0.763 |
| AQP9 | ACOD1 | 0.768 | UBAC1 | PXDN | 0.803 | FFAR2 | RFLNB | 0.749 |
| HIBCH | PXDN | 0.792 | CEMIP | CD69 | 0.775 | FPR2 | CAVIN1 | 0.788 |
| HSPA6 | LCP2 | 0.707 | CYTH4 | SELE | 0.814 | ARPIN | AMPD2 | 0.776 |
| RFLNB | NRCAM | 0.766 | CREM | TCF4 | 0.762 | BICD2 | CD69 | 0.715 |
| FILIP1L | TMEM154 | 0.805 | DPYSL3 | CD86 | 0.774 | VASN | OSM | 0.791 |
| ACOT9 | SH3BP5 | 0.769 | GLT1D1 | DOK3 | 0.727 | LCP1 | NNMT | 0.760 |
| FJX1 | OSMR | 0.777 | GAS2L3 | ANGPTL2 | 0.789 | PREX1 | BTG2 | 0.753 |
| SLC15A4 | COL15A1 | 0.787 | FCGR2B | CLEC4A | 0.806 | SNORC | HCLS1 | 0.751 |
| COQ9 | PTGS1 | 0.793 | AKAP1 | IER5 | 0.780 | ASB7 | PDE4B | 0.748 |
| CHST15 | ITPRIP | 0.803 | CCL4 | VAV1 | 0.757 | FAM20C | S100A8 | 0.808 |
| DLC1 | LRRK2 | 0.808 | TAGAP | NFAM1 | 0.703 | FZD5 | CCN4 | 0.787 |
| LGALS1 | PROK2 | 0.730 | HCLS1 | CCL2 | 0.714 | ICAM1 | CXCR2 | 0.779 |
| COL4A2 | LDLRAD3 | 0.759 | PDPN | CSF3 | 0.790 | CMTM4 | OSM | 0.811 |
| COL7A1 | BMERB1 | 0.818 | LILRB3 | TLR1 | 0.749 | RGS19 | TUBB6 | 0.748 |
| GLIPR1 | COL4A2 | 0.697 | FAP | TNF | 0.763 | OCRL | CD93 | 0.804 |
| TRIM2 | PAPPA | 0.838 | UQCRC1 | NCF4 | 0.767 | SH2D3C | CHN1 | 0.773 |
| NEO1 | LAT2 | 0.767 | PPP2R5A | SELE | 0.805 | ANKRD35 | IL1RN | 0.797 |
| THBD | SPHK1 | 0.797 | NRG1 | CEMIP | 0.788 | LILRB1 | SELP | 0.832 |
| SLC15A3 | SSH2 | 0.744 | SOD2 | ACOD1 | 0.763 | AMPD2 | PTGS2 | 0.796 |
| SLC15A3 | TNF | 0.770 | CHN1 | BCL6 | 0.716 | BPGM | CD53 | 0.741 |
| RGCC | SLA | 0.766 | TMEM273 | PHLDB2 | 0.760 | NIBAN1 | BMERB1 | 0.803 |
| CHI3L1 | S1PR1 | 0.785 | HSD11B2 | TMEM273 | 0.770 | FCGR3B | C1R | 0.831 |
| HCK | TUBB6 | 0.766 | STAP2 | CHST15 | 0.775 | RAB15 | GOLPH3L | 0.774 |
| CRIP2 | PREX1 | 0.745 | PKIB | GLT1D1 | 0.765 | CGAS | MSN | 0.761 |
| P2RY13 | FAM155A | 0.810 | MX2 | SELL | 0.804 | BCL6 | S100A8 | 0.763 |
| ZDHHC23 | SPOCK1 | 0.809 | ASB7 | CD93 | 0.802 | NDFIP2 | AQP9 | 0.801 |
| ADGRG3 | CYP26B1 | 0.786 | AKAP1 | RGS19 | 0.769 | NOD2 | SLFN11 | 0.715 |
| PPP1R18 | FAP | 0.749 | UQCRC1 | CHRDL2 | 0.772 | ACOT9 | RGS16 | 0.783 |
| TRAK1 | SERPINB9 | 0.782 | HIBCH | PTGS2 | 0.805 | PTGS1 | VAV1 | 0.774 |
| RHOH | NOD2 | 0.687 | LCP2 | IER5 | 0.714 | MYO1D | GPR137B | 0.799 |
| NDFIP2 | MAP4K4 | 0.782 | CHST15 | PRRX1 | 0.769 | PREX1 | STAT5B | 0.747 |
| NEO1 | OSM | 0.820 | CGAS | HCLS1 | 0.677 | MMP19 | PTGS1 | 0.779 |
| SH2D3C | FLT1 | 0.761 | TMEM273 | SPHK1 | 0.791 | LST1 | CD53 | 0.722 |
| LCP2 | CXCL5 | 0.757 | LRRK2 | FPR2 | 0.754 | TLR4 | SH3BP5 | 0.695 |
| TMEM106C | ENO2 | 0.766 | COTL1 | SERPINB9 | 0.768 | CSNK1G3 | NDUFA10 | 0.770 |
| AMPD2 | HS3ST3B1 | 0.791 | ZNF704 | CAVIN1 | 0.777 | ATP5IF1 | IL13RA2 | 0.874 |
| CMTM8 | VNN2 | 0.807 | CCR1 | ABL2 | 0.777 | TRPM6 | COL15A1 | 0.775 |
| NNMT | CHST11 | 0.786 | JAZF1 | TNF | 0.790 | TSPAN6 | IL1B | 0.807 |
| CTHRC1 | RNF149 | 0.796 | CREM | BCL2A1 | 0.778 | THBD | GPR137B | 0.801 |
| GNAI2 | ASAP1 | 0.750 | S100A12 | CLMP | 0.796 | COQ9 | VIM | 0.782 |
| CD300A | S100A8 | 0.820 | CSNK1G3 | DUSP4 | 0.770 | CYP26B1 | RBMS1 | 0.811 |
| MYO1D | COQ9 | 0.787 | AGFG2 | WWC2 | 0.755 | ASB7 | PTGS1 | 0.797 |
| DPYSL2 | ST3GAL5 | 0.750 | RAI14 | NRG1 | 0.794 | NR3C2 | FLT1 | 0.796 |
| SH3RF2 | G0S2 | 0.808 | DPYSL3 | SOD2 | 0.787 | GAS2L3 | WNK2 | 0.809 |
| HSPA6 | TNF | 0.761 | RAB31 | MX2 | 0.776 | IL13RA2 | PTPRC | 0.866 |
| EGR3 | P2RY13 | 0.741 | BASP1 | ARHGAP25 | 0.771 | ABL2 | PRRX1 | 0.763 |
| DLC1 | CCR1 | 0.803 | GZMB | SERPINB9 | 0.788 | TMEM273 | PPP1R18 | 0.761 |
| AGAP1 | CEP19 | 0.737 | BICD2 | PXDN | 0.783 | IL1B | MEFV | 0.791 |
| CEBPB | SPHK1 | 0.778 | CGAS | CLU | 0.773 | HSD11B2 | PRICKLE2 | 0.787 |
| GPR65 | BMERB1 | 0.834 | LCP2 | ENTPD1 | 0.780 | TNC | SLC16A6 | 0.797 |
| GPR176 | PRRX1 | 0.758 | HSD11B2 | PXDN | 0.808 | PTGS2 | FAM49A | 0.773 |
| EFNA4 | IL24 | 0.796 | FGF2 | SOD2 | 0.779 | CR1 | CYP26B1 | 0.781 |
| RAB15 | SLC35G2 | 0.830 | AGAP1 | POLDIP2 | 0.759 | CMTM4 | COL7A1 | 0.810 |
| CXCR1 | MCAM | 0.760 | ZNF704 | LCP2 | 0.770 | IL6 | NFE2 | 0.801 |
| LILRB1 | FAM155A | 0.836 | HLX | OSM | 0.760 | RGCC | TLR4 | 0.742 |
| POLDIP2 | FPR1 | 0.790 | TNF | VAV1 | 0.776 | PPP1R18 | CAVIN1 | 0.746 |
| UQCRC1 | SPOCK1 | 0.798 | ASB7 | CAVIN1 | 0.759 | CMTM8 | DPYSL3 | 0.766 |
| FPR1 | CXCL5 | 0.754 | HIBCH | CLEC4A | 0.802 | FPR1 | LOXL2 | 0.777 |
| SLC15A4 | CSTA | 0.747 | SNORC | MEFV | 0.783 | LILRA2 | FFAR2 | 0.727 |
| SERPINE1 | BPGM | 0.789 | CMTM4 | CSF3R | 0.753 | EGR3 | KCNJ15 | 0.725 |
| CHRDL2 | LST1 | 0.761 | GOLPH3L | FJX1 | 0.767 | SH2D3C | ASAP1 | 0.758 |
| LILRB3 | CEP19 | 0.711 | AMPD2 | MEFV | 0.756 | NEO1 | SH2D3C | 0.769 |
| IER5 | PHLDB2 | 0.739 | B4GALT4 | CLEC4E | 0.779 | BCL2A1 | CCN4 | 0.777 |
| COTL1 | TLR1 | 0.789 | LCP2 | ITPRIP | 0.810 | RAB31 | TMEM273 | 0.778 |
| CD300A | VNN2 | 0.823 | CGAS | STC1 | 0.791 | RALGAPA2 | FCGR2B | 0.795 |
| C2CD2 | SLC16A6 | 0.761 | CYTH4 | RGS16 | 0.752 | DSP | IL1RN | 0.804 |
| PRR16 | CXCR4 | 0.780 | GNAI2 | VNN2 | 0.768 | CHI3L2 | CYP26B1 | 0.798 |
| AGFG2 | ABL2 | 0.778 | CGAS | C1R | 0.750 | LCP2 | CCL2 | 0.766 |
| GPR137B | CD14 | 0.771 | STAP2 | LILRA2 | 0.762 | PKIB | DUSP4 | 0.766 |
| PRR16 | SPOCK1 | 0.795 | BCL2A1 | CLMP | 0.763 | DUSP2 | CD53 | 0.735 |
| DSP | TNC | 0.819 | CD93 | GMFG | 0.797 | HIBCH | NDFIP2 | 0.782 |
| SHTN1 | CLMP | 0.783 | FGF7 | SPOCK1 | 0.787 | CSF3R | CD86 | 0.746 |
| UBAC1 | ITGAM | 0.778 | ITGA5 | PHLDB2 | 0.780 | TMEM106C | PAPPA | 0.832 |
| VASN | MSN | 0.735 | PRICKLE2 | IL13RA2 | 0.864 | C2CD2 | TREM1 | 0.772 |
| GALNT12 | SH2D3C | 0.793 | CHI3L2 | ADGRG3 | 0.762 | FERMT2 | ITGAM | 0.759 |
| CBX6 | SLA | 0.720 | ATP5IF1 | KCNJ15 | 0.758 | SHTN1 | CTHRC1 | 0.794 |
| CHST15 | VIM | 0.780 | RAI14 | PRR16 | 0.802 | FCGR3B | SPP1 | 0.782 |
| JAZF1 | PTPRC | 0.766 | NFE2 | PAPPA | 0.833 | CSF2RB | EGR3 | 0.736 |
| EFNA4 | DOK3 | 0.755 | SLC15A4 | CXCR4 | 0.748 | CHST11 | PTGS1 | 0.767 |
| CSNK1G3 | TRAK1 | 0.756 | CRIP2 | DPYSL2 | 0.760 | LCP2 | VNN2 | 0.777 |
| TMEM106C | SRGN | 0.793 | LY96 | ASAP1 | 0.792 | HSD11B2 | TUBA1A | 0.769 |
| COL15A1 | TMEM273 | 0.761 | HSD11B2 | WNT5A | 0.801 | NDUFA10 | SORT1 | 0.809 |
| MMP3 | ZDHHC18 | 0.793 | TCF4 | ST3GAL5 | 0.756 | UBAC1 | JAZF1 | 0.802 |
| ASB7 | FZD5 | 0.768 | OLFM1 | CHI3L2 | 0.780 | CD93 | MSN | 0.790 |
| COX10 | CTHRC1 | 0.786 | CD86 | MAP4K4 | 0.787 | EFNA4 | TNC | 0.799 |
| PPP1R18 | PRR16 | 0.798 | SHTN1 | TLR4 | 0.731 | DPYSL2 | CHST2 | 0.775 |
| ABL2 | S100A9 | 0.828 | BASP1 | BPGM | 0.781 | BDH1 | GPR137B | 0.801 |
| PRICKLE2 | ST3GAL5 | 0.787 | SH2D3C | LILRA5 | 0.767 | FILIP1L | CD300A | 0.820 |
| PKIB | GPR137B | 0.795 | CREM | PDLIM4 | 0.789 | ITGA5 | PTPRC | 0.779 |
| CXCR1 | SLC16A6 | 0.710 | CXCR1 | MX2 | 0.718 | S100A8 | SPOCK1 | 0.817 |
| SELENBP1 | CCDC69 | 0.778 | CSTA | TIMP1 | 0.740 | FCGR2B | JAZF1 | 0.782 |
| CCDC3 | ARHGAP25 | 0.764 | TAGAP | VAV1 | 0.684 | CMTM8 | SLC2A3 | 0.773 |
| RAB15 | NFE2 | 0.785 | CREM | PCBP3 | 0.808 | C11orf96 | PDLIM4 | 0.780 |
| GPR176 | CD53 | 0.717 | TMEM71 | BMERB1 | 0.837 | EGR3 | RGS16 | 0.758 |
| CYP26B1 | TNFAIP6 | 0.801 | DPYSL3 | TNFAIP2 | 0.752 | COX10 | CHST15 | 0.794 |
| B4GALT4 | PTGS1 | 0.787 | LST1 | CREB5 | 0.782 | GLIPR1 | CLEC4A | 0.723 |
| AGFG2 | LOXL2 | 0.785 | LCP1 | S100A12 | 0.757 | ENO2 | BCL2A1 | 0.782 |
| CTHRC1 | COL7A1 | 0.788 | COL7A1 | SERPINB9 | 0.786 | ADCY6 | SLC2A3 | 0.782 |
| TIMP1 | ARHGAP25 | 0.775 | NIBAN1 | BTG2 | 0.783 | FAP | MSN | 0.760 |
| S1PR3 | OSMR | 0.778 | MIR100HG | MMP3 | 0.790 | CLEC4A | PRR16 | 0.809 |
| CEBPB | MSN | 0.778 | PPP1R18 | LCP2 | 0.753 | ADGRG3 | NFE2 | 0.739 |
| POLDIP2 | CREM | 0.788 | DUSP4 | ANGPT2 | 0.776 | RGS19 | CR1 | 0.698 |
| WNK2 | DUSP2 | 0.814 | DUSP4 | THEMIS2 | 0.790 | RILPL2 | LGALS1 | 0.798 |
| RFLNB | LCP1 | 0.745 | TLR1 | DOK3 | 0.753 | PTGS2 | GPR65 | 0.766 |
| CTHRC1 | P2RY14 | 0.794 | ZDHHC18 | SH3BP5 | 0.768 | SORT1 | SELL | 0.800 |
| FCGR3B | THBD | 0.819 | CMTM8 | HS3ST3B1 | 0.779 | AGFG2 | LILRA5 | 0.793 |
| IL1B | LCP1 | 0.763 | LILRA2 | SLC2A3 | 0.741 | FZD5 | FCGR3B | 0.806 |
| HIBCH | C11orf96 | 0.796 | S1PR1 | MSN | 0.786 | FLT1 | TCF4 | 0.759 |
| ABL2 | RGS16 | 0.755 | GLIPR1 | CBX6 | 0.684 | GLCE | CHI3L1 | 0.800 |
| CTHRC1 | C5AR1 | 0.783 | HSD11B2 | SELE | 0.810 | OLFM1 | SH3BP5 | 0.764 |
| HSD11B2 | RGS19 | 0.749 | PPP2R5A | LY96 | 0.808 | PTGS1 | BPGM | 0.771 |
| PKIB | MIR100HG | 0.771 | AGAP1 | CGAS | 0.767 | GALNT12 | CAVIN1 | 0.780 |
| S1PR3 | LILRA5 | 0.786 | PPP1R18 | LILRA5 | 0.776 | CCR1 | PDLIM4 | 0.800 |
| DLC1 | PCBP3 | 0.805 | MMP19 | TNFAIP2 | 0.761 | CSNK1G3 | CCN4 | 0.771 |
| CDX2 | TLR4 | 0.733 | POLDIP2 | ANGPTL2 | 0.775 | CCR1 | CCL4 | 0.785 |
| SELENBP1 | TMEM273 | 0.808 | CR1 | IER5 | 0.700 | ENO2 | CXCL8 | 0.793 |
| ASAP1 | CD69 | 0.757 | ZDHHC23 | GPR137B | 0.817 | S1PR1 | SPP1 | 0.754 |
| ATP5IF1 | PXDN | 0.794 | PTGS1 | RBMS1 | 0.796 | COX10 | IL13RA2 | 0.865 |
| NIPSNAP3A | CEBPB | 0.808 | POLDIP2 | TIMP1 | 0.766 | IL1RN | BTG2 | 0.775 |
| RAB31 | CHST15 | 0.784 | COQ9 | STC1 | 0.800 | CRIP2 | DPYSL3 | 0.765 |
| DUSP2 | C5AR1 | 0.775 | STAP2 | NCF2 | 0.775 | B4GALT4 | MYO1F | 0.773 |
| SERPINB9 | SPOCK1 | 0.766 | ADCY6 | OLFM1 | 0.775 | RGS16 | THEMIS2 | 0.773 |
| CAVIN3 | GZMB | 0.786 | ICAM1 | TNFAIP2 | 0.736 | IER5 | S100A12 | 0.784 |
| CHI3L2 | FLT1 | 0.761 | SORT1 | HLX | 0.751 | FCGR3B | CXCR4 | 0.793 |
| RAB15 | LCP1 | 0.762 | SLA | TCF4 | 0.742 | LILRA5 | PROK2 | 0.735 |
| UQCRC1 | TCF4 | 0.764 | PDPN | IL1RN | 0.801 | TAGAP | CRIP2 | 0.709 |
| CTHRC1 | CCR1 | 0.793 | LCP1 | WWC2 | 0.760 | COL15A1 | NRCAM | 0.766 |
| IL11 | G0S2 | 0.832 | DOK3 | GFPT2 | 0.718 | NDFIP2 | SLC2A3 | 0.771 |
| B4GALT4 | TMEM154 | 0.793 | TMEM71 | PRR16 | 0.809 | BASP1 | ANKRD35 | 0.784 |
| CLEC4E | PTGS1 | 0.761 | RIPOR3 | ACOT9 | 0.813 | TRAK1 | HLX | 0.776 |
| IFITM2 | CHN1 | 0.788 | VAV1 | SSH2 | 0.716 | FLT1 | NFE2 | 0.766 |
| LILRB1 | HCK | 0.759 | AQP9 | SLA | 0.755 | FAP | CEP19 | 0.693 |
| LILRB1 | DPYSL3 | 0.778 | MYO1F | RBMS1 | 0.739 | RHOH | NCF4 | 0.725 |
| NEO1 | CREB5 | 0.798 | IL13RA2 | GADD45B | 0.864 | LOXL2 | BCL6 | 0.710 |
| GAS2L3 | CCL2 | 0.773 | SLC11A1 | ZDHHC18 | 0.776 | WNK2 | ZDHHC18 | 0.826 |
| TRPM6 | FAP | 0.774 | SRGN | CHST2 | 0.788 | DSP | P2RY13 | 0.782 |
| FPR1 | GPR65 | 0.742 | ZNF704 | LOXL2 | 0.795 | RNF149 | C5AR1 | 0.782 |
| RGCC | BPGM | 0.785 | ARPIN | SLA | 0.746 | LGALS1 | BCL6 | 0.698 |
| SRGN | SPHK1 | 0.779 | LOXL2 | ZDHHC18 | 0.777 | IL6 | CXCL8 | 0.805 |
| DUSP4 | SERPINB9 | 0.776 | EMP3 | SWAP70 | 0.731 | S1PR1 | FAM49A | 0.759 |
| LILRB2 | CLEC4A | 0.766 | MEFV | PLEK | 0.753 | SMAP2 | CD69 | 0.740 |
| MMP3 | SOD2 | 0.784 | GPR176 | CD86 | 0.749 | SLC15A4 | PRR16 | 0.820 |
| TAGAP | SELP | 0.766 | GLIPR1 | VNN2 | 0.749 | TREM1 | CXCR4 | 0.735 |
| CREM | LY96 | 0.788 | SWAP70 | PREX1 | 0.681 | LILRB1 | PTGS2 | 0.805 |
| CTHRC1 | MMP19 | 0.788 | JAZF1 | IL11 | 0.837 | PTGS2 | TNFAIP2 | 0.781 |
| DPYSL3 | JAZF1 | 0.754 | POLDIP2 | RGCC | 0.788 | CSTA | NRG1 | 0.786 |
| NCF2 | ACOD1 | 0.742 | G0S2 | PLA1A | 0.822 | OLFM1 | SPP1 | 0.777 |
| FCGR2B | CXCL8 | 0.814 | GAS2L3 | IL13RA2 | 0.870 | OLFM1 | SELE | 0.791 |
| TRAK1 | DPYSL3 | 0.763 | CMTM8 | MSN | 0.761 | S1PR1 | RHOH | 0.719 |
| FPR1 | BMERB1 | 0.786 | TRPM6 | STC1 | 0.826 | STAT5B | ACOD1 | 0.761 |
| LRRK2 | CHST15 | 0.790 | PRRX1 | PROK2 | 0.731 | FLT1 | S100A9 | 0.812 |
| NCF2 | SH3BP5 | 0.736 | P2RY13 | CEMIP | 0.775 | HIBCH | RNF149 | 0.805 |
| KIAA1522 | LAT2 | 0.778 | TREM1 | GPR137B | 0.771 | PPP2R5A | BTG2 | 0.768 |
| LRP4 | MIR100HG | 0.774 | NIPSNAP3A | TUBB6 | 0.798 | CAVIN3 | FGF2 | 0.788 |
| AFG3L2 | RFLNB | 0.781 | FGF7 | TUBB6 | 0.783 | NNMT | VAV1 | 0.780 |
| DLC1 | PTPRC | 0.801 | LCP1 | CXCL5 | 0.742 | TRPM6 | POLDIP2 | 0.777 |
| LAT2 | CD14 | 0.759 | CSNK1G3 | SLC2A3 | 0.773 | AGFG2 | CSTA | 0.758 |
| IER5 | SLA | 0.728 | COTL1 | PXDN | 0.784 | TRAK1 | POLDIP2 | 0.792 |
| TRIM2 | CYP26B1 | 0.831 | CHST11 | SERPINB9 | 0.740 | PLEK | CXCR4 | 0.719 |
| TRAK1 | GZMB | 0.781 | NR3C2 | CHRDL2 | 0.793 | CEP19 | PHLDB2 | 0.714 |
| TSPAN6 | FPR2 | 0.786 | HIBCH | JAZF1 | 0.777 | ASB7 | PRRX1 | 0.783 |
| F5 | SLC2A3 | 0.752 | AQP9 | NCF2 | 0.757 | SNORC | SELENBP1 | 0.774 |
| MYO1F | CEMIP | 0.780 | CHI3L2 | LY96 | 0.758 | ASB7 | CLEC4A | 0.768 |
| AFG3L2 | ZDHHC23 | 0.805 | SERPINB9 | P2RY13 | 0.772 | SELENBP1 | DOK3 | 0.785 |
| CAVIN1 | MX2 | 0.765 | S1PR1 | EGR3 | 0.737 | LILRA2 | LRRK2 | 0.761 |
| GLCE | TNC | 0.812 | ENO2 | HCLS1 | 0.725 | NCF2 | SPOCK1 | 0.762 |
| LY96 | STAT5B | 0.782 | GLCE | PXDN | 0.809 | DSP | C11orf96 | 0.793 |
| ADGRG3 | PLA1A | 0.773 | IL24 | ITPRIP | 0.816 | CDX2 | FZD5 | 0.775 |
| RFLNB | PAPPA | 0.821 | S100A9 | STAT5B | 0.823 | DSP | EGR3 | 0.775 |
| FZD5 | CCDC69 | 0.768 | GPR176 | RHOH | 0.740 | IFITM2 | FGF7 | 0.778 |
| COQ9 | PREX1 | 0.774 | CTHRC1 | IL13RA2 | 0.861 | SELENBP1 | CD93 | 0.810 |
| POLDIP2 | RNF149 | 0.783 | TLR1 | CCDC3 | 0.792 | RNF149 | SNN | 0.817 |
| TAGAP | BCL2A1 | 0.709 | HCLS1 | BMERB1 | 0.749 | CHI3L2 | NRG1 | 0.785 |
| EGR3 | MEFV | 0.745 | CCDC69 | STC1 | 0.801 | FLT1 | IER5 | 0.755 |
| NIBAN1 | IL11 | 0.830 | SH2D3C | NRCAM | 0.753 | TRPM6 | S100A8 | 0.803 |
| POLDIP2 | COL4A2 | 0.785 | NR3C2 | SELP | 0.805 | TRIM2 | LILRA2 | 0.786 |
| CSF3R | SNN | 0.750 | CR1 | TLR1 | 0.754 | CLEC4A | CHST15 | 0.794 |
| STAP2 | TAGAP | 0.750 | DSP | RASSF5 | 0.780 | CMTM4 | FERMT2 | 0.795 |
| CGAS | SOD2 | 0.758 | RBMS1 | CEP19 | 0.707 | RGS19 | MX2 | 0.711 |
| MSN | MYO1F | 0.773 | WWC2 | THEMIS2 | 0.785 | MYO1F | CCDC3 | 0.774 |
| ZNF704 | CSF3R | 0.734 | CR1 | RIPOR3 | 0.770 | COQ9 | LILRB2 | 0.823 |
| NDUFA10 | ZNF704 | 0.779 | RFLNB | CCL2 | 0.761 | NEO1 | PCBP3 | 0.773 |
| CCR1 | RIPOR2 | 0.784 | SH2D3C | PI15 | 0.771 | PDLIM4 | BMERB1 | 0.819 |
| ARPIN | IL11 | 0.824 | RAB15 | TNFRSF1B | 0.768 | HCLS1 | PDLIM4 | 0.735 |
| G0S2 | CXCR4 | 0.781 | SH3BP5 | GFPT2 | 0.749 | RHOH | PHLDB2 | 0.751 |
| PRR16 | SLC35G2 | 0.803 | IL1RN | TUBA1A | 0.791 | CCR1 | COL4A2 | 0.774 |
| NIPSNAP3A | ABL2 | 0.804 | FFAR2 | ST3GAL5 | 0.758 | F5 | IL13RA2 | 0.868 |
| MYO1D | LILRB3 | 0.774 | CMTM8 | NEO1 | 0.805 | CXCR4 | SH3BP5 | 0.707 |
| NEO1 | GPR176 | 0.771 | CR1 | EGR3 | 0.709 | GLT1D1 | DPYSL3 | 0.755 |
| NFE2 | TIMP1 | 0.786 | INHBA | FAM49A | 0.790 | NDFIP2 | FPR2 | 0.801 |
| COX10 | CXCL5 | 0.758 | CLEC4A | TUBB6 | 0.805 | CSNK1G3 | SNN | 0.762 |
| PLAU | TCF4 | 0.752 | PKIB | RIPK2 | 0.788 | UBAC1 | GFPT2 | 0.757 |
| RAB31 | CREB5 | 0.770 | CMTM8 | SLC35G2 | 0.812 | CMTM8 | PLEK | 0.763 |
| PTGS1 | RIPOR2 | 0.783 | RAB15 | CSTA | 0.768 | CLEC4E | SSH2 | 0.718 |
| TAGAP | LGALS1 | 0.722 | NIBAN1 | IL6 | 0.816 | TCEA3 | C5AR1 | 0.805 |
| CAVIN1 | S100A8 | 0.812 | RALGAPA2 | ITGA5 | 0.785 | BASP1 | FILIP1L | 0.794 |
| FILIP1L | COL7A1 | 0.805 | OSM | SPP1 | 0.786 | CDX2 | VIM | 0.785 |
| PDLIM4 | MAP4K4 | 0.784 | MIR100HG | VIM | 0.734 | SWAP70 | SPP1 | 0.704 |
| TRAK1 | CYTH4 | 0.758 | NDUFA10 | MMP3 | 0.786 | NIPSNAP3A | HIBCH | 0.764 |
| FCGR3B | CXCR1 | 0.772 | PIK3AP1 | DUSP4 | 0.755 | RALGAPA2 | SLC16A6 | 0.763 |
| HLX | NCF2 | 0.731 | RALGAPA2 | GPR176 | 0.759 | ZNF704 | SPP1 | 0.762 |
| AGAP1 | MEFV | 0.779 | CCR1 | PTGS2 | 0.795 | GADD45B | VAV1 | 0.759 |
| STAP2 | CSF3R | 0.753 | CHI3L2 | LCP1 | 0.714 | NCF4 | ACOD1 | 0.763 |
| ARPIN | CSTA | 0.741 | ACOT9 | TNFRSF1B | 0.772 | RALGAPA2 | SPP1 | 0.762 |
| PRICKLE2 | HCLS1 | 0.747 | SELENBP1 | SWAP70 | 0.768 | COQ9 | DPYSL3 | 0.771 |
| DPYSL3 | MSN | 0.765 | COL4A2 | SLC2A3 | 0.723 | F5 | ZDHHC18 | 0.784 |
| ENO2 | RASSF5 | 0.778 | CCR1 | AQP9 | 0.785 | NDUFA10 | PRR16 | 0.821 |
| G0S2 | PTGS2 | 0.796 | BCL6 | TNF | 0.703 | MYO1F | PROK2 | 0.722 |
| TUBB6 | HS3ST3B1 | 0.798 | RAI14 | CD69 | 0.753 | IL24 | MSN | 0.800 |
| ASB7 | CXCR2 | 0.773 | AKAP1 | PTPRC | 0.813 | CCR1 | HCLS1 | 0.741 |
| ZNF704 | NNMT | 0.801 | S100A8 | FAM49A | 0.827 | TNF | CD53 | 0.746 |
| CTHRC1 | ST3GAL5 | 0.790 | MIR100HG | CHST2 | 0.751 | S1PR3 | SERPINE2 | 0.781 |
| CRIP2 | SMAP2 | 0.751 | AGAP1 | NRG1 | 0.791 | ASB7 | GLT1D1 | 0.710 |
| RNF149 | NOD2 | 0.735 | CEMIP | CCL4 | 0.777 | LDLRAD3 | GZMB | 0.794 |
| RIPOR3 | F5 | 0.826 | SH2D3C | ITGA5 | 0.765 | B4GALT4 | RHOH | 0.766 |
| AKAP1 | CSF2RB | 0.810 | TNFRSF1B | SLC16A6 | 0.708 | F5 | MMP3 | 0.799 |
| TSPAN6 | S1PR1 | 0.806 | PPP2R5A | GPR137B | 0.794 | HSD11B2 | ADGRG3 | 0.768 |
| CLEC4E | SELL | 0.788 | COQ9 | TCF4 | 0.754 | ABL2 | G0S2 | 0.812 |
| FAP | BPGM | 0.748 | FILIP1L | CSF3R | 0.745 | CAVIN3 | CXCR4 | 0.737 |
| CRIP2 | MX2 | 0.779 | MIR100HG | CCN4 | 0.797 | ZNF704 | VIM | 0.783 |
| CREM | GFPT2 | 0.760 | ITGA5 | CCL4 | 0.775 | CSTA | SLA | 0.705 |
| TMEM154 | NOD2 | 0.719 | RALGAPA2 | SHTN1 | 0.788 | ZNF704 | TSPAN6 | 0.799 |
| CSNK1G3 | LAT2 | 0.762 | COX10 | ATP5IF1 | 0.772 | EMP3 | FAM49A | 0.766 |
| NEO1 | GFPT2 | 0.772 | CSF2RB | IL11 | 0.828 | LILRB2 | DPYSL2 | 0.799 |
| TMEM71 | SMAP2 | 0.769 | GNAI2 | SLFN11 | 0.729 | COQ9 | MYO1F | 0.787 |
| ARPIN | CLEC4E | 0.771 | HCK | CXCR2 | 0.791 | FLT1 | STAT5B | 0.768 |
| S1PR1 | PXDN | 0.782 | FLT1 | GPR137B | 0.782 | MYO1D | PIK3AP1 | 0.722 |
| VASN | ENTPD1 | 0.759 | AQP9 | S100A8 | 0.784 | MYO1D | LGALS1 | 0.785 |
| TNC | MX2 | 0.797 | RALGAPA2 | C1R | 0.777 | RILPL2 | BTG2 | 0.794 |
| POLDIP2 | RGS16 | 0.780 | F5 | BTG2 | 0.793 | CSF3R | FAM20C | 0.728 |
| FFAR2 | ITGA5 | 0.783 | FGF7 | CEMIP | 0.799 | COL15A1 | CXCR1 | 0.733 |
| ENO2 | PREX1 | 0.770 | PRR16 | SRGN | 0.813 | MMP3 | RIPOR2 | 0.775 |
| NEO1 | CCL2 | 0.777 | S1PR3 | SPOCK1 | 0.774 | GALNT12 | ACOD1 | 0.777 |
| FCGR2B | LILRA5 | 0.789 | CXCL8 | ITPRIP | 0.805 | MCAM | GPR137B | 0.785 |
| POLDIP2 | OCRL | 0.792 | ARPIN | PLA1A | 0.779 | PFKL | NFE2 | 0.775 |
| PI15 | BCL6 | 0.714 | MMP19 | S100A9 | 0.804 | COTL1 | CD53 | 0.735 |
| TAGAP | SELE | 0.764 | SELL | RIPK2 | 0.793 | BASP1 | THBD | 0.801 |
| COL4A2 | CCDC3 | 0.763 | GADD45B | VIM | 0.765 | RAI14 | GMFG | 0.790 |
| LRP4 | ICAM1 | 0.770 | TAGAP | ENO2 | 0.728 | CEBPB | C2CD2 | 0.788 |
| FILIP1L | S100A9 | 0.842 | RALGAPA2 | MYO1F | 0.779 | FLT1 | CXCR1 | 0.737 |
| LILRB2 | PLA1A | 0.777 | CDX2 | DOK3 | 0.771 | ANGPTL2 | GMFG | 0.771 |
| FCGR3B | STAT5B | 0.802 | COX10 | TCF4 | 0.774 | TAGAP | BTG2 | 0.737 |
| HSD11B2 | G0S2 | 0.827 | CR1 | CBX6 | 0.715 | DLC1 | TLR1 | 0.809 |
| AMPD2 | CD53 | 0.746 | KIAA1522 | FGF2 | 0.785 | POLDIP2 | SOD2 | 0.782 |
| FERMT2 | CLEC4A | 0.802 | AGFG2 | RHOH | 0.762 | MSN | PDLIM4 | 0.774 |
| ARPIN | ZDHHC18 | 0.763 | AGAP1 | KCNJ15 | 0.729 | GNAI2 | TIMP1 | 0.760 |
| TMEM71 | CREM | 0.777 | SERPINE2 | FAM20C | 0.785 | PPP1R18 | VIM | 0.754 |
| ABL2 | STC1 | 0.796 | ANKRD35 | TREM1 | 0.795 | BCL6 | GPR137B | 0.716 |
| CXCR4 | ENTPD1 | 0.743 | PFKL | PKIB | 0.770 | CYP26B1 | BMERB1 | 0.812 |
| ABL2 | S100A8 | 0.829 | CSF3 | CEP19 | 0.754 | PKIB | NFAM1 | 0.764 |
| CSF3R | RILPL2 | 0.747 | SHTN1 | UQCRC1 | 0.788 | IFITM2 | VIM | 0.771 |
| CRIP2 | HS3ST3B1 | 0.779 | BTG2 | SLC35G2 | 0.803 | GNAI2 | S100A12 | 0.769 |
| PLEK | THBD | 0.793 | CHI3L1 | GMFG | 0.803 | CHN1 | CLEC4A | 0.801 |
| CLU | FPR1 | 0.790 | TLR4 | TNFRSF10C | 0.684 | ACOD1 | PDLIM4 | 0.786 |
| GLCE | BCL6 | 0.720 | ANGPT2 | CXCL5 | 0.773 | TCEA3 | RIPK2 | 0.789 |
| HSD11B2 | THEMIS2 | 0.790 | COL15A1 | PDLIM4 | 0.778 | MX2 | SLC35G2 | 0.803 |
| ZNF704 | RGS16 | 0.793 | PDPN | CREB5 | 0.783 | CXCR2 | TNFRSF10C | 0.745 |
| MEFV | CYP26B1 | 0.813 | RGS19 | CCDC3 | 0.729 | FCGR3B | CHST15 | 0.807 |
| FERMT2 | LCP1 | 0.752 | GADD45B | VNN2 | 0.820 | AFG3L2 | GLT1D1 | 0.763 |
| LILRB1 | ARHGAP25 | 0.766 | CSF3R | ANGPTL2 | 0.735 | CHRDL2 | SPHK1 | 0.760 |
| CREM | ANGPTL2 | 0.778 | CSTA | TMEM273 | 0.732 | BCL2A1 | CHST2 | 0.770 |
| CSTA | IL1B | 0.778 | RGCC | PRRX1 | 0.758 | HCK | ITPRIP | 0.786 |
| IL11 | SPOCK1 | 0.841 | SWAP70 | SELP | 0.750 | PRICKLE2 | STAT5B | 0.808 |
| GLT1D1 | SH3BP5 | 0.722 | CXCR2 | CHST11 | 0.763 | RALGAPA2 | S100A12 | 0.796 |
| FLT1 | PLA1A | 0.772 | SLC35G2 | GFPT2 | 0.776 | SOD2 | STC1 | 0.803 |
| S1PR1 | FAP | 0.764 | GPR183 | CCL4 | 0.758 | ANKRD35 | RNF149 | 0.794 |
| GPR183 | CXCR2 | 0.794 | HCK | PIP4P2 | 0.772 | NIPSNAP3A | AQP9 | 0.806 |
| PKIB | BTG2 | 0.771 | SELENBP1 | P2RY13 | 0.819 | HSD11B2 | GALNT12 | 0.748 |
| FCGR3B | P2RY14 | 0.830 | CCL2 | CXCL5 | 0.795 | GLIPR1 | PPP1R18 | 0.732 |
| NR3C2 | IL1RN | 0.791 | STC1 | SLFN11 | 0.788 | LDLRAD3 | VIM | 0.779 |
| SELL | SNN | 0.796 | ADCY6 | LY96 | 0.799 | FERMT2 | PDLIM4 | 0.773 |
| MYO1D | FGF7 | 0.785 | FCGR3B | RBMS1 | 0.832 | RGCC | INHBA | 0.791 |
| NIBAN1 | FFAR2 | 0.737 | SPP1 | ARHGAP25 | 0.751 | SLC15A3 | CCN4 | 0.757 |
| NDUFA10 | PPP1R18 | 0.773 | NDFIP2 | CLU | 0.766 | BICD2 | PTPRC | 0.731 |
| CLU | IL13RA2 | 0.858 | SMAP2 | WWC2 | 0.744 | CXCR2 | CD86 | 0.816 |
| LCP1 | PI15 | 0.755 | TCF4 | CCDC3 | 0.732 | AMPD2 | PRRX1 | 0.780 |
| ANGPT2 | C5AR1 | 0.784 | FERMT2 | FAM49A | 0.779 | CEMIP | SELP | 0.816 |
| CTHRC1 | CSF2RB | 0.793 | FLT1 | VIM | 0.757 | ASB7 | NDFIP2 | 0.777 |
| CCR1 | LDLRAD3 | 0.791 | TMEM154 | BTG2 | 0.782 | RFLNB | SLC2A3 | 0.761 |
| S100A8 | ZDHHC18 | 0.803 | FILIP1L | CXCL5 | 0.774 | GOLPH3L | CCN4 | 0.787 |
| IL1B | STC1 | 0.803 | EFNA4 | F5 | 0.791 | ICAM1 | SELP | 0.804 |
| ANGPT2 | SLC11A1 | 0.809 | VASN | ENO2 | 0.736 | MYO1D | TSPAN6 | 0.783 |
| TRAK1 | TNF | 0.795 | RNF149 | MEFV | 0.765 | LILRB1 | SWAP70 | 0.737 |
| CSNK1G3 | SERPINE2 | 0.772 | DUSP2 | FGR | 0.780 | TMEM71 | LDLRAD3 | 0.794 |
| TIMP1 | CD53 | 0.746 | OCRL | MMP19 | 0.775 | UQCRC1 | CCL4 | 0.784 |
| ARPIN | SWAP70 | 0.711 | SORT1 | LCP1 | 0.748 | SLC15A4 | MIR100HG | 0.767 |
| CSNK1G3 | PLEK | 0.763 | SORT1 | WNK2 | 0.815 | TREM1 | BPGM | 0.777 |
| FFAR2 | PLA1A | 0.774 | CR1 | GZMB | 0.778 | COQ9 | SOD2 | 0.786 |
| ANGPTL2 | CHST15 | 0.773 | GALNT12 | COL7A1 | 0.813 | MMP19 | VIM | 0.765 |
| C11orf96 | CLEC4A | 0.789 | MIR100HG | NRCAM | 0.771 | CDX2 | SPHK1 | 0.781 |
| PPP1R18 | BPGM | 0.749 | LILRA2 | CAVIN3 | 0.760 | CCR1 | PLAU | 0.788 |
| VASN | IER5 | 0.708 | NNMT | MAP4K4 | 0.788 | PIK3AP1 | CCN4 | 0.746 |
| CCDC69 | CCDC88A | 0.729 | CSF3R | GLT1D1 | 0.687 | C2CD2 | HS3ST3B1 | 0.778 |
| COL4A2 | FPR2 | 0.793 | IFITM2 | IL11 | 0.819 | JAZF1 | WNT5A | 0.799 |
| GPR176 | OSM | 0.806 | OCRL | CXCR4 | 0.722 | COTL1 | SELE | 0.807 |
| SOD2 | ZDHHC18 | 0.777 | TNC | PREX1 | 0.783 | GALNT12 | CCR1 | 0.807 |
| UBAC1 | PAPPA | 0.837 | CD300A | PIP4P2 | 0.808 | RFLNB | SPP1 | 0.752 |
| NRG1 | ENTPD1 | 0.806 | GLT1D1 | PTPRC | 0.731 | TMEM106C | NRG1 | 0.780 |
| NFAM1 | DUSP4 | 0.745 | CSTA | C5AR1 | 0.749 | TMEM106C | COL7A1 | 0.806 |
| ASAP1 | LST1 | 0.777 | NNMT | SELE | 0.802 | LDLRAD3 | CCN4 | 0.779 |
| IL24 | LY96 | 0.806 | RAB15 | WNK2 | 0.796 | COL15A1 | LDLRAD3 | 0.756 |
| CGAS | HCK | 0.744 | LGALS1 | CD69 | 0.755 | IL1B | ITGA5 | 0.786 |
| UBAC1 | CCL4 | 0.784 | RBMS1 | FAM155A | 0.794 | AGAP1 | PDE4B | 0.772 |
| EGR3 | WWC2 | 0.722 | RALGAPA2 | RGCC | 0.789 | LGALS1 | SLFN11 | 0.725 |
| CR1 | ENTPD1 | 0.773 | LILRB1 | HLX | 0.755 | B4GALT4 | CHI3L1 | 0.788 |
| LRP4 | AQP9 | 0.785 | CMTM8 | SERPINB9 | 0.776 | PFKL | PLEK | 0.778 |
| BASP1 | SLC2A3 | 0.764 | NEO1 | PLA1A | 0.790 | STAP2 | SLC15A4 | 0.772 |
| TMEM273 | MIR100HG | 0.744 | AQP9 | CYP26B1 | 0.795 | FCER1G | PRR16 | 0.793 |
| ADGRG3 | SLC2A3 | 0.727 | PPP2R5A | B4GALT4 | 0.755 | PTGS2 | SSH2 | 0.770 |
| COL4A2 | MSN | 0.771 | S100A9 | WNT5A | 0.823 | RALGAPA2 | CCL2 | 0.767 |
| RAB15 | MCAM | 0.793 | RFLNB | FAM20C | 0.766 | COQ9 | FPR1 | 0.798 |
| FILIP1L | DUSP4 | 0.799 | ITGA5 | TNF | 0.789 | ARPIN | TMEM71 | 0.804 |
| UQCRC1 | DUSP2 | 0.785 | RIPOR2 | GFPT2 | 0.745 | GPR176 | SNN | 0.764 |
| RGS19 | CEP19 | 0.675 | PPP2R5A | FCGR2B | 0.795 | TMEM273 | FPR1 | 0.777 |
| GALNT12 | PPP1R18 | 0.760 | ENO2 | BPGM | 0.804 | MMP19 | CCL4 | 0.778 |
| TCEA3 | GLT1D1 | 0.761 | ADCY6 | CHRDL2 | 0.798 | LILRB2 | MSN | 0.812 |
| ITGA5 | TNFAIP6 | 0.780 | TMEM71 | CSTA | 0.752 | MEFV | BCL2A1 | 0.759 |
| AKAP1 | NCF4 | 0.753 | ABL2 | BCL6 | 0.696 | HSD11B2 | LCP2 | 0.778 |
| ACOT9 | TUBA1A | 0.791 | OLFM1 | MMP3 | 0.802 | PDE4B | VAV1 | 0.708 |
| ERMP1 | ARHGAP25 | 0.777 | CSF3 | ICAM1 | 0.762 | NIPSNAP3A | C5AR1 | 0.826 |
| LILRA2 | OSMR | 0.792 | PDPN | SLC15A4 | 0.806 | OCRL | LGALS1 | 0.794 |
| RALGAPA2 | FPR1 | 0.784 | FZD5 | NRG1 | 0.797 | GZMB | CHST15 | 0.804 |
| RILPL2 | SPHK1 | 0.779 | FZD5 | GMFG | 0.772 | ABL2 | RIPOR2 | 0.754 |
| GALNT12 | PTPRC | 0.789 | NOD2 | SNN | 0.732 | FAM20C | PREX1 | 0.763 |
| LDLRAD3 | TLR1 | 0.792 | ADCY6 | SH2D3C | 0.786 | CBX6 | PLAU | 0.759 |
| ACOT9 | HSPA6 | 0.750 | HLX | ST3GAL5 | 0.751 | CAVIN1 | PTGS1 | 0.770 |
| RALGAPA2 | RFLNB | 0.808 | AGAP1 | SH3RF2 | 0.774 | TMEM106C | TMEM154 | 0.788 |
| EFNA4 | RASSF5 | 0.782 | C2CD2 | HSPA6 | 0.760 | TMEM106C | VIM | 0.765 |
| DPYSL2 | TIMP1 | 0.770 | CSF2RB | CEP19 | 0.723 | TSPAN6 | SLA | 0.778 |
| IL24 | IER5 | 0.797 | OLFM1 | PRRX1 | 0.771 | ANGPT2 | SPHK1 | 0.787 |
| DSP | IL1B | 0.800 | NEO1 | ANGPT2 | 0.832 | AQP9 | CD69 | 0.788 |
| CHI3L1 | FILIP1L | 0.783 | ZNF704 | CD86 | 0.796 | CHRDL2 | LGALS1 | 0.774 |
| PRICKLE2 | TNFAIP6 | 0.796 | ENO2 | CHST2 | 0.779 | GAS2L3 | SERPINE2 | 0.777 |
| S100A12 | ARHGAP25 | 0.784 | LGALS1 | ARHGAP25 | 0.763 | SH3RF2 | VIM | 0.787 |
| PIK3AP1 | RNF149 | 0.732 | COQ9 | OSM | 0.804 | NIBAN1 | PIK3AP1 | 0.718 |
| BDH1 | DLC1 | 0.803 | UBAC1 | MIR100HG | 0.771 | NDUFA10 | FCGR2B | 0.784 |
| CD86 | SH3BP5 | 0.764 | CCR1 | KCNJ15 | 0.747 | EFNA4 | NRCAM | 0.769 |
| CAVIN3 | CAVIN1 | 0.759 | ZNF704 | SELP | 0.815 | FFAR2 | ZDHHC18 | 0.765 |
| IER5 | SLC11A1 | 0.769 | LY96 | CD86 | 0.792 | LCP2 | TNFRSF10C | 0.721 |
| CSTA | TLR1 | 0.735 | CHN1 | FCGR2B | 0.787 | TNC | TCF4 | 0.779 |
| FZD5 | CEP19 | 0.725 | KIAA1522 | S100A12 | 0.800 | F5 | PLEK | 0.784 |
| CXCL8 | ASAP1 | 0.785 | C2CD2 | GMFG | 0.781 | NDFIP2 | GPR183 | 0.775 |
| CAVIN1 | LAT2 | 0.779 | AKAP1 | GLIPR1 | 0.784 | POLDIP2 | ZNF704 | 0.786 |
| LST1 | THEMIS2 | 0.767 | LILRB1 | CCL4 | 0.789 | CMTM8 | CBX6 | 0.780 |
| DPYSL2 | CXCR4 | 0.706 | RGS19 | BTG2 | 0.724 | RALGAPA2 | RIPOR3 | 0.810 |
| LILRB2 | PIK3AP1 | 0.745 | PKIB | FERMT2 | 0.791 | FLT1 | C11orf96 | 0.787 |
| FCGR2B | ANGPTL2 | 0.774 | C2CD2 | CCL4 | 0.780 | IL1RN | MSN | 0.763 |
| OLFM1 | CHRDL2 | 0.781 | SLC15A3 | SLFN11 | 0.733 | CAVIN3 | ITPRIP | 0.791 |
| CR1 | PTGS2 | 0.776 | EFNA4 | OSMR | 0.778 | SNN | CD86 | 0.781 |
| DPYSL2 | ITGA5 | 0.765 | BDH1 | IL13RA2 | 0.862 | TMEM71 | DPYSL2 | 0.792 |
| CMTM4 | S100A9 | 0.826 | ZNF704 | DPYSL2 | 0.783 | AGAP1 | CXCR4 | 0.726 |
| NIPSNAP3A | CCL4 | 0.804 | S1PR3 | ACOD1 | 0.776 | VAV1 | CD69 | 0.699 |
| HLX | CD86 | 0.730 | PLAU | FAM155A | 0.781 | CYTH4 | ITPRIP | 0.787 |
| THEMIS2 | RIPOR2 | 0.723 | TREM1 | GMFG | 0.790 | SH3RF2 | MSN | 0.779 |
| HSD11B2 | SLC15A3 | 0.770 | LY96 | PDE4B | 0.770 | GALNT12 | SLFN11 | 0.782 |
| RAB15 | NR3C2 | 0.776 | CSF3 | FGR | 0.795 | HCK | CEP19 | 0.721 |
| AFG3L2 | ARPIN | 0.760 | LOXL2 | NRCAM | 0.766 | CHI3L2 | FGF7 | 0.782 |
| FZD5 | ITPRIP | 0.788 | TRAK1 | COL7A1 | 0.807 | SWAP70 | IL11 | 0.789 |
| GPR176 | NRG1 | 0.790 | MCAM | ENTPD1 | 0.760 | S100A9 | SNN | 0.846 |
| CTHRC1 | SLFN11 | 0.766 | CR1 | BTG2 | 0.738 | TCEA3 | IL24 | 0.809 |
| FLT1 | BCAT1 | 0.765 | DPYSL2 | ADGRG3 | 0.761 | CEBPB | INHBA | 0.791 |
| DUSP4 | CXCL5 | 0.790 | RALGAPA2 | TCF4 | 0.765 | TMEM106C | IER5 | 0.742 |
| DSP | SERPINB9 | 0.784 | RALGAPA2 | PRRX1 | 0.785 | RIPOR3 | PLEK | 0.790 |
| BASP1 | COL15A1 | 0.777 | ZNF704 | CYP26B1 | 0.822 | SWAP70 | C2CD2 | 0.722 |
| RGS19 | TNFAIP6 | 0.758 | IL24 | FPR1 | 0.806 | GLIPR1 | CXCR4 | 0.707 |
| RIPOR3 | PRRX1 | 0.787 | PDPN | ACOD1 | 0.777 | CSF3 | TNFAIP6 | 0.798 |
| ARPIN | SPOCK1 | 0.787 | AGFG2 | ZDHHC18 | 0.782 | TREM1 | PDLIM4 | 0.770 |
| WNK2 | PRR16 | 0.821 | GALNT12 | CXCL8 | 0.802 | IER5 | SLC35G2 | 0.778 |
| STAP2 | S100A12 | 0.795 | ADGRG3 | PHLDB2 | 0.786 | LST1 | RIPK2 | 0.778 |
| CSNK1G3 | P2RY13 | 0.788 | PDPN | PTGS1 | 0.790 | CLU | VIM | 0.755 |
| AGFG2 | CCL4 | 0.769 | ITGAM | SLC11A1 | 0.795 | KIAA1522 | SNN | 0.784 |
| IL6 | RBMS1 | 0.817 | SMAP2 | TNF | 0.770 | SLC15A4 | PREX1 | 0.777 |
| MEFV | CHST2 | 0.795 | ICAM1 | CLMP | 0.757 | C1R | ST3GAL5 | 0.761 |
| LCP1 | C5AR1 | 0.741 | TRPM6 | SELE | 0.815 | FILIP1L | BICD2 | 0.764 |
| FLT1 | SPOCK1 | 0.779 | HLX | CLEC4A | 0.729 | DSP | PXDN | 0.795 |
| CYTH4 | CD53 | 0.728 | BASP1 | ENO2 | 0.781 | GPR183 | P2RY13 | 0.763 |
| COX10 | GFPT2 | 0.765 | NR3C2 | LCP1 | 0.777 | WNT5A | TUBA1A | 0.806 |
| NEO1 | FPR1 | 0.784 | FJX1 | PDE4B | 0.740 | CXCL8 | BPGM | 0.780 |
| LRP4 | FCGR3B | 0.806 | VASN | RAI14 | 0.743 | TMEM71 | ABL2 | 0.797 |
| BICD2 | SOD2 | 0.777 | KIAA1522 | NOD2 | 0.745 | LRP4 | ASAP1 | 0.779 |
| ATP5IF1 | NRG1 | 0.794 | PLAU | GPR65 | 0.753 | LILRB2 | P2RY13 | 0.753 |
| EFNA4 | RNF149 | 0.785 | PLAU | GMFG | 0.778 | COX10 | CGAS | 0.780 |
| KIAA1522 | SPHK1 | 0.785 | LILRB2 | CHRDL2 | 0.813 | PPP2R5A | STAP2 | 0.770 |
| NCF4 | STAT5B | 0.741 | SLC15A3 | PIP4P2 | 0.756 | SH2D3C | MIR100HG | 0.746 |
| WNK2 | COTL1 | 0.802 | LDLRAD3 | CXCR2 | 0.793 | DUSP2 | ZDHHC18 | 0.751 |
| AQP9 | BTG2 | 0.784 | DPYSL2 | EMP3 | 0.773 | EFNA4 | CXCR4 | 0.733 |
| HCK | STAT5B | 0.742 | HSD11B2 | GZMB | 0.783 | CEMIP | TIMP1 | 0.769 |
| NIPSNAP3A | ENO2 | 0.775 | LCP1 | PLEK | 0.735 | PRICKLE2 | DOK3 | 0.767 |
| LRRK2 | ENTPD1 | 0.807 | PFKL | VNN2 | 0.818 | TRIM2 | IER5 | 0.762 |
| FZD5 | RIPK2 | 0.784 | RGS19 | DUSP2 | 0.707 | GALNT12 | PLA1A | 0.784 |
| FCER1G | ACOT9 | 0.791 | PXDN | SLFN11 | 0.742 | SLC15A3 | PRR16 | 0.794 |
| GLCE | C5AR1 | 0.790 | CR1 | DPYSL3 | 0.744 | PPP2R5A | LCP1 | 0.765 |
| TMEM71 | FAP | 0.767 | GLCE | DPYSL3 | 0.795 | OLFM1 | FPR2 | 0.813 |
| RBMS1 | HS3ST3B1 | 0.798 | GLCE | ACOD1 | 0.774 | S100A12 | CCL4 | 0.793 |
| ABL2 | AQP9 | 0.800 | PDPN | C11orf96 | 0.787 | ASB7 | SHTN1 | 0.789 |
| HIBCH | SLC35G2 | 0.809 | TAGAP | S100A9 | 0.756 | TMEM106C | SPOCK1 | 0.770 |
| GLT1D1 | IL11 | 0.825 | S100A9 | SLC16A6 | 0.819 | CEMIP | WWC2 | 0.765 |
| AGFG2 | CXCR1 | 0.731 | P2RY13 | CD69 | 0.724 | CDX2 | CLMP | 0.787 |
| INHBA | CLEC4A | 0.800 | GZMB | TLR4 | 0.742 | TMEM273 | RGS16 | 0.779 |
| OCRL | TMEM71 | 0.828 | RGCC | ITGAM | 0.785 | NRCAM | P2RY14 | 0.774 |
| TNFAIP2 | BMERB1 | 0.784 | SH3RF2 | DOK3 | 0.767 | SHTN1 | GPR183 | 0.790 |
| HCLS1 | CHST2 | 0.721 | TNFAIP2 | ENTPD1 | 0.765 | CR1 | TNFAIP2 | 0.723 |
| SHTN1 | SLA | 0.763 | ACOT9 | BCL2A1 | 0.790 | LILRA2 | GPR183 | 0.775 |
| PKIB | P2RY13 | 0.826 | FERMT2 | GADD45B | 0.787 | NRG1 | S100A9 | 0.817 |
| C5AR1 | GPR65 | 0.749 | C5AR1 | BMERB1 | 0.800 | WNT5A | DOK3 | 0.774 |
| COX10 | CLEC4A | 0.797 | CDX2 | FAM20C | 0.786 | CSF2RB | FAM155A | 0.824 |
| S100A9 | SSH2 | 0.837 | CEBPB | S100A12 | 0.789 | PKIB | RBMS1 | 0.810 |
| G0S2 | CCDC3 | 0.830 | ICAM1 | SSH2 | 0.762 | IL1B | TNF | 0.784 |
| CSF3 | RASSF5 | 0.789 | ADGRG3 | PI15 | 0.763 | CCDC88A | STAT5B | 0.761 |
| NFAM1 | C2CD2 | 0.753 | NR3C2 | SLC16A6 | 0.788 | ZNF704 | GPR183 | 0.782 |
| ZDHHC23 | ANGPTL2 | 0.820 | BMERB1 | GMFG | 0.831 | MIR100HG | CHST11 | 0.750 |
| IER5 | TUBA1A | 0.737 | CMTM8 | SPHK1 | 0.771 | C2CD2 | SERPINB9 | 0.776 |
| NFE2 | ZDHHC18 | 0.791 | CHI3L2 | MMP19 | 0.738 | FILIP1L | RIPK2 | 0.795 |
| SNN | P2RY14 | 0.793 | LCP1 | GFPT2 | 0.726 | RALGAPA2 | TAGAP | 0.753 |
| GOLPH3L | DUSP2 | 0.770 | LILRB2 | CCDC3 | 0.804 | ADCY6 | SNORC | 0.787 |
| KIAA1522 | CTHRC1 | 0.792 | COTL1 | RFLNB | 0.785 | JAZF1 | PDE4B | 0.747 |
| COQ9 | CCR1 | 0.792 | RGS19 | CHN1 | 0.748 | MMP3 | MSN | 0.788 |
| GAS2L3 | FZD5 | 0.759 | S1PR1 | SERPINB9 | 0.773 | CMTM8 | SH3RF2 | 0.783 |
| MIR100HG | PTPRC | 0.750 | KCNJ15 | ZDHHC18 | 0.731 | RGCC | C1R | 0.768 |
| LCP1 | THBD | 0.772 | FILIP1L | SELE | 0.802 | S1PR1 | RIPK2 | 0.794 |
| OCRL | VASN | 0.761 | CXCL5 | LST1 | 0.755 | MCAM | TNF | 0.802 |
| PPP1R18 | IL1RN | 0.762 | CHI3L2 | RGCC | 0.735 | ERMP1 | JAZF1 | 0.804 |
| RAI14 | C5AR1 | 0.769 | RFLNB | PREX1 | 0.747 | ENO2 | PRR16 | 0.815 |
| AGAP1 | SMAP2 | 0.746 | AFG3L2 | TLR1 | 0.822 | PPP2R5A | COQ9 | 0.775 |
| RGCC | CXCR4 | 0.740 | DPYSL3 | PDLIM4 | 0.775 | CCR1 | PRICKLE2 | 0.814 |
| GLCE | ABL2 | 0.796 | INHBA | SNN | 0.797 | CSNK1G3 | DLC1 | 0.794 |
| RGS19 | SSH2 | 0.692 | MEFV | PIP4P2 | 0.740 | COQ9 | CHRDL2 | 0.772 |
| CDX2 | INHBA | 0.795 | FILIP1L | ENO2 | 0.797 | GLIPR1 | MMP19 | 0.731 |
| DPYSL3 | CHST2 | 0.774 | CSF3 | MEFV | 0.796 | CDX2 | SLFN11 | 0.777 |
| SLA | CLMP | 0.741 | RFLNB | CXCR4 | 0.731 | WNK2 | IER5 | 0.787 |
| GALNT12 | ITPRIP | 0.803 | HSD11B2 | TCF4 | 0.773 | LILRB1 | BCAT1 | 0.775 |
| CR1 | ARHGAP25 | 0.719 | ANGPTL2 | SLC2A3 | 0.740 | PDPN | SELE | 0.810 |
| CHI3L2 | FPR1 | 0.773 | TMEM273 | CD53 | 0.724 | PREX1 | CCN4 | 0.768 |
| PRRX1 | CLMP | 0.765 | CDX2 | CXCR4 | 0.757 | STC1 | CD53 | 0.779 |
| CMTM4 | ZDHHC18 | 0.785 | SNORC | GFPT2 | 0.765 | FERMT2 | ANKRD35 | 0.772 |
| UBAC1 | LCP1 | 0.758 | SOD2 | LST1 | 0.766 | ASB7 | IFITM2 | 0.776 |
| ITGAM | FAM155A | 0.776 | LILRA5 | FAM20C | 0.754 | GALNT12 | RASSF5 | 0.791 |
| FPR2 | HCLS1 | 0.708 | COL15A1 | S1PR3 | 0.763 | STAP2 | CCR1 | 0.792 |
| DSP | SERPINE2 | 0.803 | ZNF704 | SNN | 0.807 | LY96 | CD69 | 0.773 |
| PAPPA | P2RY14 | 0.823 | P2RY13 | OSMR | 0.771 | NEO1 | CYTH4 | 0.771 |
| SWAP70 | SLC35G2 | 0.751 | S100A8 | SLC35G2 | 0.828 | FCGR3B | PHLDB2 | 0.824 |
| IL13RA2 | FAM49A | 0.870 | CHRDL2 | ACOD1 | 0.788 | FCGR3B | TUBA1A | 0.805 |
| GPR183 | SSH2 | 0.755 | CCDC69 | ASAP1 | 0.776 | INHBA | KCNJ15 | 0.763 |
| CCDC88A | SELP | 0.805 | OCRL | SOD2 | 0.790 | LGALS1 | FAM155A | 0.803 |
| NEO1 | RAI14 | 0.796 | NDUFA10 | CHST15 | 0.791 | SH3RF2 | P2RY13 | 0.817 |
| G0S2 | S100A12 | 0.798 | ARPIN | ERMP1 | 0.774 | FCER1G | DOK3 | 0.753 |
| GLCE | NEO1 | 0.773 | CLU | ITGAM | 0.759 | ZDHHC23 | ST3GAL5 | 0.791 |
| NOD2 | TUBB6 | 0.735 | GPR176 | PAPPA | 0.822 | COL4A2 | PLA1A | 0.759 |
| CXCR2 | C5AR1 | 0.750 | TCEA3 | CREM | 0.777 | CYTH4 | SSH2 | 0.758 |
| FGF7 | C11orf96 | 0.792 | TMEM273 | FGR | 0.793 | TRIM2 | CHST11 | 0.768 |
| SPOCK1 | SPP1 | 0.789 | GPR176 | CCDC3 | 0.754 | FPR2 | NRCAM | 0.801 |
| CAVIN1 | CCDC88A | 0.759 | COL15A1 | WNT5A | 0.792 | LRP4 | STAP2 | 0.770 |
| ASB7 | SLC16A6 | 0.726 | GLT1D1 | TREM1 | 0.724 | AFG3L2 | SERPINB9 | 0.754 |
| SERPINE1 | DOK3 | 0.773 | PREX1 | BCL6 | 0.676 | LY96 | SPOCK1 | 0.797 |
| TRPM6 | INHBA | 0.804 | LCP2 | CD14 | 0.735 | CD93 | CLEC4E | 0.778 |
| UQCRC1 | IL11 | 0.841 | ADCY6 | S100A9 | 0.819 | HSD11B2 | MAP4K4 | 0.780 |
| GPR176 | SELP | 0.784 | JAZF1 | PRRX1 | 0.765 | DOK3 | CD86 | 0.743 |
| NIPSNAP3A | CLEC4A | 0.843 | GNAI2 | INHBA | 0.785 | TAGAP | DUSP2 | 0.732 |
| LAT2 | PDLIM4 | 0.784 | VAV1 | DOK3 | 0.711 | PLA1A | SMAP2 | 0.769 |
| ANGPT2 | HLX | 0.742 | ITGAM | GPR137B | 0.797 | NDFIP2 | RIPK2 | 0.784 |
| GPR183 | S100A12 | 0.797 | CMTM8 | SERPINE1 | 0.795 | CSNK1G3 | ZNF704 | 0.788 |
| LDLRAD3 | DPYSL2 | 0.765 | RGCC | SELL | 0.798 | VAV1 | CXCR4 | 0.694 |
| BICD2 | PDE4B | 0.725 | ENO2 | NCF2 | 0.780 | ANGPT2 | CXCR1 | 0.774 |
| VASN | BICD2 | 0.730 | TMEM71 | PCBP3 | 0.807 | PFKL | PIK3AP1 | 0.731 |
| ABL2 | LOXL2 | 0.775 | CHST15 | SOD2 | 0.795 | ITGAM | SMAP2 | 0.747 |
| FGR | IL13RA2 | 0.864 | SH3RF2 | NCF2 | 0.807 | AGAP1 | MAP4K4 | 0.778 |
| NCF4 | TNFAIP6 | 0.796 | LILRB1 | CBX6 | 0.768 | CXCR1 | NRCAM | 0.760 |
| PAPPA | CCN4 | 0.820 | ATP5IF1 | ASAP1 | 0.779 | FCGR3B | CYTH4 | 0.804 |
| GLIPR1 | SLC16A6 | 0.680 | FILIP1L | STAT5B | 0.772 | LOXL2 | P2RY13 | 0.781 |
| STAT5B | C1R | 0.777 | ITGAM | TUBA1A | 0.759 | CBX6 | CHST15 | 0.768 |
| NEO1 | ZNF704 | 0.785 | CAVIN3 | TMEM154 | 0.778 | NEO1 | CCDC69 | 0.773 |
| TCEA3 | IL1B | 0.787 | TCEA3 | CHST2 | 0.783 | DLC1 | FCGR3B | 0.834 |
| PDPN | CHI3L2 | 0.754 | LILRB2 | SLC35G2 | 0.806 | LRRK2 | FPR1 | 0.752 |
| EGR3 | NFE2 | 0.763 | ATP5IF1 | IER5 | 0.755 | LCP2 | STC1 | 0.805 |
| GLCE | LCP2 | 0.773 | BCL2A1 | CXCR4 | 0.712 | ANKRD35 | IL13RA2 | 0.888 |
| ZNF704 | LCP1 | 0.734 | CAVIN3 | F5 | 0.783 | LDLRAD3 | WNT5A | 0.801 |
| IL1B | CCL2 | 0.793 | VASN | SLC15A4 | 0.755 | RBMS1 | BCL6 | 0.703 |
| SH3RF2 | IER5 | 0.769 | EGR3 | FGF7 | 0.783 | HIBCH | PRRX1 | 0.774 |
| FERMT2 | AMPD2 | 0.776 | CHN1 | DOK3 | 0.757 | TUBB6 | PDLIM4 | 0.779 |
| CLEC4E | CHST15 | 0.769 | RBMS1 | THEMIS2 | 0.747 | TMEM273 | C11orf96 | 0.792 |
| CXCL8 | RIPK2 | 0.786 | ZNF704 | CHI3L1 | 0.794 | PIP4P2 | LST1 | 0.772 |
| PRR16 | CCDC3 | 0.806 | SRGN | TNFAIP6 | 0.787 | KIAA1522 | MMP3 | 0.798 |
| RBMS1 | SOD2 | 0.792 | CCL2 | ACOD1 | 0.761 | ADCY6 | CEP19 | 0.767 |
| UQCRC1 | PLA1A | 0.795 | DLC1 | WWC2 | 0.771 | RGS16 | TCF4 | 0.763 |
| TRPM6 | ANGPTL2 | 0.776 | PFKL | SHTN1 | 0.789 | TRAK1 | FAP | 0.763 |
| FAP | NOD2 | 0.709 | CGAS | KCNJ15 | 0.696 | ENO2 | MSN | 0.750 |
| CCDC88A | CEP19 | 0.704 | LGALS1 | MIR100HG | 0.771 | ATP5IF1 | SWAP70 | 0.725 |
| S100A9 | TIMP1 | 0.813 | MSN | CCL4 | 0.783 | CXCR2 | MCAM | 0.808 |
| CHI3L2 | SLC15A3 | 0.742 | CRIP2 | FGF7 | 0.788 | CHRDL2 | MEFV | 0.792 |
| NDFIP2 | CSF3 | 0.800 | TIMP1 | VNN2 | 0.808 | RHOH | C5AR1 | 0.723 |
| HCK | LCP2 | 0.745 | TCEA3 | RBMS1 | 0.802 | ITPRIP | THEMIS2 | 0.810 |
| C2CD2 | BMERB1 | 0.796 | CLEC4E | DOK3 | 0.732 | GPR176 | PCBP3 | 0.755 |
| FGF7 | CYP26B1 | 0.821 | INHBA | GPR137B | 0.805 | NIPSNAP3A | PXDN | 0.817 |
| EMP3 | BICD2 | 0.744 | ERMP1 | CD69 | 0.776 | UBAC1 | TSPAN6 | 0.765 |
| LRP4 | FPR1 | 0.810 | DLC1 | PLAU | 0.781 | LGALS1 | SPHK1 | 0.775 |
| TRPM6 | P2RY14 | 0.800 | LRP4 | KCNJ15 | 0.765 | PRRX1 | FAM155A | 0.796 |
| FCGR2B | ITGAM | 0.779 | GPR137B | P2RY14 | 0.785 | COL4A2 | GMFG | 0.779 |
| ITGAM | BPGM | 0.776 | TSPAN6 | CHST11 | 0.784 | UBAC1 | CYP26B1 | 0.815 |
| S100A8 | P2RY14 | 0.841 | AKAP1 | CEBPB | 0.802 | RAB15 | S1PR3 | 0.791 |
| LY96 | CXCL5 | 0.776 | LRP4 | TAGAP | 0.792 | P2RY13 | CHST2 | 0.769 |
| F5 | NRCAM | 0.772 | SORT1 | PRICKLE2 | 0.794 | SPOCK1 | ACOD1 | 0.794 |
| C2CD2 | ACOD1 | 0.754 | F5 | IER5 | 0.746 | TCEA3 | FGF2 | 0.789 |
| JAZF1 | SLA | 0.731 | CMTM8 | FFAR2 | 0.794 | F5 | HS3ST3B1 | 0.801 |
| RALGAPA2 | SLC35G2 | 0.809 | TRPM6 | NEO1 | 0.779 | WNK2 | NFAM1 | 0.789 |
| B4GALT4 | ITPRIP | 0.803 | S100A8 | FAM155A | 0.827 | LRRK2 | PI15 | 0.773 |
| NDUFA10 | CRIP2 | 0.780 | CYTH4 | RGCC | 0.781 | C1R | MAP4K4 | 0.769 |
| DSP | IL24 | 0.799 | HIBCH | FJX1 | 0.753 | RILPL2 | IL6 | 0.824 |
| CSF3 | CXCL8 | 0.786 | CTHRC1 | ACOT9 | 0.795 | NCF2 | ASAP1 | 0.778 |
| FJX1 | BTG2 | 0.759 | CGAS | HLX | 0.696 | IER5 | CCL2 | 0.746 |
| ZNF704 | CLU | 0.775 | CGAS | PXDN | 0.770 | DSP | LGALS1 | 0.794 |
| FCER1G | ITPRIP | 0.800 | ERMP1 | SSH2 | 0.778 | CMTM4 | CLU | 0.770 |
| FPR2 | BPGM | 0.792 | PRICKLE2 | PRR16 | 0.809 | CSTA | RGS16 | 0.747 |
| WWC2 | RIPK2 | 0.761 | RIPOR3 | TMEM154 | 0.807 | PPP1R18 | PDLIM4 | 0.761 |
| NDUFA10 | PREX1 | 0.778 | LRRK2 | INHBA | 0.795 | ENO2 | COTL1 | 0.759 |
| CBX6 | NCF4 | 0.739 | COQ9 | PLA1A | 0.785 | C11orf96 | ZDHHC18 | 0.786 |
| CTHRC1 | TMEM154 | 0.793 | CD300A | GADD45B | 0.791 | CMTM8 | NNMT | 0.784 |
| TAGAP | P2RY14 | 0.748 | CMTM8 | S1PR3 | 0.755 | MYO1D | LDLRAD3 | 0.798 |
| DUSP4 | TIMP1 | 0.763 | AFG3L2 | STAP2 | 0.745 | COL7A1 | CCL2 | 0.788 |
| PFKL | IL1RN | 0.776 | PPP2R5A | TUBB6 | 0.800 | FZD5 | BCL2A1 | 0.795 |
| CCR1 | TNFAIP2 | 0.764 | C5AR1 | TUBA1A | 0.759 | LILRB2 | MCAM | 0.800 |
| DUSP4 | GPR65 | 0.778 | CCR1 | WNT5A | 0.819 | FGF7 | FAM155A | 0.807 |
| TMEM154 | GADD45B | 0.790 | DUSP2 | EMP3 | 0.768 | PKIB | CHN1 | 0.799 |
| FILIP1L | FFAR2 | 0.785 | CDX2 | TRIM2 | 0.786 | OSM | CREB5 | 0.813 |
| ABL2 | CXCR1 | 0.742 | SOD2 | OSMR | 0.781 | CMTM4 | CXCR4 | 0.729 |
| CSNK1G3 | BPGM | 0.788 | AGFG2 | NOD2 | 0.728 | GOLPH3L | SOD2 | 0.802 |
| CMTM4 | SELE | 0.799 | PI15 | CD69 | 0.760 | STC1 | VNN2 | 0.830 |
| DLC1 | RIPK2 | 0.799 | NR3C2 | LDLRAD3 | 0.799 | ITGAM | THEMIS2 | 0.720 |
| LRRK2 | ENO2 | 0.795 | CLEC4A | DOK3 | 0.762 | CAVIN3 | RHOH | 0.757 |
| OCRL | ANGPTL2 | 0.781 | DLC1 | S1PR1 | 0.790 | MSN | GMFG | 0.775 |
| VASN | CD86 | 0.750 | TMEM154 | ACOD1 | 0.748 | MSN | PRR16 | 0.805 |
| AGFG2 | FERMT2 | 0.780 | S1PR1 | FAM20C | 0.776 | CEBPB | CBX6 | 0.777 |
| GAS2L3 | IL6 | 0.813 | CAVIN3 | IL1B | 0.806 | TMEM106C | COTL1 | 0.759 |
| TRPM6 | CRIP2 | 0.768 | FERMT2 | FCGR2B | 0.787 | CSF3R | NRCAM | 0.740 |
| C2CD2 | S100A8 | 0.822 | GAS2L3 | MYO1D | 0.785 | DSP | CHI3L2 | 0.750 |
| ST3GAL5 | ARHGAP25 | 0.757 | CRIP2 | RGS16 | 0.782 | JAZF1 | LCP2 | 0.766 |
| NIPSNAP3A | IL13RA2 | 0.857 | KIAA1522 | TIMP1 | 0.776 | BICD2 | GPR137B | 0.795 |
| SLC15A4 | RFLNB | 0.802 | LDLRAD3 | CD53 | 0.757 | ANGPTL2 | CHST11 | 0.760 |
| TRIM2 | CREM | 0.796 | SH3RF2 | F5 | 0.797 | SELENBP1 | G0S2 | 0.828 |
| OLFM1 | PRR16 | 0.801 | CEP19 | P2RY14 | 0.751 | ZDHHC23 | PROK2 | 0.777 |
| NIBAN1 | IL1B | 0.794 | DLC1 | CCL4 | 0.802 | CXCR2 | CHST15 | 0.814 |
| CHST15 | PHLDB2 | 0.786 | GOLPH3L | RGS19 | 0.726 | FAM49A | ENTPD1 | 0.806 |
| GOLPH3L | TIMP1 | 0.789 | NCF4 | SERPINE2 | 0.788 | CSF2RB | CXCR2 | 0.794 |
| CD93 | ITPRIP | 0.803 | WNK2 | IL1RN | 0.813 | CEBPB | BICD2 | 0.791 |
| ZDHHC23 | HSD11B2 | 0.778 | ZNF704 | CHST2 | 0.797 | FZD5 | C1R | 0.780 |
| CLU | MSN | 0.753 | ANKRD35 | NRCAM | 0.789 | CAVIN3 | GPR183 | 0.768 |
| RAI14 | WNT5A | 0.812 | S1PR3 | FCGR2B | 0.782 | GAS2L3 | BTG2 | 0.776 |
| BASP1 | G0S2 | 0.812 | NOD2 | TCF4 | 0.697 | LILRB1 | PLEK | 0.771 |
| NDUFA10 | P2RY13 | 0.801 | AGAP1 | NNMT | 0.794 | TRPM6 | SPOCK1 | 0.798 |
| TNFAIP2 | OSMR | 0.771 | SLC2A3 | SH3BP5 | 0.729 | CMTM4 | VAV1 | 0.760 |
| KCNJ15 | TLR4 | 0.696 | CHI3L2 | SLC35G2 | 0.781 | PRRX1 | TREM1 | 0.760 |
| CSF2RB | BPGM | 0.777 | GPR183 | TNFRSF10C | 0.751 | CHST15 | PLA1A | 0.795 |
| NR3C2 | TMEM71 | 0.796 | TAGAP | SLA | 0.719 | WNT5A | BTG2 | 0.797 |
| ZNF704 | RGCC | 0.814 | GALNT12 | DPYSL3 | 0.778 | CCR1 | GMFG | 0.797 |
| COQ9 | RGCC | 0.796 | GLCE | ASAP1 | 0.795 | NCF4 | NNMT | 0.781 |
| MYO1D | PDLIM4 | 0.814 | TCEA3 | TLR4 | 0.737 | LRP4 | MMP19 | 0.789 |
| ANKRD35 | LST1 | 0.780 | S1PR1 | ENTPD1 | 0.779 | RAB15 | COL7A1 | 0.802 |
| ZNF704 | CAVIN3 | 0.782 | CSTA | LY96 | 0.762 | SELP | NOD2 | 0.758 |
| CYTH4 | CEMIP | 0.788 | CEMIP | SELL | 0.789 | GADD45B | NNMT | 0.794 |
| AKAP1 | OSM | 0.809 | WNK2 | TNFRSF10C | 0.788 | TREM1 | CD14 | 0.739 |
| P2RY13 | PIP4P2 | 0.751 | NR3C2 | ST3GAL5 | 0.775 | COQ9 | LILRB1 | 0.789 |
| LRP4 | TUBA1A | 0.787 | CD300A | IL13RA2 | 0.867 | ZDHHC23 | RAB31 | 0.810 |
| IL11 | SNN | 0.835 | SMAP2 | VAV1 | 0.728 | TRPM6 | NIBAN1 | 0.820 |
| COX10 | EGR3 | 0.748 | PIK3AP1 | TNFAIP6 | 0.763 | TRIM2 | CBX6 | 0.776 |
| CLU | G0S2 | 0.807 | DUSP4 | ZDHHC18 | 0.785 | EMP3 | CD86 | 0.783 |
| GADD45B | MAP4K4 | 0.778 | IL24 | SNN | 0.805 | SH2D3C | CD93 | 0.789 |
| CR1 | ASAP1 | 0.755 | SLFN11 | RIPOR2 | 0.707 | C2CD2 | CAVIN1 | 0.755 |
| SH2D3C | TNC | 0.794 | CEBPB | SLC16A6 | 0.752 | C2CD2 | ASAP1 | 0.772 |
| FCER1G | IL1B | 0.786 | TMEM154 | CEMIP | 0.789 | CD300A | TNF | 0.806 |
| SHTN1 | CSF3 | 0.782 | FCGR3B | SERPINB9 | 0.808 | DSP | ACOD1 | 0.770 |
| MYO1D | FAP | 0.766 | RGCC | SRGN | 0.791 | PRICKLE2 | TNC | 0.794 |
| CDX2 | BPGM | 0.780 | S1PR3 | GMFG | 0.771 | TMEM71 | NFAM1 | 0.746 |
| NR3C2 | CSTA | 0.781 | SELL | CXCR4 | 0.736 | DLC1 | CEBPB | 0.806 |
| OSM | SELL | 0.820 | LILRB2 | ITGA5 | 0.805 | NFE2 | PRRX1 | 0.774 |
| ICAM1 | BPGM | 0.746 | IL1B | PI15 | 0.768 | CSF3 | SPOCK1 | 0.806 |
| FERMT2 | TMEM71 | 0.796 | PIK3AP1 | LDLRAD3 | 0.759 | HIBCH | VAV1 | 0.763 |
| SERPINE1 | GFPT2 | 0.757 | SELE | RIPK2 | 0.801 | SERPINB9 | THEMIS2 | 0.780 |
| SLC15A4 | RBMS1 | 0.821 | PKIB | DPYSL3 | 0.773 | LILRB3 | JAZF1 | 0.743 |
| COL15A1 | TLR1 | 0.782 | EMP3 | FLT1 | 0.761 | MIR100HG | MMP19 | 0.758 |
| PPP1R18 | C5AR1 | 0.763 | CCR1 | CHST11 | 0.777 | NNMT | THBD | 0.793 |
| IL24 | FFAR2 | 0.806 | TRAK1 | WNK2 | 0.818 | SELP | MAP4K4 | 0.814 |
| GZMB | SLC11A1 | 0.806 | MX2 | IER5 | 0.733 | CEBPB | RIPOR3 | 0.796 |
| FCGR2B | RIPOR2 | 0.775 | GPR183 | TNFAIP2 | 0.752 | COQ9 | MMP3 | 0.791 |
| ANGPT2 | NCF2 | 0.784 | TSPAN6 | DOK3 | 0.778 | CHN1 | DUSP2 | 0.778 |
| GALNT12 | CXCR4 | 0.761 | HSD11B2 | NNMT | 0.807 | LILRB3 | CD300A | 0.751 |
| CYP26B1 | PDLIM4 | 0.804 | AMPD2 | PTGS1 | 0.785 | COL15A1 | CLEC4E | 0.760 |
| DLC1 | BICD2 | 0.797 | S1PR3 | FAP | 0.753 | NIPSNAP3A | FCER1G | 0.816 |
| SLC11A1 | LST1 | 0.794 | ADCY6 | S100A12 | 0.785 | RALGAPA2 | S1PR3 | 0.762 |
| SLC2A3 | P2RY14 | 0.753 | ARPIN | SPP1 | 0.747 | GAS2L3 | SLA | 0.755 |
| GOLPH3L | FCER1G | 0.774 | TSPAN6 | SLC2A3 | 0.798 | SH3RF2 | CXCR2 | 0.818 |
| CMTM4 | TRAK1 | 0.800 | CMTM8 | MMP3 | 0.787 | NFAM1 | VIM | 0.765 |
| CLEC4E | GZMB | 0.762 | FZD5 | TNFAIP2 | 0.766 | AFG3L2 | AKAP1 | 0.774 |
| COL7A1 | MMP3 | 0.800 | SORT1 | SELE | 0.806 | WNK2 | SERPINE1 | 0.821 |
| LRP4 | RIPOR2 | 0.771 | RGS19 | VIM | 0.721 | TRAK1 | LCP1 | 0.747 |
| FGF7 | CCL2 | 0.774 | FPR1 | FAM155A | 0.796 | RAB31 | CXCL5 | 0.766 |
| DSP | IFITM2 | 0.773 | RIPOR3 | ANGPTL2 | 0.791 | LILRB2 | COTL1 | 0.803 |
| BASP1 | BTG2 | 0.773 | S1PR1 | HSPA6 | 0.733 | RGCC | PROK2 | 0.758 |
| RILPL2 | HCLS1 | 0.739 | SELL | SH3BP5 | 0.798 | RAI14 | PI15 | 0.767 |
| FCGR3B | RAI14 | 0.815 | BCL6 | TUBB6 | 0.705 | FJX1 | MSN | 0.752 |
| BTG2 | TNFRSF10C | 0.771 | MYO1F | ST3GAL5 | 0.755 | B4GALT4 | SLC15A3 | 0.760 |
| GLIPR1 | PDLIM4 | 0.739 | BCAT1 | S100A8 | 0.795 | STAT5B | HS3ST3B1 | 0.776 |
| PRICKLE2 | HS3ST3B1 | 0.792 | FGF2 | TCF4 | 0.751 | STC1 | ACOD1 | 0.806 |
| BCL2A1 | PROK2 | 0.716 | PI15 | SSH2 | 0.772 | WNK2 | MMP19 | 0.810 |
| VASN | TREM1 | 0.752 | ITGA5 | CLEC4A | 0.790 | COL15A1 | PLEK | 0.762 |
| GZMB | OSM | 0.807 | NNMT | STAT5B | 0.785 | S100A9 | SELL | 0.833 |
| NEO1 | CCR1 | 0.821 | SORT1 | BTG2 | 0.771 | DPYSL2 | PCBP3 | 0.766 |
| OLFM1 | P2RY13 | 0.772 | TRIM2 | SLC11A1 | 0.783 | TMEM71 | HLX | 0.719 |
| RNF149 | FAM155A | 0.816 | C11orf96 | GMFG | 0.800 | VIM | CLMP | 0.757 |
| CD300A | KCNJ15 | 0.745 | HCLS1 | PTGS2 | 0.746 | SH2D3C | LAT2 | 0.762 |
| PIP4P2 | MAP4K4 | 0.754 | LILRB1 | C2CD2 | 0.789 | FPR1 | STC1 | 0.801 |
| NR3C2 | PKIB | 0.766 | LRRK2 | C1R | 0.770 | WNK2 | LCP1 | 0.780 |
| HIBCH | OSMR | 0.786 | CHN1 | CRIP2 | 0.782 | MYO1D | PI15 | 0.770 |
| TREM1 | SPOCK1 | 0.781 | STAT5B | WWC2 | 0.759 | NDUFA10 | ARHGAP25 | 0.795 |
| GAS2L3 | PTGS2 | 0.803 | VASN | FGR | 0.757 | RIPOR3 | ST3GAL5 | 0.778 |
| ICAM1 | MX2 | 0.766 | NFAM1 | FGF7 | 0.799 | NRG1 | CHST11 | 0.785 |
| ERMP1 | GFPT2 | 0.776 | CMTM8 | PTPRC | 0.779 | ERMP1 | PTGS1 | 0.820 |
| TMEM273 | ST3GAL5 | 0.756 | CRIP2 | TLR4 | 0.717 | IFITM2 | S100A9 | 0.806 |
| AKAP1 | MX2 | 0.811 | CHST11 | PLA1A | 0.760 | GNAI2 | SERPINE2 | 0.774 |
| CGAS | CR1 | 0.714 | ITGAM | SPHK1 | 0.768 | EGR3 | CHRDL2 | 0.771 |
| AGAP1 | FERMT2 | 0.792 | RIPOR3 | TNFRSF10C | 0.790 | CCDC3 | CREB5 | 0.754 |
| PIP4P2 | SLA | 0.750 | LILRB1 | CXCL8 | 0.806 | CCL4 | TNFAIP2 | 0.749 |
| SNORC | OCRL | 0.788 | FGR | TNFRSF1B | 0.732 | TMEM273 | THBD | 0.799 |
| BASP1 | CCDC88A | 0.787 | CMTM8 | BASP1 | 0.787 | TMEM273 | MAP4K4 | 0.755 |
| CCR1 | FGF7 | 0.807 | CHST11 | BCL6 | 0.676 | CEBPB | BCL6 | 0.708 |
| PFKL | P2RY13 | 0.801 | UQCRC1 | P2RY13 | 0.806 | CHI3L1 | STAT5B | 0.787 |
| CCN4 | CD69 | 0.763 | ENO2 | G0S2 | 0.816 | SH2D3C | COL7A1 | 0.797 |
| ARPIN | WNT5A | 0.797 | NIBAN1 | FAM49A | 0.759 | TNF | BTG2 | 0.768 |
| STAP2 | RGCC | 0.772 | NDFIP2 | SOD2 | 0.789 | ZNF704 | LY96 | 0.798 |
| CBX6 | KCNJ15 | 0.710 | PDPN | GNAI2 | 0.778 | GNAI2 | GMFG | 0.773 |
| GADD45B | BCL2A1 | 0.762 | FCGR2B | CCDC88A | 0.778 | PFKL | DOK3 | 0.774 |
| MEFV | PDE4B | 0.730 | CCDC88A | GPR65 | 0.734 | CLU | WNT5A | 0.803 |
| DUSP4 | CCDC69 | 0.777 | AFG3L2 | B4GALT4 | 0.765 | MCAM | BPGM | 0.768 |
| CCL2 | LAT2 | 0.770 | NIPSNAP3A | TLR1 | 0.839 | GAS2L3 | HSD11B2 | 0.765 |
| CSNK1G3 | SELP | 0.795 | CR1 | LCP1 | 0.722 | HIBCH | RILPL2 | 0.815 |
| TREM1 | WWC2 | 0.764 | SLC15A4 | TNFRSF10C | 0.783 | EGR3 | SLFN11 | 0.703 |
| ARPIN | PREX1 | 0.772 | NR3C2 | OSM | 0.816 | IL6 | OSM | 0.814 |
| RAI14 | SELE | 0.799 | FZD5 | CHST15 | 0.776 | NIPSNAP3A | GOLPH3L | 0.760 |
| PDPN | JAZF1 | 0.781 | IL24 | IL13RA2 | 0.853 | EMP3 | SERPINE2 | 0.774 |
| GLCE | WWC2 | 0.802 | TRIM2 | CHRDL2 | 0.769 | CHST11 | GPR65 | 0.719 |
| NDUFA10 | CAVIN1 | 0.772 | GPR176 | CHST11 | 0.753 | HIBCH | CAVIN1 | 0.763 |
| CSF3R | GADD45B | 0.729 | PTGS1 | P2RY14 | 0.784 | OSM | CD86 | 0.806 |
| GOLPH3L | BASP1 | 0.788 | UQCRC1 | GMFG | 0.801 | FILIP1L | SERPINB9 | 0.793 |
| NR3C2 | CD14 | 0.805 | MYO1D | NFE2 | 0.797 | PKIB | NNMT | 0.809 |
| EFNA4 | C2CD2 | 0.774 | NR3C2 | FAP | 0.797 | EGR3 | S100A12 | 0.792 |
| OCRL | CSF2RB | 0.798 | CHI3L1 | IL11 | 0.822 | SERPINB9 | SLC16A6 | 0.735 |
| FGF7 | NRG1 | 0.803 | JAZF1 | CD53 | 0.722 | CYTH4 | G0S2 | 0.804 |
| ATP5IF1 | DPYSL2 | 0.772 | PTGS1 | LST1 | 0.777 | CCR1 | FPR1 | 0.778 |
| LILRA5 | SERPINE1 | 0.787 | NOD2 | P2RY14 | 0.736 | TAGAP | CXCR2 | 0.720 |
| C2CD2 | RAI14 | 0.758 | CCN4 | SPHK1 | 0.776 | HIBCH | NRCAM | 0.769 |
| BCL2A1 | CREB5 | 0.763 | VASN | HCK | 0.720 | FLT1 | TLR1 | 0.781 |
| OSM | IER5 | 0.797 | IL11 | VIM | 0.823 | TMEM273 | TUBA1A | 0.768 |
| FCER1G | MMP3 | 0.781 | TRAK1 | CEP19 | 0.753 | IL24 | PDE4B | 0.776 |
| NIBAN1 | RAI14 | 0.776 | RGS16 | STC1 | 0.792 | GPR176 | PLA1A | 0.763 |
| UQCRC1 | ENO2 | 0.772 | CMTM4 | ANGPTL2 | 0.789 | RAB31 | IL1RN | 0.763 |
| LCP2 | CREB5 | 0.746 | COX10 | MYO1D | 0.792 | CSNK1G3 | RFLNB | 0.773 |
| AFG3L2 | PREX1 | 0.772 | AGAP1 | DUSP2 | 0.777 | SPOCK1 | SPHK1 | 0.793 |
| CMTM4 | TMEM154 | 0.800 | NR3C2 | FZD5 | 0.776 | CCL2 | PHLDB2 | 0.759 |
| HCK | SPP1 | 0.746 | TNC | TUBA1A | 0.779 | PKIB | S100A9 | 0.832 |
| RHOH | NCF2 | 0.709 | CHST15 | TREM1 | 0.775 | CLEC4E | PCBP3 | 0.773 |
| LAT2 | LST1 | 0.760 | TCF4 | LST1 | 0.764 | PI15 | FAM20C | 0.767 |
| MCAM | BCL6 | 0.707 | F5 | CXCR4 | 0.737 | BICD2 | CHRDL2 | 0.772 |
| CMTM4 | HSD11B2 | 0.759 | TAGAP | FAM20C | 0.709 | SLC2A3 | DOK3 | 0.741 |
| BCL6 | CREB5 | 0.678 | DLC1 | FILIP1L | 0.763 | PAPPA | SELE | 0.826 |
| CCDC3 | SPHK1 | 0.777 | VASN | VNN2 | 0.776 | BASP1 | OSM | 0.812 |
| ANKRD35 | CD69 | 0.765 | BPGM | TNFRSF1B | 0.746 | HSPA6 | IL1RN | 0.748 |
| ITGAM | TNFAIP6 | 0.793 | GAS2L3 | CXCR1 | 0.752 | AFG3L2 | CYTH4 | 0.753 |
| GZMB | LST1 | 0.787 | PDPN | CCR1 | 0.792 | PPP1R18 | STC1 | 0.798 |
| RALGAPA2 | ATP5IF1 | 0.785 | COTL1 | P2RY14 | 0.774 | RAB31 | IL13RA2 | 0.857 |
| CSF3 | INHBA | 0.798 | CR1 | CLEC4A | 0.768 | NRG1 | HS3ST3B1 | 0.800 |
| SWAP70 | LY96 | 0.751 | CREM | INHBA | 0.799 | TSPAN6 | ICAM1 | 0.789 |
| NIPSNAP3A | THBD | 0.815 | COX10 | ST3GAL5 | 0.784 | ITGA5 | LST1 | 0.775 |
| GLIPR1 | GPR137B | 0.744 | FAM155A | LAT2 | 0.810 | MX2 | CCL2 | 0.768 |
| JAZF1 | SSH2 | 0.771 | DLC1 | CAVIN1 | 0.778 | STAP2 | CCDC3 | 0.777 |
| VASN | TLR4 | 0.698 | SERPINB9 | FAM49A | 0.782 | CHI3L2 | LILRA5 | 0.767 |
| TCEA3 | NRG1 | 0.796 | ANGPT2 | NFE2 | 0.787 | MCAM | PXDN | 0.770 |
| SH2D3C | TNFAIP2 | 0.749 | GLT1D1 | GZMB | 0.781 | LILRB1 | FCGR2B | 0.782 |
| GLT1D1 | VAV1 | 0.710 | NRG1 | ACOD1 | 0.795 | B4GALT4 | DPYSL2 | 0.762 |
| KCNJ15 | FAM155A | 0.760 | FILIP1L | LILRA5 | 0.786 | EGR3 | SLC15A3 | 0.733 |
| ADGRG3 | CHST2 | 0.757 | GPR183 | HCLS1 | 0.724 | SRGN | SLC35G2 | 0.808 |
| RIPOR3 | LST1 | 0.786 | MYO1D | SELL | 0.801 | GLIPR1 | BCL2A1 | 0.736 |
| CSF2RB | CHST2 | 0.790 | HSD11B2 | LGALS1 | 0.804 | NRG1 | CXCR1 | 0.739 |
| RGCC | MAP4K4 | 0.791 | CBX6 | HCLS1 | 0.672 | CAVIN3 | ITGAM | 0.773 |
| FJX1 | TUBB6 | 0.762 | DLC1 | S1PR3 | 0.788 | OLFM1 | COL7A1 | 0.814 |
| AGFG2 | DUSP4 | 0.754 | LDLRAD3 | HS3ST3B1 | 0.802 | TSPAN6 | RGS19 | 0.756 |
| LILRA5 | MEFV | 0.741 | CD69 | ARHGAP25 | 0.703 | GLT1D1 | SLC2A3 | 0.739 |
| PLAU | CCL2 | 0.763 | CEBPB | PTGS1 | 0.788 | ASAP1 | SELL | 0.792 |
| RIPOR3 | VAV1 | 0.780 | OLFM1 | GPR183 | 0.757 | CXCR1 | CCN4 | 0.751 |
| RGS19 | CD14 | 0.711 | TMEM71 | PTGS1 | 0.799 | S1PR3 | FGF2 | 0.769 |
| PXDN | PDLIM4 | 0.788 | PPP1R18 | PCBP3 | 0.747 | RIPK2 | VNN2 | 0.807 |
| AGFG2 | NRG1 | 0.784 | PLAU | RASSF5 | 0.755 | POLDIP2 | DPYSL2 | 0.748 |
| RHOH | CD86 | 0.746 | BPGM | TUBA1A | 0.756 | SH2D3C | S100A8 | 0.815 |
| CYTH4 | SLFN11 | 0.771 | MX2 | VIM | 0.765 | AKAP1 | LY96 | 0.803 |
| GALNT12 | B4GALT4 | 0.751 | ADCY6 | NNMT | 0.799 | CD300A | ANKRD35 | 0.808 |
| NDFIP2 | GALNT12 | 0.765 | FFAR2 | NCF2 | 0.719 | CRIP2 | RIPOR2 | 0.756 |
| CCDC69 | GADD45B | 0.772 | UQCRC1 | NCF2 | 0.809 | PTGS1 | SLC35G2 | 0.796 |
| FJX1 | RIPOR2 | 0.749 | GLCE | SH3BP5 | 0.783 | TLR4 | DOK3 | 0.708 |
| NCF4 | FAM20C | 0.770 | CMTM4 | PTPRC | 0.790 | CRIP2 | CCDC3 | 0.760 |
| ACOT9 | GMFG | 0.809 | ANGPT2 | CD14 | 0.781 | ITGAM | GFPT2 | 0.756 |
| FGF2 | BCAT1 | 0.764 | IFITM2 | SELL | 0.794 | FJX1 | TNFAIP2 | 0.756 |
| MSN | SRGN | 0.787 | EFNA4 | CRIP2 | 0.799 | PLA1A | SOD2 | 0.765 |
| MSN | CREB5 | 0.799 | PIK3AP1 | C2CD2 | 0.729 | SNORC | P2RY14 | 0.802 |
| LGALS1 | OSM | 0.805 | MCAM | CCDC3 | 0.762 | CYP26B1 | SMAP2 | 0.775 |
| AGAP1 | EFNA4 | 0.784 | SELL | CD69 | 0.770 | TRIM2 | VAV1 | 0.769 |
| FAP | SRGN | 0.759 | RAB15 | VASN | 0.784 | RIPOR3 | P2RY13 | 0.806 |
| PKIB | CEP19 | 0.784 | HCLS1 | TNFRSF1B | 0.679 | LCP2 | CD86 | 0.771 |
| COQ9 | PRRX1 | 0.773 | FCGR2B | SWAP70 | 0.738 | CCR1 | ACOT9 | 0.808 |
| CHN1 | CLU | 0.772 | SLC15A4 | SWAP70 | 0.752 | UBAC1 | OCRL | 0.808 |
| P2RY13 | PTPRC | 0.730 | ADGRG3 | BICD2 | 0.745 | CCL4 | STAT5B | 0.776 |
| CAVIN1 | MMP19 | 0.770 | NEO1 | FAM49A | 0.805 | COX10 | CD300A | 0.820 |
| CSNK1G3 | GPR176 | 0.744 | KIAA1522 | JAZF1 | 0.766 | BICD2 | SELL | 0.789 |
| OCRL | ITPRIP | 0.807 | LILRB2 | CD93 | 0.802 | CRIP2 | SLC15A3 | 0.754 |
| RILPL2 | PLAU | 0.784 | ARPIN | DUSP2 | 0.764 | DLC1 | DUSP4 | 0.810 |
| CHI3L2 | GPR183 | 0.724 | NR3C2 | TREM1 | 0.783 | STAP2 | BMERB1 | 0.793 |
| ZDHHC18 | CD53 | 0.740 | CXCL5 | ENTPD1 | 0.776 | NIBAN1 | PRRX1 | 0.777 |
| COQ9 | SWAP70 | 0.746 | CR1 | CREB5 | 0.744 | FPR1 | AMPD2 | 0.790 |
| NRCAM | S100A9 | 0.802 | SH3RF2 | HS3ST3B1 | 0.801 | DPYSL2 | CHST15 | 0.781 |
| GPR183 | CHRDL2 | 0.767 | GPR183 | CEP19 | 0.712 | COX10 | CLU | 0.763 |
| TAGAP | G0S2 | 0.733 | FCGR2B | LGALS1 | 0.778 | SMAP2 | TUBA1A | 0.752 |
| CSF3R | FGR | 0.726 | NR3C2 | GPR137B | 0.782 | COX10 | CSF3R | 0.761 |
| LRP4 | MEFV | 0.764 | FGR | ENTPD1 | 0.801 | TREM1 | CCN4 | 0.761 |
| CYTH4 | BCL6 | 0.730 | OCRL | GPR176 | 0.752 | LILRA2 | THEMIS2 | 0.735 |
| MYO1F | PDLIM4 | 0.784 | C1R | DOK3 | 0.745 | CREM | S100A8 | 0.807 |
| AKAP1 | CRIP2 | 0.789 | OLFM1 | NRG1 | 0.808 | S1PR1 | GFPT2 | 0.746 |
| EMP3 | SNN | 0.787 | UQCRC1 | IFITM2 | 0.791 | DPYSL3 | CHRDL2 | 0.766 |
| CDX2 | TIMP1 | 0.788 | RBMS1 | CCN4 | 0.784 | NR3C2 | KIAA1522 | 0.788 |
| GLCE | SLC15A4 | 0.801 | NEO1 | LILRB1 | 0.803 | TRIM2 | PXDN | 0.814 |
| TMEM71 | CYTH4 | 0.795 | GPR176 | RGCC | 0.758 | ZNF704 | PLEK | 0.752 |
| SLC15A4 | NOD2 | 0.723 | LRP4 | OSMR | 0.796 | FZD5 | VASN | 0.757 |
| RALGAPA2 | FILIP1L | 0.779 | SLA | WWC2 | 0.746 | TNC | MMP3 | 0.796 |
| CRIP2 | RIPK2 | 0.801 | FGR | PDLIM4 | 0.783 | CMTM4 | PDPN | 0.803 |
| LILRB2 | TLR1 | 0.761 | GOLPH3L | RIPOR3 | 0.804 | PKIB | PTGS1 | 0.795 |
| LILRB3 | HSPA6 | 0.691 | ASB7 | C1R | 0.776 | B4GALT4 | STAT5B | 0.770 |
| STAP2 | PDE4B | 0.750 | FGR | VIM | 0.773 | CSTA | BCAT1 | 0.735 |
| CRIP2 | ITPRIP | 0.808 | CAVIN1 | WNT5A | 0.807 | NEO1 | DUSP4 | 0.782 |
| CSF2RB | MCAM | 0.779 | LILRA2 | TNC | 0.792 | PRR16 | PRRX1 | 0.787 |
| SH2D3C | ACOD1 | 0.740 | LILRB2 | GADD45B | 0.807 | LOXL2 | TUBA1A | 0.773 |
| GOLPH3L | GFPT2 | 0.765 | RAB31 | OSMR | 0.766 | ZDHHC23 | CAVIN3 | 0.808 |
| WNT5A | LST1 | 0.793 | HCK | THEMIS2 | 0.753 | GADD45B | CXCR4 | 0.732 |
| SNORC | COTL1 | 0.776 | NEO1 | SLC15A3 | 0.770 | SWAP70 | INHBA | 0.745 |
| RHOH | TNFAIP2 | 0.721 | TIMP1 | TLR1 | 0.798 | BDH1 | FFAR2 | 0.777 |
| TMEM106C | IL6 | 0.813 | IL6 | STAT5B | 0.811 | AFG3L2 | THEMIS2 | 0.787 |
| PIP4P2 | CD69 | 0.724 | ARPIN | CYTH4 | 0.773 | GAS2L3 | GOLPH3L | 0.776 |
| C11orf96 | OSM | 0.814 | NDUFA10 | IER5 | 0.779 | ZNF704 | ST3GAL5 | 0.796 |
| LILRB3 | SPP1 | 0.714 | RNF149 | INHBA | 0.804 | PI15 | PLEK | 0.768 |
| FERMT2 | ASAP1 | 0.764 | IL11 | NCF2 | 0.815 | C1R | TUBA1A | 0.753 |
| GAS2L3 | GFPT2 | 0.764 | CLEC4E | WNT5A | 0.784 | PLAU | PTGS1 | 0.768 |
| TMEM106C | SERPINB9 | 0.757 | LGALS1 | S100A9 | 0.812 | TRPM6 | FCGR3B | 0.806 |
| RGS16 | CCN4 | 0.777 | SLC15A3 | SLA | 0.727 | IL1RN | LCP2 | 0.791 |
| CAVIN1 | MSN | 0.763 | STAP2 | TUBB6 | 0.776 | ERMP1 | SH2D3C | 0.779 |
| CR1 | TNF | 0.746 | IL6 | TNF | 0.803 | LILRB3 | CEMIP | 0.756 |
| MSN | SELP | 0.798 | OLFM1 | RGS16 | 0.793 | OLFM1 | PXDN | 0.791 |
| SLC35G2 | CREB5 | 0.789 | NR3C2 | GPR65 | 0.786 | CAVIN1 | HLX | 0.706 |
| TMEM106C | SLA | 0.749 | EGR3 | FAP | 0.757 | CEBPB | VASN | 0.742 |
| STAP2 | DUSP4 | 0.779 | FCGR2B | C2CD2 | 0.778 | TAGAP | CD86 | 0.734 |
| IER5 | P2RY14 | 0.742 | DUSP4 | VNN2 | 0.799 | CMTM4 | AMPD2 | 0.767 |
| MIR100HG | TNF | 0.779 | GZMB | BTG2 | 0.779 | NCF2 | CHST11 | 0.725 |
| CDX2 | RHOH | 0.762 | CSF3R | CCL2 | 0.722 | HCLS1 | PLA1A | 0.720 |
| VASN | TIMP1 | 0.719 | NR3C2 | FAM20C | 0.771 | GADD45B | C1R | 0.782 |
| CCDC69 | NFE2 | 0.734 | SPP1 | LAT2 | 0.756 | IL11 | MSN | 0.837 |
| EMP3 | CREB5 | 0.776 | PPP1R18 | SLFN11 | 0.751 | CTHRC1 | MCAM | 0.781 |
| TSPAN6 | EGR3 | 0.760 | FGR | MSN | 0.783 | FILIP1L | LAT2 | 0.781 |
| AGAP1 | GLT1D1 | 0.762 | ANGPT2 | PROK2 | 0.746 | SELP | SLC2A3 | 0.775 |
| RALGAPA2 | CSF3 | 0.809 | AFG3L2 | TCEA3 | 0.768 | HLX | SELE | 0.763 |
| RAB15 | CCDC3 | 0.778 | SLC15A4 | P2RY14 | 0.800 | CHI3L2 | GPR65 | 0.747 |
| CEMIP | TNFAIP6 | 0.787 | PPP2R5A | WNT5A | 0.819 | STAP2 | PDPN | 0.777 |
| HIBCH | CBX6 | 0.767 | STAT5B | TNF | 0.773 | AFG3L2 | SLC35G2 | 0.812 |
| UQCRC1 | SLC2A3 | 0.778 | ASB7 | BCL6 | 0.698 | CTHRC1 | GLT1D1 | 0.782 |
| RGCC | RIPK2 | 0.789 | RFLNB | GPR137B | 0.788 | DUSP2 | C2CD2 | 0.757 |
| GNAI2 | STC1 | 0.793 | TAGAP | ARHGAP25 | 0.701 | CXCR1 | MMP19 | 0.751 |
| ARPIN | ITPRIP | 0.800 | IL1B | SELE | 0.809 | CTHRC1 | PLAU | 0.768 |
| SERPINE2 | BCL2A1 | 0.777 | CDX2 | IL1RN | 0.773 | FAM155A | DOK3 | 0.776 |
| IL13RA2 | PTGS2 | 0.839 | WNK2 | HLX | 0.778 | ARPIN | SNN | 0.759 |
| PKIB | FPR1 | 0.822 | ITGAM | CD86 | 0.757 | PREX1 | TUBA1A | 0.743 |
| WWC2 | CCDC3 | 0.731 | CLEC4E | MEFV | 0.710 | DSP | CLMP | 0.785 |
| CTHRC1 | PPP1R18 | 0.769 | TMEM154 | STC1 | 0.808 | DPYSL2 | ITGAM | 0.752 |
| TMEM71 | CXCL8 | 0.788 | HIBCH | CXCR4 | 0.755 | BTG2 | FAM49A | 0.785 |
| JAZF1 | CCDC88A | 0.769 | FGF2 | SLC16A6 | 0.777 | ITGAM | SOD2 | 0.771 |
| PRICKLE2 | MSN | 0.790 | COQ9 | SELP | 0.803 | AFG3L2 | LDLRAD3 | 0.799 |
| PI15 | SOD2 | 0.761 | IL13RA2 | SLC11A1 | 0.880 | IL24 | CREB5 | 0.808 |
| NIPSNAP3A | MX2 | 0.794 | TRPM6 | RNF149 | 0.799 | HSPA6 | PREX1 | 0.694 |
| GNAI2 | GZMB | 0.769 | DUSP2 | ANGPTL2 | 0.758 | LST1 | BMERB1 | 0.803 |
| CHN1 | LCP1 | 0.753 | TMEM106C | TMEM273 | 0.759 | LILRB1 | CAVIN3 | 0.808 |
| FZD5 | ACOT9 | 0.801 | AFG3L2 | NCF2 | 0.780 | TMEM71 | BTG2 | 0.797 |
| SELENBP1 | SH3BP5 | 0.794 | RGS19 | RIPOR3 | 0.764 | TAGAP | PCBP3 | 0.728 |
| UBAC1 | ARPIN | 0.765 | MYO1D | CHI3L2 | 0.730 | MIR100HG | PIP4P2 | 0.747 |
| RAB15 | CXCR2 | 0.813 | RIPOR3 | GPR65 | 0.802 | EGR3 | CXCL8 | 0.762 |
| CHI3L2 | PPP1R18 | 0.711 | FCER1G | NNMT | 0.776 | TUBA1A | ENTPD1 | 0.785 |
| HIBCH | PPP2R5A | 0.765 | PAPPA | TUBB6 | 0.822 | PDPN | MYO1F | 0.784 |
| CSNK1G3 | NRG1 | 0.796 | RAI14 | ANGPT2 | 0.782 | GZMB | RFLNB | 0.789 |
| JAZF1 | TNFAIP6 | 0.799 | CSNK1G3 | HCK | 0.774 | AGFG2 | CGAS | 0.768 |
| SOD2 | TNFAIP2 | 0.748 | DSP | AQP9 | 0.791 | PCBP3 | TNFRSF10C | 0.779 |
| GPR183 | MYO1F | 0.752 | RILPL2 | EMP3 | 0.811 | CSNK1G3 | SH2D3C | 0.767 |
| TMEM154 | ZDHHC18 | 0.797 | TRIM2 | IL13RA2 | 0.866 | CD93 | LGALS1 | 0.790 |
| VNN2 | CD14 | 0.779 | DPYSL3 | CEMIP | 0.787 | CSF3R | FFAR2 | 0.689 |
| SERPINE1 | PROK2 | 0.741 | ICAM1 | AQP9 | 0.763 | IL13RA2 | WNT5A | 0.858 |
| GPR183 | IL11 | 0.812 | AFG3L2 | SHTN1 | 0.765 | ANGPT2 | P2RY13 | 0.786 |
| TCEA3 | FAM49A | 0.805 | RIPOR3 | CHST11 | 0.780 | SH3RF2 | IL11 | 0.835 |
| FZD5 | FPR2 | 0.802 | TRPM6 | LCP2 | 0.797 | SH3BP5 | P2RY14 | 0.772 |
| ASB7 | ACOD1 | 0.768 | RAB31 | NCF2 | 0.769 | IL6 | ZDHHC18 | 0.824 |
| COX10 | LILRB2 | 0.830 | POLDIP2 | NR3C2 | 0.783 | ADCY6 | SELL | 0.814 |
| EGR3 | FAM49A | 0.752 | CD300A | ACOD1 | 0.777 | ITGA5 | SLC11A1 | 0.780 |
| COX10 | CD93 | 0.815 | SLC35G2 | SLFN11 | 0.759 | FGF2 | MIR100HG | 0.784 |
| SORT1 | INHBA | 0.797 | KIAA1522 | FJX1 | 0.759 | F5 | NCF2 | 0.739 |
| B4GALT4 | PRR16 | 0.807 | CYTH4 | S100A8 | 0.811 | ZNF704 | IFITM2 | 0.765 |
| NNMT | CXCR4 | 0.756 | LILRB1 | PROK2 | 0.771 | SLA | SLFN11 | 0.728 |
| NFAM1 | SPHK1 | 0.731 | SNORC | EGR3 | 0.749 | TIMP1 | CCN4 | 0.765 |
| CBX6 | AMPD2 | 0.762 | PDPN | ST3GAL5 | 0.784 | DSP | MCAM | 0.782 |
| SORT1 | SLC35G2 | 0.799 | COQ9 | PHLDB2 | 0.782 | ASB7 | TNC | 0.798 |
| IL13RA2 | SLC2A3 | 0.831 | S1PR3 | SOD2 | 0.779 | DLC1 | SELP | 0.813 |
| ACOT9 | ANGPT2 | 0.786 | PDPN | S100A12 | 0.802 | GLIPR1 | CD14 | 0.728 |
| CSTA | TNF | 0.747 | TRPM6 | KCNJ15 | 0.781 | LDLRAD3 | SPHK1 | 0.792 |
| LGALS1 | SELE | 0.801 | PFKL | IFITM2 | 0.760 | GZMB | FAM155A | 0.816 |
| C2CD2 | FAM155A | 0.794 | ASAP1 | GPR65 | 0.768 | S100A12 | SLC16A6 | 0.785 |
| CAVIN1 | KCNJ15 | 0.708 | DSP | PTPRC | 0.783 | RALGAPA2 | CYTH4 | 0.767 |
| FGF7 | SLC2A3 | 0.778 | MMP19 | P2RY14 | 0.785 | IFITM2 | PLAU | 0.774 |
| HCK | HCLS1 | 0.713 | GPR176 | MEFV | 0.757 | CHN1 | CEP19 | 0.720 |
| DUSP2 | STC1 | 0.806 | RBMS1 | ZDHHC18 | 0.793 | TMEM71 | NOD2 | 0.729 |
| CGAS | TMEM154 | 0.740 | TMEM273 | HSPA6 | 0.746 | RAB31 | CGAS | 0.727 |
| GAS2L3 | VIM | 0.767 | PFKL | SH3BP5 | 0.794 | TRIM2 | STC1 | 0.806 |
| CSNK1G3 | SERPINE1 | 0.805 | CYTH4 | TNFRSF1B | 0.746 | TMEM71 | ENO2 | 0.807 |
| HSD11B2 | F5 | 0.791 | FCGR2B | PTGS2 | 0.798 | CHST15 | STC1 | 0.814 |
| CHRDL2 | SLA | 0.753 | RAB15 | HS3ST3B1 | 0.804 | COX10 | TCEA3 | 0.772 |
| FFAR2 | DOK3 | 0.742 | TREM1 | GFPT2 | 0.754 | PLAU | SOD2 | 0.764 |
| LCP1 | ITPRIP | 0.771 | EFNA4 | ATP5IF1 | 0.764 | UQCRC1 | ANGPTL2 | 0.801 |
| CSTA | JAZF1 | 0.732 | FPR2 | IL1B | 0.786 | CHST11 | CHST15 | 0.750 |
| SWAP70 | ITPRIP | 0.777 | NR3C2 | CCR1 | 0.818 | DUSP4 | LGALS1 | 0.798 |
| SERPINE2 | GPR137B | 0.808 | COL4A2 | SPP1 | 0.761 | CSF3 | ITPRIP | 0.806 |
| LOXL2 | GPR137B | 0.796 | CSNK1G3 | AMPD2 | 0.759 | GPR176 | PROK2 | 0.742 |
| RAB15 | RIPOR2 | 0.791 | C2CD2 | KCNJ15 | 0.723 | PAPPA | CCL4 | 0.818 |
| RNF149 | TNFRSF10C | 0.755 | AGAP1 | SLA | 0.753 | AMPD2 | BPGM | 0.780 |
| EGR3 | NRG1 | 0.772 | DSP | SLC2A3 | 0.767 | AKAP1 | IL11 | 0.830 |
| LILRB1 | GPR183 | 0.773 | GOLPH3L | SMAP2 | 0.756 | FAP | G0S2 | 0.796 |
| LRP4 | C1R | 0.796 | LILRA2 | SLA | 0.743 | ADCY6 | CXCL8 | 0.794 |
| CYP26B1 | SNN | 0.808 | CCDC69 | S100A9 | 0.826 | FCER1G | CBX6 | 0.758 |
| LCP2 | SELE | 0.802 | FGF7 | PIP4P2 | 0.784 | PLAU | BCL2A1 | 0.767 |
| PHLDB2 | HS3ST3B1 | 0.785 | MSN | TUBA1A | 0.770 | LCP2 | SELP | 0.805 |
| GLT1D1 | RNF149 | 0.780 | VAV1 | RASSF5 | 0.703 | FFAR2 | LAT2 | 0.777 |
| SNORC | PIP4P2 | 0.803 | DPYSL2 | C11orf96 | 0.783 | ACOT9 | PDE4B | 0.749 |
| FERMT2 | FCER1G | 0.778 | COL15A1 | BCL2A1 | 0.767 | TMEM106C | CCL2 | 0.763 |
| FCGR3B | CREB5 | 0.796 | ANGPT2 | SELP | 0.817 | LILRA2 | FCER1G | 0.756 |
| RAB31 | SNN | 0.781 | LILRB3 | CHST11 | 0.710 | LRP4 | BICD2 | 0.792 |
| SPP1 | CD53 | 0.720 | HSD11B2 | BPGM | 0.784 | GLT1D1 | AMPD2 | 0.756 |
| DPYSL3 | PDE4B | 0.743 | NFAM1 | CYTH4 | 0.748 | CXCL5 | TUBB6 | 0.774 |
| CLU | P2RY14 | 0.749 | LOXL2 | SRGN | 0.791 | BCL2A1 | RASSF5 | 0.744 |
| TNC | SPP1 | 0.785 | COX10 | PDLIM4 | 0.806 | COL4A2 | LILRA5 | 0.747 |
| IL1B | S100A12 | 0.788 | GLT1D1 | PPP1R18 | 0.749 | GAS2L3 | MEFV | 0.757 |
| NDFIP2 | TLR1 | 0.795 | CXCR1 | VAV1 | 0.696 | CHN1 | RGCC | 0.772 |
| TCF4 | SH3BP5 | 0.739 | CSF3 | NCF4 | 0.783 | PDPN | GLIPR1 | 0.732 |
| GAS2L3 | CYTH4 | 0.768 | CLU | STC1 | 0.818 | DPYSL3 | ARHGAP25 | 0.765 |
| KCNJ15 | MIR100HG | 0.744 | AFG3L2 | IL13RA2 | 0.852 | PPP2R5A | CSTA | 0.769 |
| PTGS1 | TNFAIP6 | 0.792 | LRRK2 | IER5 | 0.719 | OCRL | PKIB | 0.756 |
| RIPOR3 | PTPRC | 0.790 | NEO1 | RNF149 | 0.803 | VAV1 | RIPK2 | 0.769 |
| COTL1 | GPR137B | 0.783 | HSD11B2 | DUSP4 | 0.784 | FAP | PXDN | 0.769 |
| ADCY6 | RILPL2 | 0.816 | CSF3R | BCL6 | 0.678 | RBMS1 | TLR4 | 0.705 |
| CHN1 | LDLRAD3 | 0.783 | LILRB3 | CCR1 | 0.768 | RHOH | MMP19 | 0.741 |
| KIAA1522 | BASP1 | 0.789 | ANGPT2 | SPOCK1 | 0.776 | LILRB1 | JAZF1 | 0.778 |
| SWAP70 | SELE | 0.761 | STAP2 | SERPINE2 | 0.779 | UBAC1 | CXCR2 | 0.813 |
| GPR176 | TNF | 0.783 | TRPM6 | TSPAN6 | 0.764 | CXCR2 | SPOCK1 | 0.812 |
| FCGR2B | SOD2 | 0.789 | ZNF704 | HLX | 0.736 | CREM | CEMIP | 0.778 |
| STC1 | CD14 | 0.800 | ICAM1 | P2RY14 | 0.776 | GOLPH3L | C5AR1 | 0.797 |
| CBX6 | LY96 | 0.783 | ATP5IF1 | FGF7 | 0.798 | GPR176 | FPR2 | 0.784 |
| DUSP2 | SLC15A3 | 0.759 | EMP3 | PTGS2 | 0.787 | RALGAPA2 | CD300A | 0.820 |
| HSPA6 | SLA | 0.719 | COL15A1 | CCDC88A | 0.779 | FAP | PREX1 | 0.760 |
| FGF7 | TCF4 | 0.774 | NEO1 | CCDC88A | 0.788 | CMTM4 | NR3C2 | 0.785 |
| VASN | TMEM71 | 0.754 | CLU | SLA | 0.730 | LILRB2 | OLFM1 | 0.810 |
| RILPL2 | BPGM | 0.786 | ASAP1 | THBD | 0.800 | AMPD2 | SH3BP5 | 0.758 |
| FERMT2 | PTGS2 | 0.787 | EFNA4 | AGFG2 | 0.763 | AGFG2 | PDLIM4 | 0.789 |
| SLA | THBD | 0.772 | LY96 | SMAP2 | 0.773 | RBMS1 | ENTPD1 | 0.798 |
| GPR183 | SLC2A3 | 0.711 | CSF2RB | SLFN11 | 0.734 | COL15A1 | WWC2 | 0.748 |
| COL15A1 | RNF149 | 0.778 | CD300A | ENO2 | 0.811 | HCLS1 | RBMS1 | 0.711 |
| RALGAPA2 | BCL2A1 | 0.784 | VASN | NRG1 | 0.761 | AKAP1 | RIPOR2 | 0.782 |
| TNF | SNN | 0.801 | FGF7 | ACOT9 | 0.788 | FGR | CLEC4A | 0.784 |
| CREM | JAZF1 | 0.780 | FCGR2B | P2RY14 | 0.795 | PRICKLE2 | SSH2 | 0.815 |
| ABL2 | SPOCK1 | 0.762 | CSTA | PLEK | 0.731 | S1PR1 | OSMR | 0.789 |
| PIK3AP1 | SELL | 0.744 | FCER1G | MIR100HG | 0.763 | EFNA4 | CGAS | 0.767 |
| ITGAM | TLR4 | 0.685 | GOLPH3L | FCGR3B | 0.805 | RGCC | NFE2 | 0.808 |
| PREX1 | ITPRIP | 0.800 | CMTM4 | LDLRAD3 | 0.804 | GNAI2 | SLC16A6 | 0.703 |
| ATP5IF1 | FGR | 0.790 | RFLNB | PDE4B | 0.765 | GLCE | RGS19 | 0.733 |
| GALNT12 | FAM49A | 0.808 | CLU | SELE | 0.803 | ENO2 | PTGS1 | 0.789 |
| LCP1 | SSH2 | 0.716 | FAM20C | MAP4K4 | 0.765 | DLC1 | OSM | 0.833 |
| JAZF1 | FGR | 0.770 | PPP1R18 | SPOCK1 | 0.775 | NDFIP2 | SWAP70 | 0.751 |
| PDE4B | GMFG | 0.768 | DPYSL2 | PAPPA | 0.837 | NCF2 | VAV1 | 0.724 |
| HSPA6 | VIM | 0.740 | RIPOR3 | TUBA1A | 0.792 | LDLRAD3 | PCBP3 | 0.786 |
| FCGR2B | RGCC | 0.776 | OLFM1 | PRICKLE2 | 0.792 | CLU | SLC15A4 | 0.773 |
| NFE2 | BTG2 | 0.779 | FERMT2 | CGAS | 0.767 | F5 | GADD45B | 0.781 |
| ARPIN | FCGR2B | 0.763 | CHI3L2 | CSTA | 0.731 | GLCE | DUSP2 | 0.760 |
| CEBPB | LAT2 | 0.778 | LILRB1 | PTPRC | 0.774 | CSNK1G3 | PIP4P2 | 0.793 |
| S100A8 | HS3ST3B1 | 0.814 | FAP | PLAU | 0.756 | LILRB1 | SH3BP5 | 0.772 |
| BASP1 | TUBB6 | 0.786 | STAT5B | SLC35G2 | 0.803 | SLC16A6 | GFPT2 | 0.745 |
| SERPINB9 | ACOD1 | 0.758 | ATP5IF1 | CLEC4E | 0.779 | STAP2 | FPR1 | 0.786 |
| ANGPT2 | LAT2 | 0.786 | TRAK1 | FCER1G | 0.774 | TREM1 | PTGS1 | 0.771 |
| GALNT12 | FGF7 | 0.807 | TRAK1 | NOD2 | 0.727 | GPR176 | CLEC4E | 0.766 |
| S100A12 | BPGM | 0.801 | ANGPT2 | CD86 | 0.810 | CR1 | RGCC | 0.769 |
| FAM155A | TUBA1A | 0.790 | NIBAN1 | GADD45B | 0.782 | RAB15 | CD93 | 0.809 |
| COQ9 | DLC1 | 0.817 | COQ9 | CLU | 0.763 | SERPINE1 | SOD2 | 0.787 |
| BDH1 | BICD2 | 0.784 | S1PR1 | CHRDL2 | 0.771 | FGF7 | MMP19 | 0.784 |
| STAP2 | SELENBP1 | 0.771 | TMEM71 | LILRA5 | 0.769 | FPR1 | CLEC4E | 0.734 |
| ADCY6 | HIBCH | 0.798 | DPYSL3 | NRCAM | 0.749 | CSF2RB | F5 | 0.772 |
| IL1B | SRGN | 0.789 | TMEM273 | MMP19 | 0.782 | CLEC4A | C5AR1 | 0.760 |
| NDUFA10 | CTHRC1 | 0.795 | CYTH4 | OSMR | 0.786 | NFE2 | SPOCK1 | 0.794 |
| CREM | CD93 | 0.787 | S1PR3 | GZMB | 0.783 | BDH1 | TNFRSF10C | 0.754 |
| COL7A1 | FCGR2B | 0.792 | STAP2 | VAV1 | 0.747 | PPP2R5A | SLC11A1 | 0.803 |
| LCP2 | CCN4 | 0.768 | TMEM71 | CREB5 | 0.747 | SLC15A4 | ENO2 | 0.778 |
| FZD5 | SERPINB9 | 0.765 | UBAC1 | GOLPH3L | 0.776 | CSNK1G3 | FAM49A | 0.811 |
| PLAU | WWC2 | 0.746 | FPR2 | MMP3 | 0.809 | PLAU | ITPRIP | 0.793 |
| CREM | P2RY14 | 0.773 | SHTN1 | ATP5IF1 | 0.788 | AKAP1 | ADGRG3 | 0.771 |
| GPR137B | TNFRSF1B | 0.750 | FAM155A | WNT5A | 0.813 | RALGAPA2 | GMFG | 0.804 |
| PDPN | STAT5B | 0.786 | ASB7 | TNFAIP6 | 0.780 | ENO2 | GNAI2 | 0.777 |
| TCEA3 | LILRB3 | 0.739 | SWAP70 | CYP26B1 | 0.759 | KIAA1522 | SSH2 | 0.771 |
| DPYSL3 | P2RY14 | 0.758 | BCAT1 | SLA | 0.721 | LRRK2 | IL13RA2 | 0.866 |
| PTPRC | GPR65 | 0.730 | MYO1D | PPP2R5A | 0.784 | SNN | CCDC3 | 0.780 |
| RILPL2 | LY96 | 0.803 | ERMP1 | CCR1 | 0.812 | COL4A2 | ADGRG3 | 0.744 |
| PIK3AP1 | VNN2 | 0.744 | WNK2 | MMP3 | 0.817 | PIK3AP1 | SNN | 0.734 |
| CLU | SELL | 0.769 | EMP3 | TMEM154 | 0.762 | PIK3AP1 | S1PR1 | 0.704 |
| TCEA3 | CHN1 | 0.804 | RNF149 | CLEC4A | 0.805 | NDUFA10 | PROK2 | 0.751 |
| CAVIN3 | JAZF1 | 0.766 | RNF149 | TUBA1A | 0.792 | LILRB1 | VASN | 0.760 |
| CBX6 | PDLIM4 | 0.773 | TNFAIP2 | TNFRSF10C | 0.748 | TMEM106C | FFAR2 | 0.763 |
| COTL1 | P2RY13 | 0.785 | NDFIP2 | PDLIM4 | 0.793 | MYO1D | PRRX1 | 0.776 |
| GPR176 | SLC11A1 | 0.778 | ANGPTL2 | PHLDB2 | 0.774 | CSTA | FGR | 0.749 |
| COQ9 | G0S2 | 0.808 | FCER1G | SLA | 0.742 | NEO1 | BICD2 | 0.797 |
| TMEM106C | ZDHHC18 | 0.758 | GALNT12 | CSTA | 0.782 | ACOT9 | HCLS1 | 0.738 |
| SHTN1 | TCF4 | 0.773 | CBX6 | HCK | 0.746 | BICD2 | RBMS1 | 0.748 |
| S1PR3 | CYP26B1 | 0.803 | GADD45B | FAM20C | 0.768 | TIMP1 | SSH2 | 0.786 |
| ACOT9 | C2CD2 | 0.781 | CHRDL2 | BTG2 | 0.774 | BICD2 | RAI14 | 0.760 |
| CLU | PREX1 | 0.764 | GLIPR1 | NCF2 | 0.721 | BASP1 | C2CD2 | 0.796 |
| CCR1 | RBMS1 | 0.789 | HLX | AQP9 | 0.754 | ANKRD35 | CXCR2 | 0.818 |
| CD93 | STAT5B | 0.794 | CCDC69 | LCP2 | 0.735 | DSP | ARHGAP25 | 0.772 |
| NIBAN1 | CCDC88A | 0.746 | RALGAPA2 | FPR2 | 0.780 | CYP26B1 | THEMIS2 | 0.816 |
| IL1B | CCDC88A | 0.799 | UBAC1 | NDUFA10 | 0.798 | TNFRSF1B | LST1 | 0.725 |
| S1PR3 | ITGA5 | 0.763 | CREM | SLFN11 | 0.719 | NCF4 | CLMP | 0.783 |
| S1PR3 | RGS16 | 0.741 | SOD2 | LAT2 | 0.791 | COL15A1 | PROK2 | 0.730 |
| OLFM1 | CHST2 | 0.779 | LAT2 | GFPT2 | 0.752 | LRP4 | LILRA2 | 0.775 |
| ABL2 | ANGPT2 | 0.790 | PI15 | GMFG | 0.786 | COL4A2 | GLT1D1 | 0.759 |
| PKIB | WNT5A | 0.832 | LILRA5 | PRRX1 | 0.759 | EMP3 | CD14 | 0.762 |
| GLIPR1 | GPR65 | 0.703 | ANGPTL2 | ANGPT2 | 0.778 | PDPN | PHLDB2 | 0.788 |
| GLCE | TMEM154 | 0.770 | CSNK1G3 | BCL2A1 | 0.781 | GLT1D1 | S1PR1 | 0.734 |
| GOLPH3L | CD14 | 0.771 | NRG1 | NOD2 | 0.751 | PLAU | C5AR1 | 0.766 |
| CSNK1G3 | ARPIN | 0.773 | CDX2 | CBX6 | 0.780 | GPR183 | SRGN | 0.775 |
| CD300A | CCDC69 | 0.798 | SOD2 | PXDN | 0.788 | MX2 | GPR65 | 0.738 |
| IL6 | CXCR4 | 0.766 | B4GALT4 | TNFAIP2 | 0.747 | CCR1 | VIM | 0.773 |
| CSF3 | SLFN11 | 0.767 | HIBCH | S100A12 | 0.804 | C2CD2 | ST3GAL5 | 0.764 |
| ZDHHC18 | CHST2 | 0.771 | CREM | FLT1 | 0.771 | CCR1 | CREM | 0.791 |
| PI15 | SELE | 0.802 | CAVIN3 | ZDHHC18 | 0.777 | C11orf96 | C5AR1 | 0.783 |
| RGCC | MIR100HG | 0.757 | AKAP1 | DLC1 | 0.822 | GAS2L3 | SPHK1 | 0.769 |
| CSF3R | SPOCK1 | 0.750 | HSD11B2 | CCDC3 | 0.792 | NDUFA10 | AKAP1 | 0.828 |
| CCDC69 | ITGAM | 0.718 | NDUFA10 | SELP | 0.807 | INHBA | STC1 | 0.801 |
| PFKL | PROK2 | 0.760 | GZMB | GADD45B | 0.776 | CD93 | MEFV | 0.792 |
| CSF3 | CXCR4 | 0.766 | GOLPH3L | SRGN | 0.806 | RGS19 | CD53 | 0.668 |
| ATP5IF1 | BMERB1 | 0.809 | PI15 | PCBP3 | 0.783 | RAI14 | PCBP3 | 0.783 |
| ASB7 | ATP5IF1 | 0.770 | BCL2A1 | SLC16A6 | 0.735 | FCGR2B | RASSF5 | 0.779 |
| ARPIN | TIMP1 | 0.756 | TCEA3 | FZD5 | 0.752 | BMERB1 | ENTPD1 | 0.811 |
| JAZF1 | SLC16A6 | 0.759 | CYTH4 | SPOCK1 | 0.790 | MEFV | NNMT | 0.789 |
| CD93 | TREM1 | 0.766 | FILIP1L | RASSF5 | 0.766 | ZDHHC23 | CSTA | 0.779 |
| TNF | C1R | 0.806 | PLA1A | WNT5A | 0.797 | GALNT12 | LILRB1 | 0.781 |
| PDPN | GPR65 | 0.777 | RAB15 | NIBAN1 | 0.808 | MYO1F | PTGS1 | 0.778 |
| NCF4 | SPP1 | 0.754 | PLA1A | CD86 | 0.770 | PDPN | P2RY14 | 0.785 |
| ENO2 | FAP | 0.757 | PFKL | DUSP4 | 0.768 | PLEK | CEMIP | 0.768 |
| SERPINE2 | TUBA1A | 0.786 | EMP3 | LCP1 | 0.733 | PRR16 | CD14 | 0.777 |
| GALNT12 | VIM | 0.795 | AGAP1 | SNORC | 0.785 | IL13RA2 | THBD | 0.849 |
| CYP26B1 | MAP4K4 | 0.806 | LILRB2 | FCER1G | 0.780 | IL24 | CHST15 | 0.799 |
| GPR176 | HCLS1 | 0.700 | AKAP1 | PLEK | 0.770 | BDH1 | SMAP2 | 0.751 |
| GADD45B | TUBB6 | 0.794 | NNMT | THEMIS2 | 0.798 | NFAM1 | NNMT | 0.765 |
| ATP5IF1 | FCER1G | 0.773 | IL24 | TNFAIP6 | 0.800 | DLC1 | CCDC88A | 0.798 |
| SH2D3C | RGS19 | 0.705 | SH3RF2 | ST3GAL5 | 0.775 | PPP1R18 | SPHK1 | 0.749 |
| CSTA | LCP2 | 0.729 | ZDHHC23 | ABL2 | 0.827 | ERMP1 | C2CD2 | 0.803 |
| BCAT1 | TNFRSF10C | 0.768 | LILRB2 | MMP3 | 0.801 | AFG3L2 | RGCC | 0.770 |
| RNF149 | HLX | 0.746 | RAB31 | CXCL8 | 0.779 | ASB7 | AQP9 | 0.783 |
| ADGRG3 | FGF7 | 0.792 | TCEA3 | CXCL8 | 0.796 | LGALS1 | NOD2 | 0.731 |
| MYO1D | FAM49A | 0.810 | LRRK2 | ANKRD35 | 0.794 | SORT1 | SLA | 0.742 |
| GAS2L3 | SNN | 0.774 | OCRL | IL6 | 0.816 | SERPINE1 | LST1 | 0.793 |
| RILPL2 | CAVIN1 | 0.772 | IL11 | CYP26B1 | 0.832 | RIPOR3 | RGCC | 0.782 |
| KIAA1522 | CGAS | 0.766 | CD14 | RIPOR2 | 0.738 | GLCE | AMPD2 | 0.785 |
| OLFM1 | SLC15A3 | 0.767 | BPGM | CD86 | 0.788 | SMAP2 | PXDN | 0.768 |
| TSPAN6 | AKAP1 | 0.794 | NDUFA10 | CD86 | 0.787 | GPR176 | SPHK1 | 0.757 |
| INHBA | SLC16A6 | 0.802 | RALGAPA2 | VNN2 | 0.824 | FAM49A | GPR65 | 0.736 |
| AMPD2 | GADD45B | 0.773 | SLA | VNN2 | 0.783 | NDUFA10 | S100A9 | 0.826 |
| AFG3L2 | POLDIP2 | 0.760 | DUSP4 | HS3ST3B1 | 0.769 | CAVIN3 | CGAS | 0.772 |
| NCF4 | STC1 | 0.807 | LRRK2 | DUSP2 | 0.782 | AMPD2 | RASSF5 | 0.758 |
| TMEM273 | ABL2 | 0.777 | PDPN | GLT1D1 | 0.796 | IL1RN | TNFAIP6 | 0.790 |
| MSN | C1R | 0.778 | BDH1 | IL1B | 0.790 | SRGN | BTG2 | 0.786 |
| RILPL2 | FLT1 | 0.802 | SWAP70 | CCDC88A | 0.713 | CMTM4 | COTL1 | 0.778 |
| ICAM1 | CREB5 | 0.757 | LRP4 | CD69 | 0.782 | C11orf96 | CLMP | 0.779 |
| COX10 | COQ9 | 0.775 | S1PR1 | MEFV | 0.752 | ITGAM | SELL | 0.784 |
| ARPIN | ANGPTL2 | 0.778 | NR3C2 | DUSP2 | 0.793 | AGFG2 | CSF2RB | 0.794 |
| TCEA3 | FGF7 | 0.809 | CHI3L1 | G0S2 | 0.828 | S1PR1 | CCDC69 | 0.733 |
| ZNF704 | MIR100HG | 0.794 | SELENBP1 | LGALS1 | 0.814 | WNK2 | RIPK2 | 0.810 |
| CD93 | THBD | 0.786 | VNN2 | OSMR | 0.813 | S100A9 | CCL4 | 0.798 |
| PAPPA | S100A8 | 0.840 | AGFG2 | FGF2 | 0.772 | ICAM1 | LAT2 | 0.759 |
| LDLRAD3 | CSF3 | 0.801 | AMPD2 | IL11 | 0.832 | TSPAN6 | CHST15 | 0.793 |
| COL7A1 | TREM1 | 0.779 | NEO1 | TMEM106C | 0.786 | SH3RF2 | PRRX1 | 0.770 |
| ATP5IF1 | FJX1 | 0.767 | ACOT9 | PDLIM4 | 0.794 | CHI3L2 | DPYSL2 | 0.735 |
| AKAP1 | DUSP4 | 0.781 | AGFG2 | SPOCK1 | 0.777 | NDFIP2 | COL7A1 | 0.806 |
| TCF4 | SSH2 | 0.753 | PRICKLE2 | RHOH | 0.775 | CTHRC1 | IL6 | 0.824 |
| VASN | PLEK | 0.740 | PFKL | GLT1D1 | 0.765 | ABL2 | SMAP2 | 0.739 |
| NR3C2 | ITGA5 | 0.782 | RNF149 | GFPT2 | 0.765 | RGS16 | CCL2 | 0.768 |
| GLCE | CCDC88A | 0.779 | ZDHHC23 | FPR1 | 0.815 | BASP1 | IL1RN | 0.781 |
| LOXL2 | SLC11A1 | 0.802 | NFE2 | ACOD1 | 0.747 | OCRL | TUBA1A | 0.793 |
| PI15 | ENTPD1 | 0.772 | FGF2 | NNMT | 0.783 | AGAP1 | SELL | 0.797 |
| S100A8 | GPR65 | 0.818 | F5 | CCN4 | 0.782 | BDH1 | CEBPB | 0.812 |
| SLC15A3 | C1R | 0.759 | WNT5A | P2RY14 | 0.807 | GAS2L3 | PROK2 | 0.757 |
| IER5 | VIM | 0.736 | DSP | PROK2 | 0.733 | TIMP1 | GFPT2 | 0.742 |
| SLC15A3 | CEP19 | 0.684 | NIPSNAP3A | OSMR | 0.787 | CHI3L1 | C11orf96 | 0.795 |
| NNMT | ITPRIP | 0.801 | PXDN | SPHK1 | 0.802 | MYO1D | LY96 | 0.810 |
| FGF7 | FLT1 | 0.777 | SORT1 | SH3BP5 | 0.754 | HCLS1 | AQP9 | 0.747 |
| P2RY13 | CLMP | 0.767 | SOD2 | SPP1 | 0.764 | CMTM8 | PRR16 | 0.803 |
| IL11 | LOXL2 | 0.826 | STC1 | THEMIS2 | 0.808 | MMP3 | ACOD1 | 0.781 |
| FILIP1L | RHOH | 0.764 | SPOCK1 | TUBB6 | 0.771 | RALGAPA2 | SPOCK1 | 0.789 |
| CD93 | FLT1 | 0.780 | AMPD2 | TNFAIP2 | 0.746 | RIPOR3 | TIMP1 | 0.782 |
| ANKRD35 | SLFN11 | 0.733 | PRICKLE2 | FFAR2 | 0.807 | FPR1 | GPR137B | 0.775 |
| ITGA5 | TIMP1 | 0.761 | TSPAN6 | CHST2 | 0.784 | CEBPB | TLR1 | 0.799 |
| ENO2 | RFLNB | 0.778 | NR3C2 | LILRB2 | 0.813 | JAZF1 | ITGA5 | 0.781 |
| CMTM4 | LILRB1 | 0.783 | CHST15 | BCAT1 | 0.768 | UQCRC1 | TLR4 | 0.739 |
| BDH1 | AKAP1 | 0.800 | FCGR3B | BICD2 | 0.808 | INHBA | TUBB6 | 0.784 |
| B4GALT4 | PXDN | 0.798 | BDH1 | CREM | 0.788 | TNC | THBD | 0.793 |
| TCF4 | TNFRSF10C | 0.759 | RGCC | S100A8 | 0.819 | DSP | SELP | 0.809 |
| COL15A1 | CYP26B1 | 0.811 | SELENBP1 | ATP5IF1 | 0.765 | RALGAPA2 | CAVIN1 | 0.771 |
| CCDC69 | HCK | 0.757 | CHN1 | NRCAM | 0.770 | NIPSNAP3A | WWC2 | 0.765 |
| CSF3R | FCGR3B | 0.751 | EMP3 | PHLDB2 | 0.765 | AGAP1 | IFITM2 | 0.758 |
| LOXL2 | CD14 | 0.757 | LAT2 | RASSF5 | 0.750 | FGR | CCDC3 | 0.769 |
| CSF3R | CREB5 | 0.700 | GPR176 | BCAT1 | 0.743 | SLC15A3 | BCAT1 | 0.760 |
| CLEC4A | GPR137B | 0.806 | ARPIN | CXCR2 | 0.829 | CMTM8 | TUBB6 | 0.792 |
| OCRL | CR1 | 0.740 | EFNA4 | CEMIP | 0.779 | CSF3 | SELP | 0.824 |
| C2CD2 | ITGAM | 0.765 | TMEM273 | OSMR | 0.784 | SELP | STC1 | 0.839 |
| FGF7 | NCF4 | 0.792 | PI15 | BTG2 | 0.765 | DUSP2 | S1PR3 | 0.762 |
| WNK2 | FGF7 | 0.812 | S1PR1 | ITGA5 | 0.779 | SNORC | SRGN | 0.807 |
| DPYSL3 | SELE | 0.797 | AGFG2 | SLC2A3 | 0.763 | FPR1 | PRR16 | 0.791 |
| SELENBP1 | CCR1 | 0.824 | MX2 | TIMP1 | 0.791 | SPHK1 | CD86 | 0.770 |
| FILIP1L | CEMIP | 0.806 | SHTN1 | FGF7 | 0.780 | LCP1 | VIM | 0.739 |
| PRICKLE2 | PROK2 | 0.743 | CHST15 | PLAU | 0.780 | ANKRD35 | LAT2 | 0.758 |
| NDUFA10 | WWC2 | 0.764 | IFITM2 | NRCAM | 0.757 | GAS2L3 | RGS16 | 0.771 |
| LGALS1 | WNT5A | 0.806 | SH3RF2 | TNFAIP6 | 0.791 | HSPA6 | CCDC3 | 0.763 |
| ZNF704 | RIPOR3 | 0.807 | AKAP1 | SELP | 0.814 | TMEM106C | ATP5IF1 | 0.770 |
| CMTM4 | AKAP1 | 0.806 | MX2 | GMFG | 0.790 | SH3RF2 | ENTPD1 | 0.794 |
| TRIM2 | S1PR3 | 0.772 | RIPOR3 | NOD2 | 0.747 | SWAP70 | NCF2 | 0.710 |
| NIPSNAP3A | ZNF704 | 0.814 | OLFM1 | IL13RA2 | 0.855 | ASB7 | GLCE | 0.773 |
| CGAS | DPYSL3 | 0.762 | S1PR3 | PAPPA | 0.826 | PCBP3 | ARHGAP25 | 0.760 |
| SNORC | RILPL2 | 0.813 | AKAP1 | RNF149 | 0.799 | COL15A1 | ITGAM | 0.768 |
| OCRL | CSTA | 0.746 | RIPK2 | THEMIS2 | 0.780 | BCAT1 | VIM | 0.745 |
| EFNA4 | S1PR1 | 0.774 | FCGR3B | SELL | 0.814 | POLDIP2 | CLEC4E | 0.777 |
| GALNT12 | TLR4 | 0.758 | MMP3 | RBMS1 | 0.795 | MMP19 | TUBA1A | 0.773 |
| BDH1 | STAT5B | 0.770 | F5 | PTGS2 | 0.770 | HIBCH | FILIP1L | 0.786 |
| DUSP4 | BCAT1 | 0.798 | LRP4 | BCAT1 | 0.777 | LILRB3 | S100A12 | 0.748 |
| INHBA | MSN | 0.793 | RALGAPA2 | CD86 | 0.783 | WNK2 | FCGR3B | 0.823 |
| FILIP1L | CCN4 | 0.788 | LILRA5 | TLR1 | 0.772 | CEBPB | NOD2 | 0.729 |
| FCGR2B | ACOD1 | 0.781 | RAB15 | CSF2RB | 0.808 | ENO2 | SLA | 0.734 |
| SORT1 | SLC15A4 | 0.785 | TNFAIP6 | BMERB1 | 0.809 | SLC2A3 | TUBA1A | 0.730 |
| TRAK1 | GLIPR1 | 0.763 | ANGPT2 | FFAR2 | 0.787 | GPR183 | SOD2 | 0.775 |
| LILRB3 | SH3BP5 | 0.721 | CD93 | PRR16 | 0.815 | SELENBP1 | TLR4 | 0.756 |
| PTPRC | SLFN11 | 0.726 | ASB7 | RNF149 | 0.791 | CSNK1G3 | HCLS1 | 0.732 |
| BASP1 | CEMIP | 0.784 | TRPM6 | MSN | 0.772 | RIPK2 | MAP4K4 | 0.782 |
| ZNF704 | COL7A1 | 0.800 | DUSP4 | BCL6 | 0.699 | ANKRD35 | DUSP2 | 0.779 |
| PAPPA | PHLDB2 | 0.837 | FCGR2B | PI15 | 0.779 | HSPA6 | BPGM | 0.765 |
| ABL2 | VNN2 | 0.835 | ANGPTL2 | MMP19 | 0.760 | MMP19 | ST3GAL5 | 0.769 |
| GOLPH3L | LST1 | 0.772 | SORT1 | OSM | 0.816 | ASAP1 | SPHK1 | 0.762 |
| MMP19 | NCF4 | 0.763 | TUBA1A | SLC35G2 | 0.786 | TMEM273 | SLA | 0.751 |
| TMEM154 | CD53 | 0.723 | PPP1R18 | CHST11 | 0.737 | ASB7 | MYO1D | 0.804 |
| EMP3 | SERPINB9 | 0.778 | TNF | HS3ST3B1 | 0.788 | NIPSNAP3A | CCR1 | 0.818 |
| IFITM2 | NFAM1 | 0.738 | NOD2 | CREB5 | 0.718 | NDFIP2 | INHBA | 0.797 |
| FCGR2B | IL6 | 0.809 | IL13RA2 | RIPK2 | 0.855 | NIBAN1 | LGALS1 | 0.776 |
| TMEM71 | CCN4 | 0.784 | CEBPB | VIM | 0.778 | PTGS1 | BMERB1 | 0.808 |
| COL15A1 | NRG1 | 0.806 | FCGR3B | CCL2 | 0.797 | AGFG2 | FFAR2 | 0.762 |
| CD93 | CXCL8 | 0.794 | TRAK1 | SLC2A3 | 0.766 | SOD2 | SLC35G2 | 0.793 |
| LDLRAD3 | S100A9 | 0.813 | SNORC | S1PR1 | 0.798 | RIPOR3 | IL1RN | 0.820 |
| CYTH4 | MMP3 | 0.781 | COX10 | SERPINB9 | 0.791 | FCGR3B | CCL4 | 0.798 |
| PPP2R5A | CD69 | 0.783 | WNK2 | TMEM106C | 0.808 | CHI3L2 | FILIP1L | 0.755 |
| CCL2 | TLR1 | 0.768 | RAI14 | CXCL5 | 0.783 | LY96 | CEP19 | 0.768 |
| CAVIN1 | CYP26B1 | 0.811 | DUSP2 | WWC2 | 0.778 | PFKL | TUBB6 | 0.806 |
| TNC | TNFAIP2 | 0.781 | PIK3AP1 | TLR1 | 0.726 | SH3RF2 | NFE2 | 0.790 |
| SOD2 | MAP4K4 | 0.769 | LILRA2 | PIP4P2 | 0.772 | IER5 | SNN | 0.767 |
| FILIP1L | BCL2A1 | 0.783 | HCLS1 | IER5 | 0.667 | CCDC88A | PREX1 | 0.722 |
| IFITM2 | CGAS | 0.758 | FZD5 | CD93 | 0.795 | PDPN | GZMB | 0.787 |
| ADCY6 | PRRX1 | 0.777 | BPGM | CREB5 | 0.767 | PPP1R18 | HS3ST3B1 | 0.769 |
| CAVIN3 | FJX1 | 0.784 | IL13RA2 | MEFV | 0.868 | CXCL8 | MMP3 | 0.790 |
| ERMP1 | PI15 | 0.793 | FCGR3B | ST3GAL5 | 0.819 | ZNF704 | UQCRC1 | 0.804 |
| LY96 | TNFRSF1B | 0.757 | TNFAIP6 | RIPOR2 | 0.781 | LAT2 | MAP4K4 | 0.786 |
| ANGPT2 | IL13RA2 | 0.855 | NDUFA10 | ZDHHC18 | 0.797 | RGS19 | PAPPA | 0.803 |
| ARPIN | CD14 | 0.759 | TCF4 | OSMR | 0.771 | LRP4 | SPHK1 | 0.768 |
| CSF2RB | CCL2 | 0.769 | NR3C2 | SSH2 | 0.786 | CHN1 | ACOD1 | 0.794 |
| CXCR4 | CD53 | 0.669 | MIR100HG | TREM1 | 0.783 | JAZF1 | CD86 | 0.775 |
| RHOH | IER5 | 0.695 | CSTA | PIP4P2 | 0.724 | TLR1 | CLMP | 0.781 |
| CEBPB | TUBA1A | 0.794 | SLC15A3 | CREB5 | 0.761 | GPR176 | STC1 | 0.794 |
| ARPIN | SH2D3C | 0.775 | CMTM8 | ANKRD35 | 0.773 | PKIB | BCL2A1 | 0.799 |
| SLC15A4 | IL1B | 0.806 | AKAP1 | HS3ST3B1 | 0.816 | GADD45B | ARHGAP25 | 0.763 |
| CD300A | VIM | 0.791 | SORT1 | CXCL5 | 0.762 | CSF3R | GMFG | 0.743 |
| LILRB3 | MIR100HG | 0.734 | IL11 | SPP1 | 0.820 | FFAR2 | SLC15A3 | 0.744 |
| ADCY6 | NDUFA10 | 0.796 | TAGAP | TIMP1 | 0.727 | PTGS1 | FAM155A | 0.815 |
| GLCE | MSN | 0.787 | NNMT | TUBB6 | 0.790 | PDE4B | SPP1 | 0.732 |
| SORT1 | CR1 | 0.738 | LILRA5 | P2RY13 | 0.749 | NIBAN1 | LCP2 | 0.747 |
| GPR176 | GNAI2 | 0.736 | S1PR1 | SERPINE1 | 0.799 | CLU | TNFAIP6 | 0.792 |
| DUSP2 | FAM155A | 0.811 | ATP5IF1 | ITPRIP | 0.817 | RAB31 | PRR16 | 0.800 |
| FJX1 | C2CD2 | 0.750 | OLFM1 | CSTA | 0.730 | WNK2 | BMERB1 | 0.842 |
| FFAR2 | TNFRSF10C | 0.706 | CAVIN3 | NRG1 | 0.817 | TMEM273 | COTL1 | 0.773 |
| SORT1 | SRGN | 0.802 | ADCY6 | FPR1 | 0.806 | SHTN1 | C11orf96 | 0.779 |
| IL13RA2 | NNMT | 0.866 | EGR3 | CD86 | 0.746 | RAB15 | GPR137B | 0.797 |
| MIR100HG | LST1 | 0.758 | ABL2 | CHST11 | 0.760 | PKIB | PLA1A | 0.789 |
| GAS2L3 | CYP26B1 | 0.814 | CYTH4 | CAVIN1 | 0.768 | FGR | P2RY14 | 0.799 |
| IFITM2 | RIPOR2 | 0.767 | TMEM71 | GPR65 | 0.755 | BDH1 | CCDC69 | 0.770 |
| NRCAM | PCBP3 | 0.760 | BICD2 | IL11 | 0.825 | CMTM8 | TAGAP | 0.744 |
| EFNA4 | CHI3L1 | 0.799 | ENO2 | ASAP1 | 0.770 | CHI3L2 | IL11 | 0.822 |
| OLFM1 | PAPPA | 0.824 | WNK2 | FPR2 | 0.806 | IL24 | CRIP2 | 0.804 |
| FILIP1L | IL1B | 0.821 | MYO1F | ZDHHC18 | 0.775 | PDE4B | ENTPD1 | 0.762 |
| WNK2 | RBMS1 | 0.817 | JAZF1 | VNN2 | 0.818 | FLT1 | BTG2 | 0.769 |
| LRRK2 | PRR16 | 0.800 | AGFG2 | PPP1R18 | 0.774 | COQ9 | PIP4P2 | 0.813 |
| CSF2RB | TCF4 | 0.778 | NOD2 | CCDC3 | 0.724 | TMEM154 | RGCC | 0.793 |
| TMEM106C | BCAT1 | 0.756 | PLEK | MAP4K4 | 0.766 | ZDHHC23 | LGALS1 | 0.815 |
| AGFG2 | CXCR4 | 0.740 | DPYSL2 | TNFRSF10C | 0.762 | LILRA2 | FCGR2B | 0.795 |
| SERPINE1 | SPOCK1 | 0.809 | C2CD2 | PLA1A | 0.762 | EGR3 | TUBB6 | 0.755 |
| FILIP1L | SLC35G2 | 0.801 | COL4A2 | IL6 | 0.797 | AKAP1 | RBMS1 | 0.831 |
| GOLPH3L | CLU | 0.759 | B4GALT4 | CCL2 | 0.759 | SLC35G2 | SNN | 0.811 |
| CLEC4A | VAV1 | 0.747 | GPR176 | TNFAIP2 | 0.746 | COL15A1 | GFPT2 | 0.755 |
| GAS2L3 | SERPINE1 | 0.802 | UBAC1 | LILRA2 | 0.784 | NR3C2 | RGS19 | 0.767 |
| PAPPA | CLMP | 0.819 | OSM | THEMIS2 | 0.815 | NRG1 | TUBA1A | 0.801 |
| CHI3L1 | PPP1R18 | 0.781 | ZDHHC23 | CBX6 | 0.799 | NDUFA10 | SH2D3C | 0.788 |
| COL7A1 | BPGM | 0.791 | KIAA1522 | CYP26B1 | 0.826 | LILRB3 | NIBAN1 | 0.762 |
| LGALS1 | SLC11A1 | 0.799 | CR1 | FJX1 | 0.738 | FGF7 | PTGS1 | 0.792 |
| SRGN | CCL4 | 0.783 | PHLDB2 | GMFG | 0.792 | GALNT12 | PTGS1 | 0.807 |
| SORT1 | TMEM273 | 0.754 | LILRA5 | CEP19 | 0.742 | EGR3 | FPR2 | 0.790 |
| NIBAN1 | CSF3 | 0.815 | RAB31 | VNN2 | 0.811 | CDX2 | CXCL8 | 0.797 |
| CEBPB | EMP3 | 0.774 | PAPPA | OSMR | 0.810 | PIP4P2 | THEMIS2 | 0.751 |
| TMEM106C | SLFN11 | 0.759 | HIBCH | CHI3L2 | 0.739 | ATP5IF1 | CSF3 | 0.812 |
| FPR2 | PAPPA | 0.832 | LILRB3 | MMP19 | 0.730 | COTL1 | BCL6 | 0.731 |
| AGAP1 | BICD2 | 0.774 | SERPINE1 | S100A9 | 0.815 | ZNF704 | LILRB1 | 0.790 |
| FAP | RAI14 | 0.765 | ADGRG3 | AQP9 | 0.762 | NCF4 | P2RY13 | 0.771 |
| ENO2 | MX2 | 0.787 | LY96 | BMERB1 | 0.822 | PPP2R5A | IFITM2 | 0.780 |
| CXCL5 | SPP1 | 0.774 | FILIP1L | SLFN11 | 0.735 | AKAP1 | ANKRD35 | 0.797 |
| DSP | NEO1 | 0.777 | DUSP2 | HCLS1 | 0.719 | UQCRC1 | GPR183 | 0.782 |
| CGAS | JAZF1 | 0.767 | FPR2 | BTG2 | 0.791 | PLAU | BTG2 | 0.759 |
| SORT1 | LDLRAD3 | 0.800 | GOLPH3L | CGAS | 0.756 | ANKRD35 | NCF4 | 0.769 |
| PIK3AP1 | VAV1 | 0.676 | IL24 | THBD | 0.814 | OCRL | CXCR2 | 0.831 |
| LRP4 | CAVIN1 | 0.780 | IL11 | PCBP3 | 0.830 | ZNF704 | DUSP4 | 0.757 |
| HIBCH | ARPIN | 0.753 | HSD11B2 | ANGPT2 | 0.797 | COX10 | CAVIN3 | 0.799 |
| FAP | LGALS1 | 0.761 | P2RY13 | SPOCK1 | 0.760 | RAI14 | PTGS1 | 0.782 |
| BCL2A1 | C5AR1 | 0.750 | ARPIN | C2CD2 | 0.754 | LRRK2 | EMP3 | 0.755 |
| ITGAM | LCP1 | 0.718 | CSF3R | ITGAM | 0.716 | CSF3R | ZDHHC18 | 0.734 |
| HIBCH | TNC | 0.795 | GZMB | PTPRC | 0.774 | TLR4 | SNN | 0.720 |
| LRP4 | PHLDB2 | 0.792 | AFG3L2 | PFKL | 0.767 | CD93 | ST3GAL5 | 0.784 |
| BDH1 | MSN | 0.766 | COX10 | FCER1G | 0.782 | BCL2A1 | ENTPD1 | 0.792 |
| PIK3AP1 | TIMP1 | 0.723 | PPP2R5A | ERMP1 | 0.796 | CAVIN3 | SERPINE1 | 0.802 |
| MYO1D | TUBA1A | 0.780 | ZDHHC18 | GPR65 | 0.777 | GPR137B | PXDN | 0.787 |
| FFAR2 | SELP | 0.785 | BICD2 | NNMT | 0.787 | CCDC69 | SH3BP5 | 0.730 |
| ANGPTL2 | TREM1 | 0.770 | GNAI2 | PCBP3 | 0.759 | IL24 | HS3ST3B1 | 0.813 |
| PFKL | TCEA3 | 0.783 | TMEM154 | FCER1G | 0.778 | ANGPTL2 | TLR4 | 0.700 |
| CSNK1G3 | SWAP70 | 0.731 | LY96 | STC1 | 0.825 | SNORC | SLC11A1 | 0.800 |
| CTHRC1 | CAVIN1 | 0.769 | HCK | HSPA6 | 0.741 | PTGS2 | TNF | 0.792 |
| IL24 | PROK2 | 0.795 | NFAM1 | SLA | 0.725 | DSP | FAM20C | 0.772 |
| F5 | TNFAIP2 | 0.768 | CHN1 | RNF149 | 0.809 | DSP | DUSP2 | 0.782 |
| PTPRC | LST1 | 0.757 | CHI3L2 | CCN4 | 0.764 | ASB7 | IL1B | 0.788 |
| CSF2RB | MEFV | 0.769 | ARPIN | SERPINE1 | 0.799 | DUSP2 | THBD | 0.782 |
| SLC15A3 | CXCL5 | 0.766 | TRPM6 | FJX1 | 0.778 | FILIP1L | LCP1 | 0.751 |
| CMTM4 | IL1B | 0.791 | ITGA5 | S100A9 | 0.808 | IL13RA2 | GFPT2 | 0.831 |
| CMTM8 | RHOH | 0.769 | IL1RN | GADD45B | 0.777 | SORT1 | LILRA2 | 0.790 |
| G0S2 | VIM | 0.804 | HCK | C11orf96 | 0.778 | NR3C2 | RAB31 | 0.791 |
| HIBCH | TMEM273 | 0.777 | COQ9 | CSF2RB | 0.813 | PIK3AP1 | FGF7 | 0.776 |
| TMEM273 | SLC15A3 | 0.757 | SNORC | ST3GAL5 | 0.770 | GPR176 | THEMIS2 | 0.771 |
| TRIM2 | LDLRAD3 | 0.797 | FERMT2 | FAP | 0.760 | CLEC4E | CAVIN1 | 0.745 |
| MYO1F | STC1 | 0.804 | NDFIP2 | NFAM1 | 0.756 | SORT1 | CHST11 | 0.759 |
| DOK3 | MAP4K4 | 0.744 | SLC15A4 | FPR1 | 0.795 | IL1B | ENTPD1 | 0.805 |
| CAVIN1 | GPR65 | 0.752 | LILRB1 | IL6 | 0.822 | CMTM8 | HSPA6 | 0.759 |
| PFKL | CHI3L1 | 0.789 | PKIB | CGAS | 0.776 | CLU | CEMIP | 0.788 |
| SLC15A4 | RIPOR3 | 0.809 | CGAS | DPYSL2 | 0.762 | LDLRAD3 | S1PR1 | 0.785 |
| PCBP3 | ACOD1 | 0.771 | COX10 | PI15 | 0.763 | FZD5 | PRRX1 | 0.792 |
| SERPINE2 | CEMIP | 0.788 | GLIPR1 | FAP | 0.711 | EMP3 | SMAP2 | 0.766 |
| DSP | SPHK1 | 0.781 | CSTA | S100A8 | 0.793 | ASB7 | LILRA2 | 0.756 |
| SRGN | CEMIP | 0.779 | CEMIP | CLMP | 0.772 | PKIB | FCER1G | 0.815 |
| TNF | CCN4 | 0.797 | NRCAM | PXDN | 0.792 | ARPIN | BCL2A1 | 0.786 |
| POLDIP2 | FPR2 | 0.797 | MMP19 | THBD | 0.789 | TMEM106C | AMPD2 | 0.789 |
| IL6 | CREB5 | 0.806 | ANGPTL2 | LGALS1 | 0.764 | COX10 | ACOD1 | 0.779 |
| CSF2RB | GZMB | 0.785 | GLCE | RHOH | 0.745 | LY96 | CAVIN1 | 0.779 |
| WNK2 | ENO2 | 0.823 | CCDC69 | PI15 | 0.772 | RAB15 | CSF3 | 0.789 |
| CAVIN3 | MMP3 | 0.806 | GPR183 | PDLIM4 | 0.772 | CBX6 | RGS16 | 0.747 |
| CEBPB | COTL1 | 0.781 | OLFM1 | SWAP70 | 0.711 | COQ9 | EMP3 | 0.812 |
| OSM | GPR137B | 0.824 | GLIPR1 | FGF2 | 0.712 | DUSP2 | JAZF1 | 0.765 |
| FILIP1L | BTG2 | 0.768 | PPP1R18 | FAM155A | 0.787 | S100A12 | SNN | 0.816 |
| SORT1 | CSTA | 0.747 | JAZF1 | SERPINB9 | 0.770 | CHI3L2 | GMFG | 0.747 |
| TIMP1 | TUBA1A | 0.773 | TRAK1 | PLA1A | 0.774 | ICAM1 | ITGAM | 0.751 |
| SORT1 | COL7A1 | 0.813 | ZDHHC23 | TMEM273 | 0.833 | SORT1 | CLMP | 0.794 |
| FGF2 | PCBP3 | 0.792 | LOXL2 | ASAP1 | 0.779 | LRRK2 | SERPINB9 | 0.783 |
| SNORC | CLEC4E | 0.780 | FAM20C | BTG2 | 0.777 | SLC15A4 | ITGAM | 0.798 |
| GPR176 | PTPRC | 0.755 | PIK3AP1 | ACOT9 | 0.737 | CAVIN3 | LGALS1 | 0.762 |
| GNAI2 | PRR16 | 0.796 | FCGR2B | SLC35G2 | 0.809 | NR3C2 | CCL2 | 0.784 |
| LOXL2 | RBMS1 | 0.780 | CSNK1G3 | PRRX1 | 0.780 | AKAP1 | ATP5IF1 | 0.789 |
| PFKL | SLC16A6 | 0.762 | FZD5 | ZDHHC18 | 0.767 | DPYSL3 | LY96 | 0.789 |
| PPP2R5A | JAZF1 | 0.780 | CMTM8 | P2RY13 | 0.798 | RGCC | GPR65 | 0.784 |
| CLU | RIPOR3 | 0.785 | GPR176 | F5 | 0.790 | STAP2 | GFPT2 | 0.751 |
| POLDIP2 | NDFIP2 | 0.783 | ACOT9 | ZDHHC18 | 0.796 | HSD11B2 | S1PR1 | 0.790 |
| CCDC88A | SSH2 | 0.746 | SELENBP1 | RBMS1 | 0.813 | PKIB | B4GALT4 | 0.750 |
| KCNJ15 | NFE2 | 0.703 | CLU | RILPL2 | 0.766 | RIPOR3 | BCL6 | 0.718 |
| B4GALT4 | CSTA | 0.764 | C5AR1 | CD69 | 0.750 | CXCR2 | SOD2 | 0.787 |
| CXCL8 | SELP | 0.822 | IL11 | CEP19 | 0.777 | CSNK1G3 | TLR4 | 0.740 |
| RILPL2 | F5 | 0.791 | EFNA4 | LILRA5 | 0.770 | DPYSL2 | LY96 | 0.795 |
| COL4A2 | PRR16 | 0.791 | UBAC1 | LRRK2 | 0.810 | AFG3L2 | RASSF5 | 0.771 |
| UBAC1 | COTL1 | 0.813 | CCDC88A | TNFRSF1B | 0.728 | PDPN | EGR3 | 0.765 |
| TAGAP | CSTA | 0.711 | WNK2 | CCL2 | 0.781 | KIAA1522 | FERMT2 | 0.775 |
| FGF7 | NCF2 | 0.789 | HLX | GADD45B | 0.727 | CSF3R | LAT2 | 0.758 |
| PPP1R18 | MCAM | 0.744 | SLC2A3 | GPR65 | 0.721 | MEFV | P2RY13 | 0.746 |
| GOLPH3L | KCNJ15 | 0.753 | ARPIN | RASSF5 | 0.771 | F5 | CCDC3 | 0.785 |
| CHST15 | THBD | 0.804 | FPR1 | TNFRSF10C | 0.730 | TRAK1 | PFKL | 0.786 |
| COX10 | FCGR2B | 0.791 | HIBCH | MAP4K4 | 0.788 | COX10 | MIR100HG | 0.775 |
| CDX2 | LOXL2 | 0.795 | TAGAP | CXCR4 | 0.705 | NEO1 | CR1 | 0.767 |
| HCLS1 | BCL6 | 0.656 | RALGAPA2 | PTGS2 | 0.796 | COL7A1 | IL11 | 0.823 |
| PTGS2 | NOD2 | 0.739 | CDX2 | CEBPB | 0.811 | LRRK2 | COL15A1 | 0.773 |
| DPYSL2 | PLA1A | 0.758 | DUSP4 | FGR | 0.774 | TMEM273 | TLR4 | 0.702 |
| SLC15A4 | PXDN | 0.810 | CD300A | CHST2 | 0.800 | MYO1D | GFPT2 | 0.746 |
| PKIB | CREB5 | 0.795 | C1R | P2RY14 | 0.754 | FPR2 | SELP | 0.815 |
| WNK2 | F5 | 0.815 | ANKRD35 | CCDC3 | 0.786 | SH3RF2 | RGS16 | 0.768 |
| AKAP1 | PPP1R18 | 0.798 | TCEA3 | TSPAN6 | 0.749 | ERMP1 | IL24 | 0.804 |
| TMEM273 | CXCR1 | 0.755 | LCP2 | SRGN | 0.782 | TAGAP | STC1 | 0.764 |
| RNF149 | SLC2A3 | 0.770 | IFITM2 | MYO1F | 0.775 | ZDHHC23 | ANGPT2 | 0.825 |
| UQCRC1 | ABL2 | 0.787 | DSP | TMEM71 | 0.801 | SNORC | AQP9 | 0.799 |
| FAM20C | THBD | 0.789 | SH2D3C | VASN | 0.740 | IL6 | LCP2 | 0.804 |
| GOLPH3L | ACOT9 | 0.820 | CBX6 | STAT5B | 0.739 | RILPL2 | STC1 | 0.827 |
| MEFV | PXDN | 0.793 | C11orf96 | TNFRSF10C | 0.787 | CXCL5 | TNFAIP6 | 0.789 |
| GLIPR1 | RAB31 | 0.736 | KIAA1522 | VIM | 0.770 | POLDIP2 | STAP2 | 0.744 |
| SNN | RASSF5 | 0.784 | IL1RN | SPP1 | 0.782 | SLC15A3 | P2RY13 | 0.756 |
| RAB31 | JAZF1 | 0.755 | LILRB1 | CYTH4 | 0.785 | FGF2 | SLC11A1 | 0.792 |
| TNFAIP6 | TUBA1A | 0.778 | CEBPB | PHLDB2 | 0.795 | IL24 | SRGN | 0.798 |
| PCBP3 | ST3GAL5 | 0.753 | NIBAN1 | C5AR1 | 0.754 | ACOT9 | LY96 | 0.814 |
| INHBA | LGALS1 | 0.793 | AQP9 | SLFN11 | 0.761 | LILRA2 | CRIP2 | 0.759 |
| NNMT | PREX1 | 0.779 | GAS2L3 | DLC1 | 0.816 | FERMT2 | SLC16A6 | 0.784 |
| PPP1R18 | PLAU | 0.754 | RIPK2 | CD69 | 0.764 | GALNT12 | S1PR1 | 0.799 |
| RASSF5 | GMFG | 0.777 | NEO1 | MCAM | 0.803 | HCLS1 | IL1RN | 0.750 |
| NRG1 | MYO1F | 0.792 | ATP5IF1 | LDLRAD3 | 0.800 | GOLPH3L | HCLS1 | 0.744 |
| EFNA4 | GALNT12 | 0.767 | ITGA5 | FAM155A | 0.816 | SRGN | NOD2 | 0.751 |
| CEBPB | GMFG | 0.786 | KIAA1522 | WWC2 | 0.769 | CAVIN3 | LOXL2 | 0.769 |
| PIK3AP1 | FAM155A | 0.776 | FZD5 | OLFM1 | 0.790 | TMEM71 | MCAM | 0.801 |
| ASB7 | LCP2 | 0.762 | FCGR2B | IL1RN | 0.807 | COTL1 | ITGA5 | 0.760 |
| CLU | CD69 | 0.754 | CMTM4 | MYO1F | 0.773 | ARPIN | PRRX1 | 0.767 |
| FGF2 | SERPINB9 | 0.769 | S1PR1 | SMAP2 | 0.764 | SLC15A4 | NNMT | 0.808 |
| CXCR1 | ST3GAL5 | 0.764 | FILIP1L | ADGRG3 | 0.776 | CCDC88A | C5AR1 | 0.750 |
| NFAM1 | GPR137B | 0.744 | COX10 | BTG2 | 0.773 | LCP1 | MCAM | 0.748 |
| SH3RF2 | LCP1 | 0.754 | CAVIN3 | BMERB1 | 0.793 | HCLS1 | PCBP3 | 0.716 |
| RAB15 | MMP19 | 0.783 | CMTM8 | NCF4 | 0.747 | CCDC88A | SH3BP5 | 0.724 |
| NNMT | CD86 | 0.790 | S100A8 | ACOD1 | 0.792 | COL15A1 | ARHGAP25 | 0.769 |
| AGAP1 | GMFG | 0.779 | NCF2 | SRGN | 0.771 | ABL2 | ITGAM | 0.765 |
| MEFV | THBD | 0.792 | NRCAM | RBMS1 | 0.771 | FAP | PTPRC | 0.761 |
| ADGRG3 | STC1 | 0.793 | ERMP1 | TLR4 | 0.736 | GLIPR1 | SERPINB9 | 0.754 |
| TRIM2 | PI15 | 0.766 | AFG3L2 | NRCAM | 0.768 | SLA | SNN | 0.749 |
| PRICKLE2 | CCDC3 | 0.782 | TNC | PRRX1 | 0.781 | WNK2 | ITPRIP | 0.840 |
| FCGR2B | CREB5 | 0.789 | HIBCH | FCGR3B | 0.807 | GLCE | OCRL | 0.785 |
| ITGA5 | LAT2 | 0.783 | TRPM6 | FGF7 | 0.823 | GLT1D1 | FPR2 | 0.727 |
| UQCRC1 | THEMIS2 | 0.800 | ADCY6 | NFAM1 | 0.760 | JAZF1 | NFE2 | 0.793 |
| CRIP2 | PLAU | 0.777 | AMPD2 | MIR100HG | 0.763 | LY96 | PTPRC | 0.788 |
| DLC1 | IL11 | 0.843 | PDPN | NCF4 | 0.764 | ADGRG3 | TREM1 | 0.736 |
| SWAP70 | RHOH | 0.662 | GLT1D1 | SPP1 | 0.762 | PPP2R5A | CXCR2 | 0.832 |
| LRP4 | CCR1 | 0.803 | G0S2 | PCBP3 | 0.822 | CBX6 | CLEC4A | 0.747 |
| CSNK1G3 | CMTM4 | 0.763 | CSF2RB | NOD2 | 0.733 | GLIPR1 | BMERB1 | 0.762 |
| FCER1G | CD14 | 0.752 | SH3RF2 | S100A8 | 0.806 | AKAP1 | FCGR3B | 0.813 |
| DPYSL3 | STC1 | 0.821 | FERMT2 | PXDN | 0.777 | LDLRAD3 | JAZF1 | 0.782 |
| NRG1 | VNN2 | 0.822 | NFE2 | CXCL5 | 0.762 | RAB15 | BICD2 | 0.793 |
| DUSP2 | BCAT1 | 0.753 | ZDHHC23 | AKAP1 | 0.816 | ADGRG3 | MYO1F | 0.752 |
| GPR176 | GLT1D1 | 0.763 | CMTM8 | CHST11 | 0.748 | IL24 | TAGAP | 0.788 |
| ANGPTL2 | CCL2 | 0.767 | RGS19 | GLT1D1 | 0.693 | GADD45B | SLC2A3 | 0.735 |
| CTHRC1 | PI15 | 0.771 | PCBP3 | CYP26B1 | 0.819 | COTL1 | SLC11A1 | 0.783 |
| FPR2 | IL11 | 0.831 | FGF2 | CLEC4E | 0.767 | ARPIN | CR1 | 0.758 |
| FCGR2B | ITGA5 | 0.780 | TRAK1 | CCL2 | 0.764 | SH3RF2 | PIP4P2 | 0.816 |
| CSNK1G3 | LILRA2 | 0.787 | ATP5IF1 | CSTA | 0.765 | FAM20C | GPR65 | 0.771 |
| RHOH | TCF4 | 0.726 | ADCY6 | CSF3 | 0.811 | ENO2 | P2RY14 | 0.785 |
| TNFAIP2 | C5AR1 | 0.766 | AGFG2 | FGR | 0.788 | TCEA3 | STC1 | 0.808 |
| NEO1 | COL15A1 | 0.790 | INHBA | SERPINB9 | 0.792 | ARPIN | OSMR | 0.781 |
| NIPSNAP3A | CYP26B1 | 0.811 | PDPN | CD300A | 0.789 | FILIP1L | PROK2 | 0.726 |
| HSD11B2 | MEFV | 0.782 | GLIPR1 | SPOCK1 | 0.714 | SH2D3C | CD300A | 0.795 |
| PDE4B | PLEK | 0.745 | AGFG2 | ATP5IF1 | 0.771 | FCGR3B | FJX1 | 0.801 |
| KIAA1522 | CSF2RB | 0.784 | TRPM6 | PIP4P2 | 0.792 | LILRB1 | CHN1 | 0.799 |
| FGF2 | PDE4B | 0.748 | CDX2 | LAT2 | 0.786 | HSD11B2 | GLIPR1 | 0.759 |
| STAP2 | EGR3 | 0.752 | RAB31 | SERPINE2 | 0.780 | OCRL | IL11 | 0.836 |
| ERMP1 | TMEM154 | 0.790 | B4GALT4 | WWC2 | 0.762 | FZD5 | SLC2A3 | 0.767 |
| FPR2 | FJX1 | 0.789 | PFKL | FCER1G | 0.786 | UBAC1 | TLR1 | 0.814 |
| WNK2 | FPR1 | 0.788 | CTHRC1 | LRRK2 | 0.794 | GPR176 | CRIP2 | 0.763 |
| LRP4 | IFITM2 | 0.779 | CGAS | FJX1 | 0.754 | FCGR3B | HSPA6 | 0.787 |
| PTGS2 | CCL4 | 0.760 | NR3C2 | MYO1D | 0.783 | UBAC1 | IL1RN | 0.790 |
| BPGM | SPP1 | 0.766 | RILPL2 | TREM1 | 0.785 | TRAK1 | PHLDB2 | 0.765 |
| NR3C2 | TMEM106C | 0.774 | SWAP70 | SSH2 | 0.704 | RNF149 | VIM | 0.796 |
| TNC | HS3ST3B1 | 0.793 | AFG3L2 | FAP | 0.764 | COX10 | NIPSNAP3A | 0.757 |
| SLC15A3 | NOD2 | 0.735 | LOXL2 | SLC2A3 | 0.743 | CSF3 | S1PR1 | 0.809 |
| ERMP1 | CSF3R | 0.742 | IL1B | TCF4 | 0.793 | TRAK1 | ZNF704 | 0.787 |
| PDPN | LST1 | 0.768 | KCNJ15 | C11orf96 | 0.742 | STAP2 | EMP3 | 0.775 |
| ERMP1 | TMEM71 | 0.806 | BDH1 | FAM20C | 0.790 | AFG3L2 | CSF2RB | 0.790 |
| GNAI2 | CD53 | 0.714 | PLAU | ST3GAL5 | 0.771 | CLMP | HS3ST3B1 | 0.782 |
| CREM | CD14 | 0.760 | CSF3R | SPHK1 | 0.727 | LILRA2 | CHST2 | 0.781 |
| CXCR2 | SLC16A6 | 0.765 | PIK3AP1 | CLEC4E | 0.682 | GAS2L3 | TLR1 | 0.805 |
| SORT1 | FCER1G | 0.765 | CSNK1G3 | CHST15 | 0.773 | GALNT12 | CBX6 | 0.781 |
| NDFIP2 | LILRB1 | 0.794 | UQCRC1 | S100A12 | 0.796 | ASB7 | RGS19 | 0.707 |
| CRIP2 | CEMIP | 0.802 | LILRB2 | CEMIP | 0.788 | FGF7 | TNFAIP2 | 0.795 |
| FILIP1L | LST1 | 0.789 | GPR176 | BMERB1 | 0.780 | RAB15 | SELE | 0.814 |
| RAI14 | LGALS1 | 0.787 | COQ9 | ANGPTL2 | 0.783 | LRRK2 | SELP | 0.810 |
| KIAA1522 | TNFRSF10C | 0.775 | SNORC | SLC16A6 | 0.762 | CRIP2 | FAM49A | 0.783 |
| PI15 | P2RY14 | 0.758 | MMP3 | BCAT1 | 0.767 | TIMP1 | THEMIS2 | 0.788 |
| OLFM1 | RFLNB | 0.774 | DPYSL3 | ICAM1 | 0.760 | IL1B | SERPINB9 | 0.791 |
| IL1RN | MX2 | 0.790 | CMTM8 | TMEM71 | 0.807 | SORT1 | SH2D3C | 0.780 |
| ANGPTL2 | SSH2 | 0.780 | FERMT2 | TUBB6 | 0.765 | PAPPA | ST3GAL5 | 0.827 |
| ITGAM | TUBB6 | 0.785 | GAS2L3 | FGR | 0.791 | SH3RF2 | RGS19 | 0.761 |
| ACOD1 | VAV1 | 0.738 | LRP4 | FAM20C | 0.769 | HIBCH | FAP | 0.752 |
| PI15 | TUBA1A | 0.767 | RAB31 | HSPA6 | 0.740 | FPR1 | ACOT9 | 0.765 |
| CHI3L2 | CEMIP | 0.766 | COL4A2 | ACOT9 | 0.772 | HIBCH | UQCRC1 | 0.762 |
| ICAM1 | CHST15 | 0.764 | KIAA1522 | FZD5 | 0.773 | PIK3AP1 | PCBP3 | 0.744 |
| LRRK2 | MMP3 | 0.794 | STAP2 | MCAM | 0.767 | GZMB | CLMP | 0.788 |
| LILRB3 | CXCL8 | 0.744 | SELENBP1 | TMEM154 | 0.810 | NRG1 | CCL2 | 0.80O |
| PDPN | PIK3AP1 | 0.761 | KCNJ15 | VNN2 | 0.733 | SHTN1 | CXCL5 | 0.759 |
| PKIB | PRRX1 | 0.794 | GZMB | STAT5B | 0.781 | WNK2 | CREM | 0.808 |
| LRRK2 | TNC | 0.793 | MX2 | SH3BP5 | 0.728 | AKAP1 | LCP2 | 0.800 |
| GPR65 | CD53 | 0.709 | WNK2 | FAM20C | 0.805 | UBAC1 | BPGM | 0.803 |
| GPR183 | FFAR2 | 0.751 | CXCR1 | PROK2 | 0.728 | EFNA4 | HSPA6 | 0.735 |
| COL4A2 | SLFN11 | 0.710 | ABL2 | SRGN | 0.790 | TSPAN6 | FGR | 0.786 |
| CD300A | GPR137B | 0.808 | ZNF704 | OSMR | 0.782 | C2CD2 | PRR16 | 0.793 |
| FZD5 | NFAM1 | 0.741 | DUSP2 | MAP4K4 | 0.775 | PDPN | S100A9 | 0.809 |
| HCLS1 | CHST11 | 0.676 | LDLRAD3 | FFAR2 | 0.783 | NDUFA10 | GNAI2 | 0.781 |
| PIK3AP1 | RAI14 | 0.744 | DUSP2 | PLAU | 0.772 | CR1 | HCK | 0.736 |
| TMEM154 | FAM155A | 0.816 | FGR | S100A9 | 0.807 | MYO1D | TNF | 0.806 |
| UBAC1 | PHLDB2 | 0.808 | CDX2 | DLC1 | 0.793 | TAGAP | CXCL8 | 0.742 |
| COX10 | BMERB1 | 0.826 | TIMP1 | FAM49A | 0.792 | RFLNB | TNFAIP6 | 0.791 |
| G0S2 | ARHGAP25 | 0.809 | STAT5B | WNT5A | 0.809 | TMEM154 | TNFRSF1B | 0.725 |
| LRP4 | S1PR1 | 0.797 | SOD2 | CEP19 | 0.737 | RGS19 | IFITM2 | 0.730 |
| CD86 | CREB5 | 0.770 | CYTH4 | CXCR1 | 0.752 | PREX1 | CXCR4 | 0.680 |
| LCP1 | CCL4 | 0.738 | FCGR3B | FAM155A | 0.827 | TRPM6 | CEBPB | 0.804 |
| SHTN1 | CLU | 0.758 | WNT5A | PHLDB2 | 0.822 | FAP | PCBP3 | 0.766 |
| AFG3L2 | LRRK2 | 0.808 | RNF149 | SOD2 | 0.777 | P2RY13 | PHLDB2 | 0.775 |
| LDLRAD3 | GPR183 | 0.792 | CLEC4A | SELL | 0.811 | FGF2 | PXDN | 0.788 |
| CDX2 | LILRB2 | 0.803 | CSF3 | CCDC3 | 0.807 | DSP | NDFIP2 | 0.779 |
| GALNT12 | S100A12 | 0.797 | RBMS1 | BTG2 | 0.797 | COL7A1 | TNF | 0.789 |
| EFNA4 | SLC2A3 | 0.757 | ADCY6 | LILRB1 | 0.776 | FCER1G | RIPOR2 | 0.756 |
| PTGS2 | CEP19 | 0.740 | PLAU | SRGN | 0.776 | TNC | SMAP2 | 0.775 |
| CLEC4A | VIM | 0.782 | RHOH | TIMP1 | 0.743 | FERMT2 | TNFAIP2 | 0.764 |
| SELENBP1 | MYO1F | 0.807 | LILRB2 | SSH2 | 0.765 | RAB15 | IL1RN | 0.797 |
| ACOD1 | RIPOR2 | 0.743 | PDE4B | STAT5B | 0.739 | NEO1 | CBX6 | 0.773 |
| B4GALT4 | ICAM1 | 0.751 | SNORC | FGF2 | 0.779 | TRIM2 | PLEK | 0.770 |
| SELENBP1 | CHI3L1 | 0.808 | CSF2RB | MYO1F | 0.747 | IFITM2 | LILRA5 | 0.740 |
| NDFIP2 | CD300A | 0.795 | NFAM1 | CLEC4A | 0.731 | CR1 | NOD2 | 0.695 |
| NDFIP2 | RILPL2 | 0.786 | BCL2A1 | SELE | 0.808 | GPR176 | GFPT2 | 0.745 |
| CHRDL2 | RAI14 | 0.767 | SHTN1 | SERPINE2 | 0.799 | UBAC1 | CLMP | 0.786 |
| RIPOR3 | LCP1 | 0.770 | IL11 | CCL2 | 0.797 | CXCL8 | SPP1 | 0.770 |
| SH3RF2 | RALGAPA2 | 0.778 | FERMT2 | GPR183 | 0.760 | CSF2RB | CLEC4A | 0.764 |
| S1PR3 | RIPK2 | 0.786 | ANGPT2 | TCF4 | 0.778 | PIK3AP1 | CCDC88A | 0.695 |
| MEFV | SH3BP5 | 0.729 | VIM | ENTPD1 | 0.774 | POLDIP2 | STC1 | 0.805 |
| ADCY6 | FERMT2 | 0.791 | STC1 | WWC2 | 0.793 | CSNK1G3 | PLAU | 0.782 |
| B4GALT4 | SPP1 | 0.762 | RAB31 | SLC16A6 | 0.747 | RHOH | RGS16 | 0.741 |
| GLCE | NIBAN1 | 0.795 | FILIP1L | RGS16 | 0.760 | CHI3L2 | BCL2A1 | 0.758 |
| IL1B | TNFRSF10C | 0.776 | RAB15 | PTGS2 | 0.813 | PKIB | PAPPA | 0.832 |
| PTGS2 | SNN | 0.804 | SERPINB9 | TLR1 | 0.795 | S100A8 | TUBA1A | 0.813 |
| AFG3L2 | RAB31 | 0.753 | FZD5 | S100A12 | 0.805 | NDFIP2 | C5AR1 | 0.800 |
| CDX2 | TSPAN6 | 0.768 | CCDC69 | PAPPA | 0.824 | S1PR3 | LST1 | 0.755 |
| GLT1D1 | PTGS1 | 0.801 | CR1 | PPP1R18 | 0.733 | IER5 | CEMIP | 0.756 |
| SHTN1 | TNF | 0.795 | MYO1D | IFITM2 | 0.781 | AKAP1 | PDPN | 0.799 |
| CBX6 | GMFG | 0.767 | TMEM71 | AQP9 | 0.782 | CREM | PPP1R18 | 0.759 |
| CYP26B1 | CEMIP | 0.797 | CXCR2 | NOD2 | 0.764 | MEFV | IER5 | 0.726 |
| F5 | PROK2 | 0.733 | CSF2RB | CSF3R | 0.740 | GALNT12 | FILIP1L | 0.782 |
| CCDC69 | GZMB | 0.781 | GZMB | SMAP2 | 0.774 | BTG2 | SSH2 | 0.769 |
| STAP2 | RBMS1 | 0.774 | DUSP4 | ARHGAP25 | 0.780 | GALNT12 | INHBA | 0.819 |
| FILIP1L | MCAM | 0.766 | PROK2 | SELP | 0.746 | JAZF1 | CYP26B1 | 0.799 |
| FILIP1L | ZDHHC18 | 0.771 | TCEA3 | CSF3R | 0.771 | CEBPB | ZDHHC18 | 0.779 |
| GPR176 | SSH2 | 0.742 | GNAI2 | FAM155A | 0.791 | PIK3AP1 | SPP1 | 0.723 |
| IFITM2 | CLEC4E | 0.764 | CLU | CHST2 | 0.757 | ITPRIP | SLC16A6 | 0.782 |
| BICD2 | CEP19 | 0.679 | SELL | WNT5A | 0.814 | LDLRAD3 | ACOT9 | 0.796 |
| TMEM106C | B4GALT4 | 0.747 | CHI3L2 | SELL | 0.750 | MSN | WWC2 | 0.754 |
| MSN | PLEK | 0.745 | HIBCH | GFPT2 | 0.752 | EGR3 | TREM1 | 0.762 |
| IL6 | LCP1 | 0.776 | SNORC | TNF | 0.793 | CSF3 | DPYSL3 | 0.785 |
| FPR1 | TLR1 | 0.755 | PLEK | VIM | 0.762 | PREX1 | ACOD1 | 0.736 |
| DUSP2 | VNN2 | 0.82O | RGS19 | NNMT | 0.752 | CDX2 | CHI3L1 | 0.789 |
| EMP3 | BMERB1 | 0.798 | DPYSL2 | SLA | 0.729 | RILPL2 | SSH2 | 0.802 |
| BCL2A1 | SELL | 0.784 | OCRL | RBMS1 | 0.831 | FCGR3B | GZMB | 0.808 |
| SWAP70 | THEMIS2 | 0.705 | BMERB1 | OSMR | 0.793 | NDFIP2 | OSM | 0.817 |
| TNC | PDE4B | 0.762 | AMPD2 | LCP1 | 0.739 | FJX1 | RAI14 | 0.779 |
| CXCR2 | LAT2 | 0.818 | AFG3L2 | SELP | 0.798 | RASSF5 | CD86 | 0.768 |
| LRRK2 | ANGPT2 | 0.806 | GPR183 | SERPINE1 | 0.775 | COTL1 | TNC | 0.787 |
| COX10 | TLR1 | 0.817 | IL11 | SELL | 0.834 | LY96 | MAP4K4 | 0.783 |
| GOLPH3L | CEMIP | 0.802 | IL1RN | PIP4P2 | 0.817 | RFLNB | MCAM | 0.758 |
| GAS2L3 | NFE2 | 0.794 | UQCRC1 | CEP19 | 0.760 | AKAP1 | FAM155A | 0.839 |
| RNF149 | CXCL5 | 0.765 | CMTM4 | IL13RA2 | 0.868 | LRRK2 | GMFG | 0.788 |
| AMPD2 | ACOD1 | 0.765 | CD93 | SRGN | 0.805 | NDUFA10 | FGF2 | 0.784 |
| SWAP70 | G0S2 | 0.757 | FERMT2 | CXCL5 | 0.795 | PKIB | GMFG | 0.786 |
| RFLNB | TIMP1 | 0.770 | C2CD2 | SPP1 | 0.757 | TRAK1 | OSM | 0.806 |
| PDPN | CHRDL2 | 0.801 | SORT1 | TREM1 | 0.776 | GALNT12 | ADGRG3 | 0.768 |
| CDX2 | RAB31 | 0.792 | DLC1 | RFLNB | 0.776 | GAS2L3 | TMEM106C | 0.776 |
| SNORC | FFAR2 | 0.761 | NEO1 | PIK3AP1 | 0.748 | NFAM1 | GPR183 | 0.727 |
| S100A9 | SLA | 0.792 | CSTA | CCN4 | 0.742 | RAB31 | CD86 | 0.766 |
| CHN1 | COTL1 | 0.782 | MYO1F | CCL2 | 0.765 | DPYSL3 | C1R | 0.756 |
| AMPD2 | OSM | 0.816 | CXCR2 | THEMIS2 | 0.776 | NFAM1 | PRICKLE2 | 0.787 |
| RGCC | CREB5 | 0.798 | AKAP1 | CD93 | 0.822 | HCLS1 | TIMP1 | 0.723 |
| DLC1 | FCER1G | 0.797 | IL11 | BTG2 | 0.826 | FCER1G | TUBA1A | 0.765 |
| KIAA1522 | TNF | 0.790 | IL24 | FAM155A | 0.831 | LILRB2 | CGAS | 0.751 |
| LOXL2 | CCN4 | 0.772 | SH3RF2 | PFKL | 0.793 | LRP4 | DLC1 | 0.805 |
| RIPK2 | CD86 | 0.774 | FCGR3B | PDE4B | 0.789 | SHTN1 | PDPN | 0.782 |
| CXCL5 | THBD | 0.787 | LILRB3 | ST3GAL5 | 0.763 | CTHRC1 | WWC2 | 0.775 |
| PKIB | ITGAM | 0.803 | S1PR1 | LOXL2 | 0.784 | COTL1 | CYP26B1 | 0.789 |
| TNFAIP2 | SH3BP5 | 0.744 | G0S2 | CLMP | 0.826 | CLMP | RIPOR2 | 0.768 |
| BDH1 | C1R | 0.783 | AGFG2 | FILIP1L | 0.778 | CSF3 | CCL2 | 0.779 |
| GADD45B | RIPK2 | 0.783 | C2CD2 | TUBB6 | 0.778 | C11orf96 | ENTPD1 | 0.785 |
| STAT5B | DOK3 | 0.727 | BDH1 | EGR3 | 0.740 | IL13RA2 | PLAU | 0.835 |
| SHTN1 | RGCC | 0.792 | CDX2 | HIBCH | 0.794 | UQCRC1 | LDLRAD3 | 0.813 |
| PPP1R18 | CXCR4 | 0.692 | PFKL | PDE4B | 0.783 | LRRK2 | RASSF5 | 0.750 |
| S100A12 | STC1 | 0.819 | AGAP1 | ITPRIP | 0.793 | CLEC4A | CHST2 | 0.803 |
| RAB15 | OLFM1 | 0.807 | TLR4 | HS3ST3B1 | 0.761 | KIAA1522 | GPR183 | 0.755 |
| CMTM4 | CYTH4 | 0.778 | NRCAM | CD14 | 0.757 | MMP3 | CHST11 | 0.774 |
| DPYSL2 | FPR1 | 0.776 | TRIM2 | COL4A2 | 0.790 | ARPIN | CD53 | 0.747 |
| PLEK | PDLIM4 | 0.784 | ITGAM | MCAM | 0.775 | FGF7 | ST3GAL5 | 0.784 |
| COL4A2 | RBMS1 | 0.772 | CREM | BMERB1 | 0.816 | ZNF704 | CSF2RB | 0.802 |
| CHI3L2 | FCGR2B | 0.744 | SELL | ACOD1 | 0.787 | AGFG2 | CAVIN1 | 0.759 |
| IL11 | BCAT1 | 0.813 | LGALS1 | LST1 | 0.765 | SLA | SPOCK1 | 0.739 |
| CSF3R | COTL1 | 0.741 | RIPOR3 | S100A8 | 0.827 | PREX1 | SLC35G2 | 0.786 |
| CAVIN3 | SERPINB9 | 0.782 | ASB7 | SERPINE2 | 0.784 | CMTM4 | TRIM2 | 0.759 |
| NCF2 | BPGM | 0.775 | CAVIN3 | MAP4K4 | 0.764 | ASAP1 | PREX1 | 0.757 |
| TRAK1 | LY96 | 0.802 | LRP4 | ZNF704 | 0.793 | PFKL | FAM49A | 0.814 |
| ZDHHC23 | PIP4P2 | 0.824 | PLEK | FAM155A | 0.803 | PRR16 | SH3BP5 | 0.797 |
| HSD11B2 | CSF2RB | 0.789 | S1PR3 | IL11 | 0.824 | CDX2 | GFPT2 | 0.755 |
| TMEM71 | ADGRG3 | 0.758 | CCDC69 | OSM | 0.820 | NFAM1 | FFAR2 | 0.718 |
| DPYSL2 | PI15 | 0.772 | PI15 | CCL4 | 0.761 | NFAM1 | MMP19 | 0.746 |
| PLEK | SPOCK1 | 0.791 | CHN1 | LILRA5 | 0.792 | TMEM106C | OLFM1 | 0.774 |
| ANKRD35 | CXCR4 | 0.722 | FJX1 | CD86 | 0.769 | PFKL | PLA1A | 0.769 |
| DSP | CHST15 | 0.786 | CXCR2 | MMP19 | 0.811 | RIPOR3 | MX2 | 0.805 |
| CXCL8 | TNFRSF1B | 0.758 | IL13RA2 | VNN2 | 0.877 | CSNK1G3 | HLX | 0.769 |
| FCER1G | PCBP3 | 0.765 | SH3RF2 | FPR2 | 0.784 | C1R | VIM | 0.758 |
| LRP4 | CCDC3 | 0.796 | EFNA4 | TRIM2 | 0.783 | TNFRSF10C | PHLDB2 | 0.779 |
| TMEM106C | TAGAP | 0.756 | LY96 | CXCR2 | 0.814 | TRIM2 | CSTA | 0.753 |
| DPYSL3 | INHBA | 0.791 | LILRA2 | SLFN11 | 0.727 | MMP19 | C1R | 0.769 |
| MIR100HG | PAPPA | 0.808 | ICAM1 | PHLDB2 | 0.758 | TRIM2 | SRGN | 0.802 |
| PDPN | TREM1 | 0.774 | RIPOR3 | WWC2 | 0.773 | HSPA6 | THBD | 0.775 |
| P2RY13 | FAM49A | 0.739 | NR3C2 | STC1 | 0.799 | AGAP1 | STAT5B | 0.748 |
| CHI3L1 | ENTPD1 | 0.803 | CSF3R | PRICKLE2 | 0.762 | UQCRC1 | GALNT12 | 0.768 |
| CYP26B1 | FAM155A | 0.815 | LILRB3 | MMP3 | 0.745 | CGAS | CHST15 | 0.763 |
| TSPAN6 | TREM1 | 0.791 | ITGAM | TNFRSF1B | 0.725 | AGFG2 | MEFV | 0.781 |
| TRIM2 | FJX1 | 0.782 | TNF | ITPRIP | 0.800 | EFNA4 | GZMB | 0.787 |
| GPR183 | GPR137B | 0.791 | ITGAM | SPP1 | 0.746 | UBAC1 | CREM | 0.789 |
| SLC11A1 | HS3ST3B1 | 0.811 | NDUFA10 | PRRX1 | 0.773 | STC1 | VAV1 | 0.789 |
| COTL1 | CLEC4A | 0.774 | TLR4 | RIPK2 | 0.702 | ACOT9 | CCDC88A | 0.768 |
| POLDIP2 | SWAP70 | 0.711 | ENO2 | NRG1 | 0.792 | WWC2 | OSMR | 0.753 |
| PFKL | SELE | 0.801 | NIPSNAP3A | OSM | 0.821 | ZDHHC23 | MMP3 | 0.815 |
| SNORC | TUBA1A | 0.788 | CD93 | CHST11 | 0.774 | TMEM154 | AQP9 | 0.781 |
| PKIB | CAVIN3 | 0.789 | RAB15 | ACOD1 | 0.776 | B4GALT4 | PLEK | 0.771 |
| CAVIN1 | SOD2 | 0.781 | CD300A | RAI14 | 0.797 | TMEM71 | MMP19 | 0.791 |
| ZNF704 | IL13RA2 | 0.864 | LRP4 | DUSP4 | 0.780 | TUBA1A | CXCR4 | 0.707 |
| MYO1D | MMP19 | 0.792 | CDX2 | RFLNB | 0.792 | PRRX1 | GPR137B | 0.776 |
| NOD2 | THBD | 0.751 | ARPIN | RAB31 | 0.763 | NCF2 | CEP19 | 0.740 |
| TREM1 | CCL2 | 0.745 | LY96 | P2RY13 | 0.814 | FLT1 | RHOH | 0.752 |
| NFE2 | SELL | 0.799 | TMEM154 | TLR1 | 0.760 | MSN | CCN4 | 0.765 |
| CCDC88A | S100A8 | 0.828 | COTL1 | NRG1 | 0.789 | TRPM6 | CD93 | 0.811 |
| CTHRC1 | COL4A2 | 0.773 | SHTN1 | PPP1R18 | 0.760 | COL7A1 | TMEM273 | 0.805 |
| NNMT | C1R | 0.772 | ITGAM | LGALS1 | 0.770 | SNORC | RGS19 | 0.749 |
| FGF2 | CHST11 | 0.775 | COL15A1 | NCF4 | 0.771 | SLC15A3 | SH3BP5 | 0.735 |
| ACOT9 | FFAR2 | 0.754 | NRG1 | VAV1 | 0.784 | LILRB1 | RGS16 | 0.792 |
| TNC | CCL4 | 0.792 | TMEM273 | SOD2 | 0.813 | RGS16 | PDLIM4 | 0.778 |
| AFG3L2 | PPP2R5A | 0.776 | COX10 | VAV1 | 0.771 | BDH1 | GZMB | 0.785 |
| PFKL | BPGM | 0.779 | NFAM1 | ENO2 | 0.758 | UQCRC1 | LOXL2 | 0.813 |
| MYO1F | SSH2 | 0.737 | ANGPTL2 | CD14 | 0.754 | OCRL | STC1 | 0.815 |
| RGS19 | GPR137B | 0.760 | DPYSL3 | TUBB6 | 0.765 | S100A8 | TNFRSF1B | 0.777 |
| PPP1R18 | THEMIS2 | 0.764 | CGAS | CYTH4 | 0.758 | LILRB2 | G0S2 | 0.813 |
| MIR100HG | S100A12 | 0.818 | FGF2 | S100A8 | 0.810 | COX10 | C11orf96 | 0.803 |
| TRPM6 | PLAU | 0.774 | BDH1 | SLA | 0.761 | NDUFA10 | COQ9 | 0.781 |
| ANKRD35 | SLC2A3 | 0.764 | CCDC69 | PCBP3 | 0.778 | TMEM71 | FPR2 | 0.751 |
| ADCY6 | RNF149 | 0.787 | VIM | SLC16A6 | 0.748 | HCLS1 | TUBA1A | 0.683 |
| LILRA2 | KCNJ15 | 0.680 | LCP2 | S100A9 | 0.823 | FPR1 | SRGN | 0.765 |
| DLC1 | G0S2 | 0.840 | RBMS1 | CD14 | 0.762 | SERPINE1 | SMAP2 | 0.782 |
| RAB15 | TNFAIP6 | 0.786 | AKAP1 | CYP26B1 | 0.821 | BDH1 | PCBP3 | 0.779 |
| SPOCK1 | STC1 | 0.799 | CBX6 | CXCR4 | 0.681 | CLU | LCP1 | 0.744 |
| PKIB | TCF4 | 0.772 | TRPM6 | PAPPA | 0.816 | LRP4 | PPP1R18 | 0.775 |
| SLC11A1 | THBD | 0.817 | BCL2A1 | TCF4 | 0.767 | DSP | TUBB6 | 0.791 |
| ARPIN | CHN1 | 0.780 | SWAP70 | RBMS1 | 0.733 | EMP3 | ANGPTL2 | 0.752 |
| ERMP1 | SOD2 | 0.805 | S1PR1 | JAZF1 | 0.763 | CSF3 | HLX | 0.754 |
| FAP | CXCR1 | 0.757 | GPR137B | CLMP | 0.785 | IL24 | LILRA5 | 0.808 |
| PIP4P2 | C5AR1 | 0.761 | LDLRAD3 | VNN2 | 0.807 | IFITM2 | SWAP70 | 0.726 |
| VAV1 | BMERB1 | 0.801 | FILIP1L | GADD45B | 0.779 | SH3RF2 | CD86 | 0.788 |
| ERMP1 | SRGN | 0.817 | ABL2 | CYTH4 | 0.767 | TREM1 | CD53 | 0.733 |
| PI15 | CD53 | 0.747 | CDX2 | CAVIN3 | 0.804 | PCBP3 | HS3ST3B1 | 0.781 |
| GAS2L3 | C5AR1 | 0.788 | SLA | BPGM | 0.751 | AMPD2 | LCP2 | 0.774 |
| NFAM1 | FCGR2B | 0.777 | PDE4B | RIPK2 | 0.747 | C2CD2 | TNFRSF1B | 0.761 |
| ZDHHC23 | SLA | 0.792 | TRPM6 | FCER1G | 0.771 | ITGA5 | CD69 | 0.769 |
| GADD45B | RIPOR2 | 0.772 | HSD11B2 | CHI3L1 | 0.798 | FILIP1L | ENTPD1 | 0.788 |
| LILRA5 | THBD | 0.801 | MYO1D | DPYSL3 | 0.780 | ASAP1 | SRGN | 0.785 |
| FJX1 | GZMB | 0.765 | SERPINE2 | PROK2 | 0.735 | ARPIN | RGS19 | 0.739 |
| BDH1 | TUBB6 | 0.792 | MMP19 | PAPPA | 0.833 | AMPD2 | ENTPD1 | 0.802 |
| KIAA1522 | SH2D3C | 0.769 | TMEM106C | LILRB1 | 0.766 | CMTM4 | NIPSNAP3A | 0.760 |
| F5 | MIR100HG | 0.787 | CYTH4 | HCK | 0.747 | MX2 | VAV1 | 0.729 |
| IL13RA2 | STAT5B | 0.872 | CXCR2 | CD53 | 0.752 | S100A9 | CD14 | 0.786 |
| ASB7 | JAZF1 | 0.775 | SLC15A4 | CEP19 | 0.730 | IL1RN | CXCR2 | 0.809 |
| NDUFA10 | SPOCK1 | 0.789 | CMTM8 | TNC | 0.795 | RAB15 | RILPL2 | 0.806 |
| LDLRAD3 | PTPRC | 0.783 | NDUFA10 | RFLNB | 0.802 | TRAK1 | NR3C2 | 0.785 |
| RHOH | SNN | 0.767 | PTGS2 | ITPRIP | 0.824 | LY96 | NFE2 | 0.787 |
| GALNT12 | BASP1 | 0.797 | RNF149 | ST3GAL5 | 0.783 | CMTM4 | GPR176 | 0.764 |
| TNFAIP6 | LAT2 | 0.792 | MYO1F | SPOCK1 | 0.774 | FCER1G | SRGN | 0.781 |
| LILRA2 | CYP26B1 | 0.802 | SLC15A4 | PRICKLE2 | 0.803 | LRP4 | CEBPB | 0.804 |
| BDH1 | TNC | 0.803 | HSD11B2 | MMP3 | 0.812 | RGS19 | HS3ST3B1 | 0.746 |
| WNK2 | ASAP1 | 0.811 | EFNA4 | WWC2 | 0.765 | LILRB1 | SLC11A1 | 0.809 |
| CAVIN3 | PRICKLE2 | 0.787 | TNFRSF1B | SPHK1 | 0.748 | OLFM1 | FAP | 0.778 |
| CHRDL2 | RBMS1 | 0.768 | SWAP70 | SNN | 0.733 | AGFG2 | SERPINE2 | 0.779 |
| GLCE | CCL4 | 0.777 | PRICKLE2 | SPOCK1 | 0.791 | S100A12 | SPHK1 | 0.790 |
| STAP2 | FAP | 0.768 | CAVIN1 | PTGS2 | 0.788 | S1PR3 | INHBA | 0.787 |
| LILRB3 | RIPOR3 | 0.746 | LRRK2 | WNT5A | 0.816 | SERPINB9 | SLC2A3 | 0.759 |
| TNFAIP6 | ST3GAL5 | 0.791 | S1PR1 | C5AR1 | 0.762 | FZD5 | FPR1 | 0.794 |
| RGS19 | CYP26B1 | 0.777 | AGAP1 | TMEM273 | 0.786 | TMEM106C | PLEK | 0.759 |
| CR1 | BCAT1 | 0.741 | NEO1 | SLC2A3 | 0.782 | ENO2 | TNFAIP2 | 0.760 |
| KIAA1522 | FPR2 | 0.798 | FPR1 | PXDN | 0.767 | DSP | TCEA3 | 0.779 |
| IL13RA2 | SSH2 | 0.872 | MMP3 | NRCAM | 0.790 | GLCE | IL13RA2 | 0.879 |
| CCR1 | CLEC4A | 0.798 | CD93 | NRCAM | 0.763 | LY96 | TCF4 | 0.792 |
| C11orf96 | LCP2 | 0.782 | CAVIN3 | CCN4 | 0.791 | LCP1 | NRCAM | 0.734 |
| IL11 | STAT5B | 0.831 | DPYSL2 | S1PR3 | 0.754 | LOXL2 | PIP4P2 | 0.784 |
| CLEC4A | SMAP2 | 0.783 | CRIP2 | VAV1 | 0.755 | ZNF704 | TIMP1 | 0.760 |
| AGFG2 | LILRB2 | 0.815 | CXCR2 | IL11 | 0.838 | COL15A1 | LY96 | 0.778 |
| LRRK2 | CD93 | 0.808 | HIBCH | CD300A | 0.818 | MYO1F | PDE4B | 0.720 |
| CSNK1G3 | COTL1 | 0.750 | TSPAN6 | LILRA2 | 0.779 | TCEA3 | CXCR4 | 0.744 |
| COL7A1 | GFPT2 | 0.773 | FAM155A | THEMIS2 | 0.800 | SHTN1 | SOD2 | 0.778 |
| AKAP1 | DUSP2 | 0.779 | CD86 | CHST2 | 0.780 | IL11 | MAP4K4 | 0.826 |
| RALGAPA2 | NCF4 | 0.763 | ATP5IF1 | PRICKLE2 | 0.818 | SLA | ENTPD1 | 0.749 |
| NDFIP2 | TUBA1A | 0.782 | DLC1 | TNF | 0.817 | PLA1A | CEP19 | 0.718 |
| OSM | BCL2A1 | 0.796 | VASN | COL7A1 | 0.745 | CBX6 | MSN | 0.764 |
| NDUFA10 | PDLIM4 | 0.802 | TRPM6 | IL1B | 0.790 | GLT1D1 | CLMP | 0.770 |
| RGCC | HS3ST3B1 | 0.790 | ANGPTL2 | WNT5A | 0.797 | JAZF1 | STC1 | 0.811 |
| COX10 | TMEM273 | 0.788 | CMTM8 | MAP4K4 | 0.788 | DUSP4 | PTGS1 | 0.804 |
| LOXL2 | CLMP | 0.773 | COL15A1 | RAI14 | 0.765 | ARPIN | BMERB1 | 0.804 |
| LILRA2 | FAM49A | 0.754 | CAVIN1 | CD53 | 0.734 | PREX1 | SSH2 | 0.712 |
| CCR1 | ZDHHC18 | 0.789 | NRCAM | STAT5B | 0.757 | VASN | SELP | 0.772 |
| AGFG2 | COL15A1 | 0.764 | COL15A1 | NFE2 | 0.771 | ZNF704 | FAM155A | 0.808 |
| FGF2 | CREB5 | 0.772 | ENO2 | ANGPT2 | 0.789 | S1PR1 | CD53 | 0.715 |
| ZNF704 | RIPOR2 | 0.763 | KIAA1522 | CRIP2 | 0.785 | WNK2 | MIR100HG | 0.819 |
| EGR3 | CLEC4E | 0.745 | RAI14 | VNN2 | 0.806 | COL7A1 | PDE4B | 0.776 |
| CAVIN3 | RIPOR2 | 0.759 | C1R | BTG2 | 0.784 | ITGAM | P2RY14 | 0.771 |
| CXCR1 | TNFRSF1B | 0.706 | COX10 | MMP3 | 0.803 | PAPPA | CCDC3 | 0.813 |
| CBX6 | CREB5 | 0.743 | FGF2 | CCDC69 | 0.776 | COQ9 | STAT5B | 0.763 |
| TUBB6 | ITPRIP | 0.799 | CGAS | RGS16 | 0.741 | LRP4 | OCRL | 0.779 |
| FZD5 | TNFRSF1B | 0.761 | SERPINB9 | SELE | 0.815 | AMPD2 | CEMIP | 0.817 |
| ABL2 | DOK3 | 0.737 | AGAP1 | COL4A2 | 0.780 | CCDC88A | CCL4 | 0.773 |
| WNT5A | SNN | 0.812 | HLX | PRR16 | 0.745 | SERPINE1 | GPR65 | 0.791 |
| FFAR2 | IER5 | 0.719 | TMEM106C | TMEM71 | 0.800 | TRPM6 | C5AR1 | 0.796 |
| CSNK1G3 | HIBCH | 0.753 | LILRB2 | FAM49A | 0.763 | RALGAPA2 | PLEK | 0.775 |
| ERMP1 | CGAS | 0.771 | CSNK1G3 | BDH1 | 0.782 | ASAP1 | RASSF5 | 0.758 |
| CMTM4 | NEO1 | 0.781 | ASB7 | FFAR2 | 0.721 | CHI3L1 | TNF | 0.811 |
| EMP3 | PRRX1 | 0.751 | CSF2RB | SERPINB9 | 0.777 | SMAP2 | SSH2 | 0.747 |
| DPYSL3 | NFE2 | 0.781 | GLIPR1 | PLAU | 0.704 | OCRL | PTPRC | 0.792 |
| CCDC69 | NOD2 | 0.716 | CEBPB | RGCC | 0.795 | NRG1 | RFLNB | 0.783 |
| ZDHHC23 | CEBPB | 0.829 | NEO1 | IL11 | 0.826 | RILPL2 | PXDN | 0.798 |
| CYTH4 | BPGM | 0.789 | HSD11B2 | OLFM1 | 0.807 | LILRB2 | CHST2 | 0.808 |
| KIAA1522 | SELL | 0.800 | PDPN | TMEM273 | 0.789 | MYO1D | CD14 | 0.783 |
| TNFRSF10C | CREB5 | 0.715 | CHRDL2 | PLEK | 0.779 | FCGR3B | MYO1F | 0.812 |
| SLA | PDLIM4 | 0.749 | BCAT1 | SPP1 | 0.760 | TMEM154 | TNC | 0.800 |
| SWAP70 | ASAP1 | 0.726 | FPR2 | MSN | 0.800 | G0S2 | S100A9 | 0.807 |
| TLR4 | CD69 | 0.690 | TMEM273 | INHBA | 0.808 | RILPL2 | PRRX1 | 0.771 |
| PPP2R5A | FGR | 0.800 | CSF2RB | TMEM154 | 0.768 | FAM20C | SOD2 | 0.767 |
| AGFG2 | PRR16 | 0.805 | TNFAIP2 | GPR65 | 0.741 | NEO1 | CCDC3 | 0.784 |
| FCER1G | SELE | 0.815 | CEBPB | RIPOR2 | 0.776 | NIPSNAP3A | SWAP70 | 0.759 |
| NDUFA10 | PCBP3 | 0.767 | FILIP1L | HCLS1 | 0.701 | ENO2 | HS3ST3B1 | 0.773 |
| SHTN1 | SLFN11 | 0.761 | PCBP3 | SLC16A6 | 0.785 | TSPAN6 | HS3ST3B1 | 0.809 |
| NCF2 | BCL2A1 | 0.740 | IFITM2 | CCL2 | 0.766 | IL1B | PDE4B | 0.766 |
| HSD11B2 | SLA | 0.769 | GLCE | RFLNB | 0.783 | CXCR2 | ITPRIP | 0.849 |
| NNMT | SLC11A1 | 0.810 | COL15A1 | SERPINE1 | 0.780 | AFG3L2 | COL15A1 | 0.786 |
| RAB31 | TLR1 | 0.781 | HLX | TNF | 0.738 | DLC1 | SPHK1 | 0.792 |
| NDFIP2 | CLEC4A | 0.802 | GALNT12 | RAB31 | 0.794 | NFAM1 | NOD2 | 0.719 |
| SPP1 | MAP4K4 | 0.754 | AGFG2 | MMP19 | 0.779 | SLC15A3 | ARHGAP25 | 0.738 |
| OLFM1 | COTL1 | 0.765 | PDE4B | SLC16A6 | 0.699 | RAB31 | CTHRC1 | 0.775 |
| HCK | NFE2 | 0.767 | TMEM273 | RILPL2 | 0.808 | PDPN | C5AR1 | 0.788 |
| CBX6 | OSMR | 0.778 | HSD11B2 | LILRB3 | 0.764 | GALNT12 | NCF4 | 0.769 |
| IFITM2 | FAM49A | 0.794 | IL1B | NFE2 | 0.783 | ITPRIP | HS3ST3B1 | 0.811 |
| SELP | GMFG | 0.809 | CBX6 | CCDC88A | 0.721 | IL1RN | RFLNB | 0.780 |
| CD300A | MX2 | 0.802 | ITGA5 | P2RY14 | 0.794 | NFAM1 | CCDC88A | 0.729 |
| COL7A1 | LCP1 | 0.765 | CYP26B1 | TUBA1A | 0.803 | AQP9 | ITGA5 | 0.775 |
| COX10 | CHN1 | 0.801 | SLC15A4 | ITPRIP | 0.805 | GOLPH3L | RHOH | 0.757 |
| CD300A | SLC16A6 | 0.780 | EFNA4 | TCEA3 | 0.778 | GAS2L3 | NEO1 | 0.758 |
| TCEA3 | CSF2RB | 0.798 | ERMP1 | GPR65 | 0.787 | PPP1R18 | TNFAIP6 | 0.783 |
| CAVIN3 | DUSP4 | 0.805 | TRAK1 | PRICKLE2 | 0.815 | AGAP1 | CHN1 | 0.797 |
| FZD5 | PDLIM4 | 0.790 | FGF2 | CHST2 | 0.771 | NIPSNAP3A | SELENBP1 | 0.763 |
| TRIM2 | CEBPB | 0.790 | FPR2 | TIMP1 | 0.787 | HSPA6 | STC1 | 0.791 |
| CHI3L2 | CLMP | 0.759 | CHRDL2 | TUBA1A | 0.756 | CD93 | CREB5 | 0.783 |
| ITGAM | ASAP1 | 0.760 | RHOH | MCAM | 0.766 | AGFG2 | BASP1 | 0.792 |
| SNORC | PTGS1 | 0.797 | NFAM1 | ABL2 | 0.745 | HCLS1 | LCP2 | 0.690 |
| CCDC69 | TREM1 | 0.743 | CXCL8 | TREM1 | 0.755 | NEO1 | FERMT2 | 0.817 |
| AKAP1 | C1R | 0.796 | ANKRD35 | RHOH | 0.764 | GAS2L3 | PXDN | 0.797 |
| ZNF704 | SLC15A3 | 0.768 | GLIPR1 | MMP3 | 0.740 | CSTA | CCL2 | 0.736 |
| COL4A2 | RFLNB | 0.758 | SLC15A4 | HCLS1 | 0.743 | TAGAP | LAT2 | 0.741 |
| C5AR1 | CCN4 | 0.773 | SRGN | S100A9 | 0.824 | NFAM1 | RBMS1 | 0.741 |
| OCRL | CSF3R | 0.748 | COL4A2 | WNT5A | 0.788 | GAS2L3 | SERPINB9 | 0.774 |
| ST3GAL5 | P2RY14 | 0.770 | BDH1 | SH3BP5 | 0.778 | COX10 | C1R | 0.793 |
| GLIPR1 | SOD2 | 0.741 | TCEA3 | GADD45B | 0.772 | TRAK1 | TUBB6 | 0.801 |
| STAT5B | GPR65 | 0.759 | ST3GAL5 | SLFN11 | 0.759 | CLU | PTGS2 | 0.787 |
| ZDHHC23 | TNFRSF1B | 0.793 | RFLNB | BCAT1 | 0.760 | LILRB1 | BTG2 | 0.783 |
| SERPINB9 | FAM155A | 0.808 | NDUFA10 | DUSP4 | 0.760 | CAVIN1 | CD69 | 0.748 |
| RNF149 | CXCR4 | 0.767 | CSNK1G3 | TIMP1 | 0.764 | FCGR3B | STC1 | 0.828 |
| TRIM2 | NEO1 | 0.773 | CHI3L1 | CAVIN1 | 0.779 | GAS2L3 | S1PR3 | 0.767 |
| CMTM4 | TIMP1 | 0.776 | FILIP1L | PDLIM4 | 0.781 | CCR1 | AMPD2 | 0.805 |
| SERPINE1 | PCBP3 | 0.799 | CSTA | FAP | 0.728 | LCP1 | CD86 | 0.742 |
| STAP2 | CHI3L1 | 0.783 | AFG3L2 | SH2D3C | 0.765 | FJX1 | CHST2 | 0.753 |
| CSNK1G3 | S100A12 | 0.794 | ADGRG3 | THEMIS2 | 0.763 | SLC15A3 | ACOD1 | 0.751 |
| THEMIS2 | ENTPD1 | 0.801 | INHBA | MAP4K4 | 0.790 | UQCRC1 | KCNJ15 | 0.770 |
| TMEM71 | TLR4 | 0.709 | IL1B | MCAM | 0.810 | RAB31 | CSF3 | 0.784 |
| TMEM273 | BTG2 | 0.786 | FILIP1L | TMEM71 | 0.809 | DPYSL3 | CYTH4 | 0.771 |
| CHST15 | TNF | 0.789 | PDPN | COL15A1 | 0.787 | AGFG2 | PAPPA | 0.823 |
| EGR3 | SELP | 0.783 | CSF2RB | DOK3 | 0.753 | NIBAN1 | GPR183 | 0.756 |
| CAVIN3 | VAV1 | 0.757 | SNORC | SMAP2 | 0.764 | ITGA5 | MSN | 0.766 |
| C2CD2 | GFPT2 | 0.741 | BCAT1 | GFPT2 | 0.741 | BICD2 | PRRX1 | 0.769 |
| F5 | TIMP1 | 0.787 | SLC15A4 | FAP | 0.774 | CSNK1G3 | MSN | 0.748 |
| DPYSL3 | GZMB | 0.785 | SH2D3C | TREM1 | 0.744 | CCR1 | PLEK | 0.784 |
| RNF149 | LOXL2 | 0.822 | RHOH | SERPINE1 | 0.762 | NIBAN1 | FPR2 | 0.739 |
| NR3C2 | LILRA2 | 0.787 | PIK3AP1 | CXCL8 | 0.759 | FCER1G | NRCAM | 0.772 |
| TMEM106C | EMP3 | 0.782 | MX2 | TLR1 | 0.764 | OCRL | CCN4 | 0.789 |
| ITPRIP | CD86 | 0.802 | MYO1D | IL11 | 0.831 | LOXL2 | PCBP3 | 0.784 |
| SNORC | FJX1 | 0.775 | C2CD2 | HLX | 0.711 | FCGR2B | C5AR1 | 0.799 |
| S100A8 | GFPT2 | 0.779 | DPYSL2 | BPGM | 0.768 | COL15A1 | TCF4 | 0.749 |
| FAM20C | STAT5B | 0.776 | CEMIP | RIPOR2 | 0.768 | CGAS | MAP4K4 | 0.719 |
| ANGPT2 | ACOD1 | 0.785 | ABL2 | IER5 | 0.758 | CSF3R | CLMP | 0.746 |
| ASB7 | LAT2 | 0.772 | ATP5IF1 | IL11 | 0.837 | GOLPH3L | S100A12 | 0.787 |
| LGALS1 | PLA1A | 0.778 | AFG3L2 | PDLIM4 | 0.774 | RNF149 | OSM | 0.807 |
| SWAP70 | LGALS1 | 0.724 | TRPM6 | CR1 | 0.782 | TNC | SPHK1 | 0.797 |
| CHST11 | PHLDB2 | 0.759 | TRPM6 | RIPK2 | 0.782 | MYO1D | LILRA2 | 0.793 |
| ADCY6 | GMFG | 0.781 | C5AR1 | VAV1 | 0.740 | CHRDL2 | NNMT | 0.792 |
| UBAC1 | RGS16 | 0.787 | LILRB3 | SLA | 0.729 | CCR1 | SLFN11 | 0.769 |
| SELENBP1 | CD86 | 0.819 | FGR | BCAT1 | 0.763 | SLA | BMERB1 | 0.783 |
| MYO1F | THEMIS2 | 0.733 | GNAI2 | CAVIN1 | 0.740 | RAB31 | S100A12 | 0.787 |
| GZMB | PI15 | 0.771 | RGS19 | LRRK2 | 0.719 | CXCR2 | KCNJ15 | 0.732 |
| NCF2 | SERPINE1 | 0.776 | ICAM1 | RFLNB | 0.755 | ITGAM | DOK3 | 0.722 |
| FJX1 | ARHGAP25 | 0.752 | NDFIP2 | CGAS | 0.770 | RAB31 | CLU | 0.759 |
| HIBCH | SLFN11 | 0.777 | PDPN | MMP3 | 0.781 | TAGAP | RNF149 | 0.732 |
| KCNJ15 | PTPRC | 0.709 | NDFIP2 | G0S2 | 0.825 | ARPIN | NCF2 | 0.786 |
| GPR176 | GPR65 | 0.762 | S1PR1 | RIPOR2 | 0.734 | FCGR2B | HCLS1 | 0.745 |
| HSD11B2 | CAVIN1 | 0.787 | TMEM273 | RAI14 | 0.771 | LILRB2 | BCL2A1 | 0.766 |
| CSNK1G3 | SLC11A1 | 0.797 | TAGAP | CCDC69 | 0.702 | PFKL | LOXL2 | 0.804 |
| PTGS2 | CHST2 | 0.791 | RFLNB | PXDN | 0.782 | FLT1 | FPR1 | 0.755 |
| SRGN | SLC2A3 | 0.747 | CXCL5 | THEMIS2 | 0.774 | CCDC88A | CXCL5 | 0.759 |
| SORT1 | IER5 | 0.746 | DPYSL3 | LCP2 | 0.772 | GADD45B | OSMR | 0.802 |
| ADGRG3 | ASAP1 | 0.758 | TNFAIP2 | CCDC3 | 0.765 | MX2 | PHLDB2 | 0.789 |
| FGF7 | GPR65 | 0.790 | CMTM4 | SELP | 0.809 | B4GALT4 | MMP19 | 0.759 |
| MCAM | TREM1 | 0.766 | CMTM4 | CSTA | 0.755 | FPR1 | MEFV | 0.744 |
| S100A12 | TNFAIP6 | 0.798 | LILRA5 | RGS16 | 0.759 | CMTM8 | ZNF704 | 0.798 |
| CSTA | TNC | 0.767 | CSNK1G3 | CXCL5 | 0.764 | GZMB | SSH2 | 0.780 |
| CSNK1G3 | SELE | 0.791 | CCDC69 | HS3ST3B1 | 0.793 | ADCY6 | LGALS1 | 0.786 |
| CSF3R | PDLIM4 | 0.745 | DSP | INHBA | 0.811 | ANKRD35 | RBMS1 | 0.780 |
| LOXL2 | CCL2 | 0.770 | TRAK1 | CYP26B1 | 0.831 | ASB7 | CCDC69 | 0.729 |
| COL15A1 | TMEM154 | 0.767 | RAB31 | S1PR1 | 0.766 | PFKL | CAVIN1 | 0.782 |
| NDFIP2 | ATP5IF1 | 0.773 | NFE2 | C5AR1 | 0.743 | NFE2 | PIP4P2 | 0.756 |
| FCGR2B | OSMR | 0.794 | PDLIM4 | SPHK1 | 0.779 | COL7A1 | TMEM154 | 0.810 |
| CHST11 | ASAP1 | 0.747 | NDUFA10 | RNF149 | 0.797 | ACOD1 | SLC16A6 | 0.740 |
| CTHRC1 | CLU | 0.771 | SLC15A3 | CCL2 | 0.759 | PRRX1 | PTGS1 | 0.773 |
| NFAM1 | FGF2 | 0.757 | PDE4B | LAT2 | 0.771 | ZDHHC23 | NCF4 | 0.790 |
| ZDHHC23 | SORT1 | 0.833 | NDFIP2 | CYP26B1 | 0.810 | IL1B | PTPRC | 0.795 |
| CREM | FAM20C | 0.786 | TRPM6 | CHST11 | 0.786 | RIPOR3 | VIM | 0.779 |
| GALNT12 | CHST2 | 0.781 | DLC1 | C1R | 0.778 | TRIM2 | SOD2 | 0.772 |
| LILRA5 | VIM | 0.785 | AGAP1 | CREB5 | 0.776 | MYO1D | RAI14 | 0.783 |
| IFITM2 | CHST2 | 0.763 | BCL6 | SPHK1 | 0.706 | RILPL2 | CCDC69 | 0.794 |
| TRPM6 | CLMP | 0.783 | COX10 | CCDC69 | 0.780 | FAP | ST3GAL5 | 0.751 |
| RAB15 | PIP4P2 | 0.808 | CXCL8 | SERPINE2 | 0.800 | CCL2 | DOK3 | 0.750 |
| CLEC4A | WWC2 | 0.791 | IFITM2 | LILRA2 | 0.752 | CCR1 | FCGR3B | 0.819 |
| PKIB | PRR16 | 0.817 | EGR3 | P2RY14 | 0.749 | EGR3 | C11orf96 | 0.747 |
| UBAC1 | BTG2 | 0.797 | CGAS | ARHGAP25 | 0.713 | GAS2L3 | OLFM1 | 0.804 |
| RFLNB | CYP26B1 | 0.788 | PIK3AP1 | BPGM | 0.727 | GLT1D1 | IL13RA2 | 0.862 |
| CCN4 | ENTPD1 | 0.776 | PREX1 | SPHK1 | 0.765 | RFLNB | SELE | 0.802 |
| NIBAN1 | VIM | 0.771 | STAP2 | GPR65 | 0.762 | P2RY13 | SELE | 0.794 |
| ZNF704 | PRRX1 | 0.776 | CMTM8 | CXCL5 | 0.774 | RAB15 | CD14 | 0.767 |
| DPYSL3 | HCK | 0.751 | DPYSL3 | S100A8 | 0.814 | PROK2 | ZDHHC18 | 0.727 |
| CHST11 | TCF4 | 0.735 | RGCC | LCP2 | 0.787 | PI15 | NOD2 | 0.738 |
| UBAC1 | DLC1 | 0.809 | CTHRC1 | PDE4B | 0.768 | LDLRAD3 | SLC15A3 | 0.777 |
| RAB15 | ITGA5 | 0.793 | SLC11A1 | TNFRSF10C | 0.784 | CMTM4 | VASN | 0.785 |
| MX2 | SPP1 | 0.758 | ARPIN | MMP19 | 0.768 | AGFG2 | SNN | 0.800 |
| NIBAN1 | COTL1 | 0.785 | IFITM2 | ZDHHC18 | 0.770 | RILPL2 | HSPA6 | 0.755 |
| PFKL | CSF3R | 0.761 | PPP2R5A | CXCR4 | 0.735 | CGAS | RASSF5 | 0.719 |
| TRIM2 | RGS16 | 0.762 | LST1 | ST3GAL5 | 0.767 | CSF3 | NOD2 | 0.762 |
| CCR1 | P2RY14 | 0.799 | OCRL | CHST2 | 0.778 | B4GALT4 | BMERB1 | 0.789 |
| OCRL | NFAM1 | 0.745 | HIBCH | CSF3R | 0.759 | HIBCH | PHLDB2 | 0.786 |
| BDH1 | CLMP | 0.793 | SERPINE1 | SLC11A1 | 0.813 | TCEA3 | LCP1 | 0.756 |
| AQP9 | CHST2 | 0.774 | CXCL8 | CCL2 | 0.775 | TSPAN6 | P2RY13 | 0.805 |
| IL6 | LGALS1 | 0.807 | PPP2R5A | STAT5B | 0.753 | PDPN | BPGM | 0.771 |
| C11orf96 | FAM155A | 0.821 | SNORC | CCDC88A | 0.786 | NR3C2 | CTHRC1 | 0.814 |
| S1PR1 | PLEK | 0.772 | LDLRAD3 | RNF149 | 0.800 | PI15 | BMERB1 | 0.784 |
| C11orf96 | PXDN | 0.789 | HSD11B2 | VASN | 0.768 | TSPAN6 | CD86 | 0.807 |
| BASP1 | IFITM2 | 0.770 | SH3RF2 | BCAT1 | 0.764 | BASP1 | DOK3 | 0.755 |
| TLR1 | SNN | 0.817 | CEBPB | SMAP2 | 0.777 | LRP4 | TNC | 0.790 |
| NIPSNAP3A | BCL2A1 | 0.816 | ANGPT2 | LCP2 | 0.793 | COL4A2 | PHLDB2 | 0.756 |
| MSN | TNFRSF1B | 0.744 | SELENBP1 | VNN2 | 0.822 | OCRL | PAPPA | 0.848 |
| NFE2 | PDLIM4 | 0.792 | FPR1 | MCAM | 0.773 | WNK2 | CHST11 | 0.784 |
| B4GALT4 | FGF2 | 0.778 | MYO1D | INHBA | 0.798 | WNK2 | PIP4P2 | 0.815 |
| CR1 | ITPRIP | 0.784 | AKAP1 | MYO1F | 0.792 | LILRA2 | TMEM273 | 0.778 |
| KCNJ15 | CXCL5 | 0.728 | NIBAN1 | SELL | 0.810 | DUSP4 | IL1RN | 0.767 |
| BPGM | STC1 | 0.823 | CLEC4A | CEMIP | 0.794 | VASN | ACOT9 | 0.757 |
| ARPIN | SERPINB9 | 0.769 | GOLPH3L | C1R | 0.779 | CBX6 | SLFN11 | 0.712 |
| FGF7 | LGALS1 | 0.792 | PRICKLE2 | FJX1 | 0.779 | AGFG2 | SLC15A3 | 0.766 |
| ERMP1 | MMP3 | 0.812 | FERMT2 | MX2 | 0.786 | HIBCH | WNK2 | 0.817 |
| ERMP1 | RGS16 | 0.783 | NCF2 | PLA1A | 0.768 | PIP4P2 | STC1 | 0.806 |
| SNORC | NNMT | 0.818 | CHN1 | NCF2 | 0.776 | C11orf96 | GADD45B | 0.792 |
| PFKL | SERPINE2 | 0.804 | TMEM106C | CGAS | 0.754 | ADGRG3 | CREB5 | 0.736 |
| CXCL8 | IL11 | 0.813 | SLA | LAT2 | 0.750 | PDPN | CXCR4 | 0.754 |
| TCEA3 | CD53 | 0.756 | GLT1D1 | WWC2 | 0.772 | BDH1 | SWAP70 | 0.749 |
| FZD5 | LDLRAD3 | 0.791 | RAI14 | THBD | 0.788 | LOXL2 | BMERB1 | 0.814 |
| LRRK2 | RILPL2 | 0.807 | GNAI2 | PHLDB2 | 0.770 | RAI14 | RHOH | 0.750 |
| SLC15A3 | SMAP2 | 0.746 | ANKRD35 | CBX6 | 0.745 | RNF149 | SPOCK1 | 0.799 |
| FCGR2B | SELP | 0.805 | GOLPH3L | TNFAIP6 | 0.792 | CHN1 | PLAU | 0.773 |
| CHRDL2 | ABL2 | 0.761 | MX2 | CHST2 | 0.787 | BDH1 | LGALS1 | 0.793 |
| FFAR2 | SPOCK1 | 0.787 | TMEM273 | PAPPA | 0.838 | CSF3R | S1PR3 | 0.753 |
| CSF3R | PDE4B | 0.690 | SNORC | GMFG | 0.790 | COX10 | PPP1R18 | 0.775 |
| DLC1 | CHN1 | 0.802 | PKIB | CYP26B1 | 0.812 | DPYSL3 | ZDHHC18 | 0.767 |
| COX10 | CD69 | 0.783 | DUSP4 | CYTH4 | 0.779 | TMEM154 | CCDC88A | 0.742 |
| SELE | SMAP2 | 0.795 | GPR137B | ST3GAL5 | 0.802 | NCF4 | CCDC3 | 0.774 |
| TMEM154 | RIPOR2 | 0.730 | GOLPH3L | CTHRC1 | 0.804 | LILRB2 | PROK2 | 0.740 |
| PAPPA | TNFAIP2 | 0.822 | AKAP1 | CSTA | 0.779 | FGR | TCF4 | 0.772 |
| FAP | SLC16A6 | 0.773 | CHI3L1 | SLC15A4 | 0.811 | MMP19 | SPOCK1 | 0.788 |
| CCR1 | S1PR1 | 0.800 | ZDHHC23 | GMFG | 0.806 | GNAI2 | ST3GAL5 | 0.761 |
| GAS2L3 | SORT1 | 0.776 | IL11 | PDE4B | 0.803 | SNN | CD53 | 0.750 |
| NIBAN1 | COL7A1 | 0.807 | MYO1D | CHN1 | 0.793 | NR3C2 | FCGR3B | 0.803 |
| PAPPA | RGS16 | 0.832 | NIBAN1 | HCLS1 | 0.716 | CLEC4E | NFE2 | 0.720 |
| NDUFA10 | PTGS2 | 0.816 | LRP4 | RAB31 | 0.794 | ARPIN | TMEM154 | 0.796 |
| SORT1 | PROK2 | 0.736 | DUSP2 | ARHGAP25 | 0.754 | IL24 | SPHK1 | 0.796 |
| CD300A | NIBAN1 | 0.791 | LILRA5 | DOK3 | 0.764 | LRP4 | MCAM | 0.792 |
| CYTH4 | CXCL5 | 0.773 | CAVIN1 | BCL2A1 | 0.767 | CEBPB | CXCL5 | 0.765 |
| COL4A2 | CREM | 0.780 | SELENBP1 | CREM | 0.812 | NFE2 | NRCAM | 0.778 |
| FGF7 | TNFRSF10C | 0.804 | RAB31 | CLMP | 0.761 | CCDC88A | RASSF5 | 0.724 |
| PPP1R18 | CLMP | 0.768 | HIBCH | PKIB | 0.775 | UBAC1 | SLC35G2 | 0.820 |
| DSP | RGS19 | 0.738 | PAPPA | PRRX1 | 0.815 | ERMP1 | GPR137B | 0.799 |
| TMEM106C | PLAU | 0.770 | TRIM2 | SLC15A3 | 0.764 | PCBP3 | SELP | 0.803 |
| BICD2 | BCAT1 | 0.754 | DPYSL3 | IL13RA2 | 0.872 | RGS19 | MCAM | 0.720 |
| BCL2A1 | GMFG | 0.782 | NRG1 | RGS16 | 0.784 | DUSP2 | CD86 | 0.762 |
| CHI3L1 | DPYSL3 | 0.786 | CDX2 | ICAM1 | 0.760 | ADGRG3 | RIPOR2 | 0.736 |
| GADD45B | PTGS2 | 0.778 | LRP4 | PRICKLE2 | 0.805 | FZD5 | SOD2 | 0.791 |
| COX10 | GMFG | 0.784 | HCK | SLFN11 | 0.749 | DOK3 | SH3BP5 | 0.728 |
| CRIP2 | BPGM | 0.781 | TRIM2 | SERPINE2 | 0.794 | PKIB | CSF3 | 0.823 |
| CMTM4 | TMEM71 | 0.804 | ZDHHC23 | RBMS1 | 0.845 | BASP1 | S1PR1 | 0.799 |
| OCRL | GPR183 | 0.783 | CSTA | SNN | 0.734 | CREM | LILRA5 | 0.764 |
| LILRB2 | LCP1 | 0.748 | FZD5 | RGS19 | 0.728 | BDH1 | LOXL2 | 0.796 |
| NIPSNAP3A | CCL2 | 0.807 | F5 | SOD2 | 0.778 | NIBAN1 | ACOD1 | 0.763 |
| EMP3 | IL6 | 0.799 | HCK | WNT5A | 0.773 | ITGA5 | VNN2 | 0.813 |
| UQCRC1 | CD14 | 0.781 | MYO1D | CXCL8 | 0.796 | CDX2 | CHI3L2 | 0.743 |
| COQ9 | CHN1 | 0.790 | FZD5 | RIPOR2 | 0.758 | FCER1G | CEP19 | 0.739 |
| TMEM154 | HCLS1 | 0.695 | PFKL | CCDC88A | 0.791 | GPR137B | HS3ST3B1 | 0.800 |
| RGS19 | FCGR2B | 0.749 | SH3RF2 | CCDC3 | 0.764 | FGF7 | PROK2 | 0.785 |
| MX2 | WNT5A | 0.806 | TRIM2 | CCL2 | 0.777 | C2CD2 | HCLS1 | 0.718 |
| VASN | SERPINE2 | 0.737 | IFITM2 | HCK | 0.747 | RGS19 | CXCL5 | 0.727 |
| RALGAPA2 | FCER1G | 0.776 | CCR1 | TMEM273 | 0.801 | CTHRC1 | MX2 | 0.786 |
| TNFAIP2 | VAV1 | 0.739 | SERPINB9 | CCL2 | 0.770 | EFNA4 | HCK | 0.759 |
| AGFG2 | KIAA1522 | 0.771 | COL7A1 | CEMIP | 0.799 | BICD2 | BTG2 | 0.767 |
| EMP3 | ITGA5 | 0.789 | FILIP1L | GPR65 | 0.786 | PTGS2 | SLC16A6 | 0.755 |
| ERMP1 | SPHK1 | 0.768 | SLC11A1 | TNFAIP6 | 0.792 | PKIB | COL15A1 | 0.779 |
| SOD2 | VIM | 0.783 | CMTM4 | LAT2 | 0.764 | PTGS1 | SMAP2 | 0.763 |
| EFNA4 | PPP2R5A | 0.779 | IL24 | GNAI2 | 0.778 | CHN1 | FCGR3B | 0.815 |
| CHRDL2 | PTPRC | 0.768 | HCLS1 | CEP19 | 0.654 | MX2 | VNN2 | 0.789 |
| S1PR3 | PTGS2 | 0.790 | DLC1 | TMEM273 | 0.790 | ZDHHC23 | WNK2 | 0.832 |
| COL15A1 | FCGR2B | 0.784 | DPYSL3 | HCLS1 | 0.706 | CLEC4E | PTGS2 | 0.752 |
| COL15A1 | FFAR2 | 0.770 | SELP | TLR1 | 0.823 | TMEM154 | CXCR2 | 0.765 |
| CD300A | PLA1A | 0.785 | ADCY6 | BDH1 | 0.778 | ERMP1 | COL15A1 | 0.787 |
| COX10 | RAI14 | 0.800 | COL7A1 | CLMP | 0.796 | FZD5 | SMAP2 | 0.755 |
| CTHRC1 | SNN | 0.796 | TSPAN6 | DUSP4 | 0.766 | CBX6 | CHST2 | 0.763 |
| DUSP4 | PAPPA | 0.807 | RILPL2 | SLC35G2 | 0.813 | BASP1 | CD93 | 0.801 |
| FZD5 | PAPPA | 0.833 | LCP2 | BCL6 | 0.688 | LCP1 | PIP4P2 | 0.734 |
| PKIB | CRIP2 | 0.781 | NDUFA10 | SLFN11 | 0.782 | AGAP1 | CLEC4A | 0.805 |
| ANGPT2 | SLC35G2 | 0.792 | GADD45B | ZDHHC18 | 0.778 | RGS16 | SLC16A6 | 0.752 |
| FAP | STC1 | 0.787 | CDX2 | PDE4B | 0.788 | MIR100HG | SOD2 | 0.767 |
| PTPRC | VAV1 | 0.714 | CMTM8 | SLC11A1 | 0.781 | SHTN1 | CBX6 | 0.770 |
| NRCAM | CLEC4A | 0.790 | CBX6 | NCF2 | 0.732 | PLAU | BCAT1 | 0.761 |
| POLDIP2 | PCBP3 | 0.768 | AKAP1 | SLC2A3 | 0.785 | TAGAP | IL1RN | 0.735 |
| CEBPB | CCN4 | 0.784 | ACOT9 | CCN4 | 0.779 | COX10 | SLC35G2 | 0.827 |
| B4GALT4 | SERPINE2 | 0.787 | STAT5B | FAM49A | 0.761 | IL6 | PIP4P2 | 0.818 |
| CTHRC1 | ENO2 | 0.797 | TCEA3 | RHOH | 0.766 | FGF2 | ANGPTL2 | 0.786 |
| ACOT9 | SPP1 | 0.757 | MX2 | BTG2 | 0.781 | COL15A1 | HCK | 0.764 |
| FGR | ANGPTL2 | 0.771 | MYO1F | WWC2 | 0.773 | SH2D3C | SLC15A3 | 0.741 |
| LRP4 | IL1B | 0.795 | LDLRAD3 | CAVIN1 | 0.763 | UQCRC1 | CLEC4A | 0.810 |
| COTL1 | MMP3 | 0.778 | LRRK2 | FLT1 | 0.777 | CHI3L1 | ICAM1 | 0.781 |
| RIPOR3 | JAZF1 | 0.795 | RAB31 | CRIP2 | 0.770 | NRCAM | TNFRSF1B | 0.734 |
| CHST11 | PCBP3 | 0.764 | LST1 | OSMR | 0.788 | CMTM4 | SELENBP1 | 0.791 |
| GAS2L3 | ABL2 | 0.798 | GPR183 | SLC16A6 | 0.726 | CLU | SLC35G2 | 0.790 |
| TNFAIP6 | SNN | 0.792 | SHTN1 | PCBP3 | 0.764 | TMEM71 | AMPD2 | 0.789 |
| DPYSL3 | LAT2 | 0.768 | ATP5IF1 | PTGS2 | 0.800 | FCGR2B | ANGPT2 | 0.796 |
| FFAR2 | IL11 | 0.825 | B4GALT4 | THBD | 0.789 | IL24 | SPP1 | 0.780 |
| FJX1 | NRG1 | 0.800 | MCAM | LST1 | 0.773 | DUSP2 | MYO1F | 0.769 |
| NRG1 | CD14 | 0.770 | C1R | FAM49A | 0.771 | ATP5IF1 | BCL6 | 0.740 |
| EGR3 | PDE4B | 0.720 | CMTM4 | VNN2 | 0.815 | PFKL | CEBPB | 0.807 |
| CSNK1G3 | BMERB1 | 0.785 | SLC15A4 | DUSP2 | 0.784 | ZDHHC23 | CCL4 | 0.794 |
| NR3C2 | CXCR2 | 0.817 | ATP5IF1 | RGS19 | 0.738 | CAVIN1 | LST1 | 0.762 |
| GADD45B | S100A8 | 0.812 | RALGAPA2 | PIK3AP1 | 0.741 | RALGAPA2 | BICD2 | 0.790 |
| RIPOR3 | GNAI2 | 0.796 | IL24 | ITGAM | 0.797 | COL15A1 | MYO1F | 0.771 |
| HIBCH | MYO1D | 0.803 | PKIB | NFE2 | 0.790 | MMP3 | STC1 | 0.806 |
| SWAP70 | MX2 | 0.702 | CMTM4 | IL1RN | 0.785 | RGS16 | TNF | 0.778 |
| SH3RF2 | CD93 | 0.802 | LILRB3 | S1PR3 | 0.741 | SNORC | PREX1 | 0.764 |
| AGAP1 | UQCRC1 | 0.772 | CHI3L1 | NOD2 | 0.768 | COL15A1 | S100A12 | 0.792 |
| SPP1 | GPR65 | 0.755 | RILPL2 | PIP4P2 | 0.816 | COL4A2 | RIPOR3 | 0.775 |
| SH2D3C | CXCR4 | 0.706 | AGAP1 | HS3ST3B1 | 0.792 | GZMB | FAM49A | 0.789 |
| NIBAN1 | CLEC4A | 0.755 | RAB31 | RILPL2 | 0.797 | PDPN | IL6 | 0.798 |
| SELE | BPGM | 0.813 | PIK3AP1 | TREM1 | 0.716 | RAB31 | MAP4K4 | 0.767 |
| S1PR3 | C2CD2 | 0.751 | FGF2 | STAT5B | 0.777 | ARPIN | WWC2 | 0.756 |
| COL7A1 | CD69 | 0.789 | CXCR2 | IL13RA2 | 0.866 | RAB31 | GNAI2 | 0.760 |
| PFKL | DPYSL2 | 0.770 | AKAP1 | FGR | 0.792 | CHST11 | PXDN | 0.779 |
| NFAM1 | FPR1 | 0.746 | BDH1 | COL15A1 | 0.779 | RGS19 | BMERB1 | 0.772 |
| GLCE | FAP | 0.775 | CLU | CCDC69 | 0.762 | HLX | MEFV | 0.721 |
| HSD11B2 | STC1 | 0.793 | POLDIP2 | MYO1D | 0.792 | CSF3R | PHLDB2 | 0.757 |
| SLC2A3 | LST1 | 0.738 | AFG3L2 | SLA | 0.752 | CMTM4 | TMEM273 | 0.798 |
| TNFAIP6 | ENTPD1 | 0.799 | FGF2 | S100A9 | 0.808 | PRRX1 | FAM20C | 0.777 |
| NR3C2 | ANGPTL2 | 0.786 | CSTA | MIR100HG | 0.741 | CHN1 | PDE4B | 0.760 |
| SLC15A4 | IL13RA2 | 0.869 | ERMP1 | NNMT | 0.825 | GLIPR1 | PIP4P2 | 0.707 |
| ADGRG3 | CXCL5 | 0.743 | PIP4P2 | GFPT2 | 0.752 | NDFIP2 | GMFG | 0.785 |
| ASB7 | RIPK2 | 0.803 | CCL4 | SPOCK1 | 0.788 | CLU | SH3BP5 | 0.752 |
| NDFIP2 | RNF149 | 0.813 | CXCL8 | SERPINE1 | 0.792 | ARPIN | CCN4 | 0.774 |
| COX10 | WNK2 | 0.824 | EMP3 | FJX1 | 0.747 | PPP1R18 | LCP1 | 0.714 |
| ADCY6 | ERMP1 | 0.792 | RILPL2 | HS3ST3B1 | 0.800 | LGALS1 | PIP4P2 | 0.770 |
| MYO1D | OSM | 0.811 | PLA1A | P2RY14 | 0.771 | JAZF1 | HCLS1 | 0.695 |
| CSF3R | SLC35G2 | 0.755 | C11orf96 | TNFAIP6 | 0.798 | CLU | NRG1 | 0.806 |
| JAZF1 | LST1 | 0.763 | FILIP1L | RAI14 | 0.759 | TRIM2 | ZDHHC23 | 0.789 |
| NFAM1 | IL1B | 0.773 | ICAM1 | GPR65 | 0.738 | TRIM2 | ATP5IF1 | 0.770 |
| CR1 | SELE | 0.791 | ANGPTL2 | ITPRIP | 0.785 | NIBAN1 | PDE4B | 0.733 |
| GLIPR1 | RHOH | 0.676 | S1PR1 | ENO2 | 0.776 | HIBCH | COL15A1 | 0.770 |
| CHI3L1 | GNAI2 | 0.775 | DLC1 | HSPA6 | 0.805 | ATP5IF1 | CXCL5 | 0.756 |
| NDUFA10 | CLU | 0.759 | TREM1 | TNFRSF10C | 0.720 | NCF2 | CCN4 | 0.752 |
| LRRK2 | PLEK | 0.770 | COL15A1 | SLC2A3 | 0.730 | FPR2 | HS3ST3B1 | 0.794 |
| IL24 | PRR16 | 0.823 | SWAP70 | GPR137B | 0.734 | GLCE | IER5 | 0.753 |
| NCF2 | ITPRIP | 0.821 | FCGR2B | CXCR4 | 0.737 | ITGAM | PDE4B | 0.713 |
| CDX2 | MMP19 | 0.772 | TMEM106C | NCF4 | 0.746 | FCGR3B | PDLIM4 | 0.817 |
| PREX1 | SPOCK1 | 0.775 | NFAM1 | TNFRSF10C | 0.725 | PCBP3 | PIP4P2 | 0.804 |
| RAI14 | WWC2 | 0.755 | CMTM4 | DOK3 | 0.747 | HLX | LOXL2 | 0.719 |
| DPYSL3 | FGF2 | 0.784 | G0S2 | BMERB1 | 0.844 | ABL2 | LST1 | 0.781 |
| PDLIM4 | SH3BP5 | 0.776 | LGALS1 | CCL2 | 0.752 | C2CD2 | PAPPA | 0.829 |
| ENO2 | MCAM | 0.772 | WNK2 | SSH2 | 0.804 | IER5 | CCDC3 | 0.745 |
| LOXL2 | OSMR | 0.782 | TNFAIP6 | ACOD1 | 0.783 | SERPINE1 | SPHK1 | 0.790 |
| PKIB | CD300A | 0.816 | RGS19 | ACOD1 | 0.705 | PFKL | GNAI2 | 0.795 |
| TSPAN6 | PPP1R18 | 0.781 | FGF2 | STC1 | 0.781 | MMP3 | CLEC4A | 0.786 |
| CLU | CCL2 | 0.773 | LCP1 | MX2 | 0.722 | DLC1 | PTGS2 | 0.807 |
| S100A8 | TCF4 | 0.823 | HSPA6 | SMAP2 | 0.729 | GALNT12 | FAP | 0.791 |
| SHTN1 | HSPA6 | 0.765 | CHI3L1 | TUBA1A | 0.786 | COL7A1 | SWAP70 | 0.743 |
| EMP3 | FPR1 | 0.771 | ITGAM | ZDHHC18 | 0.772 | TRIM2 | SH3BP5 | 0.785 |
| IFITM2 | TMEM273 | 0.781 | POLDIP2 | LRP4 | 0.762 | SPOCK1 | SSH2 | 0.786 |
| TRPM6 | NDFIP2 | 0.764 | GALNT12 | FPR2 | 0.804 | TMEM154 | WWC2 | 0.782 |
| ATP5IF1 | FPR1 | 0.787 | GPR176 | COTL1 | 0.749 | IL1B | TUBB6 | 0.810 |
| CSF3R | PLAU | 0.728 | MYO1D | WWC2 | 0.760 | ZDHHC18 | ITPRIP | 0.787 |
| FAM49A | GFPT2 | 0.756 | FGR | PI15 | 0.772 | AMPD2 | SLC35G2 | 0.809 |
| GPR137B | PDLIM4 | 0.796 | CD300A | SH3BP5 | 0.803 | CSF3R | FPR2 | 0.712 |
| COL15A1 | BMERB1 | 0.774 | KIAA1522 | GLIPR1 | 0.735 | FGF2 | TNFRSF10C | 0.779 |
| DUSP2 | PCBP3 | 0.772 | PIK3AP1 | SERPINE1 | 0.773 | PRR16 | CEP19 | 0.751 |
| FAM155A | GFPT2 | 0.778 | TREM1 | SELE | 0.790 | SH3RF2 | THBD | 0.813 |
| TMEM106C | CTHRC1 | 0.798 | RAB15 | CLU | 0.779 | GOLPH3L | RAB31 | 0.792 |
| CYTH4 | CYP26B1 | 0.807 | KIAA1522 | FLT1 | 0.777 | RAB31 | VIM | 0.762 |
| PPP2R5A | CHN1 | 0.793 | GPR183 | MMP3 | 0.769 | FPR2 | CXCL8 | 0.781 |
| PROK2 | ARHGAP25 | 0.722 | SELENBP1 | S100A12 | 0.799 | TMEM106C | CYTH4 | 0.758 |
| TNFAIP2 | SSH2 | 0.743 | MIR100HG | GPR65 | 0.753 | AKAP1 | CREB5 | 0.819 |
| MCAM | FAM155A | 0.783 | AGFG2 | DUSP2 | 0.779 | CD300A | LRRK2 | 0.798 |
| TMEM154 | BPGM | 0.775 | GLCE | JAZF1 | 0.782 | COQ9 | GPR176 | 0.765 |
| CEBPB | S100A9 | 0.808 | CMTM4 | SLC11A1 | 0.787 | WNK2 | HS3ST3B1 | 0.825 |
| NDFIP2 | PHLDB2 | 0.781 | ACOT9 | RBMS1 | 0.790 | SRGN | PTGS2 | 0.790 |
| GPR183 | COTL1 | 0.769 | FCGR2B | SPHK1 | 0.792 | THBD | CREB5 | 0.783 |
| TRAK1 | FAM20C | 0.766 | OLFM1 | CCN4 | 0.785 | FCER1G | FGF7 | 0.803 |
| CTHRC1 | VIM | 0.771 | NDFIP2 | CHST15 | 0.776 | CDX2 | PHLDB2 | 0.772 |
| AGAP1 | PLAU | 0.766 | PRRX1 | BCAT1 | 0.755 | AMPD2 | HSPA6 | 0.732 |
| CCDC69 | CLEC4E | 0.719 | SNORC | OSM | 0.814 | ABL2 | TNFAIP2 | 0.757 |
| PKIB | CLU | 0.785 | FCGR3B | MX2 | 0.798 | RIPOR3 | SPP1 | 0.772 |
| TMEM273 | ITPRIP | 0.808 | PLAU | PRRX1 | 0.763 | PDE4B | TNFAIP2 | 0.730 |
| EFNA4 | POLDIP2 | 0.773 | CHI3L1 | CD14 | 0.793 | SNORC | SNN | 0.793 |
| NFE2 | SLC11A1 | 0.801 | PDLIM4 | CD86 | 0.780 | CSF3R | S100A9 | 0.742 |
| NFAM1 | MMP3 | 0.771 | CBX6 | ENTPD1 | 0.771 | COL7A1 | PREX1 | 0.808 |
| PCBP3 | SLC11A1 | 0.778 | RGS19 | SLC11A1 | 0.762 | AGFG2 | PTPRC | 0.783 |
| WNK2 | FAM49A | 0.819 | CHI3L2 | CAVIN1 | 0.772 | CLEC4E | S100A12 | 0.779 |
| UBAC1 | LILRB1 | 0.803 | GPR137B | BTG2 | 0.798 | PIK3AP1 | FPR1 | 0.725 |
| CDX2 | SNORC | 0.774 | RAB31 | PREX1 | 0.749 | OLFM1 | RIPK2 | 0.783 |
| AGAP1 | CD86 | 0.783 | CMTM4 | TNFRSF10C | 0.769 | LILRA2 | S100A12 | 0.761 |
| SELENBP1 | SPOCK1 | 0.812 | CSTA | DPYSL3 | 0.731 | AGAP1 | PTPRC | 0.779 |
| TNFRSF10C | CCN4 | 0.765 | IL1RN | FAM49A | 0.811 | FGR | SELP | 0.813 |
| FLT1 | SNN | 0.806 | LGALS1 | SH3BP5 | 0.755 | DUSP4 | RAI14 | 0.799 |
| STAP2 | ENO2 | 0.761 | NDFIP2 | RALGAPA2 | 0.766 | TRPM6 | LILRB1 | 0.795 |
| RAB15 | KIAA1522 | 0.799 | ARPIN | LILRB2 | 0.816 | CREM | AMPD2 | 0.777 |
| PDPN | FAM155A | 0.814 | CDX2 | CD93 | 0.797 | GPR183 | MMP19 | 0.759 |
| TMEM71 | CLEC4E | 0.718 | RILPL2 | MEFV | 0.813 | TMEM154 | PRRX1 | 0.773 |
| SELENBP1 | NFAM1 | 0.775 | TAGAP | ICAM1 | 0.696 | COL4A2 | SPHK1 | 0.768 |
| PI15 | RGS16 | 0.770 | COTL1 | LY96 | 0.788 | GZMB | PRRX1 | 0.762 |
| TNFAIP6 | ZDHHC18 | 0.788 | MSN | TUBB6 | 0.774 | CHI3L1 | KCNJ15 | 0.772 |
| IL24 | FGR | 0.806 | LILRB1 | ADGRG3 | 0.793 | TNC | TNFAIP6 | 0.798 |
| OCRL | GMFG | 0.786 | SELE | STAT5B | 0.799 | KCNJ15 | PLEK | 0.748 |
| SERPINE1 | SERPINB9 | 0.798 | TRPM6 | PLEK | 0.768 | PKIB | RAI14 | 0.802 |
| DLC1 | CD53 | 0.771 | ERMP1 | ENTPD1 | 0.824 | LRRK2 | RGS16 | 0.764 |
| CMTM4 | HS3ST3B1 | 0.804 | KIAA1522 | AMPD2 | 0.783 | CXCL8 | BCL6 | 0.736 |
| TRPM6 | HCK | 0.773 | NRCAM | ACOD1 | 0.796 | CXCR1 | SERPINB9 | 0.733 |
| UBAC1 | LILRB3 | 0.749 | EFNA4 | RAI14 | 0.781 | SELE | PXDN | 0.816 |
| ANGPT2 | PXDN | 0.791 | IL6 | PHLDB2 | 0.825 | LCP1 | BTG2 | 0.728 |
| GAS2L3 | CD53 | 0.748 | FAP | PDLIM4 | 0.758 | LCP2 | MCAM | 0.780 |
| DUSP2 | PTGS2 | 0.776 | IL13RA2 | SELP | 0.871 | MSN | SLC11A1 | 0.789 |
| CSNK1G3 | AGFG2 | 0.758 | LRP4 | HS3ST3B1 | 0.777 | CD300A | TREM1 | 0.750 |
| BASP1 | RIPK2 | 0.794 | PFKL | CXCR2 | 0.823 | S1PR3 | SELP | 0.794 |
| OCRL | SORT1 | 0.779 | CSF3 | BPGM | 0.806 | AMPD2 | FAM155A | 0.800 |
| FGF7 | TLR4 | 0.765 | NCF2 | SELP | 0.799 | UQCRC1 | MAP4K4 | 0.791 |
| EFNA4 | FAP | 0.784 | IL1B | CCL4 | 0.778 | BICD2 | PI15 | 0.764 |
| BASP1 | SPOCK1 | 0.787 | BASP1 | TAGAP | 0.756 | FERMT2 | GZMB | 0.788 |
| ANGPT2 | OSMR | 0.790 | PCBP3 | THEMIS2 | 0.798 | PRRX1 | TNFRSF10C | 0.777 |
| SLC15A4 | SLC35G2 | 0.800 | PAPPA | SERPINE2 | 0.831 | BICD2 | CYTH4 | 0.767 |
| PRICKLE2 | RNF149 | 0.836 | MMP3 | CEP19 | 0.746 | CEMIP | ACOD1 | 0.782 |
| CSNK1G3 | CHST2 | 0.758 | TCEA3 | NIBAN1 | 0.813 | SNORC | CEMIP | 0.813 |
| NDUFA10 | OCRL | 0.799 | FAP | TLR1 | 0.773 | TMEM154 | TREM1 | 0.750 |
| SERPINE2 | GFPT2 | 0.765 | SELENBP1 | VASN | 0.783 | IL1RN | TIMP1 | 0.769 |
| PRICKLE2 | FLT1 | 0.786 | SLC11A1 | SLFN11 | 0.787 | DSP | FAP | 0.772 |
| SNORC | ICAM1 | 0.765 | ASAP1 | THEMIS2 | 0.777 | FGR | LAT2 | 0.789 |
| FCER1G | CYTH4 | 0.777 | TRIM2 | PRRX1 | 0.777 | ITGAM | CXCR4 | 0.688 |
| AGFG2 | C11orf96 | 0.782 | AKAP1 | ENTPD1 | 0.805 | NCF4 | NOD2 | 0.730 |
| SLA | PXDN | 0.751 | TCEA3 | WNT5A | 0.808 | TSPAN6 | TAGAP | 0.792 |
| RALGAPA2 | PDPN | 0.813 | ZNF704 | TREM1 | 0.759 | TRIM2 | CREB5 | 0.802 |
| MEFV | NCF2 | 0.738 | AGFG2 | SLC11A1 | 0.787 | SERPINE2 | WNT5A | 0.811 |
| SERPINE1 | ENTPD1 | 0.794 | CHST15 | STAT5B | 0.763 | IFITM2 | MAP4K4 | 0.780 |
| DSP | NR3C2 | 0.793 | CHRDL2 | MAP4K4 | 0.780 | THBD | PXDN | 0.793 |
| NEO1 | COL7A1 | 0.809 | NR3C2 | PCBP3 | 0.773 | FGR | VNN2 | 0.797 |
| FAP | BICD2 | 0.759 | PPP2R5A | CYTH4 | 0.768 | SH2D3C | CLEC4E | 0.717 |
| RGS19 | CHI3L2 | 0.691 | ZNF704 | CHI3L2 | 0.724 | TCF4 | C5AR1 | 0.776 |
| SELE | SPOCK1 | 0.807 | PROK2 | TNF | 0.718 | ITGAM | CCN4 | 0.771 |
| NDFIP2 | MCAM | 0.772 | CRIP2 | IL1RN | 0.825 | DPYSL2 | RBMS1 | 0.759 |
| ADCY6 | SLA | 0.772 | RAB15 | PTPRC | 0.801 | DLC1 | SLC11A1 | 0.827 |
| RGS19 | IL13RA2 | 0.840 | KIAA1522 | SELENBP1 | 0.807 | DSP | NNMT | 0.805 |
| DUSP4 | SH3BP5 | 0.767 | LILRA2 | LY96 | 0.785 | PFKL | CCL4 | 0.780 |
| AKAP1 | DPYSL3 | 0.785 | TCEA3 | HLX | 0.776 | COQ9 | SSH2 | 0.779 |
| CEP19 | CD14 | 0.730 | CD93 | RIPOR2 | 0.789 | CLU | C5AR1 | 0.799 |
| LCP1 | TUBB6 | 0.756 | FGF7 | WWC2 | 0.780 | CD300A | COL15A1 | 0.770 |
| PFKL | CCL2 | 0.768 | CXCL8 | DOK3 | 0.774 | ZDHHC23 | NFE2 | 0.809 |
| NDUFA10 | CSF3 | 0.799 | LY96 | PTGS2 | 0.794 | TRAK1 | SELE | 0.800 |
| GALNT12 | MMP3 | 0.810 | HIBCH | TLR1 | 0.808 | GALNT12 | CLEC4A | 0.823 |
| AFG3L2 | MCAM | 0.778 | NIPSNAP3A | NEO1 | 0.771 | S100A12 | SMAP2 | 0.780 |
| TMEM71 | SLC15A3 | 0.762 | TMEM71 | ENTPD1 | 0.813 | RASSF5 | BMERB1 | 0.822 |
| HSD11B2 | INHBA | 0.815 | FLT1 | FJX1 | 0.765 | GPR183 | FLT1 | 0.779 |
| RGS16 | S100A12 | 0.791 | ANGPT2 | TNFAIP6 | 0.801 | PRR16 | S100A8 | 0.829 |
| DUSP2 | FGF7 | 0.785 | SH3RF2 | CBX6 | 0.758 | WNK2 | COL4A2 | 0.800 |
| RAB15 | RGCC | 0.803 | CR1 | PROK2 | 0.731 | SH3RF2 | CCR1 | 0.790 |
| DUSP2 | IL1B | 0.785 | RAB31 | PLAU | 0.759 | B4GALT4 | MMP3 | 0.804 |
| VIM | GFPT2 | 0.735 | PDPN | AMPD2 | 0.813 | ICAM1 | SOD2 | 0.741 |
| RAB15 | RIPK2 | 0.793 | PTPRC | SLC11A1 | 0.805 | COL7A1 | CD93 | 0.803 |
| VASN | AMPD2 | 0.763 | TMEM106C | SSH2 | 0.756 | TMEM154 | NCF2 | 0.749 |
| PKIB | TUBB6 | 0.797 | S1PR3 | MAP4K4 | 0.776 | RAB15 | ADGRG3 | 0.769 |
| TRAK1 | HCK | 0.770 | ARPIN | MYO1F | 0.792 | TMEM154 | CCL2 | 0.766 |
| TSPAN6 | ANGPT2 | 0.833 | TSPAN6 | TNC | 0.824 | CR1 | TMEM71 | 0.756 |
| GLIPR1 | ABL2 | 0.716 | SH2D3C | CLEC4A | 0.759 | TNFAIP2 | SPHK1 | 0.759 |
| SNORC | FCGR2B | 0.788 | ERMP1 | HS3ST3B1 | 0.812 | COX10 | ANGPT2 | 0.806 |
| COTL1 | MIR100HG | 0.752 | ADCY6 | FAM20C | 0.777 | CBX6 | CEP19 | 0.673 |
| LGALS1 | CLEC4A | 0.786 | PIK3AP1 | VIM | 0.716 | TLR1 | SH3BP5 | 0.745 |
| ZDHHC23 | BASP1 | 0.804 | FJX1 | HCLS1 | 0.719 | PPP2R5A | TNFAIP2 | 0.751 |
| FFAR2 | CHST15 | 0.773 | ITGAM | PLAU | 0.768 | CEBPB | HLX | 0.735 |
| CREM | CSF3R | 0.731 | PPP2R5A | DLC1 | 0.796 | VASN | GNAI2 | 0.718 |
| CDX2 | ACOT9 | 0.774 | FJX1 | HLX | 0.722 | PPP1R18 | PI15 | 0.767 |
| GOLPH3L | CSTA | 0.752 | CSF2RB | RBMS1 | 0.784 | DSP | LILRB1 | 0.799 |
| GLIPR1 | SH3BP5 | 0.704 | STAP2 | CREM | 0.753 | CCL2 | THEMIS2 | 0.775 |
| SELL | CREB5 | 0.806 | PIP4P2 | ARHGAP25 | 0.742 | INHBA | PTPRC | 0.800 |
| RILPL2 | LST1 | 0.780 | PAPPA | CD69 | 0.819 | HCLS1 | NNMT | 0.740 |
| CHRDL2 | IL13RA2 | 0.861 | ASB7 | CBX6 | 0.733 | LILRB1 | RNF149 | 0.794 |
| FZD5 | COL7A1 | 0.809 | LRP4 | SLC35G2 | 0.816 | ERMP1 | TIMP1 | 0.813 |
| TNC | LOXL2 | 0.782 | SNORC | IL1B | 0.799 | LILRB3 | CD69 | 0.711 |
| PTGS2 | RGS16 | 0.784 | NCF2 | TLR4 | 0.706 | CTHRC1 | SLA | 0.753 |
| B4GALT4 | LILRB1 | 0.790 | PCBP3 | BPGM | 0.789 | CD69 | HS3ST3B1 | 0.757 |
| TCF4 | FAM49A | 0.767 | CMTM8 | BICD2 | 0.758 | CCDC88A | CD14 | 0.734 |
| FLT1 | MX2 | 0.774 | AKAP1 | SLC35G2 | 0.822 | IL11 | LGALS1 | 0.831 |
| RNF149 | CD69 | 0.775 | CTHRC1 | NFAM1 | 0.793 | ANKRD35 | RGS16 | 0.758 |
| CCR1 | SPOCK1 | 0.799 | CYTH4 | SRGN | 0.776 | CXCR2 | BCAT1 | 0.806 |
| FCGR3B | ABL2 | 0.826 | LILRA2 | CSTA | 0.729 | CAVIN3 | WWC2 | 0.755 |
| CHST15 | TNFAIP6 | 0.793 | B4GALT4 | ASAP1 | 0.773 | SMAP2 | CCDC3 | 0.735 |
| PIK3AP1 | RIPOR3 | 0.763 | MYO1D | SHTN1 | 0.787 | STAT5B | CXCR4 | 0.708 |
| GAS2L3 | C2CD2 | 0.789 | ZNF704 | CYTH4 | 0.780 | NDUFA10 | RAB31 | 0.776 |
| CSF3 | SELE | 0.804 | RIPOR3 | GLT1D1 | 0.800 | SLC15A3 | SELE | 0.807 |
| CHRDL2 | TNC | 0.798 | SPOCK1 | ST3GAL5 | 0.767 | EMP3 | SPHK1 | 0.757 |
| PPP2R5A | SWAP70 | 0.752 | MMP19 | VAV1 | 0.748 | EMP3 | ENTPD1 | 0.780 |
| SNORC | SLC2A3 | 0.790 | NRG1 | PTGS2 | 0.793 | RALGAPA2 | CAVIN3 | 0.783 |
| DUSP4 | EMP3 | 0.760 | NRCAM | BCAT1 | 0.772 | FGF7 | SWAP70 | 0.746 |
| UBAC1 | FCGR3B | 0.805 | LDLRAD3 | THEMIS2 | 0.791 | RNF149 | NCF4 | 0.764 |
| LDLRAD3 | IER5 | 0.768 | TMEM154 | IER5 | 0.722 | TIMP1 | WWC2 | 0.736 |
| DPYSL2 | P2RY14 | 0.758 | NFE2 | SNN | 0.813 | TAGAP | NNMT | 0.743 |
| GLIPR1 | LCP2 | 0.701 | NFAM1 | C11orf96 | 0.759 | GPR137B | LAT2 | 0.790 |
| CD300A | TLR4 | 0.726 | BCL6 | P2RY14 | 0.738 | PDPN | DPYSL3 | 0.788 |
| PCBP3 | CD14 | 0.748 | FAM20C | SSH2 | 0.760 | PIK3AP1 | LILRA5 | 0.723 |
| SH2D3C | COL4A2 | 0.756 | GMFG | CD69 | 0.772 | POLDIP2 | GALNT12 | 0.771 |
| HIBCH | CEMIP | 0.787 | FPR2 | P2RY14 | 0.789 | NRG1 | PDE4B | 0.772 |
| CSF3 | ABL2 | 0.802 | VAV1 | OSMR | 0.781 | OSM | SERPINE2 | 0.824 |
| EFNA4 | FCGR2B | 0.791 | LGALS1 | MCAM | 0.758 | F5 | WNT5A | 0.807 |
| CHI3L1 | PTGS2 | 0.788 | SH3RF2 | C2CD2 | 0.798 | TMEM154 | ENO2 | 0.791 |
| GOLPH3L | RFLNB | 0.797 | RNF149 | CCL4 | 0.786 | ACOT9 | OSMR | 0.783 |
| ATP5IF1 | EGR3 | 0.752 | RAI14 | IL6 | 0.803 | LCP1 | CCN4 | 0.751 |
| DPYSL3 | SPHK1 | 0.764 | CHI3L1 | NRG1 | 0.789 | B4GALT4 | LST1 | 0.767 |
| AGAP1 | FILIP1L | 0.789 | TRAK1 | TCF4 | 0.760 | CHN1 | S100A8 | 0.823 |
| GPR183 | SWAP70 | 0.715 | TRPM6 | RIPOR3 | 0.791 | TRPM6 | BASP1 | 0.797 |
| SHTN1 | PIK3AP1 | 0.749 | DLC1 | THEMIS2 | 0.790 | PXDN | GPR65 | 0.779 |
| NRCAM | PTPRC | 0.768 | BTG2 | ST3GAL5 | 0.758 | NFAM1 | FLT1 | 0.741 |
| CSNK1G3 | LRP4 | 0.797 | PFKL | LCP2 | 0.801 | PDPN | SERPINE1 | 0.785 |
| SELP | TIMP1 | 0.796 | LRRK2 | CEMIP | 0.779 | UQCRC1 | FERMT2 | 0.808 |
| RGS19 | RAB31 | 0.725 | PRICKLE2 | TLR4 | 0.738 | HIBCH | THBD | 0.811 |
| PLA1A | C5AR1 | 0.777 | HSD11B2 | DUSP2 | 0.795 | SPP1 | P2RY14 | 0.757 |
| DPYSL3 | DUSP4 | 0.804 | PRR16 | CLMP | 0.788 | TMEM273 | TCF4 | 0.745 |
| ACOD1 | DOK3 | 0.736 | GLCE | PRRX1 | 0.773 | BDH1 | CD300A | 0.821 |
| GZMB | NOD2 | 0.758 | NRG1 | LCP1 | 0.762 | MEFV | ARHGAP25 | 0.724 |
| PKIB | THEMIS2 | 0.810 | TMEM71 | IL6 | 0.813 | SERPINE1 | CD14 | 0.779 |
| ITGA5 | VIM | 0.783 | ACOT9 | LCP2 | 0.787 | UQCRC1 | CHI3L1 | 0.792 |
| ADCY6 | ABL2 | 0.798 | ARPIN | PDLIM4 | 0.776 | JAZF1 | G0S2 | 0.826 |
| NIPSNAP3A | SELP | 0.807 | STAT5B | TIMP1 | 0.765 | IFITM2 | IL1RN | 0.765 |
| LRP4 | G0S2 | 0.820 | CTHRC1 | S100A8 | 0.809 | P2RY13 | RBMS1 | 0.750 |
| DUSP2 | SRGN | 0.774 | DOK3 | CD69 | 0.714 | BASP1 | SLC11A1 | 0.796 |
| HIBCH | GADD45B | 0.787 | CSNK1G3 | G0S2 | 0.814 | THBD | RIPK2 | 0.798 |
| AGFG2 | RNF149 | 0.775 | FZD5 | CXCR2 | 0.821 | PAPPA | TNFRSF10C | 0.815 |
| CAVIN1 | CREB5 | 0.755 | ARPIN | PDPN | 0.782 | SH3RF2 | PREX1 | 0.773 |
| FGR | RASSF5 | 0.766 | ZDHHC23 | LILRA2 | 0.798 | RILPL2 | NNMT | 0.800 |
| CCDC88A | ENTPD1 | 0.795 | HCLS1 | PLAU | 0.721 | GNAI2 | RFLNB | 0.751 |
| CD93 | LST1 | 0.792 | EGR3 | BCL2A1 | 0.751 | TSPAN6 | CLMP | 0.790 |
| CDX2 | SH2D3C | 0.780 | FGF2 | SLC15A3 | 0.761 | DUSP4 | LST1 | 0.792 |
| GLIPR1 | TNFRSF1B | 0.681 | ERMP1 | PRICKLE2 | 0.838 | WNK2 | ENTPD1 | 0.821 |
| RGCC | CD86 | 0.794 | RAB31 | CREM | 0.759 | AGFG2 | GPR65 | 0.784 |
| S100A9 | FAM155A | 0.839 | BMERB1 | CD69 | 0.793 | G0S2 | THBD | 0.824 |
| RGS16 | ST3GAL5 | 0.756 | ZDHHC23 | ZDHHC18 | 0.802 | BICD2 | CHST2 | 0.770 |
| GLIPR1 | HCLS1 | 0.675 | CSNK1G3 | HS3ST3B1 | 0.775 | RAB31 | FAM20C | 0.768 |
| PIP4P2 | TNF | 0.810 | TNFAIP2 | MAP4K4 | 0.747 | VASN | CD14 | 0.723 |
| CDX2 | HCLS1 | 0.762 | BPGM | PDLIM4 | 0.776 | FILIP1L | IL11 | 0.834 |
| PIK3AP1 | F5 | 0.705 | RAB15 | RGS16 | 0.787 | GLCE | SH2D3C | 0.766 |
| LILRA5 | FAM155A | 0.791 | IL13RA2 | PI15 | 0.835 | IL13RA2 | TUBA1A | 0.864 |
| FAP | ITGA5 | 0.759 | TNC | LAT2 | 0.803 | FZD5 | CLEC4A | 0.802 |
| IL24 | TMEM154 | 0.811 | RAB15 | NCF2 | 0.794 | LST1 | GMFG | 0.777 |
| WNK2 | GFPT2 | 0.790 | PPP2R5A | BICD2 | 0.800 | MX2 | PCBP3 | 0.783 |
| BCAT1 | HS3ST3B1 | 0.757 | ZNF704 | LST1 | 0.767 | CSF3R | CXCL8 | 0.722 |
| CMTM8 | RALGAPA2 | 0.766 | IL11 | NCF4 | 0.834 | MX2 | ST3GAL5 | 0.785 |
| GPR183 | ZDHHC18 | 0.769 | PFKL | STAT5B | 0.758 | SELENBP1 | FAP | 0.790 |
| FFAR2 | CCDC88A | 0.739 | GLT1D1 | P2RY13 | 0.726 | CMTM8 | AMPD2 | 0.781 |
| FCGR2B | RBMS1 | 0.791 | ASB7 | PKIB | 0.753 | WWC2 | GFPT2 | 0.735 |
| FAP | SNN | 0.766 | BDH1 | C11orf96 | 0.785 | SRGN | CEP19 | 0.735 |
| SMAP2 | ZDHHC18 | 0.745 | ADGRG3 | FPR2 | 0.755 | CSNK1G3 | SPOCK1 | 0.789 |
| TNC | ITPRIP | 0.802 | AFG3L2 | FZD5 | 0.756 | FCGR3B | MMP3 | 0.812 |
| GZMB | RASSF5 | 0.772 | COQ9 | CREB5 | 0.794 | ASAP1 | TNF | 0.784 |
| FCER1G | ITGAM | 0.753 | PROK2 | SMAP2 | 0.746 | C11orf96 | LOXL2 | 0.792 |
| SRGN | CCDC88A | 0.798 | TMEM106C | G0S2 | 0.812 | CHN1 | IER5 | 0.763 |
| ZNF704 | SELE | 0.808 | GALNT12 | DUSP2 | 0.789 | PKIB | FILIP1L | 0.775 |
| GALNT12 | DLC1 | 0.789 | TRPM6 | PPP1R18 | 0.778 | CGAS | WNT5A | 0.790 |
| OLFM1 | ANKRD35 | 0.798 | FAM20C | SLFN11 | 0.728 | ERMP1 | SLC2A3 | 0.768 |
| ADCY6 | PXDN | 0.786 | G0S2 | CHST15 | 0.815 | MCAM | MSN | 0.758 |
| SNORC | CSF3 | 0.817 | LCP2 | CYP26B1 | 0.812 | MEFV | PROK2 | 0.723 |
| VASN | RIPOR3 | 0.764 | FCGR2B | MMP3 | 0.782 | COQ9 | SLC2A3 | 0.770 |
| TLR1 | PHLDB2 | 0.786 | CHRDL2 | PROK2 | 0.764 | EMP3 | PLA1A | 0.778 |
| FLT1 | ABL2 | 0.757 | NIBAN1 | CCDC69 | 0.753 | ANGPT2 | TNC | 0.807 |
| COX10 | EMP3 | 0.807 | VIM | ARHGAP25 | 0.757 | LOXL2 | ENTPD1 | 0.782 |
| C5AR1 | ENTPD1 | 0.799 | VASN | AQP9 | 0.787 | FCGR3B | FLT1 | 0.814 |
| CAVIN3 | CHST15 | 0.779 | PRICKLE2 | S100A9 | 0.837 | TMEM273 | STC1 | 0.821 |
| LY96 | PIP4P2 | 0.801 | GALNT12 | PLAU | 0.788 | ANGPT2 | SPP1 | 0.762 |
| TAGAP | SLC11A1 | 0.756 | TAGAP | CLMP | 0.734 | NEO1 | LY96 | 0.817 |
| NIPSNAP3A | EGR3 | 0.779 | C2CD2 | IL6 | 0.805 | RGCC | CHST11 | 0.758 |
| HSPA6 | SLC2A3 | 0.709 | CREM | TNFRSF1B | 0.739 | NIBAN1 | TMEM273 | 0.783 |
| RIPK2 | GMFG | 0.783 | PPP2R5A | STC1 | 0.815 | GPR183 | S100A9 | 0.822 |
| RHOH | NFE2 | 0.726 | ANKRD35 | IL6 | 0.829 | IL24 | TNFAIP2 | 0.791 |
| CRIP2 | LST1 | 0.766 | FLT1 | LY96 | 0.779 | CBX6 | RHOH | 0.695 |
| LILRA2 | HLX | 0.714 | WNK2 | CBX6 | 0.799 | TUBB6 | RIPOR2 | 0.781 |
| F5 | LAT2 | 0.798 | VASN | CLU | 0.737 | HLX | SLC16A6 | 0.717 |
| EFNA4 | FAM155A | 0.790 | OSM | SLA | 0.774 | CSNK1G3 | TCF4 | 0.759 |
| SH2D3C | PREX1 | 0.713 | COL7A1 | SLFN11 | 0.750 | NR3C2 | SMAP2 | 0.769 |
| CHI3L2 | S1PR1 | 0.748 | GZMB | TNC | 0.804 | BDH1 | LILRB2 | 0.822 |
| FAM20C | TCF4 | 0.765 | ACOT9 | LOXL2 | 0.785 | DSP | RHOH | 0.766 |
| FGR | CCN4 | 0.776 | BICD2 | NOD2 | 0.687 | RILPL2 | MCAM | 0.792 |
| CRIP2 | CCL2 | 0.759 | NIBAN1 | TREM1 | 0.736 | TMEM154 | WNT5A | 0.818 |
| TMEM273 | MYO1F | 0.751 | UQCRC1 | MSN | 0.801 | ASAP1 | RIPOR2 | 0.759 |
| CLU | RIPOR2 | 0.757 | FJX1 | RHOH | 0.741 | SORT1 | CD53 | 0.742 |
| NEO1 | ATP5IF1 | 0.790 | IL6 | PTPRC | 0.804 | CDX2 | WNT5A | 0.818 |
| P2RY13 | SMAP2 | 0.773 | FGR | PHLDB2 | 0.787 | BDH1 | IL24 | 0.803 |
| TRIM2 | CCDC3 | 0.776 | CLEC4E | CXCL5 | 0.758 | BDH1 | RFLNB | 0.793 |
| CEBPB | PAPPA | 0.827 | CSF3R | ENO2 | 0.755 | ZNF704 | PCBP3 | 0.769 |
| CSF3R | KCNJ15 | 0.673 | FAP | TNFRSF1B | 0.742 | FERMT2 | LILRA2 | 0.765 |
| PIK3AP1 | LST1 | 0.709 | MYO1D | NDUFA10 | 0.816 | GALNT12 | CLU | 0.772 |
| CHI3L1 | NNMT | 0.793 | OLFM1 | ARHGAP25 | 0.764 | GZMB | CCDC3 | 0.788 |
| TCF4 | BMERB1 | 0.779 | NDFIP2 | ICAM1 | 0.762 | CSF3R | ANKRD35 | 0.745 |
| AGAP1 | GALNT12 | 0.775 | S1PR1 | FLT1 | 0.766 | TSPAN6 | LILRB3 | 0.766 |
| IL24 | MIR100HG | 0.795 | CSF2RB | PTPRC | 0.759 | JAZF1 | SPP1 | 0.755 |
| MMP19 | CREB5 | 0.778 | BICD2 | MYO1F | 0.730 | RALGAPA2 | CLEC4E | 0.778 |
| COL4A2 | CD93 | 0.765 | TMEM71 | MAP4K4 | 0.753 | DPYSL3 | LCP1 | 0.737 |
| MMP19 | GPR65 | 0.767 | RAB31 | FGF2 | 0.764 | ITPRIP | CD69 | 0.798 |
| CYP26B1 | SH3BP5 | 0.806 | LILRB1 | IER5 | 0.762 | LRP4 | RBMS1 | 0.796 |
| GNAI2 | HCK | 0.742 | GOLPH3L | PIP4P2 | 0.802 | ADCY6 | CTHRC1 | 0.798 |
| RGCC | LGALS1 | 0.779 | PIP4P2 | CCN4 | 0.782 | LDLRAD3 | FLT1 | 0.773 |
| FAM20C | HS3ST3B1 | 0.784 | EMP3 | PROK2 | 0.750 | KIAA1522 | FGR | 0.796 |
| CHI3L2 | COL7A1 | 0.778 | LCP2 | RASSF5 | 0.718 | RGS19 | GADD45B | 0.722 |
| LY96 | AQP9 | 0.791 | F5 | CD14 | 0.767 | NOD2 | LAT2 | 0.734 |
| SELENBP1 | PLAU | 0.795 | TMEM154 | NCF4 | 0.771 | MX2 | PLAU | 0.773 |
| ZDHHC23 | SELE | 0.810 | CLU | SPP1 | 0.756 | GLIPR1 | BPGM | 0.737 |
| PIK3AP1 | THEMIS2 | 0.708 | COL7A1 | CREB5 | 0.805 | SHTN1 | TMEM71 | 0.807 |
| DSP | RIPOR2 | 0.776 | FZD5 | FLT1 | 0.783 | LILRB1 | VIM | 0.778 |
| NOD2 | TUBA1A | 0.709 | TCEA3 | BICD2 | 0.780 | CYTH4 | FAM49A | 0.779 |
| STAT5B | TCF4 | 0.741 | LILRA2 | ENTPD1 | 0.794 | STAP2 | CSF2RB | 0.790 |
| SNORC | S100A9 | 0.838 | ERMP1 | SELL | 0.828 | PXDN | CHST2 | 0.789 |
| COL4A2 | RIPOR2 | 0.765 | CMTM4 | HLX | 0.759 | CLEC4A | PXDN | 0.801 |
| ADGRG3 | TNF | 0.762 | PI15 | TNF | 0.780 | NFE2 | S100A8 | 0.800 |
| KIAA1522 | GNAI2 | 0.753 | UQCRC1 | PIK3AP1 | 0.741 | SELL | ITPRIP | 0.806 |
| EFNA4 | PRICKLE2 | 0.796 | VAV1 | HS3ST3B1 | 0.764 | PIK3AP1 | LOXL2 | 0.749 |
| FAM20C | TUBA1A | 0.764 | RHOH | ZDHHC18 | 0.745 | ADGRG3 | SELP | 0.775 |
| FCGR3B | ARHGAP25 | 0.806 | ABL2 | NRCAM | 0.765 | TRAK1 | PI15 | 0.766 |
| C11orf96 | VNN2 | 0.809 | CBX6 | RASSF5 | 0.719 | FAP | VNN2 | 0.792 |
| CDX2 | SERPINB9 | 0.767 | RALGAPA2 | RAI14 | 0.795 | LILRB3 | DPYSL2 | 0.743 |
| P2RY13 | SELL | 0.786 | IFITM2 | HSPA6 | 0.742 | VASN | FAM49A | 0.749 |
| ANGPT2 | CHST11 | 0.769 | FAP | CCN4 | 0.761 | RAB31 | BCL6 | 0.700 |
| SERPINE1 | CHST2 | 0.798 | EFNA4 | SELE | 0.800 | DUSP2 | S100A9 | 0.814 |
| SHTN1 | EGR3 | 0.771 | TCF4 | ZDHHC18 | 0.752 | PKIB | FAM49A | 0.816 |
| FLT1 | COTL1 | 0.778 | CHN1 | SELP | 0.801 | CYP26B1 | CD86 | 0.805 |
| CHI3L1 | SERPINB9 | 0.803 | RGS19 | P2RY14 | 0.749 | MX2 | PTGS1 | 0.803 |
| NFAM1 | S100A9 | 0.781 | C11orf96 | SLC11A1 | 0.812 | COX10 | MSN | 0.789 |
| SH2D3C | DOK3 | 0.728 | FPR1 | PTPRC | 0.750 | SNORC | TNC | 0.806 |
| S100A8 | STAT5B | 0.806 | CHI3L2 | RAI14 | 0.756 | AMPD2 | TNC | 0.803 |
| ENO2 | FGF2 | 0.779 | ZNF704 | HCLS1 | 0.723 | HCK | SLC16A6 | 0.744 |
| CHST15 | SERPINB9 | 0.766 | SHTN1 | SERPINB9 | 0.780 | FGR | IER5 | 0.762 |
| BCL6 | S100A9 | 0.741 | GNAI2 | ITPRIP | 0.807 | TCF4 | HS3ST3B1 | 0.766 |
| HSD11B2 | CCR1 | 0.818 | FGF2 | CHRDL2 | 0.795 | SELENBP1 | MMP19 | 0.792 |
| TRAK1 | COTL1 | 0.776 | FAP | RBMS1 | 0.767 | CEBPB | CAVIN1 | 0.769 |
| GPR176 | HSPA6 | 0.740 | CXCR1 | TREM1 | 0.714 | NFAM1 | ANGPT2 | 0.772 |
| ANGPTL2 | ABL2 | 0.762 | FILIP1L | S100A12 | 0.814 | PIP4P2 | ST3GAL5 | 0.778 |
| BPGM | TCF4 | 0.764 | LDLRAD3 | OSMR | 0.782 | NCF2 | SLC35G2 | 0.776 |
| PCBP3 | FAM155A | 0.800 | AKAP1 | FAM20C | 0.785 | FGR | IL1B | 0.788 |
| PIK3AP1 | AQP9 | 0.761 | SELL | TCF4 | 0.775 | CHRDL2 | HS3ST3B1 | 0.794 |
| GAS2L3 | NCF4 | 0.757 | NIPSNAP3A | CTHRC1 | 0.820 | OLFM1 | NCF2 | 0.769 |
| MMP19 | SH3BP5 | 0.768 | AGFG2 | DPYSL2 | 0.760 | S1PR1 | AMPD2 | 0.780 |
| LILRB3 | BTG2 | 0.737 | AGAP1 | PHLDB2 | 0.784 | LST1 | TNFRSF10C | 0.765 |
| TRIM2 | CXCR4 | 0.734 | CCR1 | GADD45B | 0.791 | FLT1 | S100A8 | 0.807 |
| NR3C2 | TUBB6 | 0.794 | NDUFA10 | IL1RN | 0.781 | CLEC4E | RBMS1 | 0.728 |
| CSNK1G3 | IL13RA2 | 0.871 | C5AR1 | WWC2 | 0.763 | LILRA2 | CXCR1 | 0.690 |
| NIPSNAP3A | SLC15A4 | 0.792 | RHOH | MSN | 0.748 | SMAP2 | CHST2 | 0.766 |
| IER5 | TUBB6 | 0.744 | GZMB | IL1B | 0.785 | LY96 | ANGPT2 | 0.801 |
| CXCR2 | CEMIP | 0.797 | BCL6 | CD86 | 0.708 | ITGAM | PRRX1 | 0.766 |
| PPP2R5A | EMP3 | 0.801 | SERPINB9 | NOD2 | 0.738 | AFG3L2 | FERMT2 | 0.781 |
| ZDHHC23 | GADD45B | 0.802 | GAS2L3 | RIPK2 | 0.793 | CMTM8 | C11orf96 | 0.775 |
| VASN | CCL2 | 0.724 | NNMT | CHST15 | 0.796 | SH2D3C | MX2 | 0.730 |
| BASP1 | GLIPR1 | 0.752 | NRG1 | BPGM | 0.811 | SORT1 | IL11 | 0.833 |
| DLC1 | CHST11 | 0.789 | GZMB | PXDN | 0.795 | G0S2 | WWC2 | 0.804 |
| CSF2RB | TNC | 0.793 | NDUFA10 | ICAM1 | 0.758 | ANGPT2 | CREB5 | 0.783 |
| ANGPT2 | RFLNB | 0.799 | LILRA2 | CSF3R | 0.697 | ARPIN | NEO1 | 0.776 |
| LILRB2 | C1R | 0.782 | CTHRC1 | FPR1 | 0.789 | COTL1 | BTG2 | 0.765 |
| ASB7 | TMEM106C | 0.763 | NEO1 | PRRX1 | 0.794 | BASP1 | FGR | 0.786 |
| COL7A1 | P2RY14 | 0.788 | MCAM | NOD2 | 0.727 | BDH1 | CD93 | 0.802 |
| ANGPTL2 | FFAR2 | 0.777 | ACOT9 | SPHK1 | 0.802 | SH2D3C | PDPN | 0.784 |
| CXCR2 | GPR137B | 0.808 | FZD5 | RHOH | 0.763 | EMP3 | PTPRC | 0.755 |
| UBAC1 | HCK | 0.761 | IFITM2 | CREB5 | 0.789 | PCBP3 | TREM1 | 0.777 |
| IL24 | CXCR4 | 0.780 | CBX6 | FJX1 | 0.740 | TNF | ARHGAP25 | 0.790 |
| CSNK1G3 | KIAA1522 | 0.781 | AFG3L2 | BCAT1 | 0.763 | CXCL8 | ITGA5 | 0.782 |
| LILRB2 | SPHK1 | 0.806 | KIAA1522 | HCLS1 | 0.735 | CLU | HS3ST3B1 | 0.784 |
| FLT1 | FAM20C | 0.769 | ERMP1 | F5 | 0.787 | HIBCH | HS3ST3B1 | 0.797 |
| RALGAPA2 | SELP | 0.795 | FCGR2B | NCF2 | 0.791 | FILIP1L | FGR | 0.796 |
| TUBA1A | GMFG | 0.771 | MYO1D | SH2D3C | 0.778 | MYO1D | IL13RA2 | 0.860 |
| SELE | SSH2 | 0.800 | CDX2 | RAB15 | 0.794 | ATP5IF1 | THBD | 0.798 |
| AGAP1 | PDLIM4 | 0.777 | LILRB3 | TREM1 | 0.711 | LILRA5 | IL1B | 0.770 |
| CSF3 | TUBA1A | 0.789 | PKIB | G0S2 | 0.826 | CXCL5 | PDLIM4 | 0.785 |
| PDPN | TLR1 | 0.802 | C11orf96 | CCDC88A | 0.789 | BASP1 | ACOD1 | 0.786 |
| NCF4 | TLR1 | 0.776 | ARPIN | HCLS1 | 0.735 | CR1 | PRRX1 | 0.746 |
| FAM20C | PDLIM4 | 0.775 | AFG3L2 | PLEK | 0.749 | PREX1 | CLMP | 0.772 |
| COL4A2 | GADD45B | 0.765 | CYTH4 | ST3GAL5 | 0.754 | CREM | SPOCK1 | 0.770 |
| FGF7 | PI15 | 0.788 | LILRB3 | TNFAIP2 | 0.712 | BCAT1 | CCL4 | 0.766 |
| MYO1D | RASSF5 | 0.784 | PLA1A | CXCL5 | 0.768 | AGAP1 | ITGAM | 0.779 |
| F5 | CEP19 | 0.702 | NIPSNAP3A | RAB15 | 0.761 | CD93 | CD69 | 0.783 |
| COX10 | DUSP2 | 0.790 | NDFIP2 | CAVIN1 | 0.758 | TSPAN6 | RIPK2 | 0.812 |
| VASN | S100A12 | 0.760 | ZDHHC23 | MYO1F | 0.808 | ASB7 | WWC2 | 0.763 |
| GAS2L3 | BMERB1 | 0.810 | WNK2 | NRCAM | 0.790 | DPYSL2 | FCGR3B | 0.821 |
| PDPN | NFE2 | 0.796 | BASP1 | MCAM | 0.803 | PRICKLE2 | MCAM | 0.784 |
| AQP9 | MEFV | 0.787 | CLMP | P2RY14 | 0.776 | IER5 | GMFG | 0.764 |
| DUSP2 | FCGR3B | 0.813 | CHI3L1 | ACOD1 | 0.792 | SERPINE1 | HS3ST3B1 | 0.801 |
| CHI3L2 | PROK2 | 0.758 | SH2D3C | HCK | 0.745 | SH3RF2 | FERMT2 | 0.785 |
| ZNF704 | IL1B | 0.784 | GADD45B | TIMP1 | 0.774 | LRP4 | CSF3 | 0.806 |
| FGR | THEMIS2 | 0.781 | MMP3 | VIM | 0.777 | VNN2 | MAP4K4 | 0.771 |
| CXCL5 | RIPK2 | 0.769 | SERPINE2 | CCL2 | 0.775 | AGAP1 | FPR2 | 0.776 |
| FILIP1L | FAM20C | 0.761 | PROK2 | CREB5 | 0.715 | TMEM273 | RIPOR2 | 0.748 |
| FCER1G | LAT2 | 0.767 | TREM1 | NOD2 | 0.728 | ICAM1 | TNFRSF10C | 0.742 |
| GLIPR1 | WWC2 | 0.719 | SH2D3C | CHST15 | 0.768 | STAP2 | SORT1 | 0.742 |
| CDX2 | CTHRC1 | 0.798 | LGALS1 | PAPPA | 0.832 | GPR183 | IL1B | 0.788 |
| CEBPB | SERPINE1 | 0.794 | RALGAPA2 | HSPA6 | 0.764 | CHST11 | CXCL5 | 0.759 |
| UQCRC1 | STAT5B | 0.777 | EFNA4 | SOD2 | 0.775 | ITPRIP | SLFN11 | 0.803 |
| LILRA5 | SELP | 0.805 | MYO1F | TNFAIP2 | 0.741 | DLC1 | ANKRD35 | 0.791 |
| GPR176 | S100A12 | 0.800 | SMAP2 | BMERB1 | 0.778 | LILRA5 | RBMS1 | 0.773 |
| NNMT | P2RY14 | 0.791 | COQ9 | ITPRIP | 0.807 | S1PR3 | NFE2 | 0.785 |
| IL24 | COL4A2 | 0.786 | CHN1 | TLR4 | 0.725 | C5AR1 | CHST2 | 0.783 |
| RASSF5 | P2RY14 | 0.777 | F5 | KCNJ15 | 0.715 | CSF2RB | SSH2 | 0.769 |
| SLC11A1 | TUBB6 | 0.792 | NR3C2 | OCRL | 0.779 | CSNK1G3 | PI15 | 0.767 |
| CSTA | BPGM | 0.766 | FCGR3B | INHBA | 0.822 | CLEC4E | CCL4 | 0.738 |
| STAT5B | CREB5 | 0.768 | EFNA4 | PFKL | 0.760 | ADCY6 | CMTM8 | 0.795 |
| PRICKLE2 | PTPRC | 0.807 | GAS2L3 | RHOH | 0.762 | DPYSL2 | LGALS1 | 0.765 |
| SELL | ENTPD1 | 0.803 | AFG3L2 | BASP1 | 0.773 | EGR3 | ANGPT2 | 0.751 |
| HSD11B2 | COL4A2 | 0.796 | MYO1D | LILRB2 | 0.824 | TCEA3 | BASP1 | 0.799 |
| HSD11B2 | CCDC69 | 0.778 | SPP1 | CXCR4 | 0.727 | SLC15A4 | GLT1D1 | 0.788 |
| CCDC69 | CXCR2 | 0.762 | SH2D3C | C11orf96 | 0.775 | ENO2 | LY96 | 0.785 |
| NDFIP2 | C1R | 0.774 | FCER1G | CXCR4 | 0.735 | FFAR2 | IL6 | 0.814 |
| CAVIN1 | BTG2 | 0.752 | WNK2 | VIM | 0.800 | VAV1 | MAP4K4 | 0.725 |
| ANGPT2 | SNN | 0.820 | ANGPT2 | SLC2A3 | 0.764 | TAGAP | SMAP2 | 0.736 |
| ASB7 | CCN4 | 0.793 | NDFIP2 | AMPD2 | 0.771 | LILRB1 | RAB31 | 0.790 |
| CRIP2 | THEMIS2 | 0.773 | LRP4 | WNK2 | 0.811 | COL7A1 | F5 | 0.816 |
| NIPSNAP3A | SLC15A3 | 0.789 | CGAS | BTG2 | 0.750 | TMEM106C | FGF2 | 0.770 |
| WNT5A | SPHK1 | 0.798 | ARPIN | FCER1G | 0.765 | PRICKLE2 | PDLIM4 | 0.795 |
| ERMP1 | TLR1 | 0.811 | MCAM | SRGN | 0.788 | ATP5IF1 | TLR1 | 0.799 |
| CCR1 | MX2 | 0.791 | AGFG2 | WNK2 | 0.815 | AQP9 | MSN | 0.783 |
| SELENBP1 | AQP9 | 0.793 | GZMB | SPP1 | 0.762 | CSF2RB | IL1B | 0.792 |
| INHBA | RBMS1 | 0.799 | COL7A1 | CCL4 | 0.800 | CREM | LDLRAD3 | 0.787 |
| NCF2 | PCBP3 | 0.772 | NIBAN1 | PXDN | 0.778 | COL7A1 | CHST2 | 0.796 |
| FCGR2B | PROK2 | 0.752 | PCBP3 | MAP4K4 | 0.779 | AGFG2 | ZNF704 | 0.777 |
| SHTN1 | CXCL8 | 0.791 | BASP1 | FJX1 | 0.769 | PAPPA | SLC15A3 | 0.826 |
| CTHRC1 | S100A12 | 0.802 | SELE | CCN4 | 0.807 | GZMB | SELL | 0.804 |
| VASN | ST3GAL5 | 0.741 | STAP2 | IL1B | 0.784 | FAM49A | GMFG | 0.792 |
| B4GALT4 | CD86 | 0.779 | COQ9 | WNT5A | 0.814 | ANKRD35 | SLC11A1 | 0.817 |
| VNN2 | GMFG | 0.817 | FERMT2 | DPYSL2 | 0.763 | ANGPTL2 | PLA1A | 0.777 |
| FZD5 | LRRK2 | 0.801 | PPP2R5A | PRRX1 | 0.782 | SLC15A3 | S100A12 | 0.781 |
| TSPAN6 | SELENBP1 | 0.768 | SH2D3C | TNF | 0.794 | CD300A | CLMP | 0.790 |
| FILIP1L | CCDC69 | 0.759 | ACOD1 | MAP4K4 | 0.750 | FILIP1L | CLMP | 0.766 |
| UQCRC1 | RGCC | 0.809 | AQP9 | STC1 | 0.804 | PPP1R18 | PTGS1 | 0.772 |
| NDUFA10 | LDLRAD3 | 0.805 | SH3RF2 | CSF2RB | 0.807 | ANGPTL2 | PLEK | 0.772 |
| CCL2 | SPOCK1 | 0.780 | RFLNB | S100A8 | 0.812 | FGF2 | CXCR4 | 0.759 |
| TMEM273 | TIMP1 | 0.783 | SELENBP1 | FFAR2 | 0.792 | GLT1D1 | GPR65 | 0.723 |
| IER5 | PTGS1 | 0.765 | NIBAN1 | JAZF1 | 0.779 | FCGR2B | MYO1F | 0.785 |
| SWAP70 | PTGS2 | 0.739 | LDLRAD3 | PRR16 | 0.797 | SORT1 | RILPL2 | 0.796 |
| LILRB1 | CXCR2 | 0.822 | COL4A2 | PRRX1 | 0.781 | CREM | SH3BP5 | 0.761 |
| SLFN11 | HS3ST3B1 | 0.775 | TUBB6 | ENTPD1 | 0.790 | LRRK2 | PXDN | 0.794 |
| FGF7 | STAT5B | 0.784 | TNF | RIPK2 | 0.793 | CBX6 | LCP2 | 0.724 |
| DUSP4 | MSN | 0.760 | NDUFA10 | DPYSL2 | 0.760 | LRP4 | CSTA | 0.781 |
| INHBA | PREX1 | 0.792 | DSP | HLX | 0.744 | NIBAN1 | C2CD2 | 0.780 |
| FFAR2 | NRCAM | 0.778 | HLX | CCL4 | 0.712 | DUSP2 | PROK2 | 0.751 |
| PLAU | GPR137B | 0.773 | RFLNB | LAT2 | 0.772 | HSD11B2 | GPR176 | 0.764 |
| FGR | CYP26B1 | 0.803 | TNFRSF1B | GFPT2 | 0.723 | SHTN1 | FFAR2 | 0.787 |
| PREX1 | VNN2 | 0.785 | CDX2 | AKAP1 | 0.785 | LRRK2 | SERPINE1 | 0.802 |
| PDPN | LILRB1 | 0.800 | PKIB | MYO1F | 0.787 | SH3RF2 | FAP | 0.769 |
| SLA | CREB5 | 0.754 | CHST11 | C5AR1 | 0.741 | MSN | PCBP3 | 0.756 |
| AKAP1 | WNT5A | 0.822 | GPR176 | TLR1 | 0.781 | NIPSNAP3A | TNF | 0.801 |
| MYO1F | GPR65 | 0.730 | TMEM273 | SLFN11 | 0.722 | GPR183 | FCER1G | 0.762 |
| VIM | PXDN | 0.759 | HIBCH | IL13RA2 | 0.851 | TMEM106C | PRR16 | 0.799 |
| DSP | COQ9 | 0.784 | GPR176 | STAT5B | 0.742 | FAP | TNFAIP6 | 0.764 |
| SHTN1 | THBD | 0.791 | OSM | VAV1 | 0.798 | TRAK1 | SELL | 0.803 |
| TNC | CLMP | 0.785 | CHI3L1 | NCF4 | 0.795 | SNN | ZDHHC18 | 0.778 |
| STAP2 | WWC2 | 0.742 | GLIPR1 | BICD2 | 0.701 | PIK3AP1 | FAM49A | 0.702 |
| SLC15A3 | ITPRIP | 0.802 | CXCR2 | SERPINB9 | 0.811 | PPP1R18 | MAP4K4 | 0.767 |
| HCLS1 | TNF | 0.733 | C11orf96 | TNFAIP2 | 0.783 | GLT1D1 | LCP2 | 0.730 |
| HCLS1 | THEMIS2 | 0.696 | DSP | SWAP70 | 0.726 | FCER1G | LCP2 | 0.757 |
| JAZF1 | ANGPTL2 | 0.761 | NDFIP2 | COTL1 | 0.766 | NFE2 | PROK2 | 0.720 |
| SERPINE1 | P2RY14 | 0.800 | COQ9 | BICD2 | 0.784 | JAZF1 | AQP9 | 0.803 |
| TRIM2 | GPR176 | 0.755 | SWAP70 | RIPOR2 | 0.688 | COTL1 | GADD45B | 0.772 |
| IL11 | CLMP | 0.819 | UBAC1 | KIAA1522 | 0.779 | AFG3L2 | CAVIN1 | 0.775 |
| FCER1G | BCL2A1 | 0.761 | RIPOR3 | SELL | 0.803 | NFE2 | BCAT1 | 0.764 |
| SLC15A4 | PI15 | 0.781 | CSTA | GPR183 | 0.721 | RILPL2 | FAM49A | 0.810 |
| AGAP1 | CD53 | 0.744 | EFNA4 | LST1 | 0.788 | CBX6 | C1R | 0.755 |
| TNC | SRGN | 0.792 | TSPAN6 | RGS16 | 0.797 | MEFV | SERPINB9 | 0.765 |
| LOXL2 | SERPINE2 | 0.795 | ADGRG3 | C5AR1 | 0.743 | COL4A2 | FJX1 | 0.759 |
| GLIPR1 | NRCAM | 0.726 | ABL2 | P2RY13 | 0.770 | ASB7 | SNN | 0.807 |
| SPOCK1 | FAM49A | 0.789 | NIBAN1 | S100A8 | 0.820 | DLC1 | CLMP | 0.781 |
| PFKL | RFLNB | 0.792 | LOXL2 | CXCR4 | 0.741 | C2CD2 | ARHGAP25 | 0.774 |
| FGF7 | SLC15A3 | 0.799 | TNF | CEP19 | 0.735 | DPYSL3 | ITGA5 | 0.765 |
| ATP5IF1 | F5 | 0.778 | COL4A2 | LOXL2 | 0.762 | LGALS1 | BMERB1 | 0.816 |
| CXCR1 | ITGA5 | 0.768 | CHI3L1 | SWAP70 | 0.749 | CSF3 | TLR4 | 0.749 |
| TMEM273 | TREM1 | 0.768 | IL6 | S100A12 | 0.812 | LRP4 | LRRK2 | 0.807 |
| ATP5IF1 | PLA1A | 0.774 | SH3RF2 | FCER1G | 0.777 | TIMP1 | TNFRSF1B | 0.751 |
| FILIP1L | SLC15A3 | 0.759 | WNK2 | CD86 | 0.800 | GOLPH3L | FAM155A | 0.799 |
| NDFIP2 | NOD2 | 0.751 | THBD | ARHGAP25 | 0.792 | ARPIN | CHST15 | 0.768 |
| IL6 | ACOD1 | 0.799 | CTHRC1 | TCF4 | 0.781 | NR3C2 | PTPRC | 0.793 |
| GPR183 | PREX1 | 0.742 | CHN1 | JAZF1 | 0.769 | COL4A2 | SLC11A1 | 0.787 |
| ARPIN | PTGS1 | 0.785 | WNK2 | ITGA5 | 0.810 | HIBCH | FCER1G | 0.781 |
| FAM155A | SLFN11 | 0.789 | CSF3 | RILPL2 | 0.804 | SNORC | MSN | 0.785 |
| TMEM273 | CYP26B1 | 0.815 | AMPD2 | CHST11 | 0.757 | HIBCH | BMERB1 | 0.790 |
| SLC16A6 | CHST2 | 0.756 | EFNA4 | CLEC4E | 0.763 | LILRB3 | TIMP1 | 0.734 |
| GNAI2 | CHST11 | 0.711 | MX2 | CHST15 | 0.792 | EFNA4 | HSD11B2 | 0.753 |
| LILRA5 | MMP3 | 0.786 | NR3C2 | LAT2 | 0.798 | COX10 | ZDHHC23 | 0.800 |
| CTHRC1 | CHST15 | 0.806 | FZD5 | PIK3AP1 | 0.743 | CGAS | EGR3 | 0.733 |
| SPHK1 | CD14 | 0.751 | LILRA5 | SERPINB9 | 0.781 | TRAK1 | TMEM106C | 0.777 |
| S1PR3 | JAZF1 | 0.760 | CDX2 | NFE2 | 0.772 | FERMT2 | CCN4 | 0.783 |
| JAZF1 | TNC | 0.790 | CCR1 | RGS16 | 0.781 | FJX1 | SOD2 | 0.770 |
| STAP2 | P2RY14 | 0.764 | KIAA1522 | TNC | 0.795 | LCP2 | TIMP1 | 0.776 |
| COTL1 | BCL2A1 | 0.762 | NRG1 | SH3BP5 | 0.794 | CGAS | CD14 | 0.732 |
| LILRB1 | LGALS1 | 0.792 | GPR137B | CCN4 | 0.788 | PPP2R5A | PRICKLE2 | 0.813 |
| SELE | CD53 | 0.788 | COQ9 | HCLS1 | 0.739 | TMEM106C | CEMIP | 0.786 |
| BMERB1 | HS3ST3B1 | 0.845 | CHI3L2 | ITGA5 | 0.740 | DSP | OSMR | 0.790 |
| PFKL | FILIP1L | 0.787 | DSP | RAB31 | 0.786 | CEBPB | HSPA6 | 0.739 |
| DLC1 | DUSP2 | 0.779 | STC1 | FAM155A | 0.822 | ATP5IF1 | PROK2 | 0.754 |
| LILRA2 | SPOCK1 | 0.780 | NEO1 | STAP2 | 0.780 | CCR1 | CCDC88A | 0.793 |
| MMP3 | BCL2A1 | 0.777 | NIPSNAP3A | FAP | 0.790 | CSNK1G3 | CBX6 | 0.764 |
| LILRB2 | PDPN | 0.802 | GAS2L3 | HLX | 0.753 | HLX | IL6 | 0.761 |
| B4GALT4 | COL4A2 | 0.778 | RILPL2 | ARHGAP25 | 0.795 | IL6 | CHST11 | 0.791 |
| TREM1 | C5AR1 | 0.725 | CHI3L1 | CREB5 | 0.784 | SLC2A3 | VAV1 | 0.722 |
| RGCC | TUBA1A | 0.786 | SORT1 | SNN | 0.803 | TSPAN6 | MMP3 | 0.820 |
| ADGRG3 | SLFN11 | 0.750 | PXDN | GFPT2 | 0.761 | MMP19 | PIP4P2 | 0.788 |
| COL15A1 | ADGRG3 | 0.757 | PIP4P2 | CEMIP | 0.791 | LRRK2 | MMP19 | 0.782 |
| RAB15 | SH2D3C | 0.781 | PRRX1 | SLFN11 | 0.733 | FERMT2 | ADGRG3 | 0.789 |
| RAB15 | LDLRAD3 | 0.823 | OLFM1 | FAM49A | 0.776 | LILRA2 | NRG1 | 0.779 |
| JAZF1 | PROK2 | 0.742 | CLMP | VNN2 | 0.811 | SPOCK1 | TLR1 | 0.793 |
| TAGAP | LOXL2 | 0.734 | DLC1 | LAT2 | 0.799 | GZMB | OSMR | 0.786 |
| TRIM2 | LGALS1 | 0.779 | NNMT | LST1 | 0.776 | IFITM2 | NIBAN1 | 0.798 |
| IL1B | WWC2 | 0.790 | THBD | HS3ST3B1 | 0.812 | SH3RF2 | IL24 | 0.807 |
| AGFG2 | OSMR | 0.772 | TRIM2 | LILRB2 | 0.826 | S1PR3 | ANGPTL2 | 0.749 |
| CSF3 | S100A9 | 0.816 | WNT5A | RIPOR2 | 0.800 | HCK | ACOD1 | 0.770 |
| HSD11B2 | RFLNB | 0.779 | RIPOR3 | S1PR3 | 0.785 | CSF2RB | RHOH | 0.723 |
| PFKL | FZD5 | 0.773 | SNORC | LST1 | 0.788 | UQCRC1 | IER5 | 0.760 |
| CHI3L1 | CHST2 | 0.788 | LILRB2 | HLX | 0.728 | NRG1 | CD53 | 0.778 |
| TAGAP | RAI14 | 0.720 | CHN1 | PROK2 | 0.745 | ADGRG3 | G0S2 | 0.778 |
| B4GALT4 | MIR100HG | 0.756 | BICD2 | CLMP | 0.771 | PKIB | SNN | 0.786 |
| LILRB2 | LILRB1 | 0.808 | S100A9 | VAV1 | 0.824 | CLEC4A | PI15 | 0.771 |
| CHN1 | CREB5 | 0.788 | TMEM71 | DUSP4 | 0.783 | LILRB1 | BPGM | 0.804 |
| AGFG2 | TSPAN6 | 0.759 | PPP2R5A | PHLDB2 | 0.774 | ANKRD35 | MEFV | 0.789 |
| GNAI2 | CYP26B1 | 0.800 | KIAA1522 | S1PR1 | 0.775 | GALNT12 | CCL4 | 0.792 |
| NCF4 | CXCR4 | 0.705 | ACOT9 | FAM49A | 0.797 | CAVIN3 | CCL2 | 0.776 |
| GALNT12 | TNF | 0.802 | VASN | IL11 | 0.806 | COX10 | F5 | 0.815 |
| TSPAN6 | NRG1 | 0.812 | CHRDL2 | SLC16A6 | 0.792 | SORT1 | GFPT2 | 0.755 |
| NDUFA10 | ENTPD1 | 0.801 | MSN | GPR65 | 0.781 | ACOD1 | GPR65 | 0.736 |
| IL6 | MCAM | 0.820 | FGR | RIPOR2 | 0.769 | PPP1R18 | NNMT | 0.776 |
| CSNK1G3 | CCL4 | 0.780 | CD93 | NFE2 | 0.808 | COL7A1 | TNC | 0.809 |
| TIMP1 | GMFG | 0.777 | CBX6 | S100A12 | 0.798 | PDE4B | BCAT1 | 0.728 |
| NCF4 | WWC2 | 0.769 | EMP3 | WNT5A | 0.782 | MMP3 | CHST15 | 0.795 |
| TMEM154 | MAP4K4 | 0.742 | CLU | BCAT1 | 0.755 | FGF2 | P2RY14 | 0.769 |
| COL4A2 | CXCR4 | 0.733 | LILRB2 | LILRA5 | 0.763 | CCDC3 | CHST2 | 0.771 |
| RGCC | TNFAIP2 | 0.772 | CXCR1 | FAM155A | 0.773 | PREX1 | CD69 | 0.702 |
| CCL2 | PXDN | 0.767 | JAZF1 | PLA1A | 0.777 | PTGS2 | RIPOR2 | 0.760 |
| NEO1 | WNT5A | 0.829 | COX10 | FGF7 | 0.791 | CEBPB | LST1 | 0.781 |
| CHN1 | COL15A1 | 0.769 | ADGRG3 | PDE4B | 0.728 | CTHRC1 | PXDN | 0.791 |
| GPR183 | JAZF1 | 0.754 | NIPSNAP3A | PLA1A | 0.787 | LILRB1 | SPP1 | 0.781 |
| BDH1 | MCAM | 0.783 | FAM20C | RIPOR2 | 0.766 | DPYSL3 | RASSF5 | 0.759 |
| CAVIN3 | NOD2 | 0.750 | STAT5B | TNFRSF1B | 0.729 | CSTA | ENTPD1 | 0.749 |
| SNORC | DLC1 | 0.780 | CRIP2 | CHRDL2 | 0.772 | FAM20C | S100A12 | 0.789 |
| MEFV | CLMP | 0.775 | LILRA2 | CHI3L1 | 0.779 | RALGAPA2 | NRG1 | 0.828 |
| CAVIN1 | WWC2 | 0.744 | ITGAM | NCF4 | 0.762 | COL4A2 | STAT5B | 0.760 |
| CHN1 | RFLNB | 0.776 | CSNK1G3 | PTGS1 | 0.791 | CMTM8 | DPYSL2 | 0.786 |
| CTHRC1 | RIPOR3 | 0.795 | CD300A | CLEC4A | 0.795 | SERPINE1 | SSH2 | 0.809 |
| PFKL | MIR100HG | 0.761 | CXCR1 | SPP1 | 0.741 | TMEM273 | NRG1 | 0.814 |
| CD300A | MMP3 | 0.796 | SH3RF2 | CD300A | 0.801 | DSP | CSF2RB | 0.803 |
| COL4A2 | MEFV | 0.766 | KIAA1522 | CREB5 | 0.788 | SLC11A1 | FAM155A | 0.825 |
| CDX2 | BMERB1 | 0.799 | COTL1 | ANGPT2 | 0.805 | CD93 | GNAI2 | 0.782 |
| SERPINE1 | C5AR1 | 0.794 | FJX1 | SSH2 | 0.765 | GPR176 | ENTPD1 | 0.769 |
| SPOCK1 | PXDN | 0.790 | RGCC | SERPINB9 | 0.795 | C11orf96 | CXCR4 | 0.757 |
| S1PR1 | BCL2A1 | 0.761 | CDX2 | BCL2A1 | 0.779 | PCBP3 | CHST2 | 0.773 |
| AGFG2 | MCAM | 0.775 | DPYSL2 | PTGS1 | 0.788 | CSNK1G3 | CR1 | 0.741 |
| NOD2 | WWC2 | 0.721 | CSF2RB | FLT1 | 0.782 | HCK | MAP4K4 | 0.764 |
| LILRB2 | RNF149 | 0.799 | MIR100HG | SMAP2 | 0.713 | CCL2 | SLC35G2 | 0.773 |
| BICD2 | MMP3 | 0.784 | SERPINE1 | ASAP1 | 0.798 | THBD | ST3GAL5 | 0.796 |
| DSP | CHRDL2 | 0.774 | PPP2R5A | TMEM71 | 0.831 | GAS2L3 | BCL6 | 0.733 |
| CXCL8 | NNMT | 0.795 | S100A12 | TUBB6 | 0.800 | RHOH | SLC11A1 | 0.776 |
| FILIP1L | RFLNB | 0.788 | CRIP2 | SELE | 0.802 | COL4A2 | TUBA1A | 0.750 |
| CD300A | CCL4 | 0.780 | S1PR3 | PDE4B | 0.754 | F5 | CCL4 | 0.760 |
| FPR1 | S100A9 | 0.773 | TRPM6 | SOD2 | 0.786 | S1PR3 | HCK | 0.758 |
| FAP | PIP4P2 | 0.758 | LRP4 | LILRB1 | 0.787 | PI15 | PDLIM4 | 0.778 |
| GLT1D1 | SERPINE2 | 0.775 | RAB15 | HCK | 0.760 | CAVIN3 | FAM49A | 0.772 |
| PRR16 | RBMS1 | 0.806 | TCF4 | BTG2 | 0.744 | SERPINE1 | C1R | 0.783 |
| PIK3AP1 | FPR2 | 0.715 | PPP1R18 | CCDC69 | 0.747 | B4GALT4 | SERPINE1 | 0.805 |
| HIBCH | CR1 | 0.758 | PRICKLE2 | BPGM | 0.792 | PFKL | COTL1 | 0.804 |
| LILRB1 | ITGA5 | 0.788 | NDUFA10 | KIAA1522 | 0.786 | FCER1G | ITGA5 | 0.770 |
| ACOT9 | PLA1A | 0.779 | ARPIN | CEMIP | 0.782 | NFAM1 | SERPINB9 | 0.742 |
| RAB31 | CR1 | 0.744 | CYP26B1 | CLMP | 0.806 | COQ9 | FAM20C | 0.778 |
| LDLRAD3 | PIP4P2 | 0.791 | RAB15 | ITPRIP | 0.816 | IL13RA2 | OSMR | 0.862 |
| TMEM106C | IFITM2 | 0.777 | COQ9 | JAZF1 | 0.755 | NDFIP2 | SLC11A1 | 0.801 |
| C2CD2 | BPGM | 0.750 | RIPOR3 | RASSF5 | 0.790 | POLDIP2 | CD300A | 0.807 |
| LDLRAD3 | RILPL2 | 0.809 | AFG3L2 | AQP9 | 0.794 | KIAA1522 | LY96 | 0.786 |
| PAPPA | SLC11A1 | 0.842 | CXCL5 | ZDHHC18 | 0.759 | SHTN1 | MSN | 0.777 |
| CDX2 | GLT1D1 | 0.762 | IL11 | MCAM | 0.834 | CSNK1G3 | MIR100HG | 0.727 |
| SORT1 | TLR4 | 0.719 | TRIM2 | TNFRSF1B | 0.753 | GOLPH3L | CYTH4 | 0.755 |
| AMPD2 | IL13RA2 | 0.888 | VIM | CD14 | 0.754 | LILRA2 | CHI3L2 | 0.759 |
| FILIP1L | SLA | 0.751 | TLR4 | WWC2 | 0.701 | S1PR3 | TLR1 | 0.783 |
| SHTN1 | TIMP1 | 0.762 | CXCR4 | PHLDB2 | 0.724 | ATP5IF1 | LCP1 | 0.756 |
| TREM1 | GPR65 | 0.736 | ATP5IF1 | SRGN | 0.802 | CSNK1G3 | LILRA5 | 0.795 |
| STAP2 | CD53 | 0.739 | NDUFA10 | IL24 | 0.820 | AGFG2 | HSPA6 | 0.743 |
| CXCR2 | G0S2 | 0.816 | FGF7 | PLAU | 0.800 | DSP | CLEC4A | 0.797 |
| CEBPB | C1R | 0.776 | SELENBP1 | PREX1 | 0.791 | NR3C2 | EGR3 | 0.794 |
| SERPINE2 | STC1 | 0.812 | CLU | COL7A1 | 0.793 | CHI3L2 | FAP | 0.747 |
| FFAR2 | MIR100HG | 0.785 | SH2D3C | GNAI2 | 0.720 | COL4A2 | NRG1 | 0.801 |
| BASP1 | PTGS1 | 0.797 | COQ9 | TLR1 | 0.814 | CBX6 | CLMP | 0.759 |
| CHI3L1 | CHST11 | 0.780 | PPP2R5A | TMEM273 | 0.794 | BCAT1 | VAV1 | 0.746 |
| FAP | SMAP2 | 0.735 | DPYSL2 | CXCR2 | 0.805 | FERMT2 | HLX | 0.731 |
| RAB31 | FGR | 0.771 | PLEK | BTG2 | 0.769 | OCRL | LILRB1 | 0.793 |
| ASAP1 | CHST2 | 0.774 | ITGA5 | BCL6 | 0.729 | FGR | GFPT2 | 0.752 |
| AMPD2 | NNMT | 0.799 | BCAT1 | WWC2 | 0.755 | GLT1D1 | RFLNB | 0.759 |
| CSNK1G3 | IL1B | 0.797 | CEBPB | COL7A1 | 0.792 | WNK2 | CD93 | 0.819 |
| FFAR2 | SOD2 | 0.759 | SLA | TUBA1A | 0.735 | AGAP1 | BASP1 | 0.786 |
| PIK3AP1 | GMFG | 0.740 | GAS2L3 | LILRB3 | 0.738 | PPP1R18 | LGALS1 | 0.764 |
| CD93 | FAM20C | 0.793 | NRG1 | RBMS1 | 0.801 | FZD5 | SERPINE1 | 0.818 |
| COX10 | ITPRIP | 0.805 | COX10 | STC1 | 0.817 | CEP19 | ST3GAL5 | 0.724 |
| OLFM1 | IL24 | 0.799 | IL13RA2 | TNFRSF10C | 0.854 | GZMB | HCK | 0.772 |
| AFG3L2 | TCF4 | 0.757 | LST1 | CHST2 | 0.765 | ASAP1 | TNFRSF10C | 0.755 |
| ZNF704 | HS3ST3B1 | 0.783 | GAS2L3 | NFAM1 | 0.743 | UBAC1 | TAGAP | 0.754 |
| PRR16 | BPGM | 0.810 | GPR176 | BTG2 | 0.745 | TSPAN6 | FGF7 | 0.813 |
| RASSF5 | PDLIM4 | 0.784 | RALGAPA2 | FLT1 | 0.803 | CDX2 | GAS2L3 | 0.795 |
| DSP | HSPA6 | 0.768 | FILIP1L | DUSP2 | 0.771 | ASB7 | LILRB2 | 0.773 |
| SERPINE2 | PXDN | 0.783 | FERMT2 | CHST15 | 0.779 | F5 | SLC16A6 | 0.748 |
| MEFV | ST3GAL5 | 0.788 | LILRB2 | LCP2 | 0.758 | LCP1 | TNFRSF1B | 0.709 |
| MCAM | ST3GAL5 | 0.768 | AGAP1 | FAM20C | 0.782 | ARPIN | F5 | 0.801 |
| TRIM2 | CCDC88A | 0.797 | PI15 | FAM49A | 0.772 | FCGR2B | NRCAM | 0.776 |
| GZMB | TUBA1A | 0.776 | AKAP1 | TNFRSF1B | 0.785 | B4GALT4 | TMEM71 | 0.816 |
| FCGR3B | IL1B | 0.801 | FCGR2B | SSH2 | 0.791 | B4GALT4 | SELL | 0.804 |
| FCGR3B | ITGAM | 0.819 | POLDIP2 | IL1B | 0.779 | RAI14 | CHST2 | 0.776 |
| SNORC | LGALS1 | 0.800 | AGAP1 | MMP3 | 0.788 | CLEC4A | SPP1 | 0.764 |
| SMAP2 | STAT5B | 0.729 | NCF2 | S100A12 | 0.767 | AKAP1 | PIP4P2 | 0.814 |
| PCBP3 | BCL6 | 0.695 | RAB15 | AKAP1 | 0.803 | NIPSNAP3A | SNORC | 0.782 |
| ASAP1 | P2RY13 | 0.779 | NEO1 | S100A12 | 0.795 | HIBCH | ACOD1 | 0.793 |
| VASN | MYO1F | 0.740 | CDX2 | B4GALT4 | 0.764 | PREX1 | CCL2 | 0.755 |
| DPYSL3 | THBD | 0.784 | PREX1 | SLC2A3 | 0.723 | NRG1 | BCL6 | 0.718 |
| WNK2 | RNF149 | 0.811 | CMTM4 | TSPAN6 | 0.777 | S100A9 | SOD2 | 0.801 |
| HSPA6 | SLC35G2 | 0.787 | LILRB3 | BCL2A1 | 0.724 | SORT1 | PIK3AP1 | 0.723 |
| CCDC88A | ZDHHC18 | 0.780 | CHST11 | TNFAIP2 | 0.736 | ADCY6 | CHST2 | 0.774 |
| TSPAN6 | SH2D3C | 0.795 | TRIM2 | DUSP2 | 0.760 | S1PR3 | TNFAIP2 | 0.750 |
| OLFM1 | PI15 | 0.772 | SPP1 | RIPOR2 | 0.749 | RIPOR3 | CLEC4E | 0.774 |
| ASB7 | TREM1 | 0.750 | PPP1R18 | SELP | 0.790 | CSTA | C11orf96 | 0.752 |
| TNFRSF1B | WNT5A | 0.766 | AKAP1 | NRCAM | 0.768 | DLC1 | ZDHHC18 | 0.811 |
| GOLPH3L | RASSF5 | 0.779 | FGF2 | ITGA5 | 0.765 | AGFG2 | RGS19 | 0.743 |
| CLU | MMP19 | 0.765 | CHI3L1 | TMEM273 | 0.793 | LRP4 | IL11 | 0.825 |
| CSNK1G3 | PAPPA | 0.830 | DOK3 | RIPK2 | 0.752 | HCK | MSN | 0.752 |
| ENO2 | MIR100HG | 0.751 | GALNT12 | SLA | 0.772 | CSTA | F5 | 0.737 |
| COQ9 | NRCAM | 0.763 | CD300A | HLX | 0.735 | RNF149 | SPHK1 | 0.785 |
| FZD5 | RILPL2 | 0.776 | CYP26B1 | WNT5A | 0.836 | CAVIN3 | VNN2 | 0.810 |
| TSPAN6 | GALNT12 | 0.764 | IL1RN | NFE2 | 0.788 | RAI14 | DOK3 | 0.741 |
| ICAM1 | PLEK | 0.744 | OLFM1 | ITGA5 | 0.777 | ANKRD35 | ABL2 | 0.785 |
| PDPN | RFLNB | 0.790 | PKIB | SH3BP5 | 0.777 | CSF3 | CLEC4A | 0.809 |
| CBX6 | GPR137B | 0.794 | ZDHHC18 | SLC16A6 | 0.767 | DSP | NCF2 | 0.793 |
| PIP4P2 | PDLIM4 | 0.789 | BASP1 | SERPINE1 | 0.805 | TMEM273 | SPP1 | 0.763 |
| NDUFA10 | ASAP1 | 0.807 | PKIB | KCNJ15 | 0.790 | CHI3L1 | GLT1D1 | 0.792 |
| PFKL | THEMIS2 | 0.800 | CGAS | MIR100HG | 0.764 | CCDC69 | IL6 | 0.811 |
| CEBPB | TAGAP | 0.740 | CYTH4 | SLC15A3 | 0.759 | PFKL | AMPD2 | 0.786 |
| INHBA | LCP2 | 0.792 | ZDHHC18 | CD69 | 0.767 | ZDHHC23 | NEO1 | 0.793 |
| IER5 | SPOCK1 | 0.757 | SELL | TLR4 | 0.753 | BASP1 | BCL2A1 | 0.792 |
| FLT1 | PDE4B | 0.736 | CDX2 | IL13RA2 | 0.857 | TCEA3 | COTL1 | 0.760 |
| TRPM6 | SLC35G2 | 0.806 | CTHRC1 | ZDHHC18 | 0.778 | ATP5IF1 | SMAP2 | 0.760 |
| ANGPT2 | SMAP2 | 0.786 | CD300A | LOXL2 | 0.792 | HIBCH | TSPAN6 | 0.786 |
| CD300A | SELE | 0.813 | ATP5IF1 | CCDC88A | 0.792 | COX10 | PRRX1 | 0.777 |
| IER5 | PDE4B | 0.710 | COL7A1 | SSH2 | 0.811 | DUSP4 | TNFRSF10C | 0.766 |
| COL4A2 | P2RY14 | 0.753 | CSNK1G3 | CHI3L2 | 0.726 | FPR2 | C1R | 0.793 |
| CEBPB | CLEC4A | 0.799 | GALNT12 | C5AR1 | 0.815 | SELENBP1 | C2CD2 | 0.788 |
| AGAP1 | FGF7 | 0.794 | AQP9 | C5AR1 | 0.760 | PDPN | PTPRC | 0.790 |
| COL15A1 | CEP19 | 0.698 | SLC16A6 | CD69 | 0.719 | SPOCK1 | THBD | 0.787 |
| TRIM2 | FLT1 | 0.793 | SERPINE1 | FAM155A | 0.813 | SH2D3C | PROK2 | 0.721 |
| MX2 | THBD | 0.794 | MMP3 | TIMP1 | 0.773 | PPP1R18 | P2RY14 | 0.775 |
| SORT1 | GALNT12 | 0.774 | FAM155A | ZDHHC18 | 0.803 | PPP2R5A | IER5 | 0.787 |
| IL13RA2 | CREB5 | 0.862 | FPR2 | ANGPT2 | 0.807 | MSN | ZDHHC18 | 0.757 |
| C11orf96 | C1R | 0.772 | FAP | SOD2 | 0.766 | TRPM6 | AMPD2 | 0.781 |
| PFKL | KIAA1522 | 0.779 | PI15 | SLC15A3 | 0.769 | CR1 | ACOT9 | 0.780 |
| GOLPH3L | SSH2 | 0.771 | ANGPTL2 | SNN | 0.793 | GLT1D1 | VNN2 | 0.777 |
| NCF2 | GPR137B | 0.780 | TNC | VAV1 | 0.781 | NFE2 | GPR65 | 0.741 |
| HSD11B2 | ATP5IF1 | 0.752 | GLT1D1 | PRICKLE2 | 0.808 | SNORC | PAPPA | 0.831 |
| PLA1A | CCDC3 | 0.770 | SLC16A6 | THEMIS2 | 0.730 | CGAS | TUBB6 | 0.765 |
| COQ9 | PDE4B | 0.789 | NRG1 | CXCL5 | 0.804 | COL15A1 | FPR2 | 0.784 |
| RALGAPA2 | OLFM1 | 0.777 | RALGAPA2 | HCK | 0.768 | CREM | DUSP4 | 0.788 |
| F5 | FGF7 | 0.804 | DLC1 | GZMB | 0.788 | SPP1 | RASSF5 | 0.749 |
| RIPK2 | CHST2 | 0.780 | PKIB | SHTN1 | 0.794 | SNORC | CBX6 | 0.771 |
| LILRA2 | ACOT9 | 0.780 | MMP19 | NFE2 | 0.783 | S1PR3 | BCL2A1 | 0.778 |
| POLDIP2 | ANKRD35 | 0.774 | SPOCK1 | C5AR1 | 0.793 | DPYSL3 | CD53 | 0.731 |
| FERMT2 | RIPOR3 | 0.791 | DUSP4 | GMFG | 0.797 | AGAP1 | CREM | 0.786 |
| PIK3AP1 | FCGR3B | 0.785 | GAS2L3 | F5 | 0.797 | SLC11A1 | SPOCK1 | 0.811 |
| FILIP1L | CREM | 0.774 | CMTM4 | CEP19 | 0.756 | NRG1 | MMP19 | 0.785 |
| VASN | ABL2 | 0.730 | S1PR3 | CXCR1 | 0.765 | CMTM8 | CHST15 | 0.781 |
| GLIPR1 | CCR1 | 0.732 | INHBA | GFPT2 | 0.778 | C11orf96 | CHST11 | 0.770 |
| MX2 | ASAP1 | 0.768 | SMAP2 | SLC35G2 | 0.795 | PDPN | CSF2RB | 0.789 |
| PPP1R18 | CREB5 | 0.777 | G0S2 | PREX1 | 0.811 | PRRX1 | SH3BP5 | 0.768 |
| DPYSL2 | GADD45B | 0.758 | COTL1 | TREM1 | 0.771 | PLA1A | S100A8 | 0.822 |
| DUSP2 | CXCL5 | 0.776 | LY96 | ACOD1 | 0.796 | GPR176 | TNFRSF10C | 0.777 |
| GLT1D1 | PLA1A | 0.776 | GADD45B | SERPINB9 | 0.773 | ICAM1 | ARHGAP25 | 0.745 |
| CLEC4A | PLA1A | 0.785 | NDFIP2 | EGR3 | 0.780 | VASN | MAP4K4 | 0.743 |
| DLC1 | FGF2 | 0.798 | ACOT9 | GPR65 | 0.784 | TNF | CHST2 | 0.785 |
| LRRK2 | TMEM71 | 0.761 | COL4A2 | TLR1 | 0.778 | VASN | C2CD2 | 0.726 |
| JAZF1 | INHBA | 0.799 | HIBCH | GPR65 | 0.784 | TSPAN6 | COL4A2 | 0.799 |
| PIP4P2 | SPHK1 | 0.792 | PPP1R18 | GMFG | 0.766 | ADGRG3 | INHBA | 0.776 |
| CLMP | PHLDB2 | 0.767 | COL7A1 | TLR1 | 0.821 | ASB7 | SNORC | 0.768 |
| RAI14 | SH3BP5 | 0.748 | SNORC | LCP1 | 0.757 | FCER1G | SH3BP5 | 0.759 |
| SELL | STC1 | 0.812 | SH3RF2 | FAM49A | 0.802 | MSN | ENTPD1 | 0.773 |
| AGFG2 | HLX | 0.762 | IL11 | ACOD1 | 0.807 | CDX2 | TCEA3 | 0.770 |
| POLDIP2 | PROK2 | 0.737 | PRRX1 | BTG2 | 0.762 | CTHRC1 | CCL2 | 0.776 |
| NCF2 | SELE | 0.787 | TRPM6 | HSPA6 | 0.757 | NR3C2 | MEFV | 0.787 |
| DUSP2 | IL11 | 0.810 | ASAP1 | TLR1 | 0.788 | TMEM273 | PXDN | 0.799 |
| NR3C2 | OLFM1 | 0.802 | FGR | CD14 | 0.749 | PAPPA | PTPRC | 0.840 |
| SH3BP5 | HS3ST3B1 | 0.781 | SRGN | SPP1 | 0.757 | PLAU | CD53 | 0.741 |
| TRAK1 | PIP4P2 | 0.795 | TRPM6 | CHRDL2 | 0.809 | BTG2 | ARHGAP25 | 0.758 |
| TRIM2 | CD69 | 0.782 | P2RY13 | RIPOR2 | 0.726 | ANGPTL2 | HCLS1 | 0.712 |
| NR3C2 | PFKL | 0.795 | ADGRG3 | KCNJ15 | 0.739 | ATP5IF1 | CR1 | 0.748 |
| LOXL2 | SSH2 | 0.783 | RIPOR3 | ICAM1 | 0.786 | LILRA5 | RFLNB | 0.775 |
| CSNK1G3 | VNN2 | 0.843 | ACOT9 | GADD45B | 0.786 | CCDC69 | SPHK1 | 0.779 |
| FZD5 | DOK3 | 0.744 | NDFIP2 | IL11 | 0.818 | MYO1D | RIPK2 | 0.779 |
| MMP19 | MX2 | 0.781 | GOLPH3L | CRIP2 | 0.767 | TUBA1A | MAP4K4 | 0.759 |
| CAVIN1 | SLC15A3 | 0.735 | FPR1 | LY96 | 0.799 | NDUFA10 | SH3BP5 | 0.798 |
| LRRK2 | CD69 | 0.729 | AMPD2 | TIMP1 | 0.798 | HLX | CD14 | 0.711 |
| GPR137B | GFPT2 | 0.767 | CHRDL2 | CHST2 | 0.774 | NFE2 | SPHK1 | 0.786 |
| TNC | INHBA | 0.796 | DSP | CGAS | 0.781 | ASB7 | PPP1R18 | 0.758 |
| COQ9 | INHBA | 0.800 | SLC15A4 | PPP1R18 | 0.781 | EFNA4 | VASN | 0.770 |
| CMTM4 | PDE4B | 0.775 | HIBCH | PLEK | 0.771 | LILRB3 | PPP1R18 | 0.745 |
| TRIM2 | G0S2 | 0.815 | LRP4 | LDLRAD3 | 0.799 | HIBCH | SLC16A6 | 0.783 |
| ARPIN | TCF4 | 0.749 | NRG1 | CXCR2 | 0.810 | FJX1 | LAT2 | 0.755 |
| DUSP2 | SNN | 0.768 | ANGPTL2 | PI15 | 0.767 | CHI3L2 | SLC15A4 | 0.732 |
| GLCE | PROK2 | 0.748 | HCK | PDLIM4 | 0.773 | ADCY6 | ICAM1 | 0.755 |
| TRPM6 | NFAM1 | 0.756 | DLC1 | NCF4 | 0.797 | BICD2 | C5AR1 | 0.750 |
| LRP4 | SLC15A4 | 0.793 | CSTA | SERPINB9 | 0.749 | TSPAN6 | CCN4 | 0.794 |
| CD93 | SLC11A1 | 0.796 | PRR16 | CEMIP | 0.804 | PLA1A | VAV1 | 0.751 |
| CSNK1G3 | GMFG | 0.780 | CR1 | FPR2 | 0.756 | SORT1 | NFAM1 | 0.761 |
| CCDC69 | ICAM1 | 0.757 | SERPINE2 | CHST2 | 0.801 | IL24 | NOD2 | 0.766 |
| FFAR2 | PDLIM4 | 0.788 | HLX | LILRA5 | 0.750 | PREX1 | BCAT1 | 0.748 |
| ASB7 | TMEM154 | 0.762 | LRP4 | CR1 | 0.780 | CD300A | CXCR2 | 0.815 |
| SMAP2 | SLC2A3 | 0.750 | AQP9 | LST1 | 0.775 | FGF7 | OSM | 0.821 |
| TUBA1A | GPR65 | 0.763 | ZDHHC23 | CR1 | 0.785 | LILRA5 | ST3GAL5 | 0.805 |
| NDUFA10 | VAV1 | 0.776 | CYTH4 | TNFAIP6 | 0.796 | COL7A1 | PLEK | 0.784 |
| TMEM154 | PTGS1 | 0.796 | TREM1 | SLC35G2 | 0.779 | CHN1 | GPR183 | 0.781 |
| DPYSL3 | ST3GAL5 | 0.759 | CD300A | NRCAM | 0.777 | LILRB1 | MAP4K4 | 0.794 |
| LRRK2 | HLX | 0.721 | ANKRD35 | CLEC4A | 0.807 | LDLRAD3 | CEMIP | 0.789 |
| CYTH4 | TIMP1 | 0.767 | CR1 | CCL2 | 0.753 | CLU | PTGS1 | 0.781 |
| NEO1 | MX2 | 0.777 | CSF2RB | CREB5 | 0.774 | LILRB3 | CD14 | 0.735 |
| NR3C2 | ADGRG3 | 0.762 | RIPOR3 | GFPT2 | 0.774 | RNF149 | PROK2 | 0.728 |
| CMTM8 | SOD2 | 0.765 | UQCRC1 | PRRX1 | 0.778 | ADCY6 | ASB7 | 0.780 |
| CD300A | OSM | 0.828 | LRRK2 | SPP1 | 0.765 | UQCRC1 | CHST11 | 0.772 |
| CDX2 | STAP2 | 0.744 | SORT1 | C1R | 0.764 | IFITM2 | ITGAM | 0.781 |
| GAS2L3 | LRRK2 | 0.798 | S100A9 | TNFRSF1B | 0.787 | AKAP1 | LRRK2 | 0.812 |
| TMEM71 | RBMS1 | 0.770 | LILRB2 | CBX6 | 0.754 | BICD2 | SLC16A6 | 0.710 |
| PRICKLE2 | CAVIN1 | 0.776 | PLAU | P2RY14 | 0.768 | BICD2 | PCBP3 | 0.779 |
| LILRB3 | VAV1 | 0.705 | ARPIN | PDE4B | 0.762 | ADCY6 | SH3RF2 | 0.792 |
| GOLPH3L | CLEC4A | 0.798 | PTGS1 | CREB5 | 0.795 | NFAM1 | BPGM | 0.760 |
| ACOT9 | MIR100HG | 0.759 | FPR1 | CYP26B1 | 0.785 | SH2D3C | NRG1 | 0.787 |
| GPR65 | SLC16A6 | 0.705 | S1PR3 | RASSF5 | 0.759 | NRCAM | PTGS2 | 0.796 |
| GLCE | DOK3 | 0.745 | LILRB1 | HS3ST3B1 | 0.794 | ASAP1 | TLR4 | 0.734 |
| CLEC4A | SLC16A6 | 0.744 | TAGAP | EGR3 | 0.693 | ACOT9 | GPR137B | 0.803 |
| SWAP70 | BPGM | 0.721 | S1PR1 | CREB5 | 0.775 | ZDHHC23 | TNFAIP2 | 0.790 |
| LRP4 | ABL2 | 0.799 | LRP4 | VAV1 | 0.773 | CHN1 | CAVIN3 | 0.776 |
| CXCL8 | PRR16 | 0.803 | PDPN | MCAM | 0.796 | TLR1 | BTG2 | 0.801 |
| UQCRC1 | FLT1 | 0.781 | GAS2L3 | CEBPB | 0.789 | CSF2RB | TUBA1A | 0.778 |
| GAS2L3 | FGF7 | 0.818 | FPR1 | GADD45B | 0.754 | CEBPB | IFITM2 | 0.769 |
| SELE | WWC2 | 0.787 | OCRL | TLR1 | 0.824 | MCAM | THBD | 0.784 |
| TNFRSF10C | CD53 | 0.703 | AGAP1 | PIP4P2 | 0.787 | SERPINE2 | FAM155A | 0.808 |
| RIPOR3 | S100A12 | 0.808 | ICAM1 | SELE | 0.790 | ADGRG3 | GFPT2 | 0.744 |
| CD300A | AQP9 | 0.784 | CLMP | SH3BP5 | 0.762 | SERPINB9 | OSMR | 0.787 |
| HCLS1 | ENTPD1 | 0.734 | LCP2 | GADD45B | 0.773 | TRAK1 | BDH1 | 0.785 |
| IL11 | BCL6 | 0.781 | RBMS1 | PROK2 | 0.730 | IL24 | IL6 | 0.807 |
| CLEC4E | TLR1 | 0.727 | NFAM1 | IER5 | 0.695 | DLC1 | PIP4P2 | 0.796 |
| RAI14 | VIM | 0.762 | RILPL2 | RBMS1 | 0.825 | ACOT9 | VNN2 | 0.806 |
| LCP1 | CHST2 | 0.737 | TAGAP | CHST2 | 0.738 | PRICKLE2 | C5AR1 | 0.810 |
| CXCL8 | LST1 | 0.772 | SELL | TNFAIP2 | 0.768 | LRRK2 | GADD45B | 0.784 |
| NFE2 | STAT5B | 0.771 | CREM | RHOH | 0.742 | S100A12 | TLR1 | 0.800 |
| NIBAN1 | IER5 | 0.742 | GLCE | CLMP | 0.772 | HIBCH | HSD11B2 | 0.764 |
| IFITM2 | SERPINB9 | 0.772 | GALNT12 | RIPOR2 | 0.788 | FPR2 | SELL | 0.801 |
| JAZF1 | CAVIN1 | 0.755 | F5 | SERPINE1 | 0.806 | GLIPR1 | CAVIN1 | 0.702 |
| CAVIN3 | MEFV | 0.776 | RALGAPA2 | IER5 | 0.766 | PI15 | RIPOR2 | 0.763 |
| EMP3 | RHOH | 0.727 | S1PR3 | GFPT2 | 0.742 | SORT1 | ITPRIP | 0.800 |
| PI15 | SNN | 0.785 | FCGR3B | LILRA5 | 0.803 | CGAS | OSMR | 0.778 |
| RILPL2 | SWAP70 | 0.744 | COL7A1 | MSN | 0.788 | COL15A1 | CLMP | 0.763 |
| GALNT12 | SSH2 | 0.776 | CRIP2 | CHST11 | 0.760 | IL6 | SOD2 | 0.814 |
| ITGA5 | G0S2 | 0.805 | FCER1G | CCDC69 | 0.764 | IL24 | F5 | 0.803 |
| SELENBP1 | FAM20C | 0.781 | SORT1 | IL1RN | 0.798 | TMEM154 | LCP2 | 0.745 |
| ANGPTL2 | CLEC4A | 0.799 | ENO2 | GMFG | 0.796 | SSH2 | VNN2 | 0.802 |
| FGF2 | CD53 | 0.747 | LOXL2 | SPOCK1 | 0.786 | OLFM1 | OSMR | 0.806 |
| TSPAN6 | BASP1 | 0.814 | FPR1 | THBD | 0.773 | SLA | ARHGAP25 | 0.725 |
| HSD11B2 | CD69 | 0.785 | WNK2 | ST3GAL5 | 0.796 | KCNJ15 | CEP19 | 0.732 |
| RAB31 | FLT1 | 0.766 | SHTN1 | PTGS2 | 0.799 | ASB7 | LRRK2 | 0.780 |
| FCER1G | HCK | 0.751 | SELENBP1 | FILIP1L | 0.806 | TRIM2 | CRIP2 | 0.788 |
| RNF149 | CYP26B1 | 0.815 | SH3RF2 | STC1 | 0.819 | AFG3L2 | CCDC3 | 0.772 |
| RAB31 | PLA1A | 0.772 | COL4A2 | BCL6 | 0.684 | S1PR1 | RBMS1 | 0.756 |
| SHTN1 | BMERB1 | 0.807 | PKIB | RALGAPA2 | 0.759 | PAPPA | ACOD1 | 0.822 |
| BICD2 | TLR4 | 0.687 | GZMB | SELP | 0.808 | IFITM2 | SLC11A1 | 0.773 |
| CMTM4 | NRG1 | 0.807 | PRR16 | CD86 | 0.802 | CBX6 | BCL6 | 0.676 |
| PPP1R18 | CCDC88A | 0.752 | ANGPTL2 | VNN2 | 0.817 | CREM | BPGM | 0.762 |
| ANGPTL2 | CAVIN1 | 0.763 | RGS16 | TLR4 | 0.709 | PPP2R5A | PLA1A | 0.776 |
| PPP1R18 | S100A12 | 0.776 | C5AR1 | GFPT2 | 0.765 | IL6 | CD69 | 0.790 |
| BDH1 | LILRA5 | 0.800 | RALGAPA2 | TIMP1 | 0.787 | RGCC | S100A9 | 0.821 |
| GNAI2 | ITGA5 | 0.760 | RAI14 | CEMIP | 0.779 | CRIP2 | RFLNB | 0.764 |
| FGF2 | HLX | 0.730 | TSPAN6 | FZD5 | 0.764 | CMTM4 | HCLS1 | 0.753 |
| SNORC | LILRA5 | 0.796 | FGF7 | THBD | 0.787 | CAVIN3 | CD14 | 0.770 |
| SWAP70 | ANGPT2 | 0.739 | CHST15 | OSMR | 0.790 | ERMP1 | EGR3 | 0.780 |
| SELENBP1 | FERMT2 | 0.807 | BDH1 | NFE2 | 0.795 | LILRA2 | SELL | 0.805 |
| NCF4 | MAP4K4 | 0.771 | RAB31 | MMP19 | 0.768 | NRG1 | CREB5 | 0.784 |
| BDH1 | LAT2 | 0.775 | PDPN | LOXL2 | 0.774 | TREM1 | CCDC3 | 0.762 |
| GAS2L3 | COL15A1 | 0.796 | ATP5IF1 | CAVIN1 | 0.762 | CSNK1G3 | ANGPTL2 | 0.777 |
| KCNJ15 | TNFAIP2 | 0.725 | ITGAM | SRGN | 0.789 | NIPSNAP3A | ADGRG3 | 0.771 |
| COL15A1 | THEMIS2 | 0.782 | RGCC | SPOCK1 | 0.771 | GZMB | PRR16 | 0.796 |
| LY96 | PREX1 | 0.775 | MYO1D | C5AR1 | 0.798 | TCEA3 | SERPINE2 | 0.796 |
| OCRL | IL24 | 0.804 | SHTN1 | SWAP70 | 0.750 | NIPSNAP3A | BTG2 | 0.765 |
| WNK2 | FGR | 0.799 | LILRB1 | MEFV | 0.797 | PKIB | FCGR3B | 0.805 |
| UQCRC1 | MCAM | 0.791 | CEBPB | RHOH | 0.750 | AGFG2 | TMEM154 | 0.802 |
| CLU | CXCR4 | 0.717 | ATP5IF1 | SLC2A3 | 0.768 | RFLNB | SSH2 | 0.765 |
| NEO1 | PPP1R18 | 0.776 | COX10 | TREM1 | 0.789 | CYTH4 | SLC16A6 | 0.777 |
| AMPD2 | TREM1 | 0.778 | RAB15 | PHLDB2 | 0.819 | ADCY6 | SRGN | 0.815 |
| IL11 | PTPRC | 0.827 | CXCR2 | AQP9 | 0.781 | HLX | SLC2A3 | 0.715 |
| ANGPTL2 | STAT5B | 0.765 | SPP1 | ACOD1 | 0.780 | GZMB | C5AR1 | 0.784 |
| CSF3 | ASAP1 | 0.814 | PRRX1 | CCDC3 | 0.779 | NR3C2 | CBX6 | 0.779 |
| ARPIN | S1PR3 | 0.750 | ADCY6 | AGFG2 | 0.773 | TCEA3 | LILRA5 | 0.790 |
| RNF149 | SERPINB9 | 0.782 | HCK | WWC2 | 0.770 | NR3C2 | IL11 | 0.822 |
| WNK2 | SNN | 0.829 | AQP9 | TUBB6 | 0.793 | DUSP4 | SRGN | 0.783 |
| WWC2 | CD14 | 0.759 | RIPOR3 | CCDC88A | 0.796 | ANKRD35 | C11orf96 | 0.763 |
| CCL2 | CREB5 | 0.761 | INHBA | VIM | 0.785 | TCEA3 | SH3BP5 | 0.781 |
| TMEM273 | NOD2 | 0.716 | SHTN1 | VNN2 | 0.832 | ANGPTL2 | ITGAM | 0.773 |
| SNORC | CXCR1 | 0.748 | DPYSL3 | ANGPT2 | 0.788 | VASN | PCBP3 | 0.716 |
| NR3C2 | STAT5B | 0.777 | IL6 | ITGA5 | 0.807 | DLC1 | COL4A2 | 0.782 |
| TRAK1 | B4GALT4 | 0.757 | ATP5IF1 | CHN1 | 0.799 | LILRB3 | GPR176 | 0.723 |
| MIR100HG | G0S2 | 0.823 | NNMT | BMERB1 | 0.822 | CCR1 | CRIP2 | 0.814 |
| CGAS | TLR4 | 0.681 | UBAC1 | SERPINE2 | 0.788 | JAZF1 | THBD | 0.801 |
| LRRK2 | BPGM | 0.780 | CD300A | ARHGAP25 | 0.795 | DPYSL3 | PTGS1 | 0.788 |
| TRAK1 | RASSF5 | 0.781 | EMP3 | FGR | 0.772 | HSD11B2 | OSMR | 0.780 |
| TMEM106C | TNFAIP6 | 0.790 | MSN | OSM | 0.808 | PPP2R5A | TNC | 0.805 |
| CXCR2 | NRCAM | 0.796 | COX10 | COL7A1 | 0.809 | AKAP1 | DPYSL2 | 0.787 |
| FGF7 | HLX | 0.759 | LILRB1 | CHST15 | 0.784 | GOLPH3L | OSMR | 0.799 |
| CSF2RB | TNFRSF1B | 0.731 | RAB15 | NDUFA10 | 0.786 | RNF149 | RIPK2 | 0.765 |
| MSN | RBMS1 | 0.803 | RFLNB | SMAP2 | 0.759 | IL24 | PCBP3 | 0.791 |
| NOD2 | ST3GAL5 | 0.718 | SLC15A4 | LCP1 | 0.769 | TLR1 | CHST2 | 0.793 |
| WNK2 | NCF4 | 0.788 | IFITM2 | PRR16 | 0.802 | CEBPB | ANKRD35 | 0.792 |
| ITGA5 | CXCL5 | 0.774 | CGAS | NFAM1 | 0.712 | TNF | LST1 | 0.765 |
| CXCR2 | FAM155A | 0.837 | LRRK2 | S100A12 | 0.793 | C2CD2 | CHST2 | 0.763 |
| CYTH4 | CCDC3 | 0.760 | SELENBP1 | COL4A2 | 0.800 | IL6 | P2RY13 | 0.804 |
| DLC1 | LILRB1 | 0.811 | GPR176 | TMEM273 | 0.765 | COL7A1 | SPOCK1 | 0.808 |
| FJX1 | FAM49A | 0.766 | CTHRC1 | ANKRD35 | 0.795 | BPGM | CHST2 | 0.774 |
| IL24 | FCER1G | 0.804 | FGF7 | SNN | 0.799 | RHOH | CD53 | 0.672 |
| CHN1 | NFAM1 | 0.786 | FCGR3B | SNN | 0.818 | LOXL2 | CCDC88A | 0.786 |
| FERMT2 | SPOCK1 | 0.779 | ASAP1 | VNN2 | 0.807 | TNC | TNFRSF10C | 0.811 |
| ACOT9 | S100A9 | 0.830 | S1PR1 | WWC2 | 0.741 | FJX1 | IL13RA2 | 0.860 |
| GLCE | P2RY14 | 0.808 | B4GALT4 | ACOD1 | 0.792 | IER5 | BCL6 | 0.662 |
| SLC16A6 | SLFN11 | 0.718 | F5 | LILRA5 | 0.761 | FAP | SELP | 0.787 |
| CR1 | HS3ST3B1 | 0.771 | CXCR1 | AQP9 | 0.745 | FPR2 | ASAP1 | 0.789 |
| LILRA2 | IER5 | 0.726 | NR3C2 | SELE | 0.819 | CGAS | P2RY14 | 0.779 |
| COL7A1 | RHOH | 0.773 | PDE4B | TNF | 0.765 | CRIP2 | PAPPA | 0.830 |
| ANKRD35 | NCF2 | 0.775 | PIP4P2 | DOK3 | 0.750 | RAI14 | SERPINE2 | 0.790 |
| PRICKLE2 | CD53 | 0.776 | CDX2 | P2RY13 | 0.800 | RNF149 | FFAR2 | 0.756 |
| PPP1R18 | GPR137B | 0.781 | LY96 | CEMIP | 0.797 | LOXL2 | CEMIP | 0.777 |
| COQ9 | SLC11A1 | 0.796 | BASP1 | ITGA5 | 0.789 | ABL2 | MSN | 0.772 |
| CSNK1G3 | CXCR4 | 0.724 | RAI14 | SMAP2 | 0.752 | CHI3L2 | HCK | 0.738 |
| SWAP70 | TUBA1A | 0.726 | PROK2 | ST3GAL5 | 0.754 | PROK2 | TNFAIP2 | 0.717 |
| SNN | CREB5 | 0.810 | HSD11B2 | STAT5B | 0.750 | ADCY6 | S1PR1 | 0.797 |
| CXCR1 | PLAU | 0.743 | VIM | THEMIS2 | 0.760 | KCNJ15 | PCBP3 | 0.738 |
| GZMB | TNFAIP6 | 0.795 | TRPM6 | TNFAIP6 | 0.779 | NIBAN1 | TUBA1A | 0.770 |
| TRPM6 | ANGPT2 | 0.788 | TLR1 | FAM49A | 0.758 | MIR100HG | OSM | 0.814 |
| PIK3AP1 | CHST2 | 0.735 | CBX6 | CLEC4E | 0.712 | COX10 | OCRL | 0.789 |
| SELP | BMERB1 | 0.813 | MYO1F | SLC35G2 | 0.792 | SH3RF2 | S1PR1 | 0.794 |
| ADGRG3 | VNN2 | 0.768 | BICD2 | IER5 | 0.708 | CHI3L2 | DUSP4 | 0.804 |
| FJX1 | RASSF5 | 0.754 | TAGAP | CD14 | 0.688 | OCRL | CLEC4E | 0.792 |
| LILRA2 | LOXL2 | 0.773 | SELP | PDLIM4 | 0.804 | SWAP70 | MIR100HG | 0.695 |
| CAVIN3 | MX2 | 0.778 | ACOT9 | CD53 | 0.754 | MYO1D | DUSP2 | 0.774 |
| VASN | ZDHHC18 | 0.733 | RAB15 | VAV1 | 0.770 | OLFM1 | PTGS2 | 0.791 |
| CEBPB | CHST2 | 0.789 | SLFN11 | GFPT2 | 0.712 | S1PR3 | CXCL8 | 0.795 |
| DUSP4 | PDE4B | 0.755 | PPP2R5A | PDLIM4 | 0.794 | JAZF1 | CXCL5 | 0.765 |
| CHST15 | TUBB6 | 0.790 | S1PR3 | C11orf96 | 0.782 | RAI14 | PDLIM4 | 0.788 |
| MAP4K4 | CD53 | 0.717 | PLEK | PXDN | 0.793 | RFLNB | NOD2 | 0.716 |
| COL4A2 | CLEC4A | 0.791 | SLC15A4 | FGF7 | 0.795 | UQCRC1 | RILPL2 | 0.818 |
| CLEC4E | LST1 | 0.746 | RGCC | SOD2 | 0.798 | RFLNB | PRRX1 | 0.761 |
| CMTM4 | BASP1 | 0.789 | NFAM1 | RILPL2 | 0.777 | CXCL8 | LOXL2 | 0.781 |
| NCF2 | TREM1 | 0.728 | GPR183 | CCL2 | 0.761 | IL1RN | SELP | 0.817 |
| CR1 | DOK3 | 0.720 | DUSP4 | FCGR3B | 0.801 | OCRL | FGR | 0.788 |
| SH3RF2 | FPR1 | 0.813 | LILRB1 | IL1RN | 0.804 | RAB15 | FCGR2B | 0.793 |
| BICD2 | PIP4P2 | 0.734 | FCER1G | MYO1F | 0.764 | ADCY6 | ZNF704 | 0.777 |
| MEFV | PI15 | 0.773 | TRAK1 | ICAM1 | 0.757 | P2RY13 | BCL6 | 0.730 |
| NIBAN1 | TNFRSF10C | 0.736 | HIBCH | GPR176 | 0.756 | DPYSL2 | GFPT2 | 0.743 |
| LRRK2 | CR1 | 0.756 | PRICKLE2 | FCER1G | 0.796 | SOD2 | FAM155A | 0.820 |
| CRIP2 | PDE4B | 0.755 | STC1 | RIPOR2 | 0.796 | RGS19 | ST3GAL5 | 0.714 |
| MMP3 | BPGM | 0.778 | NR3C2 | LOXL2 | 0.814 | S1PR1 | NRG1 | 0.805 |
| GLIPR1 | FAM20C | 0.723 | ASB7 | P2RY14 | 0.777 | OCRL | LILRB3 | 0.755 |
| TRPM6 | JAZF1 | 0.791 | TMEM273 | LST1 | 0.771 | RAB31 | SPOCK1 | 0.759 |
| ADCY6 | ENO2 | 0.799 | IL11 | LAT2 | 0.825 | DLC1 | SLC2A3 | 0.749 |
| FJX1 | NFE2 | 0.772 | PROK2 | SLC2A3 | 0.696 | SERPINB9 | HS3ST3B1 | 0.773 |
| PTPRC | SLC2A3 | 0.733 | ASAP1 | GMFG | 0.783 | GPR176 | C1R | 0.756 |
| PPP2R5A | ACOD1 | 0.796 | MYO1D | GPR176 | 0.764 | TMEM154 | MMP3 | 0.790 |
| STAT5B | TLR4 | 0.681 | MMP19 | BMERB1 | 0.806 | IER5 | PTGS2 | 0.748 |
| ZNF704 | NRCAM | 0.762 | AKAP1 | COL4A2 | 0.785 | PDE4B | BCL2A1 | 0.733 |
| LRP4 | MYO1F | 0.791 | CHN1 | PRR16 | 0.803 | PREX1 | PHLDB2 | 0.757 |
| CAVIN3 | AMPD2 | 0.794 | ZNF704 | PI15 | 0.761 | MX2 | CD14 | 0.754 |
| LRP4 | TSPAN6 | 0.765 | FGR | PDE4B | 0.753 | SOD2 | FAM49A | 0.786 |
| IL11 | NRCAM | 0.828 | FAP | BTG2 | 0.758 | AMPD2 | SERPINE1 | 0.809 |
| CEMIP | SLC11A1 | 0.789 | FLT1 | SERPINB9 | 0.779 | CCDC88A | WNT5A | 0.817 |
| FPR1 | CD53 | 0.736 | RAB15 | GLT1D1 | 0.776 | WWC2 | TNFRSF10C | 0.749 |
| FGR | MEFV | 0.774 | DSP | TNFAIP6 | 0.792 | C2CD2 | LCP2 | 0.781 |
| COL4A2 | VIM | 0.747 | RHOH | HS3ST3B1 | 0.747 | GNAI2 | PTPRC | 0.736 |
| B4GALT4 | P2RY14 | 0.784 | PCBP3 | DOK3 | 0.736 | TMEM71 | DPYSL3 | 0.785 |
| F5 | BCL6 | 0.695 | FCGR3B | VAV1 | 0.815 | S100A8 | CD86 | 0.831 |
| NIBAN1 | CLMP | 0.775 | OSM | CCDC3 | 0.809 | PRRX1 | S100A12 | 0.801 |
| CXCR2 | DOK3 | 0.778 | EGR3 | FAM155A | 0.776 | ADCY6 | LRRK2 | 0.802 |
| PDPN | VNN2 | 0.820 | PKIB | C11orf96 | 0.808 | GALNT12 | ATP5IF1 | 0.748 |
| PDPN | CEMIP | 0.777 | MCAM | FAM49A | 0.793 | F5 | FGR | 0.777 |
| OSM | CD69 | 0.802 | OSM | ENTPD1 | 0.813 | GOLPH3L | MYO1F | 0.772 |
| SWAP70 | PIP4P2 | 0.719 | CRIP2 | CD69 | 0.766 | NCF2 | PROK2 | 0.703 |
| CAVIN3 | SELL | 0.787 | SLC2A3 | SLC16A6 | 0.715 | CCR1 | CREB5 | 0.797 |
| LRP4 | GOLPH3L | 0.760 | BDH1 | PRICKLE2 | 0.814 | TNF | CD69 | 0.760 |
| SERPINE2 | CD69 | 0.771 | FCER1G | CYP26B1 | 0.798 | IL6 | VNN2 | 0.831 |
| SH2D3C | ST3GAL5 | 0.751 | TAGAP | COL4A2 | 0.707 | PLEK | SLC35G2 | 0.807 |
| FPR1 | CHST11 | 0.734 | SH2D3C | PPP1R18 | 0.753 | NOD2 | SLC16A6 | 0.715 |
| FCGR3B | FAM20C | 0.800 | AMPD2 | NRG1 | 0.807 | GAS2L3 | LST1 | 0.763 |
| STAP2 | RFLNB | 0.768 | TMEM106C | F5 | 0.791 | HSD11B2 | ICAM1 | 0.772 |
| CLEC4E | SERPINE2 | 0.747 | CCL4 | NOD2 | 0.746 | CGAS | GNAI2 | 0.714 |
| ACOT9 | CD14 | 0.782 | TAGAP | F5 | 0.711 | MSN | SPOCK1 | 0.772 |
| NIPSNAP3A | SH3BP5 | 0.773 | FCGR3B | CCDC3 | 0.812 | PRICKLE2 | CXCR2 | 0.844 |
| OLFM1 | CAVIN3 | 0.786 | THBD | CEP19 | 0.742 | NIBAN1 | CHST2 | 0.788 |
| CSNK1G3 | TNFAIP6 | 0.789 | NRCAM | GFPT2 | 0.751 | BICD2 | GMFG | 0.780 |
| PTGS1 | LAT2 | 0.784 | RASSF5 | SSH2 | 0.725 | ANGPTL2 | LCP1 | 0.737 |
| CEBPB | ENO2 | 0.781 | NR3C2 | NFE2 | 0.780 | AGAP1 | ABL2 | 0.788 |
| BDH1 | CRIP2 | 0.788 | COTL1 | SMAP2 | 0.753 | CD93 | CEP19 | 0.734 |
| NRG1 | ITGA5 | 0.790 | TSPAN6 | COL15A1 | 0.798 | TCEA3 | CD93 | 0.804 |
| SELE | CD14 | 0.802 | FERMT2 | CTHRC1 | 0.780 | PREX1 | RIPOR2 | 0.699 |
| AQP9 | VAV1 | 0.778 | DLC1 | CBX6 | 0.773 | GZMB | VAV1 | 0.764 |
| CMTM4 | NNMT | 0.805 | UBAC1 | RALGAPA2 | 0.785 | PRR16 | GFPT2 | 0.788 |
| TRAK1 | CHRDL2 | 0.760 | SELL | MAP4K4 | 0.809 | B4GALT4 | FPR2 | 0.800 |
| C11orf96 | ITPRIP | 0.806 | CCR1 | FAM49A | 0.797 | CLU | RNF149 | 0.785 |
| UQCRC1 | WNT5A | 0.819 | SERPINB9 | LAT2 | 0.761 | ENO2 | LST1 | 0.781 |
| TMEM106C | PIK3AP1 | 0.705 | MCAM | CHST11 | 0.769 | GAS2L3 | CD300A | 0.781 |
| NEO1 | PTPRC | 0.788 | NDFIP2 | CEMIP | 0.797 | BCAT1 | STAT5B | 0.745 |
| CHST11 | GPR137B | 0.775 | CGAS | C11orf96 | 0.769 | LILRB3 | PLAU | 0.733 |
| PKIB | SPP1 | 0.804 | TCEA3 | S1PR3 | 0.778 | LILRB2 | TUBA1A | 0.785 |
| SLC11A1 | WNT5A | 0.817 | LILRA2 | MMP3 | 0.785 | CTHRC1 | IL1RN | 0.802 |
| SWAP70 | S100A9 | 0.776 | B4GALT4 | DUSP2 | 0.766 | S100A12 | C1R | 0.797 |
| GLCE | CXCL8 | 0.796 | NR3C2 | STAP2 | 0.769 | CAVIN1 | CXCR4 | 0.736 |
| MIR100HG | FAM155A | 0.791 | CR1 | CD53 | 0.687 | CCDC88A | VIM | 0.759 |
| TMEM71 | SLA | 0.768 | FERMT2 | TLR4 | 0.718 | CD300A | P2RY14 | 0.799 |
| LILRB3 | MX2 | 0.729 | RGCC | IL1RN | 0.791 | LILRB3 | GZMB | 0.751 |
| S1PR3 | TNC | 0.782 | TMEM71 | TUBA1A | 0.788 | TMEM106C | CSF3 | 0.786 |
| ERMP1 | PCBP3 | 0.765 | DSP | C2CD2 | 0.791 | PFKL | CYP26B1 | 0.825 |
| HSD11B2 | MYO1F | 0.781 | LCP2 | PRRX1 | 0.767 | BASP1 | CXCR4 | 0.730 |
| DSP | ITGAM | 0.779 | SELENBP1 | SRGN | 0.816 | HSPA6 | CCDC88A | 0.714 |
| CLU | TREM1 | 0.778 | SHTN1 | RFLNB | 0.783 | TMEM71 | SERPINE2 | 0.799 |
| CHST11 | SERPINE2 | 0.780 | NFE2 | S100A12 | 0.778 | CTHRC1 | RGCC | 0.781 |
| MCAM | THEMIS2 | 0.796 | FILIP1L | BPGM | 0.782 | LILRB2 | SMAP2 | 0.787 |
| GLCE | CHI3L2 | 0.743 | FJX1 | CYTH4 | 0.768 | DPYSL2 | VAV1 | 0.743 |
| VASN | CCR1 | 0.770 | CMTM8 | PIK3AP1 | 0.736 | STAP2 | HS3ST3B1 | 0.765 |
| AKAP1 | BASP1 | 0.804 | CLEC4E | FFAR2 | 0.720 | ZDHHC23 | RGS19 | 0.769 |
| S100A9 | CXCR4 | 0.797 | TNFAIP2 | LAT2 | 0.753 | PFKL | SELL | 0.812 |
| CR1 | PLEK | 0.722 | FCER1G | TLR1 | 0.781 | SNORC | CHST11 | 0.776 |
| GZMB | PDE4B | 0.758 | SNORC | PIK3AP1 | 0.732 | ATP5IF1 | SPOCK1 | 0.780 |
| CSNK1G3 | NR3C2 | 0.777 | SNORC | SPHK1 | 0.772 | IL6 | TUBA1A | 0.817 |
| NR3C2 | PIK3AP1 | 0.772 | FAP | CREB5 | 0.757 | P2RY13 | VNN2 | 0.786 |
| NDUFA10 | PIK3AP1 | 0.735 | MCAM | ZDHHC18 | 0.775 | STAP2 | BCAT1 | 0.758 |
| ENO2 | BCAT1 | 0.764 | PLEK | TREM1 | 0.749 | CLEC4A | CD69 | 0.742 |
| IER5 | SLC16A6 | 0.694 | COL7A1 | HSPA6 | 0.791 | DLC1 | DPYSL2 | 0.760 |
| TRAK1 | HCLS1 | 0.731 | ENO2 | MEFV | 0.784 | CCDC69 | FFAR2 | 0.725 |
| CCR1 | SMAP2 | 0.777 | FGR | SOD2 | 0.771 | FAP | P2RY14 | 0.769 |
| IFITM2 | ITGA5 | 0.755 | OSM | THBD | 0.819 | FCGR3B | LCP2 | 0.804 |
| CLEC4A | TCF4 | 0.792 | CHST11 | CD14 | 0.729 | CMTM4 | SLFN11 | 0.769 |
| RILPL2 | ACOT9 | 0.808 | LILRB2 | TMEM273 | 0.785 | SELP | DOK3 | 0.780 |
| IFITM2 | S1PR1 | 0.795 | NIBAN1 | GLT1D1 | 0.740 | NFE2 | TNFRSF10C | 0.715 |
| OLFM1 | CD86 | 0.771 | NNMT | PHLDB2 | 0.780 | CSTA | CD53 | 0.677 |
| OCRL | CAVIN3 | 0.783 | MSN | PHLDB2 | 0.770 | TCEA3 | DPYSL3 | 0.759 |
| IFITM2 | G0S2 | 0.798 | RHOH | WWC2 | 0.738 | AKAP1 | ARHGAP25 | 0.782 |
| FCER1G | BICD2 | 0.768 | TAGAP | DPYSL2 | 0.721 | CSNK1G3 | SPP1 | 0.752 |
| SNORC | PKIB | 0.748 | AFG3L2 | ACOD1 | 0.764 | NR3C2 | CCDC88A | 0.779 |
| FAM49A | ST3GAL5 | 0.779 | NCF4 | S100A8 | 0.812 | TNFRSF1B | LAT2 | 0.744 |
| SWAP70 | PRRX1 | 0.715 | STAT5B | BMERB1 | 0.820 | CDX2 | RIPK2 | 0.777 |
| MEFV | SNN | 0.788 | CREM | CCDC3 | 0.790 | GNAI2 | WWC2 | 0.748 |
| IL13RA2 | HS3ST3B1 | 0.875 | CBX6 | PI15 | 0.770 | MYO1F | BMERB1 | 0.807 |
| PDPN | CCL4 | 0.770 | FGR | OSMR | 0.790 | CSF3 | SERPINE1 | 0.803 |
| TMEM154 | CEP19 | 0.717 | SELENBP1 | GMFG | 0.809 | GPR183 | CD93 | 0.800 |
| SH2D3C | INHBA | 0.791 | CYTH4 | LCP1 | 0.741 | AGAP1 | SWAP70 | 0.709 |
| WWC2 | SLFN11 | 0.736 | CHN1 | CSF2RB | 0.789 | RHOH | WNT5A | 0.772 |
| GALNT12 | GFPT2 | 0.770 | GPR176 | PXDN | 0.785 | NCF4 | TREM1 | 0.773 |
| DLC1 | CREM | 0.790 | CLEC4A | GFPT2 | 0.783 | HIBCH | CLEC4E | 0.792 |
| ASB7 | BCL2A1 | 0.752 | HSD11B2 | FCGR2B | 0.796 | MEFV | CEP19 | 0.704 |
| GLT1D1 | CLEC4E | 0.713 | PRR16 | VAV1 | 0.803 | CXCL8 | NCF4 | 0.789 |
| CXCL8 | RHOH | 0.768 | CSF2RB | HCLS1 | 0.717 | S1PR3 | CD14 | 0.755 |
| CLEC4E | INHBA | 0.783 | CSF3 | CCDC88A | 0.803 | F5 | RGCC | 0.804 |
| ITGA5 | TNFRSF1B | 0.759 | PIK3AP1 | GPR183 | 0.715 | PPP2R5A | SELP | 0.813 |
| MEFV | SLA | 0.743 | CCDC69 | PLAU | 0.767 | IL1RN | TNF | 0.795 |
| ZDHHC23 | BICD2 | 0.817 | OLFM1 | PREX1 | 0.758 | PPP2R5A | MMP19 | 0.758 |
| CYTH4 | DOK3 | 0.738 | GLT1D1 | FAM155A | 0.782 | HIBCH | CHST2 | 0.771 |
| TMEM71 | THBD | 0.802 | NIBAN1 | FAP | 0.767 | IL24 | SH3BP5 | 0.811 |
| SORT1 | PAPPA | 0.831 | S100A12 | VAV1 | 0.781 | PAPPA | DOK3 | 0.809 |
| MX2 | S100A12 | 0.789 | LRP4 | WNT5A | 0.807 | SMAP2 | THEMIS2 | 0.759 |
| RAB15 | GNAI2 | 0.776 | ADGRG3 | HCLS1 | 0.727 | AGFG2 | LCP1 | 0.750 |
| SLC15A4 | CCL4 | 0.792 | IL1RN | RIPOR2 | 0.776 | STC1 | TNFAIP6 | 0.813 |
| PI15 | TNFAIP2 | 0.761 | LILRB1 | SLC2A3 | 0.762 | ARPIN | COL7A1 | 0.796 |
| NEO1 | HLX | 0.754 | PAPPA | TIMP1 | 0.821 | NRG1 | SLC2A3 | 0.762 |
| FPR1 | SLC11A1 | 0.788 | SERPINB9 | CCDC88A | 0.778 | CMTM4 | VIM | 0.788 |
| LILRA5 | TNFRSF1B | 0.732 | SORT1 | FJX1 | 0.768 | CAVIN1 | FFAR2 | 0.762 |
| CCN4 | MAP4K4 | 0.774 | BDH1 | LCP1 | 0.749 | RHOH | SELP | 0.785 |
| CRIP2 | C2CD2 | 0.768 | GALNT12 | PRRX1 | 0.790 | TRIM2 | S1PR1 | 0.801 |
| KIAA1522 | COTL1 | 0.780 | CAVIN3 | STAT5B | 0.778 | CSF3 | CSF3R | 0.760 |
| KIAA1522 | FAM20C | 0.783 | COX10 | IL6 | 0.814 | RGCC | CD14 | 0.776 |
| TMEM106C | ARHGAP25 | 0.746 | FGF2 | LST1 | 0.778 | COX10 | LOXL2 | 0.807 |
| HCK | GPR137B | 0.772 | SH2D3C | TLR4 | 0.703 | PIK3AP1 | NRG1 | 0.760 |
| CLU | MCAM | 0.765 | LILRA2 | LILRA5 | 0.737 | PI15 | ARHGAP25 | 0.762 |
| VASN | ACOD1 | 0.740 | SHTN1 | PXDN | 0.813 | LCP1 | LCP2 | 0.717 |
| GLCE | PLAU | 0.784 | DPYSL3 | CXCL5 | 0.782 | RNF149 | VAV1 | 0.771 |
| HLX | INHBA | 0.738 | BPGM | MAP4K4 | 0.750 | COL15A1 | ENO2 | 0.778 |
| POLDIP2 | BASP1 | 0.790 | EGR3 | ADGRG3 | 0.751 | SNORC | BCAT1 | 0.769 |
| ATP5IF1 | GZMB | 0.779 | DLC1 | PXDN | 0.806 | CHI3L2 | CD69 | 0.755 |
| VAV1 | TNFRSF10C | 0.713 | FPR2 | PTGS2 | 0.771 | GPR183 | OSMR | 0.774 |
| DSP | COL4A2 | 0.783 | GAS2L3 | ZDHHC18 | 0.772 | TRPM6 | PCBP3 | 0.760 |
| TRPM6 | OCRL | 0.772 | CGAS | MCAM | 0.752 | FERMT2 | PDE4B | 0.740 |
| PKIB | CHRDL2 | 0.780 | ACOT9 | ASAP1 | 0.767 | IFITM2 | GLT1D1 | 0.762 |
| FAP | LILRA5 | 0.757 | COL15A1 | FAM49A | 0.782 | TAGAP | C1R | 0.711 |
| MYO1D | CCR1 | 0.799 | SPOCK1 | TNFRSF1B | 0.752 | SORT1 | WWC2 | 0.767 |
| CMTM8 | SLC15A3 | 0.774 | COL7A1 | NRG1 | 0.818 | LILRB2 | TNFAIP2 | 0.778 |
| AGFG2 | C2CD2 | 0.776 | HSPA6 | SERPINE2 | 0.753 | SH3RF2 | ABL2 | 0.778 |
| FJX1 | CXCR4 | 0.716 | VASN | C5AR1 | 0.752 | NFAM1 | SLFN11 | 0.719 |
| TMEM106C | MYO1F | 0.770 | ADGRG3 | LCP1 | 0.732 | CLEC4A | BCAT1 | 0.770 |
| COL7A1 | ANGPTL2 | 0.800 | CREM | FCGR3B | 0.812 | B4GALT4 | RBMS1 | 0.785 |
| HCLS1 | RGS16 | 0.713 | CTHRC1 | GMFG | 0.795 | GPR137B | LST1 | 0.780 |
| DLC1 | DOK3 | 0.770 | SORT1 | GPR183 | 0.757 | S1PR3 | SSH2 | 0.762 |
| STAP2 | CBX6 | 0.745 | GLT1D1 | SMAP2 | 0.738 | PDPN | WWC2 | 0.774 |
| RAB15 | FILIP1L | 0.806 | ANGPT2 | LILRA5 | 0.793 | SNORC | RALGAPA2 | 0.772 |
| RFLNB | SLC11A1 | 0.800 | RNF149 | S100A12 | 0.792 | JAZF1 | C11orf96 | 0.786 |
| NEO1 | PXDN | 0.826 | HLX | CXCR2 | 0.750 | FPR2 | VNN2 | 0.761 |
| STC1 | MAP4K4 | 0.802 | MYO1F | BCAT1 | 0.757 | TCF4 | FAM155A | 0.782 |
| CXCR1 | THEMIS2 | 0.731 | F5 | IL1RN | 0.785 | DPYSL3 | TLR1 | 0.789 |
| HLX | C11orf96 | 0.739 | STAP2 | SSH2 | 0.754 | DLC1 | CSF3 | 0.814 |
| TRAK1 | LILRB1 | 0.779 | IL24 | DUSP2 | 0.784 | SRGN | ARHGAP25 | 0.780 |
| LCP2 | SLC2A3 | 0.724 | VASN | ITPRIP | 0.769 | ENO2 | PHLDB2 | 0.789 |
| CYP26B1 | STAT5B | 0.812 | NFAM1 | RNF149 | 0.755 | NFE2 | GFPT2 | 0.762 |
| PDPN | RIPOR3 | 0.806 | CLU | FPR2 | 0.795 | LOXL2 | HS3ST3B1 | 0.776 |
| JAZF1 | LCP1 | 0.727 | PI15 | SLC16A6 | 0.768 | ACOD1 | ST3GAL5 | 0.770 |
| FAP | P2RY13 | 0.763 | UBAC1 | NDFIP2 | 0.789 | STAP2 | ACOD1 | 0.766 |
| CYP26B1 | CD69 | 0.795 | CEBPB | PTGS2 | 0.791 | LGALS1 | RBMS1 | 0.791 |
| ZNF704 | LILRA5 | 0.782 | BDH1 | FERMT2 | 0.797 | PRICKLE2 | PHLDB2 | 0.781 |
| MX2 | PLEK | 0.773 | RIPOR3 | SWAP70 | 0.748 | PIK3AP1 | IL13RA2 | 0.840 |
| SELENBP1 | GZMB | 0.803 | GLT1D1 | CSTA | 0.715 | SMAP2 | RIPK2 | 0.761 |
| DLC1 | CXCR2 | 0.817 | NR3C2 | MCAM | 0.796 | S100A8 | TNFAIP2 | 0.800 |
| RAI14 | FFAR2 | 0.767 | MYO1F | P2RY13 | 0.726 | CCL2 | SNN | 0.787 |
| TMEM154 | CCDC69 | 0.749 | PTGS2 | SELP | 0.815 | CDX2 | AMPD2 | 0.779 |
| ATP5IF1 | TNFRSF10C | 0.760 | CHST11 | STAT5B | 0.743 | TSPAN6 | CLU | 0.774 |
| C11orf96 | TCF4 | 0.764 | MIR100HG | MYO1F | 0.764 | TSPAN6 | NCF4 | 0.783 |
| OLFM1 | TNFAIP2 | 0.775 | TRIM2 | CD14 | 0.767 | TRPM6 | BDH1 | 0.765 |
| RAB31 | GPR137B | 0.791 | PCBP3 | PREX1 | 0.763 | ASAP1 | ZDHHC18 | 0.776 |
| VASN | SPP1 | 0.730 | PIP4P2 | CCDC3 | 0.761 | GNAI2 | TLR1 | 0.755 |
| ZNF704 | PTGS2 | 0.800 | CSTA | ITPRIP | 0.786 | GLCE | P2RY13 | 0.785 |
| CLU | CD93 | 0.771 | CCDC69 | SLC16A6 | 0.723 | SRGN | TIMP1 | 0.784 |
| AFG3L2 | PXDN | 0.805 | RFLNB | NCF2 | 0.784 | JAZF1 | NCF4 | 0.747 |
| SLC15A4 | FLT1 | 0.787 | CSF3R | NFE2 | 0.709 | THBD | SLFN11 | 0.765 |
| LILRB3 | AQP9 | 0.737 | RFLNB | ARHGAP25 | 0.766 | LGALS1 | CEMIP | 0.781 |
| PPP1R18 | SERPINE2 | 0.767 | TLR4 | TUBB6 | 0.720 | FAP | HCLS1 | 0.714 |
| NDFIP2 | WNT5A | 0.823 | CEBPB | JAZF1 | 0.789 | LRRK2 | CCDC3 | 0.790 |
| PLAU | MAP4K4 | 0.753 | DUSP4 | VIM | 0.779 | BICD2 | SLA | 0.731 |
| MCAM | TUBA1A | 0.749 | RAB31 | PRRX1 | 0.760 | NIPSNAP3A | SHTN1 | 0.776 |
| PFKL | GADD45B | 0.818 | COL7A1 | AQP9 | 0.783 | SOD2 | TIMP1 | 0.770 |
| PKIB | SERPINB9 | 0.789 | COX10 | DPYSL3 | 0.766 | UQCRC1 | S100A9 | 0.835 |
| FJX1 | HSPA6 | 0.752 | LRRK2 | TREM1 | 0.752 | CXCR1 | PDE4B | 0.702 |
| ATP5IF1 | CLEC4A | 0.795 | LRRK2 | TNFRSF1B | 0.727 | MMP19 | TREM1 | 0.764 |
| DUSP2 | IL6 | 0.792 | IL1B | BCL2A1 | 0.774 | SORT1 | CXCR1 | 0.758 |
| NFE2 | CD69 | 0.732 | TRIM2 | NFAM1 | 0.737 | SLC15A4 | TMEM154 | 0.808 |
| RHOH | NNMT | 0.768 | SORT1 | CREB5 | 0.798 | GPR176 | C11orf96 | 0.773 |
| LILRB3 | NOD2 | 0.692 | PREX1 | VIM | 0.746 | CSF2RB | PRRX1 | 0.768 |
| TREM1 | TCF4 | 0.775 | WNK2 | TREM1 | 0.775 | THBD | BMERB1 | 0.818 |
| OCRL | CREM | 0.784 | CTHRC1 | ICAM1 | 0.785 | PLAU | SLC16A6 | 0.774 |
| GAS2L3 | LCP2 | 0.789 | NEO1 | PROK2 | 0.751 | HIBCH | CEBPB | 0.815 |
| SLA | SH3BP5 | 0.735 | PCBP3 | CEMIP | 0.797 | HCK | RFLNB | 0.755 |
| PPP2R5A | LILRA2 | 0.806 | PKIB | RAB31 | 0.797 | CSF3 | CCN4 | 0.795 |
| MMP3 | THBD | 0.796 | SHTN1 | CAVIN3 | 0.773 | LGALS1 | BCAT1 | 0.754 |
| LILRA5 | LAT2 | 0.805 | ARPIN | PXDN | 0.796 | BPGM | RIPOR2 | 0.754 |
| NDFIP2 | TUBB6 | 0.785 | CSTA | NCF2 | 0.720 | TAGAP | ANKRD35 | 0.759 |
| PI15 | PLA1A | 0.781 | FLT1 | KCNJ15 | 0.718 | CAVIN3 | SLC11A1 | 0.800 |
| NDFIP2 | BCL6 | 0.727 | CCDC69 | RFLNB | 0.766 | LOXL2 | CHST2 | 0.783 |
| TNFAIP6 | C5AR1 | 0.784 | CGAS | CREB5 | 0.737 | OLFM1 | CBX6 | 0.764 |
| ANGPT2 | CLEC4A | 0.805 | NRG1 | LST1 | 0.791 | ZNF704 | OSM | 0.809 |
| C11orf96 | TLR4 | 0.729 | ZDHHC18 | GMFG | 0.761 | NIBAN1 | OSM | 0.818 |
| AGAP1 | NR3C2 | 0.772 | BDH1 | SERPINB9 | 0.783 | TNFRSF1B | CLMP | 0.746 |
| TNC | NFE2 | 0.800 | CXCR2 | SSH2 | 0.774 | TMEM273 | CHST15 | 0.781 |
| SHTN1 | SELP | 0.807 | AKAP1 | CCL2 | 0.764 | GLCE | ZNF704 | 0.794 |
| ARPIN | IL1RN | 0.783 | KIAA1522 | FILIP1L | 0.750 | LILRA5 | LST1 | 0.771 |
| PI15 | GFPT2 | 0.746 | S1PR1 | C11orf96 | 0.773 | PPP1R18 | DPYSL3 | 0.746 |
| PIK3AP1 | IL11 | 0.805 | G0S2 | SELP | 0.847 | PRRX1 | THEMIS2 | 0.775 |
| NIPSNAP3A | C11orf96 | 0.783 | IL24 | PIK3AP1 | 0.763 | BASP1 | FAM49A | 0.786 |
| CLEC4A | ACOD1 | 0.751 | PRICKLE2 | RGCC | 0.800 | COQ9 | LY96 | 0.814 |
| CRIP2 | TNFAIP2 | 0.769 | NIPSNAP3A | ANKRD35 | 0.755 | POLDIP2 | PTPRC | 0.782 |
| P2RY13 | TNFAIP6 | 0.786 | LRP4 | KIAA1522 | 0.765 | COL7A1 | LOXL2 | 0.798 |
| RAI14 | ITPRIP | 0.801 | NFAM1 | CCDC3 | 0.757 | SLC15A3 | PDE4B | 0.731 |
| CHI3L1 | PLEK | 0.796 | FAP | LCP1 | 0.738 | GLCE | GLT1D1 | 0.763 |
| CDX2 | PPP2R5A | 0.769 | ATP5IF1 | MCAM | 0.776 | DLC1 | IL1RN | 0.821 |
| DLC1 | KCNJ15 | 0.728 | UBAC1 | PCBP3 | 0.783 | BASP1 | CSF3 | 0.785 |
| MYO1F | SH3BP5 | 0.723 | LILRA5 | TNFAIP2 | 0.765 | DPYSL3 | S100A9 | 0.827 |
| FJX1 | DOK3 | 0.748 | PFKL | NRCAM | 0.778 | MIR100HG | SLC16A6 | 0.756 |
| TRAK1 | RAI14 | 0.797 | SERPINE2 | CD53 | 0.749 | CHST11 | ITPRIP | 0.788 |
| GPR183 | LILRA5 | 0.763 | COX10 | TUBA1A | 0.793 | ZDHHC23 | SRGN | 0.831 |
| COX10 | JAZF1 | 0.769 | ITGA5 | SLC16A6 | 0.758 | RAB15 | ACOT9 | 0.788 |
| RFLNB | MMP19 | 0.771 | SORT1 | TLR1 | 0.785 | OCRL | CHI3L1 | 0.797 |
| RHOH | CCDC88A | 0.716 | GAS2L3 | SLC15A3 | 0.766 | ATP5IF1 | FAP | 0.764 |
| COX10 | PIP4P2 | 0.823 | RGS19 | SELP | 0.766 | TMEM273 | FCGR3B | 0.828 |
| LGALS1 | PCBP3 | 0.790 | SH3RF2 | IL1RN | 0.768 | CMTM4 | WNK2 | 0.810 |
| CCR1 | PCBP3 | 0.805 | POLDIP2 | MMP19 | 0.763 | AMPD2 | CCL4 | 0.793 |
| RGS16 | THBD | 0.794 | NRG1 | VIM | 0.780 | RIPOR3 | CCL2 | 0.776 |
| IL1B | CHST11 | 0.781 | SOD2 | VNN2 | 0.800 | OCRL | RGS16 | 0.772 |
| PDE4B | PTGS2 | 0.745 | FPR2 | PRRX1 | 0.792 | CXCL5 | FAM155A | 0.788 |
| IER5 | SPHK1 | 0.749 | TSPAN6 | SRGN | 0.814 | NIPSNAP3A | CEMIP | 0.804 |
| ANKRD35 | CXCL8 | 0.821 | LY96 | LAT2 | 0.784 | EMP3 | SLC35G2 | 0.793 |
| TMEM71 | LCP1 | 0.741 | LGALS1 | RIPK2 | 0.786 | S1PR1 | CXCR1 | 0.732 |
| GLIPR1 | COL15A1 | 0.710 | CGAS | P2RY13 | 0.727 | HSPA6 | PRR16 | 0.792 |
| NDUFA10 | DUSP2 | 0.767 | FAP | GPR65 | 0.758 | AKAP1 | PDE4B | 0.785 |
| ANKRD35 | PPP1R18 | 0.741 | G0S2 | TNFAIP6 | 0.792 | SOD2 | CD86 | 0.786 |
| UQCRC1 | MX2 | 0.784 | PROK2 | S100A12 | 0.744 | SELENBP1 | AMPD2 | 0.793 |
| FZD5 | GFPT2 | 0.772 | DUSP2 | IL1RN | 0.797 | SRGN | TNFRSF10C | 0.781 |
| BDH1 | WNK2 | 0.820 | FJX1 | IL11 | 0.823 | LILRA2 | GADD45B | 0.767 |
| CDX2 | LRRK2 | 0.801 | TMEM71 | CHST11 | 0.747 | ENO2 | HCK | 0.758 |
| SMAP2 | LST1 | 0.763 | CR1 | ZDHHC18 | 0.742 | AGAP1 | PPP2R5A | 0.786 |
| SWAP70 | OSMR | 0.729 | DUSP2 | TIMP1 | 0.766 | ASB7 | RALGAPA2 | 0.783 |
| PDE4B | SMAP2 | 0.734 | NIPSNAP3A | VNN2 | 0.832 | PI15 | PXDN | 0.774 |
| LRP4 | MSN | 0.782 | UQCRC1 | LRRK2 | 0.806 | TRPM6 | THEMIS2 | 0.789 |
| FERMT2 | SELE | 0.798 | PLEK | C5AR1 | 0.765 | AGAP1 | SLC35G2 | 0.815 |
| TNC | SH3BP5 | 0.790 | MMP3 | GMFG | 0.786 | NIPSNAP3A | ATP5IF1 | 0.768 |
| CSF3 | SLC2A3 | 0.767 | RAB31 | VAV1 | 0.746 | FERMT2 | NCF2 | 0.762 |
| CREM | HCK | 0.771 | PPP1R18 | VAV1 | 0.734 | ATP5IF1 | LY96 | 0.802 |
| ANKRD35 | BMERB1 | 0.782 | NNMT | TNFRSF10C | 0.797 | LILRB1 | OSMR | 0.805 |
| F5 | PIP4P2 | 0.774 | UQCRC1 | NNMT | 0.829 | RGS16 | BMERB1 | 0.801 |
| BDH1 | HSPA6 | 0.765 | CMTM4 | CHI3L2 | 0.732 | CMTM4 | SLC2A3 | 0.771 |
| TSPAN6 | EMP3 | 0.809 | SMAP2 | VNN2 | 0.794 | LCP1 | CYP26B1 | 0.779 |
| GADD45B | RASSF5 | 0.779 | TRIM2 | TMEM106C | 0.757 | BDH1 | PIK3AP1 | 0.733 |
| S1PR1 | FGR | 0.788 | SHTN1 | ASAP1 | 0.786 | SH3RF2 | CD69 | 0.770 |
| FAM20C | VIM | 0.762 | NNMT | S100A8 | 0.821 | BDH1 | TMEM154 | 0.808 |
| LGALS1 | RASSF5 | 0.770 | POLDIP2 | PTGS2 | 0.821 | GPR137B | SSH2 | 0.786 |
| CSNK1G3 | NEO1 | 0.787 | SH2D3C | S100A12 | 0.782 | CYTH4 | RHOH | 0.750 |
| COX10 | CCR1 | 0.810 | TMEM71 | RGS16 | 0.795 | SELP | C5AR1 | 0.802 |
| PIK3AP1 | CHRDL2 | 0.750 | KIAA1522 | LILRB1 | 0.775 | MCAM | C1R | 0.757 |
| AGAP1 | TUBB6 | 0.789 | ANGPTL2 | PTPRC | 0.768 | BCAT1 | GPR65 | 0.754 |
| RIPOR3 | CXCR1 | 0.769 | TMEM71 | S100A9 | 0.821 | F5 | PHLDB2 | 0.779 |
| CHN1 | MX2 | 0.791 | UQCRC1 | CSF3R | 0.760 | NR3C2 | IFITM2 | 0.786 |
| TNC | SERPINE2 | 0.802 | C11orf96 | GPR65 | 0.790 | OCRL | CCDC3 | 0.765 |
| GNAI2 | MCAM | 0.750 | HIBCH | CSF3 | 0.789 | COX10 | SH2D3C | 0.793 |
| KIAA1522 | DPYSL3 | 0.763 | MYO1F | PTPRC | 0.717 | EMP3 | BCL2A1 | 0.745 |
| RGS19 | P2RY13 | 0.732 | SLFN11 | P2RY14 | 0.737 | ITGAM | RHOH | 0.705 |
| INHBA | SPP1 | 0.784 | CSF2RB | BTG2 | 0.779 | PIP4P2 | FAM155A | 0.800 |
| RALGAPA2 | ANGPT2 | 0.821 | COL4A2 | S100A12 | 0.793 | THBD | CD14 | 0.782 |
| COQ9 | CCN4 | 0.774 | LRRK2 | PLAU | 0.767 | CMTM8 | ACOD1 | 0.774 |
| LILRB3 | TMEM273 | 0.762 | ATP5IF1 | AQP9 | 0.791 | PDPN | CD93 | 0.805 |
| PLA1A | SSH2 | 0.760 | TSPAN6 | CSTA | 0.775 | SH3RF2 | MYO1F | 0.781 |
| CCR1 | SLC16A6 | 0.791 | S1PR1 | TLR4 | 0.704 | CSF3R | MCAM | 0.738 |
| FZD5 | NOD2 | 0.740 | IL24 | TREM1 | 0.779 | PDPN | RAI14 | 0.791 |
| RBMS1 | CCL4 | 0.772 | CHI3L1 | TNFRSF10C | 0.782 | UBAC1 | SELE | 0.805 |
| SERPINE2 | S100A9 | 0.836 | TMEM273 | S100A12 | 0.792 | LILRA2 | ADGRG3 | 0.727 |
| ADCY6 | B4GALT4 | 0.769 | FGF7 | FFAR2 | 0.804 | | | |
| CLU | MYO1F | 0.763 | SLC15A4 | SELL | 0.805 | | | |

Importantly, we determined the performance of all 52,326 [324*323/2] 2-gene combinations possible from the 324 significant DE genes identified from the multicohort analysis (FIG. 4D). First, to show that the gene-pairs are associated with response and not partial to other factors, we evaluated the performance of 10,000 randomly selected gene-pairs out of the 52,326 possible pairs using 50 permutations of scrambled patient response adjudications wherein the labels (responders [1] or non-responders [0]) were randomly permuted while maintaining the original ratio of responders to non-responders for each dataset). This resulted in a mean AUC distribution around 0.5 which represents absence of signal (FIG. 4E). AUCs for the 10,000 randomly selected gene-pairs of the 324 signature biomarkers are given in Table 4. Second, to show that the 52,326 gene-pairs have clear discriminatory capability over randomly selected gene-pairs from all genes measured, we randomly sampled 10,000 gene-pairs from 15,116 genes and calculated AUCs to characterize background gene-pairs (FIG. 4F). As expected, in each study as well as in pooled results, the mean AUCs for 2-gene pairs derived from the 324 DE genes were greater compared to the mean AUCs for 10,000 randomly sampled gene-pairs from all measured genes (background). We used Welch's t-test (unequal variance t-test) to calculate significance for the difference in mean distributions, resulting in a t-value >130 with corresponding p-values <10-150 for all comparisons: dataset by dataset as well as the pooled results.

### 5. Discussion

Our goal was to identify a clear predictive factor of therapeutic response to anti-TNF alpha in the hopes that with such a baseline gene expression-based test it would greatly improve the efficacy and cost-to-benefit ratio of the biologics. We used our established LOSO multicohort analysis framework on four mucosal biopsy datasets to identify significant DE genes of interest that would differentiate anti-TNF alpha responders from non-responders. We identified with moderate thresholds a set of 324 DE genes that have acceptable discriminatory performance with a pooled AUROC of 0.82.

To illustrate that we can translate the set of 324 genes to a more clinically useful signature, we used a feature down-selection method known as greedy forward search to identify a parsimonious 7-gene signature with an improved AUROC of 0.88.

In addition to testing the performance of the 324 DE genes and 7-gene signature, we demonstrated that it is possible to select a subset of 324 DE mRNAs either as single marker or as a gene pair to achieve a robust level of performance for the purposes of discriminating between responders and non-responders. Specifically, we emphasize that we tested the significance of the AUC distributions in FIG. 4D for all 52,326 possible 2-gene pairs from 324 DE genes against two different null hypotheses. The first null hypothesis was used to test the notion that these 324 DE genes are associated with TNF alpha treatment response and not other factors as represented by the AUC distributions in FIG. 4E obtained from label-permuted datasets. The second null hypothesis was employed to test the notion that the 52,326 gene-pairs perform better than background as represented by the AUC distributions in FIG. 4F obtained for 10,000 gene-pairs randomly selected from all the 15,116 genes measured in the datasets. Against both null hypotheses, the performance characterization of all 52,326 2-gene pairs that are exhaustively possible from our 324 signature DE genes were highly significant in discriminating responders from non-responders.

Lastly, we tested the feasibility of translating these gene sets into a clinical test by leveraging our Inflammatix Machine Learning platform. With samples from all datasets integrated as one cohort, we used the 324 DE genes and 7-gene signature as inputs to evaluate a gamut of machine learning models. These gene sets had remarkable performance in a wide variety of classifiers, suggesting that the genes we have discovered represent true underlying molecular differences associated with treatment response with high odds that they will validate in independent patient cohorts as well as translate well to a target diagnostic platform for a clinical prognostic test. These findings need to be validated via retrospective clinical studies prior to initiating prospective clinical trials and subsequent implementation in patient care.

The above description of example embodiments of the present disclosure has been presented for the purposes of illustration and description. It is not intended to be exhaustive or to limit the disclosure to the precise form described, and many modifications and variations are possible in light of the teaching above.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art. Where a conflict exists between the instant application and a reference provided herein, the instant application shall dominate.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “1′ or ‘2’ or ‘3’ or ‘1 and 2’ or ‘1 and 3’ or ‘2 and 3’ or ‘1, 2 and 3’”. Whenever a range is given in the specification, for example, a temperature range, a time range, or a composition range, all intermediate ranges and subranges, as well as all individual values included in the ranges given are intended to be included in the disclosure.

1. A method of administering medical care to a subject presenting one or more symptoms of Inflammatory Bowel Disease (IBD), the method comprising:
(i) receiving a biological sample obtained from the subject;
(ii) measuring expression levels of one or more biomarkers in the sample, wherein the one or more biomarkers comprise at least one biomarker from Table 3 or one pair of biomarkers from Table 4; and
(iii) generating a TNF alpha prognostic score based on the measured expression levels of the biomarkers in the sample, wherein a TNF alpha prognostic score that exceeds a threshold value indicates that the subject is a candidate for anti-TNF alpha therapy.

2. The method of claim 1, further comprising:
(iv) determining the subject is a candidate for anti-TNF alpha therapy based on the TNF alpha prognostic score exceeding the threshold value; and
(v) administering anti-TNF alpha therapy to the subject to treat the IBD.

3. The method of claim 1, further comprising:
(iv) determining the subject is not a candidate for anti-TNF alpha therapy based on the TNF alpha prognostic score not exceeding the threshold.

4. The method of claim 1, wherein the biological sample is an intestinal biopsy, buccal swab, blood, or stool.

5. The method of claim 1, wherein the IBD is one of Crohn's Disease (CD), ulcerative colitis (UC), and an unclassified IBD.

6. (canceled)

7. (canceled)

8. The method of claim 1, wherein the expression of the biomarkers is detected using qRT-PCR or isothermal amplification.

9. The method of claim 8, wherein the isothermal amplification method is qRT-LAMP.

10. (canceled)

11. The method of claim 1, wherein the method comprises measuring the expression of a set of biomarkers comprising seven biomarkers in the sample, and wherein the set of biomarkers comprise WNK2, OCRL, ASB7, PCBP3, AMPD2, FAM155A, and IL13RA2.

12. (canceled)

13. The method of claim 1, wherein the TNF alpha prognostic score exceeds the threshold value and wherein the measured expression levels of at least one of WNK2, OCRL, and ASB7 are elevated relative to a reference level representative of an individual with IBD who is not a candidate for anti-TNF alpha therapy.

14. The method of claim 1, wherein the TNF alpha prognostic score exceeds the threshold value and wherein the measured expression levels of at least one of PCBP3, AMPD2, FAM155A, and IL13RA2 are reduced relative to a reference level representative of an individual with IBD who is not a candidate for anti-TNF alpha therapy.

15. (canceled)

16. A test kit for detecting the expression levels of one or more biomarkers in a biological sample from a subject with Inflammatory Bowel Disease (IBD), wherein the biomarkers comprise at least one biomarker from Table 3 or one pair of biomarkers from Table 4.

17. The test kit of claim 16, wherein the kit comprises a microarray.

18. The test kit of claim 16, wherein the kit comprises an oligonucleotide for each of the one or more biomarkers, wherein each oligonucleotide hybridizes to one of the biomarkers.

19. The test kit of claim 16, wherein the biomarkers comprise WNK2, OCRL, ASB7, PCBP3, AMPD2, FAM155A, and IL13RA2.

20. The test kit of claim 19, wherein the kit comprises an oligonucleotide that hybridizes to WNK2, an oligonucleotide that hybridizes to OCRL, an oligonucleotide that hybridizes to ASB7, an oligonucleotide that hybridizes to PCBP3, an oligonucleotide that hybridizes to AMPD2, an oligonucleotide that hybridizes to FAM155A, and an oligonucleotide that hybridizes to IL13RA2.

21. (canceled)

22. The test kit of claim 16, wherein the IBD is Crohn's Disease (CD).

23. The test kit of claim 16, wherein the IBD is Ulcerative Colitis (UC).

24. The test kit of claim 16, wherein the IBD is an unclassified IBD.

25. The test kit of claim 16, wherein the biological sample is an intestinal biopsy, buccal swab, blood, or stool.

26. The test kit of claim 16, further comprising instructions to calculate a TNF alpha prognostic score based on the levels of expression of the biomarkers in the biological sample from the subject, the score correlating with the likelihood that the subject will respond to an anti-TNF alpha therapy.

27. (canceled)
